(19) United States
(12) Patent Application Publication
(10) Pub. No.: US 2025/0122550 A1
(43) Pub. Date: Apr. 17, 2025

(54) METHODS AND COMPOSITIONS FOR MAKING BACTERIOCINS AND ANTIMICROBIAL PEPTIDES

(71) Applicant: Syngulon SA, Seraing (BE)

(72) Inventor: Philippe Gabant, Ottignies Louvain-La-Neuve (BE)

(21) Appl. No.: 18/984,698

(22) Filed: Dec. 17, 2024

Related U.S. Application Data
(62) Division of application No. 18/052,519, filed on Nov. 3, 2022, now Pat. No. 12,195,780.
Application No. 18/052,519 is a division of application No. 16/642,342, filed on Feb. 26, 2020, now Pat. No. 11,492,651.
(60) Provisional application No. 62/720,804, filed on Aug. 21, 2018.
(60) Provisional application No. 62/552,835, filed on Aug. 31, 2017.

(51) Int. Cl. C12P 21/02; A01N 63/50; B01L 3/00; C07K 14/195

(52) U.S. Cl. CPC C12P 21/02; A01N 63/50; B01L 3/502738; C07K 14/195

(57) ABSTRACT

Methods and compositions for making bacteriocins are described in some embodiments herein. In some embodiments, pro-polypeptide comprising the bacteriocins in the desired ratios in cis, and separated by cleavage sited can be produced by a microbial cell comprising a nucleic acid encoding the pro-polypeptide. In some embodiments microfluidic devices and methods for making specified mixtures of antimicrobial peptides and/or bacteriocins are described.

### RELATED APPLICATIONS

This application is a divisional application of U.S. application Ser. No. 18/052,519, filed Nov. 3, 2022, which is a divisional application of U.S. application Ser. No. 16/642,342, filed Feb. 26, 2020, now U.S. Pat. No. 11,492,651, issued Nov. 8, 2022, which is a U.S. national phase application under 35 U.S.C. § 371 of International Application No. PCT/US2018/048846, filed on Aug. 30, 2018, which claims the benefit of U.S. Provisional Application No. 62/720,804, filed Aug. 21, 2018, and U.S. Provisional Application No. 62/552,835, filed Aug. 31, 2017. The contents of the aforementioned applications are expressly incorporated herein by reference in their entirety.

### REFERENCE TO SEQUENCE LISTING, TABLE, OR COMPUTER PROGRAM LISTING

The present application is being filed along with a Sequence Listing in electronic format. The Sequence Listing is provided a file entitled SeqListingSYNG003D2.xml, created on Dec. 3, 2024, which is 865,276 bytes in size. The information in the electronic format of the Sequence Listing is incorporated herein by reference in its entirety.

### BACKGROUND

Microbial organism-mediated processes can be used in a variety of industrial processes for the manufacture of products of interest, for example for fermentation in a feedstock. Additionally, microbial organisms can be used to manufacture products in sterile environments, such as in the manufacture of pharmaceuticals, biologics, and cosmetics. Additionally, populations of microbial organisms are involved in maintaining the health and metabolic functions of multicellular organisms, for example as cultures of microbial flora in the gut and skin of humans, or the roots of the plants. The efficiency and efficacy of these processes can be affected by culture conditions, as well as the phenotypic characteristics of microbial organisms present in the culture.

Tuning populations of microbial organisms, for example to reduce or eliminate undesired microbial organisms can be useful for maintaining the industrial processes and maintaining the health of tissues that comprise microbial organisms.

### FIELD

Embodiments herein relate generally to producing gene products for the control of growth of microorganisms. More particularly, some embodiments relate to methods, reagents, and microfluidic devices for making bacteriocins. The bacteriocins can be produced in a composition, such as a specified mixture of bacteriocins, which can be useful for controlling the growth of populations of microbial organisms. In some embodiments antimicrobial peptides and/or bacteriocins are produced in desired ratios in a composition.

### SUMMARY

Some embodiments include a method of making bacteriocins. The method can comprise expressing a nucleic acid comprising a bacteriocin coding sequence and a second polypeptide coding sequence in a single reading frame, in which the second polypeptide comprises, consists essentially of, or consists of a bacteriocin or signal molecule. The nucleic acid can further comprise cleavage site coding sequences disposed between the bacteriocin coding sequence and the second polypeptide coding sequence in the single reading frame. Thus, a pro-polypeptide comprising the bacteriocin, second polypeptide, and cleavage sites disposed between the bacteriocin and second polypeptide can be generated. In some embodiments, the method further comprises cleaving the cleavage site, thus separating the bacteriocin and second polypeptide from each other. The method can thus produce a composition comprising the bacteriocin and the second polypeptide. In some embodiments, the second polypeptide comprises, consists essentially of, or consists of a bacteriocin. In some embodiments, the second polypeptide comprises, consists essentially of, or consists of an antimicrobial peptide. In some embodiments, the second polypeptide comprises, consists essentially of, or consists of the signal molecule. In some embodiments, the expressing is performed by a microbial cell that does not produce a functional immunity modulator for at least one of the bacteriocins. In some embodiments, the microbial cell does not produce a functional immunity modulator for any of the bacteriocins. In some embodiments, the expressing is performed in vitro. In some embodiments, at least one of the bacteriocins is inactive when it is part of the pro-polypeptide. In some embodiments, the method further comprises isolating the pro-polypeptide prior to the cleaving. In some embodiments, the isolating comprises affinity purifying the pro-polypeptide, in which the affinity purification comprises binding an affinity tag encoded by the nucleic acid. In some embodiments, the nucleic acid comprises three bacteriocin coding sequences in the single reading frame. In some embodiments, at least two of the bacteriocins are different from each other. In some embodiments, the composition comprises a desired ratio of bacteriocins, or a desired ratio of signal molecules and bacteriocins. In some embodiments, at least a portion the desired ratio is achieved by a ratio of bacteriocin coding sequences, or bacteriocin and signal molecule coding sequences in the single reading frame of the nucleic acid. In some embodiments, the desired ratio is further achieved by a second nucleic acid comprising a ratio of bacteriocins coding sequences and further comprising cleavage sites between the bacteriocin coding sequences. In some embodiments, desired ratio is achieved by a ratio of bacteriocin coding sequences in the single reading frame of the nucleic acid. In some embodiments, the desired ratio of bacteriocins is selected to target an undesired microbial organism or population of undesired microbial organisms, and/or the desired ratio of bacteriocins is selected to balance a population of a microbiome of an animal, a human organ, a plant, a plant root, and/or soil (e.g. in the context of a plant root and soil). In some embodiments, the desired ratio of bacteriocins and signal molecules is selected to control genetic drift of a target microbial cell, and stimulate growth or production of a producing cell. In some embodiments, the desired ratio comprises a ratio of a first bacteriocin to a second bacteriocin of 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, or 9:10, in which the first bacteriocin is different from the second bacteriocin. In some embodiments, the cleavage sites are for a wild-type, variant, or synthetic cleavage enzyme, such as an endopeptidase. In some embodiments, the cleavage sites are for a cleavage enzyme selected from the group consisting of: Arg-C proteinase, Asp-N endopeptidase, BNPS-Skatole, Caspase 1, Caspase 2, Caspase 3, Caspase 4, Caspase 5, Caspase 6, Caspase 7, Caspase 8, Caspase 9, Caspase 10, Chymotrypsin-high specificity, Clostripain (Clostridiopeptidase B), CNBr, Enterokinase, Factor Xa, Formic acid, Glutamyl endopeptidase, GranzymeB, Hydroxylamine, lodosobenzoic acid, LysC, Neutrophil elastase, NTCB (2-nitro-5-thiocyanobenzoic acid), Pepsin (pH1.3), Pepsin (pH>2), Proline-endopeptidase, Proteinase K, Staphylococcal peptidase I, Thermolysin, Thrombin, or Trypsin. In some embodiments, the cleavage sites are for a single cleavage enzyme, and wherein the cleavage enzyme does not cleave within the bacteriocins. In some embodiments, at least one cleavage site is for a first cleavage enzyme, and another cleavage site is for a second cleavage enzyme, and neither the first nor the second cleavage enzyme cleaves within the bacteriocins. In some embodiments, the composition produced by the method further comprises a signal molecule (in which the nucleotide further comprises: a coding sequence for a signal molecule in the single reading frame), and a cleavage site sequence disposed between the signal molecule and a bacteriocin coding sequence. In some embodiments, the signal molecule is selected from the group consisting of: quorum sensing molecules, signal transduction receptor ligands, growth factors, hormones, and cytokines, and wherein the signal molecule can be wild-type, mutant, or synthetic. In some embodiments, the signal molecule comprises, consists of, or consists essentially of a quorum sensing peptide. In some embodiments, the pro-polypeptide has a length of no more than about 2000 amino acids. In some embodiments, the method further comprises expressing a second nucleic acid encoding a second pro-polypeptide comprising two bacteriocins and cleavage sites disposed therebetween, wherein the second pro-polypeptide is different from the first pro-polypeptide. In some embodiments, cleaving the pro-polypeptide comprises physical treatment of a peptide linker comprised by the cleavage site, wherein the peptide linker is chemical-sensitive or pH sensitive. In some embodiments, the method further comprises chemically modifying the bacteriocins. In some embodiments, the bacteriocins are chemically modified co-translationally. In some embodiments, the bacteriocins are chemically modified following the cleaving. In some embodiments, a pro-polypeptide as described herein comprises two or more antimicrobial peptides instead of bacteriocins, and upon cleaveage, a composition comprising antimicrobial peptides is produced. In some embodiments, a pro-polypeptide as described herein comprises one or more antimicrobial peptides and one or more bacteriocins, and upon cleaveage, a composition comprising a mixture of antimicrobial peptides and bacteriocins is produced.

Some embodiments include an isolated nucleic acid comprising a bacteriocin coding sequence and a second polypeptide coding sequence in a single reading frame, in which the second polypeptide comprises, consists essentially of, or consists of a bacteriocin or a signal molecule. The isolated nucleic acid can comprise cleavage site coding sequences disposed between the bacteriocin coding sequences and in the single reading frame. In some embodiments, the second polypeptide comprises, consists essentially of, or consists of the bacteriocin. In some embodiments, the second polypeptide comprises, consists essentially of, or consists of an antimicrobial peptide. In some embodiments, the second polypeptide comprises, consists essentially of, or consists of the signal molecule. In some embodiments, the cleavage site coding sequences encode cleavage sites for a cleavage enzyme, and in which the bacteriocin coding sequences do not comprise cleavage sites for the cleavage enzyme. In some embodiments, the nucleic acid comprises three bacteriocin coding sequences in the single reading frame. In some embodiments, the nucleic acid comprises at least 5, 10, 15, or 20 bacteriocin sequences in the single reading frame. In some embodiments, a cleavage site coding sequence is disposed in frame between any two adjacent bacteriocin and/or signal molecule coding sequences. In some embodiments, at least two of the bacteriocin sequences encode different bacteriocins from each other. In some embodiments, the three bacteriocin sequences are present in a desired ratio or portion of a desired ratio. In some embodiments, the desired ratio is selected to target an undesired microbial organism or population of undesired microbial organisms. In some embodiments, the desired ratio of bacteriocins is selected to balance a population of a microbiome of an animal, a human organ, a plant, a plant root, and/or soil (e.g., in the context of a plant root and soil). In some embodiments, the cleavage sites are for a wild-type, variant, or synthetic cleavage enzyme, such as an endopeptidase. In some embodiments, the cleavage sites are for a cleavage enzyme selected from the group consisting of: Arg-C proteinase, Asp-N endopeptidase, BNPS-Skatole, Caspase 1, Caspase 2, Caspase 3, Caspase 4, Caspase 5, Caspase 6, Caspase 7, Caspase 8, Caspase 9, Caspase 10, Chymotrypsin-high specificity, Clostripain (Clostridiopeptidase B), CNBr, Enterokinase, Factor Xa, Formic acid, Glutamyl endopeptidase, GranzymeB, Hydroxylamine, lodosobenzoic acid, LysC, Neutrophil elastase, NTCB (2-nitro-5-thiocyanobenzoic acid), Pepsin (pH1.3), Pepsin (pH>2), Proline-endopeptidase, Proteinase K, Staphylococcal peptidase I, Thermolysin, Thrombin, or Trypsin. In some embodiments, a first cleavage site coding sequence encodes a first cleavage site is for a first cleavage enzyme, and wherein a second cleavage site coding sequence encodes a cleavage site for a second cleavage enzyme that is different from the first cleavage enzyme, and wherein the bacteriocins do not comprise a cleavage site for any of the first or second cleavage enzyme. In some embodiments, the cleavage sites comprise a pH- or chemically-sensitive linker. In some embodiments, the isolated nucleic acid further comprises a coding sequence for a signal molecule in the single reading frame, in which a cleavage site coding sequences is disposed between the coding sequence for signal molecule and an adjacent bacteriocin coding sequence. In some embodiments, the signal molecule is selected from the group consisting of: quorum sensing molecules, signal transduction receptor ligands, growth factors, hormones, and cytokines, and in which the signal molecule can be wild-type, mutant, or synthetic. In some embodiments, an isolated nucleic acid as described herein comprises two or more antimicrobial peptide coding sequences instead of bacteriocin coding sequences, and encodes a pro-polypeptide comprising the antimicrobial peptides. In some embodiments, an isolated nucleic acid as described herein comprises one or more antimicrobial peptide coding sequences and one or more bacteriocin coding sequences, and encodes a pro-polypeptide comprising one or more antimicrobial peptides and one or more bacteriocins.

Some embodiments include a microbial cell, comprising a promoter operably linked to the isolated nucleic acid of any of the embodiments described herein, in which the isolated microbial cell does not produce a functional immunity modulator for a bacteriocin encoded by the isolated nucleic acid. In some embodiments, the cell does not produce a functional immunity modulator for any of the bacteriocins encoded by the isolated nucleic acid.

Some embodiments include an isolated pro-polypeptide comprising two bacteriocins, and/or a bacteriocin and a signal molecule, cleavage sites disposed between the bacteriocins and/or the bacteriocin and the signal molecule; and an affinity tag. In some embodiments, the pro-polypeptide comprises the two bacteriocins. In some embodiments, the pro-polypeptide comprises the bacteriocin and the signal molecule. In some embodiments, the pro-polypeptide comprises three bacteriocins. In some embodiments, the pro-polypeptide comprises at least 5, 10, 15, or 20 bacteriocins. In some embodiments, the pro-polypeptide comprises a signal molecule. In some embodiments, the cleavage sites are for a cleavage enzyme, and wherein the bacteriocin coding sequences do not comprise cleavage sites for the cleavage enzyme. In some embodiments, a cleavage site is for a first cleavage enzyme, in which another cleavage site is for a second cleavage enzyme different from the first cleavage enzyme, and in which the bacteriocins do not comprise a cleavage site for any of the first or second cleavage enzymes. In some embodiments, the isolated pro-polypeptide further comprises a co-translational or post-translational modification. In some embodiments, an isolated pro-polypeptide as described herein comprises two or more antimicrobial peptides instead of bacteriocins. In some embodiments, an isolated pro-polypeptide as described herein comprises one or more antimicrobial peptides and one or more bacteriocins.

In some embodiments, a composition comprising two more bacteriocins in a ratio selected to target a microbial cell or populations of microbial cells is described. Each of the bacteriocins of the composition can comprise, at its N-terminus, C-terminus, or N-terminus and C-terminus, a portion of a cleavage sequence that has been cleaved, in which the portions of cleavage sequences at the N-, C-, or N- and C-termini of the bacteriocins are for cleavage sites of the same or different cleavage enzyme. In some embodiments, at least some of the bacteriocins further comprise a tag. In some embodiments, the tag is selected from the group consisting of affinity tags, a signal sequence, or a stability tag. In some embodiments, the composition further comprises a signal molecule in a desired ratio with the bacteriocins, wherein the signal molecule comprises, at its N-terminus, C-terminus, or N-terminus and C-terminus, a portion of a cleavage sequence that has been cleaved, wherein the portions of cleavage sequences at the N-, C-, or N- and C-termini of the signal molecule are for cleavage sites of the same or different cleavage enzymes. In some embodiments, the ratio of bacteriocins is selected to target an undesired microbial organism or population of undesired microbial organisms. In some embodiments, the ratio of bacteriocins is selected to balance a population of a microbiome of an animal, a human organ, a plant, a plant root, and/or soil (e.g., in the context of a plant root and soil). In some embodiments, the composition is formulated for topical or oral administration to a human subject. In some embodiments, the composition is for use in balancing a population of a microbiome, for example, that of an animal, a human organ (such as a gut or skin), a plant, a plant root, and/or soil. In some embodiments, the isolated pro-polypeptide as described herein comprises one or more antimicrobial peptides. The pro-polypeptide can be cleaved, and the composition can comprise one or more antimicrobial peptides that comprise portions of cleavage sequences at the N-, C-, or N- and C-termini.

Some embodiments include a method for producing a specified mixture of bacteriocins and/or antimicrobial proteins, for example. The method can comprise selecting the mixture to comprise two or more different bacteriocins and/or antimicrobial proteins. The method can comprise, in a microfluidic device comprising discrete coding substrates that each encode an antimicrobial peptide or bacteriocin: placing discrete coding substrates that encode the antimicrobial peptides and/or bacteriocins of the specified mixture in fluid communication with an in vitro transcription/translation solution; incubating the discrete coding substrates with the in vitro transcription/translation solution, thus generating antimicrobial peptides and/or bacteriocins encoded by the discrete coding substrates; and mixing the antimicrobial peptides and/or bacteriocins in the microfluidic device, thus producing the specified mixture of antimicrobial peptides and/or bacteriocins. In some embodiments, the method further comprises producing two or more submixtures each comprising a subset of the specified mixture of antimicrobial peptides and/or bacteriocins and combining the submixtures to produce the specified mixture of antimicrobial peptides and/or bacteriocins. In some embodiments, selecting further comprises selecting a stoichiometry of the two or more different antimicrobial peptides and/or bacteriocins of the specified mixture. In some embodiments, the specified mixture of antimicrobial peptides and/or bacteriocins comprises a specified stoichiometry, and combining the submixtures results in the specified stoichiometry. In some embodiments, the discrete coding substrates are comprised within separate chambers. In some embodiments, the discrete coding substrates comprise nucleic acids immobilized thereon. In some embodiments, discrete coding substrates encoding antimicrobial peptides and/or bacteriocins of the specified mixture, but not other discrete coding substrates, are placed in fluidic communication with the in vitro transcription/translation solution. In some embodiments, incubating the discrete coding substrates with the in vitro transcription/translation solution comprises flowing the in vitro transcription/translation solution into each chamber. In some embodiments, the in vitro transcription/translation solution comprises an in vitro transcription reagent and/or an in vitro translation reagent. In some embodiments, placing the discrete coding substrates that encode the antimicrobial peptides and/or bacteriocins of the specified mixture in fluid communication with an in vitro transcription/translation solution comprises (i) opening valves so as to place a source of the in vitro transcription/translation solution in fluid communication with the discrete coding substrates; (ii) closing valves so as to inhibit fluid communication between the source of the in vitro transcription/translation solution and the other discrete coding substrates, or a combination of (i) and (ii). In some embodiments, mixing the antimicrobial peptides and/or bacteriocins in the microfluidic device comprises opening a valve to place the discrete coding substrates in fluid communication with a fluidic reservoir, in which the antimicrobial peptides and/or bacteriocins are mixed in the fluidic reservoir. In some embodiments, the method further comprises screening the mixture of antimicrobial peptides and/or bacteriocins in situ for a desired effect. In some embodiments, the screening is for inhibition of the growth or reproduction of a pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract. In some embodiments, the screening is for an absence of deleterious effects of the mixture of antimicrobial peptides and/or bacteriocins on a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract. In some embodiments, the screening is performed in real time. For example, the screening can be performed within 120 minutes, 60 minutes, 30 minutes, 15 minutes, 10 minutes, 5 minute, or 1 minute of the specified mixture of antimicrobial peptides and/or bacteriocins being generated. In some embodiments, the screening is for stabilization of an antimicrobial peptide and/or bacteriocin or for destruction of a microbial biofilm. In some embodiments, one or more of the discrete coding substrates encodes an auxiliary protein with anti-protease activity. In some embodiments, the screening is for enhancement of growth or reproduction of a non-pathogenic microbial organism in a microbiome of a subject, such as a skin, gut, ovarian follicle, lung, saliva, oral cavity, mucosa, conjunctiva, or biliary tract. In some embodiments, one or more of the discrete coding substrates encodes an auxiliary protein that attracts the non-pathogenic microbial organism, or that enhances growth or reproduction of the non-pathogenic microbial organism in the microbiome of a subject. In some embodiments, the method further comprises delivering the specified mixture of antimicrobial peptides and/or bacteriocins to a wound via a tubing or membrane, thereby cleaning or dressing the wound. In some embodiments, the desired effect comprises antimicrobial activity. In some embodiments, the method further comprises screening 1, 2, 3, 4, 5, 6, 7, 8, 9, or 10 different mixtures against a microbial infection. In some embodiments, the method further comprises delivering the mixture of specified antimicrobial peptides and/or bacteriocins, in combination with a chemical antibiotic and/or a phage antibiotic, to a subject. In some embodiments, the in vitro transcription/translation solution is lyophyilized, and further comprising adding water to the in vitro transcription/translation solution. In some embodiments, the method comprises making a specified mixture of bacteriocins. As such the method can comprise, in a microfluidic device comprising discrete coding substrates that each encode a bacteriocin: placing discrete coding substrates that encode the bacteriocins of the specified mixture in fluid communication with an in vitro transcription/translation solution; incubating the discrete coding substrates with the in vitro transcription/translation solution, thus generating bacteriocins encoded by the discrete coding substrates; and mixing the bacteriocins in the microfluidic device, thus producing the specified mixture of bacteriocins. In some embodiments, the method comprises making a specified mixture of bacteriocins that does not comprise antimicrobial peptides. In some embodiments, the method comprises making a specified mixture of antimicrobial peptides. In some embodiments, the method comprises making a specified mixture of antimicrobial peptides that does not comprise bacteriocins. In some embodiments, the method comprises making a specified mixture of bacteriocins and antimicrobial peptides. It will be understood that if a method, microfluidic device or system as described herein is for making a specified mixture of bacteriocins that does not comprise antimicrobial peptides, such a method, microfluidic device or system need not comprise selecting antimicrobial peptides as part of a specified mixture, need not comprise discrete coding substrates encoding antimicrobial peptides, and need not produce or flow any antimicrobial peptides. It will be understood that if a method, microfluidic device or system as described herein is for making a specified mixture of antimicrobial peptides that does not comprise bacteriocins, such a method, microfluidic device or system need not comprise selecting bacteriocins as part of a specified mixture, need not comprise discrete coding substrates encoding bacteriocins, and need not produce or flow any bacteriocins.

Some embodiments include a microfluidic device for producing a specified mixture of antimicrobial peptides and/or bacteriocins. The device can comprise discrete coding substrates that each encode an antimicrobial peptide and/or bacteriocin; an in vitro transcription/translation solution; a fluidic reservoir; and valves each disposed on a fluidic path between a discrete coding substrate and the fluidic reservoir, each valve configured to regulate flow between the discrete coding substrate and the fluidic reservoir. The device can be configured to be placed in data communication with a processor configured to: based on the specified mixture of antimicrobial peptides and/or bacteriocins, configure the valves to place the discrete coding substrates that encode the antimicrobial peptides and/or bacteriocins of the mixture, but not other discrete coding substrates, in fluidic communication with the fluidic reservoir; permit incubation of the in vitro transcription/translation solution with the discrete coding substrates that encode the antimicrobial peptides and/or bacteriocins of the specified mixture, so that the antimicrobial peptides and/or bacteriocins of the specified mixture are produced; permit flow of the antimicrobial peptides and/or bacteriocins through the valves into the fluidic reservoir; and control flow of fluid in the fluidic reservoir, in which the flow comprises movement of the antimicrobial peptides and/or bacteriocins in the fluidic reservoir, thereby producing the specified mixture of antimicrobial peptides and/or bacteriocins in the fluidic reservoir. In some embodiments, the specified mixture of antimicrobial peptides and/or bacteriocins comprises two or more submixtures each comprising, consisting essentially of, or consisting of a subset of antimicrobial peptides and/or bacteriocins; and the processor is configured to permit flow of each submixture into the fluidic reservoir, thereby producing the specified mixture of antimicrobial peptides and/or bacteriocins. In some embodiments, the specified mixture of antimicrobial peptides and/or bacteriocins comprises a sum of the subsets of antimicrobial peptides and/or bacteriocins in a specified stoichiometry, and wherein combination of the submixtures yields the specified stoichiometry. In some embodiments, the discrete coding substrates are comprised within separate chambers. In some embodiments, the discrete coding substrates comprise nucleic acids immobilized thereon. In some embodiments, the discrete coding substrates comprise a material or product selected from the group consisting of a chip, bead, nanoparticle, well, membrane, matrix, plastic, metal, glass, polymer, polysaccharide, and paramagnetic compound. In some embodiments, the in vitro transcription/translation solution comprises an in vitro transcription reagent and/or an in vitro translation reagent. In some embodiments, the device is portable. In some embodiments, one or more of the discrete coding substrates encodes an auxiliary protein comprising, consisting essentially of, or consisting of a protein for stabilization of antimicrobial peptides and/or bacteriocins, a protein with anti-protease activity, or a protein for destruction of a microbial biofilm. In some embodiments, one or more of the discrete coding substrates encodes an auxiliary protein that attracts a non-pathogenic microbial organism, or that enhances growth or reproduction of the non-pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract. In some embodiments, the fluidic reservoir is configured to be placed in fluid communication with a tissue of a subject. In some embodiments, the tissue comprises a microbiome, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract. In some embodiments, the fluidic reservoir is configured to be placed in fluid communication with a wound of a subject. In some embodiments, the microfluidic device further comprises a fluidic passage such as a tube or membrane through which the fluidic reservoir is capable of being placed in fluid communication with the microbiome or wound, through which the specified mixture of antimicrobial peptides and/or bacteriocins is capable of being delivered to the microbiome or wound. In some embodiments, wherein each discrete coding substrate encodes a different bacteriocin. In some embodiments, a discrete coding substrate comprises the isolated nucleic acid comprising, consisting essentially of, or consisting of an antimicrobial peptide and/or bacteriocin coding sequence and a second polypeptide coding sequence in a single reading frame, as described herein. In some embodiments, a discrete coding substrate encodes an isolated pro-polypeptide as described herein. In embodiments, the microfluidic device further comprises the processor. In some embodiments, the microfluidic device further comprises a reservoir of chemical or phage antibiotics configured to mix with the mixture of specified antimicrobial peptides and/or bacteriocins. In some embodiments, the microfluidic device further comprise an antibiotic comprising, consisting essentially of, or consisting of chemical antibiotic and/or a phage antibiotic. In some embodiments, the in vitro transcription/translation solution is lyophyilized. In some embodiments, the microfluidic device is for producing a specified mixture of bacteriocins. As such, the device can comprise discrete coding substrates that each encode a bacteriocin; an in vitro transcription/translation solution; a fluidic reservoir; and valves each disposed on a fluidic path between a discrete coding substrate and the fluidic reservoir, each valve configured to regulate flow between the discrete coding substrate and the fluidic reservoir. In some embodiments, the microfluidic device is for producing a specified mixture of bacteriocins that does not comprise antimicrobial peptides. In some embodiments, the microfluidic device is for producing a specified mixture of antimicrobial peptides. In some embodiments, the microfluidic device is for producing a specified mixture of antimicrobial peptides that does not comprise bacteriocins. In some embodiments, the microfluidic device is for producing a specified mixture of bacteriocins and antimicrobial peptides.

Some embodiments include a system. The system can comprise a microfluidic device described herein, and a processor configured to: based on a specified mixture of antimicrobial peptides and/or bacteriocins (as described herein), configure the valves to place the discrete coding substrates that encode the antimicrobial peptides and/or bacteriocins of the mixture, but not other discrete coding substrates, in fluidic communication with the fluidic reservoir; permit incubation of the in vitro transcription/translation solution with the discrete coding substrates that encode the antimicrobial peptides and/or bacteriocins of the specified mixture, so that the antimicrobial peptides and/or bacteriocins of the specified mixture are produced; permit flow of the antimicrobial peptides and/or bacteriocins through the valves into the fluidic reservoir; and control flow of fluid in the fluidic reservoir, wherein the flow comprises movement of the antimicrobial peptides and/or bacteriocins in the fluidic reservoir, thus producing the specified mixture of antimicrobial peptides and/or bacteriocins in the fluidic reservoir. In some embodiments, the microfluidic device is comprised by a cartridge, and the system comprises a coupling for placing the cartridge in data communication with the processor. In some embodiments, the system further comprises a reservoir of in vitro transcription/translation solution. In some embodiments, the system further comprise a reservoir of chemical and/or phage antibiotics configured to mix with the mixture of specified antimicrobial peptides and/or bacteriocins. In some embodiments, the system further comprise an antibiotic comprising, consisting essentially of, or consisting of a chemical antibiotic and/or a phage. In some embodiments, the in vitro transcription/translation solution is lyophyilized. In some embodiments, the system is for producing a specified mixture of bacteriocins. As such, the processor can be configured to, based on a specified mixture of antimicrobial peptides and/or bacteriocins (as described herein), configure the valves to place the discrete coding substrates that encode the bacteriocins of the mixture, but not other discrete coding substrates, in fluidic communication with the fluidic reservoir; permit incubation of the in vitro transcription/translation solution with the discrete coding substrates that encode the antimicrobial peptides and/or bacteriocins of the specified mixture, so that the bacteriocins of the specified mixture are produced. In some embodiments, the system is for producing a specified mixture of bacteriocins that does not comprise antimicrobial peptides. In some embodiments, the system is for producing a specified mixture of antimicrobial peptides. In some embodiments, the system is for producing a specified mixture of antimicrobial peptides. In some embodiments, the system is for producing a specified mixture of antimicrobial peptides that does not comprise bacteriocins. In some embodiments, the system is for producing a specified mixture of bacteriocins and antimicrobial peptides.

Some embodiments include a microfluidic device for producing a specified mixture of antimicrobial peptides and/or bacteriocins. The device can comprises discrete coding substrates that each encode an antimicrobial peptide and/or bacteriocin, and valves each disposed on a fluidic path connected to a discrete coding substrate. Each valve can be configured to regulate flow to or from the discrete coding substrate. The device can be configured to be placed in fluid communication with a fluidic reservoir and/or an in vitro transcription/translation solution. In some embodiments, the device further comprises a fluidic reservoir or an in vitro transcription/translation solution. Some embodiments include a microfluidic device for producing a specified mixture of bacteriocins. The device can comprise discrete coding substrates that each encode a bacteriocin, and valves each disposed on a fluidic path to or from a discrete coding substrate, configured to place the discrete coding substrate in fluid communication with a fluidic reservoir and/or an in vitro transcription/translation solution.

### BRIEF DESCRIPTION OF THE DRAWINGS

FIG. 1 is a schematic diagram of a conventional method for making a composition comprising bacteriocins. As shown, there are multiple strains for the production of a bacteriocin mixture. However, it is contemplated that there is a very high likelihood that the different bacteriocins will be of different concentrations ([A]≠[B]≠[C]≠[D]). Additionally, it is contemplated that the bacteriocins produced may be toxic to the host so as to cause production problems

FIG. 2 is a schematic diagram of an embodiment of a nucleic acid encoding a pro-polypeptide comprising bacteriocins and cleavage sites in accordance with some embodiments herein. As shown, a single bacteria may produce a pro-polypeptide encoded by a synthetic gene. It is contemplated that the molecular ratio of one bacteriocin can be changed by putting multiple copies of it in the pro-polypeptide.

FIG. 3 is a schematic diagram of an embodiment of a composition comprising bacteriocins and cleavage sites in accordance with some embodiments herein.

FIG. 4A is a schematic diagram of an embodiment of a pro-polypeptide comprising bacteriocins, cleavage sites, and a signal molecule in accordance with some embodiments herein.

FIG. 4B is a schematic diagram of an embodiment of a composition comprising bacteriocins and a signal molecule following cleavage of a pro-polypeptide in accordance with some embodiments herein.

FIG. 5 is a schematic diagram depicting a microfluidic device in accordance with some embodiments herein.

FIG. 6 is a schematic diagram depicting a microfluidic device in accordance with some embodiments herein.

FIG. 7 is a flow diagram illustrating a method for producing a specified mixture of antimicrobial peptides and/or bacteriocins according to some embodiments herein.

FIG. 8 is a flow diagram illustrating a method for making antimicrobial peptides and/or bacteriocins according to some embodiments herein.

### DETAILED DESCRIPTION

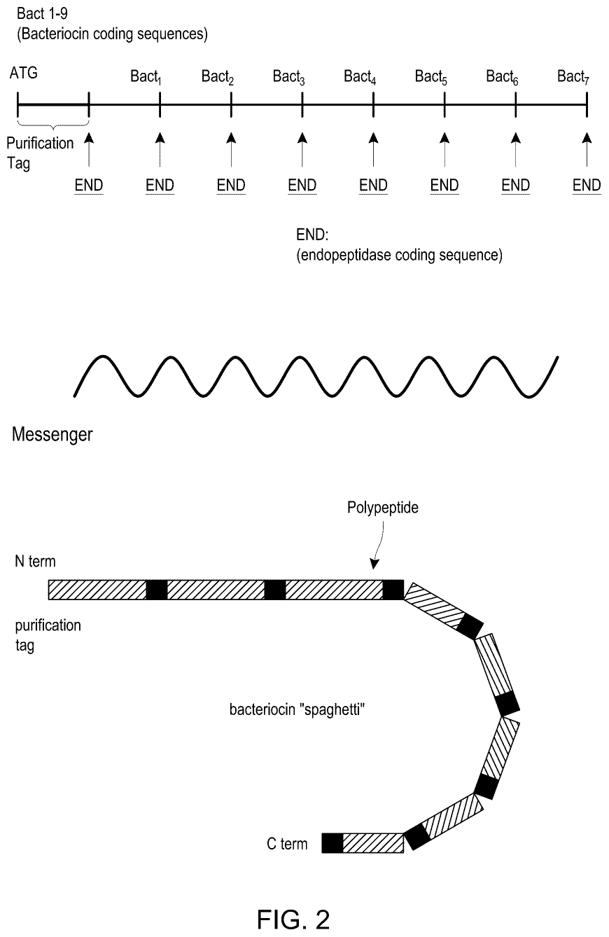

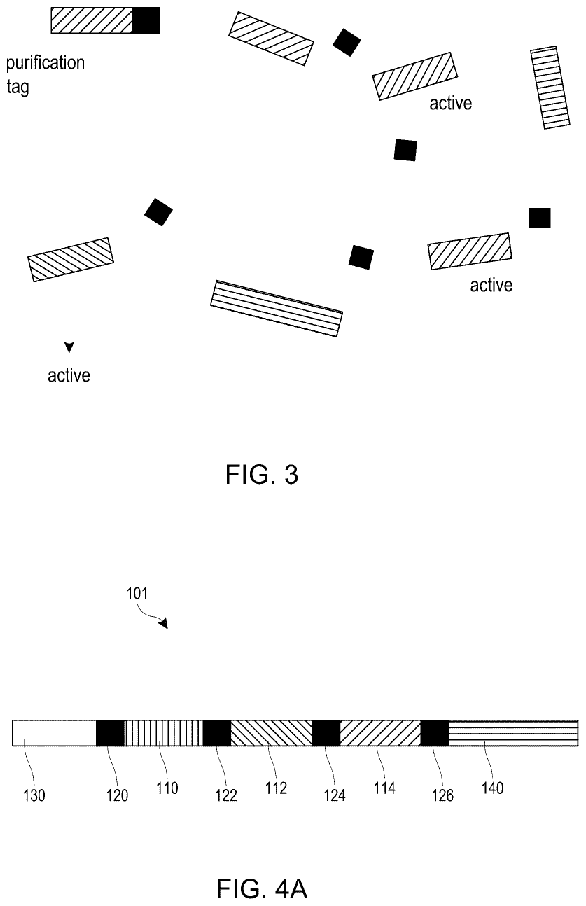

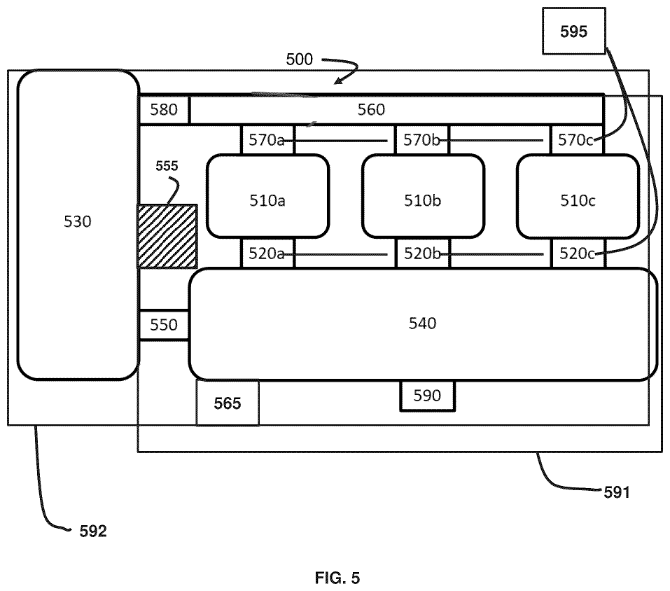

Described in accordance with some embodiments herein are methods and compositions for making bacteriocins in a desired ratio. Some embodiments include compositions comprising bacteriocins in precise ratios or stoichiometries, which can be useful for tuning the population of microbial organisms in a number of applications, for example in industrial biotechnology manufacturing processes, pharmaceutical, biologic, and cosmetic manufacturing, and medical applications. In some embodiments, a single pro-polypeptide containing multiple bacteriocins separated by cleavage sites is encoded by a nucleic acid, and produced (see, for example, FIG. 2) by a genetically-engineered microbial cell containing the nucleic acid. In some embodiments, the pro-polypeptide can further comprise signal molecules that can modulate the growth of microbial cells and/or cells of a multicellular host. The bacteriocins in the pro-polypeptide can be in an inactive form, and thus the microbial cell does not require immunity against these bacteriocins. The pro-polypeptide can be isolated, for example by affinity purification. The cleavage sites of the pro-polypeptide can be cleaved (see, for example, FIG. 3), thus producing a mixture of active bacteriocins. Conceptually, the pro-polypeptide can be envisioned as a strand of “spaghetti,” containing multiple component bacteriocins, which can then be cleaved into the individual bacteriocins. It is noted that in this Application, for brevity, a pro-polypeptide may also be referred to as “spaghetti.” Some embodiments include microfluidic devices and methods for making specified mixtures of bacteriocins. The “specified mixtures” of bacteriocins of methods and microfluidic devices of some embodiments herein can be specified according to parameters that include, but are not limited to, the composition of bacteriocins (so that the comprise specified bacteriocins and/or do not comprise other bacteriocins), the stoichiometry of the bacteriocins of the mixture, and optionally, the presence of auxiliary proteins. The microfluidic device can comprise discrete coding substrates that each encode a bacteriocin (See, for example, FIG. 5). Discrete coding substrates encoding the bacteriocins of the specified mixture can be placed in fluid communication with an in vitro transcription/translation solution, so that the discrete coding substrates encoding the bacteriocins of the specified mixture are incubated with the in vitro transcription/translation solution, thus producing the bacteriocins of the specified mixture. The bacteriocins of the specified mixture can then be placed in fluid communication with a fluidic reservoir of the microfluidic device, and the bacteriocins fluidically move to the fluidic reservoir, thus producing the specified mixture of antimicrobial peptides and/or bacteriocins in the fluidic reservoir. In some embodiments, each discrete coding substrate encodes a different bacteriocin. In some embodiments, a discrete coding substrate encodes two or more different bacteriocins. In some embodiments, at least one or all of the discrete coding substrates encode a “spaghetti” pro-polypeptide as described herein. Optionally, the discrete coding substrate can comprise or encode a protease as described herein, which can cleave the cleavage sites of the pro-polypeptide so that the discrete coding substrate can produce two or more bacteriocins in a specified stoichiometry. The microfluidic device can comprise an outlet, configured to place the specified mixture of antimicrobial peptides and/or bacteriocins in fluidic communication with a tissue, a wound, a host microbiome, or a vessel. The specified mixture of bacteriocins can be used, for example, as a point-of-care antimicrobial composition, and/or to test the effects of the specified mixture on a host tissue and/or host microbiome, for example one comprising an undesired microbial organism.

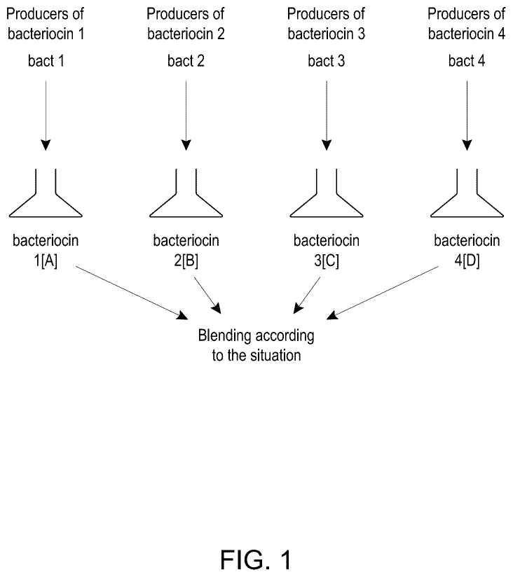

Advantageously, the relative quantities of different bacteriocins in the pro-polypeptide will precisely determine the ratio of bacteriocins in the mixture after cleavage, and this ratio will be maintained regardless of the rate of expression of the polypeptide within or among different microbial cells. In contrast, conventional methods in which bacteriocins are translated as separate peptides or produced by different cells (see FIG. 1) can be subject to variations in transformation efficiency, genetic engineering efficiency, and/or gene expression efficiency (for example if some bacteriocin-encoding plasmids replicate and/or initiate transcription more efficiently than others), so that the final ratios of bacteriocins will be less precise than embodiments herein. As another advantage, since each nucleic acid can encode many bacteriocins, some embodiments herein provide for a much more efficient genetic modification of a host cell than conventional approaches that would involve introducing multiple constructs each encoding a particular bacteriocin. As another advantage, since the bacteriocins can be produced in their inactive form in the pro-polypeptide, the “workhorse” microbial organism that produces the bacteriocins does not need to be configured with immunity to all, some, or any of these bacteriocins, and thus can readily be used to produce mixtures of any of a number of different bacteriocins. Although in some embodiments, the producing microbial organism can naturally or artificially be configured with immunity to one or more or all of the bacteriocins included in the pro-polypeptide.

Populations of microbial organisms targeted by bacteriocin compositions of some embodiments herein can exist in a number in commercially useful environments such as industrial cultures, fermenters, pharmaceutical, biological, and cosmetic manufacturing, in microbiomes, such as human organs, animals, and plants (e.g. on the roots or soil in which the plant grows), and in products, such as foods (for human and/or animals), drug products, and cosmetic products. Without being limited by theory, it is contemplated that populations of microbial organisms in any of these environments do not necessarily co-exist at a steady state. For example, as reviewed in Jaramillo et al., Nature Microbiology 2:17119 (2017), as populations of microbial organisms interact with each other (directly or indirectly), they can affect each other in positive or negative ways, but are not expected to exist at steady state, which conventionally lead to challenges in maintaining a co-culture. In some embodiments, when certain populations of microbial organisms reach a quantity or density above a certain threshold, a mixture of bacteriocins produced according to methods, and/or through the use or pro-polypeptides and/or nucleic acids and/or microbial cells of some embodiments herein can be added to the environment so as to target the microbial cells of this population. For example, if a population contains a large excess of microbial organism #1, and a smaller excess of microbial organism #2, in accordance with some embodiments, a mixture comprising a relatively high ratio of bacteriocin #1 (targeting microbial organism #1) to bacteriocin #2 (targeting microbial organism #2) can be administered to the population, so as to reduce the growth of microbial organisms #1 and #2, with a greater reduction targeted to microbial organism #1.

### Bacteriocins and Antimicrobial Peptides

As used herein, “bacteriocin,” and variations of this root term, has its customary and ordinary meaning as understood by one of skill in the art in view of this disclosure. It refers to a polypeptide that is secreted by a host cell and can neutralize at least one cell other than the individual host cell in which the polypeptide is made, including cells clonally related to the host cell and other microbial cells. “Bacteriocin” also encompasses a cell-free or chemically synthesized version of such a polypeptide. A cell that expresses a particular “immunity modulator” (discussed in more detail herein) is immune to the neutralizing effects of a particular bacteriocin or group of bacteriocins. As such, bacteriocins can neutralize a cell producing the bacteriocin and/or other microbial cells, so long as these cells do not produce an appropriate immunity modulator. As such, a host cell can exert cytotoxic or growth-inhibiting effects on a plurality of other microbial organisms by secreting bacteriocins. Detailed descriptions of bacteriocins, including methods and compositions for using bacteriocins to control the growth of microbial cells can be found, for example, in U.S. Pat. No. 9,333,227, which is hereby incorporated by reference in its entirety. “Bacteriocin” is not limited by the origin of the polypeptide, and by way of example is contemplated to encompass any bacteriocin, such as naturally-occurring bacteriocins, synthetic bacteriocins, and variants and combinations thereof.

The bacteriocins of some embodiments are initially produced in a pro-polypeptide, which can then be cleaved as described herein to produce the individual bacteriocins. In some embodiments, the pro-polypeptide is produced by the translational machinery (e.g. a ribosome, etc.) of a microbial cell. In some embodiments, the pro-polypeptide is produced by an in vitro expression system. The pro-polypeptide can undergo cleavage (for example processing by a cleavage enzyme such as a naturally-occurring or synthetic protease) to yield the polypeptide of the bacteriocin itself. As such, in some embodiments, a bacteriocin is produced from a precursor polypeptide. In some embodiments, a bacteriocin comprises a polypeptide that has undergone post-translational modifications, for example cleavage, or the addition of one or more functional groups. In some embodiments, a pro-polypeptide comprising, consisting essentially of, or consisting of bacteriocins and cleavage sites as described herein is chemically synthesized.

“Antibiotic,” and variations of this root term, has its customary and ordinary meaning as understood by one of skill in the art in view of this disclosure. It refers to a metabolite, or an intermediate of a metabolic pathway which can kill or arrest the growth of at least one microbial cell. Some antibiotics can be produced by microbial cells, for example bacteria. Some antibiotics can be synthesized chemically. It is understood that bacteriocins are distinct from antibiotics, at least in that bacteriocins refer to gene products (which, in some embodiments, undergo additional post-translational processing) or synthetic analogs of the same, while antibiotics refer to intermediates or products of metabolic pathways or synthetic analogs of the same.

Neutralizing activity of bacteriocins can include, for example, arrest of microbial reproduction, or cytotoxicity. Some bacteriocins have cytotoxic activity (e.g. “bacteriocide” effects), and thus can kill microbial organisms, for example bacteria, yeast, algae, synthetic microorganisms, and the like. Some bacteriocins can inhibit the reproduction of microbial organisms (e.g. “bacteriostatic” effects), for example bacteria, yeast, algae, synthetic microorganisms, and the like, for example by arresting the cell cycle. In some neutralizing comprises, consists of, or consists essentially of arrest of microbial reproduction, inhibition of reproduction of microbial organisms, and/or cytotoxicity.

In some embodiments, a particular neutralizing activity or ranges of activities (e.g. cytoxicity or arrest of microbial reproduction) is selected based on the type of microbial regulation that is desired and the particular strains or species of microbial organisms being targeted. As such, in some embodiments, particular bacteriocins or combinations of bacteriocins are selected. For example, in some embodiments, microbial cells are engineered to express particular bacteriocins based on the cells being regulated. In some embodiments, for example if contaminating cells are to be killed, at least one cytotoxic bacteriocin is provided. In some embodiments, a bacteriocin or combination of bacteriocins which is effective against contaminants which commonly occur in a particular culture, or a particular geographic location, or a particular type of culture grown in a particular geographic location are selected. In some embodiments, for example embodiments in which reversible regulation of microbial cell ratios is desired, a bacteriocin that inhibits microbial reproduction is provided. Without being limited by any particular theory, many bacteriocins can have neutralizing activity against microbial organisms that typically occupy the same ecological niche as the species that produces the bacteriocin. As such, in some embodiments, when a particular spectrum of bacteriocin activity is desired, a bacteriocin is selected from a host species that occupies the same (or similar) ecological niche as the microbial organism or organisms targeted by the bacteriocin. In some embodiments, a particular mixture and/or ratio is selected to target a single microbial organism (which can include targeting one or more than one microbial organisms of that type, for example clonally related microbial organisms). For example a particular type of microbial organism may be targeted more efficiently by a predetermined mixture and/or ratio of bacteriocins than by a single bacteriocin.

In some embodiments, one or more bacteriocin activities are selected in advance of culture growth, and a pro-polypeptide comprising the bacteriocins in a desired stoichiometry is prepared. A polynucleotide encoding the pro-polypeptide can be prepared, for example using nucleic acid synthesis and/or molecular cloning, and can be used to produce the pro-polypeptide. The polynucleotide can be transcribed (if a DNA) and translated using a number of suitable systems, for example in a microbial cell, or in an in vitro expression system. In some embodiments, bacteriocins (and ratios thereof) may be selected based on their ability to neutralize one or more invading organisms which are likely to attempt to grow in a particular culture. In some embodiments, bacteriocins (and ratios thereof) may be selected based on their ability to limit the growth of particular useful microbial strains in an environment, for example in an industrial feedstock, or in a fermenter, or in a food, pharmaceutical, or cosmetic manufacturing environment, or in a tissue environment such as a gut or skin microbiome, or in maintaining or tuning a microbial population in a plant, a plant root, and/or soil, or in preserving or maintaining the quality of a food, drug or cosmetic product. In some embodiments, one or more bacteriocin activities (and/or ratios) are selected based on one or more microbial strains or a population of microbial strains an existing environment. For example, in some embodiments, if particular invaders are identified in an environment, a panel of neutralizing bacteriocins (and ratios thereof) can be selected to neutralize the identified invaders. In some embodiments, the bacteriocins are selected to neutralize all or substantially all of the microbial cells in an environment, for example to eliminate an industrial culture in a culture environment so that a new industrial culture can be introduced to the culture environment, or to prevent or inhibit contamination of a pharmaceutical or cosmetic manufacturing environment, or to prevent or minimize contamination or spoilage of a food, drug, or cosmetic product.

For example, in some embodiments, an anti-fungal activity (such as anti-yeast activity) is desired. A number of bacteriocins with anti-fungal activity have been identified. For example, bacteriocins from Bacillus have been shown to have neutralizing activity against yeast strains (see Adetunji and Olaoye (2013) Malaysian Journal of Microbiology 9:130-13, hereby incorporated by reference in its entirety), an Enterococcus faecalis peptide (WLPPAGLLGRCGRWFRPWLLWLQ SGAQY KWLGNLFGLGPK, SEQ ID NO: 1) has been shown to have neutralizing activity against Candida species (see Shekh and Roy (2012) BMC Microbiology 12:132, hereby incorporated by reference in its entirety), and bacteriocins from Pseudomonas have been shown to have neutralizing activity against fungi such as Curvularia lunata, Fusarium species, Helminthosporium species, and Biopolaris species (Shalani and Srivastava (2008) The Internet Journal of Microbiology. Volume 5 Number 2. DOI: 10.5580/27dd-accessible on the worldwide web at archive.ispub.com/journal/the-internet-journal-of-microbiology/volume-5-number-2/screening-for-antifungal-activity-of-pseudomonas-fluorescens-against-phytopathogenic-fungi.html #sthash.d0Ys03UO.1DKuT1US.dpuf, hereby incorporated by reference in its entirety). By way of example, botrycidin AJ1316 (see Zuber, P et al. (1993) Peptide Antibiotics. In Bacillus subtilis and Other Gram-Positive Bacteria: Biochemistry, Physiology, and Molecular Genetics ed Sonenshein et al., pp. 897-916, American Society for Microbiology, hereby incorporated by reference in its entirety) and alirin B1 (see Shenin et al. (1995) Antibiot Khimioter 50:3-7, hereby incorporated by reference in its entirety) from B. subtilis have been shown to have antifungal activities. As such, in some embodiments, for example embodiments in which neutralization of a fungal microbial organism is desired, a bacteriocin comprises at least one of botrycidin AJ1316 or alirin B1.

For example, in some embodiments, bacteriocin activity in a culture of a particular microorganism (or collection of different microorganisms) is desirable, and bacteriocins are selected in predetermined ratios in order to neutralize microorganisms other than the desired microorganism(s). Bacteriocins typically produced by the desired microorganisms can be selected, as the desired microbial organisms can already produce the relevant immunity modulators against these bacteriocins, or can readily be engineered to produce the immunity modulators. As such, the selected bacteriocins can target undesired microbial cells, while causing little or no neutralization of the desired microbial organisms. For example, in some embodiments, bacteriocins are selected in particular ratios in order to neutralize invading microbial organisms typically found in a cyanobacteria culture environment, while preserving the cyanobacteria. Clusters of conserved bacteriocin polypeptides have been identified in a wide variety of cyanobacteria species. For example, at least 145 putative bacteriocin gene clusters have been identified in at least 43 cyanobacteria species, as reported in Wang et al. (2011), Genome Mining Demonstrates the Widespread Occurrence of Gene Clusters Encoding Bacteriocins in Cyanobacteria. PLOS ONE 6 (7): e22384, hereby incorporated by reference in its entirety. Exemplary cyanobacteria bacteriocins are shown in Table 1.2, as SEQ ID NO's 420, 422, 424, 426, 428, 430, 432, 434, 436, 438, 440, 442, 444, 446, 448, and 450.

In some embodiments, a microbial cell produces bacteriocins. In some embodiments, bacteriocins neutralize cells of a different species or strain from the microbial cell that produced bacteriocins. In some embodiments, bacteriocins neutralize cells of the same species or strain as the host cell if these cells lack an appropriate immunity modulator. As bacteriocins can mediate neutralization of both host and non-host microbial organisms, the skilled artisan will readily appreciate that a bacteriocin is distinct from poison-antidote systems, which involve an endogenous mechanism by which a host microorganism can neutralize only itself. In other words, bacteriocins can neutralize cells other than the cell in which they are produced (for example, bacteriocins can be selected and/or engineered to act as an ecological niche protector), while poison molecules kill only the individual cell in which they are produced (for example, to act as suicidal systems).

A number of bacteriocins have been identified and characterized. Without being limited by theory, exemplary bacteriocins can be classified as “class I” bacteriocins, which typically undergo post-translational modification, and “class II” bacteriocins, which are typically unmodified. Additionally, exemplary bacteriocins in each class can be categorized into various subgroups, as summarized in Table 1.1, which is adapted from Cotter, P. D. et al. “Bacteriocins—a viable alternative to antibiotics” Nature Reviews Microbiology (2013) 11:95-105, hereby incorporated by reference in its entirety.

Without being limited by theory, bacteriocins can effect neutralization of a target microbial cell in a variety of ways. For example, a bacteriocin can permeabilize a cell wall, thus depolarizing the cell wall and interfering with respiration.

TABLE 1.1
Classification of Exemplary Bacteriocins

| | | |
|---|---|---|
| Group | Distinctive feature | Examples |

Class I (typically modified)

| | | |
|---|---|---|
| MccC7-C51-type | Is covalently attached to a carboxy- | MccC7-C51 |
| bacteriocins | terminal aspartic acid | |
| Lasso peptides | Have a lasso structure | MccJ25 |
| Linear azole- or | Possess heterocycles but not other | MccB17 |
| azoline-containing | modifications | |
| peptides | | |
| Lantibiotics | Possess lanthionine bridges | Nisin, planosporicin, |
| | | mersacidin, actagardine, |
| | | mutacin 1140 |
| Linaridins | Have a linear structure and contain | Cypemycin |
| | dehydrated amino acids | |
| Proteusins | Contain multiple hydroxylations, | Polytheonamide A |
| | epimerizations and methylations | |
| Sactibiotics | Contain sulphur-α-carbon linkages | Subtilosin A, thuricin CD |
| Patellamide-like | Possess heterocycles and undergo | Patellamide A |
| cyanobactins | macrocyclization | |
| Anacyclamide-like | Cyclic peptides consisting of | Anacyclamide A10 |
| cyanobactins | proteinogenic amino acids with prenyl | |
| | attachments | |
| Thiopeptides | Contain a central pyridine, | Thiostrepton, nocathiacin |
| | dihydropyridine or piperidine ring as | I, GE2270 A, philipimycin |
| | well as heterocycles | |
| Bottromycins | Contain macrocyclic amidine, a | Bottromycin A2 |
| | decarboxylated carboxy-terminal | |
| | thiazole and carbon-methylated | |
| | amino acids | |
| Glycocins | Contain S-linked glycopeptides | Sublancin 168 |

Class II (typically unmodified or cyclic)

| | | |
|---|---|---|
| IIa peptides (pediocin | Possess a conserved YGNGV motif | Pediocin PA-1, enterocin |
| PA-1-like | (in which N represents any amino | CRL35, carnobacteriocin |
| bacteriocins) | acid) | BM1 |
| IIb peptides | Two unmodified peptides are | ABP118, lactacin F |
| | required for activity | |
| IIc peptides | Cyclic peptides | Enterocin AS-48 |
| IId peptides | Unmodified, linear, non-pediocin-like, | MccV, MccS, epidermicin |
| | single-peptide bacteriocins | NI01, lactococcin A |
| IIe peptides | Contain a serine-rich carboxy- | MccE492, MccM |
| | terminal region with a non-ribosomal | |
| | siderophore-type modification | |

A number of bacteriocins can be used in accordance with embodiments herein. Exemplary bacteriocins are shown in Table 1.2. In some embodiments, at least one bacteriocin comprising, consisting essentially of, or consisting of a polypeptide sequence of Table 1.2 is provided. As shown in Table 1.2, some bacteriocins function as pairs of molecules. As such, it will be understood that unless explicitly stated otherwise, when a functional “bacteriocin” or “providing a bacteriocin,” or the like is discussed herein, functional bacteriocin pairs are included along with bacteriocins that function individually. With reference to Table 1.2, “organisms of origin” listed in parentheses indicate alternative names and/or strain information for organisms known the produce the indicated bacteriocin. However, in some embodiments herein, bacteriocins are produced by a desired microbial organisms, and thus are not limited to the examples of organisms known to produce the bacteriocins shown in Table 1.2

Embodiments herein also include peptides and proteins with identity to bacteriocins described in Table 1.2. The term “identity” has its customary and ordinary meaning as understood by one of skill in the art in view of this disclosure. It is meant to include nucleic acid or protein sequence homology or three-dimensional homology. As used herein, a “variant” of a polypeptide, such as a bacteriocin, signal molecule, immunity modulator, tag (or any other component peptide of a pro-polypeptide as described herein) has its customary and ordinary meaning as understood by one of skill in the art in view of this disclosure. It will be understood to include identity as described herein to the reference sequence of at least about 70%, for example, at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, including ranges between any two of the listed values, for example 70%-100%, 75%-100%, 80%-100%, 85%-100%, 90%-100%, 95%-100%, 97%-100%, 99%-100%, 70%-99%, 75%-99%, 80%-99%, 85%-99%, 90%-99%, 95%-99%, 97%-99%, 70%-95%, 75%-95%, 80%-95%, 85%-95%, 90%-95%, 70%-90%, 75%-90%, 80%-90%, and 85%-90%. Several techniques exist to determine nucleic acid or polypeptide sequence homology and/or three-dimensional homology to polypeptides. These methods are routinely employed to discover the extent of identity that one sequence, domain, or model has to a target sequence, domain, or model. Percent identity may be determined using the BLAST software (Altschul, S. F., et al. (1990) “Basic local alignment search tool.” J. Mol. Biol. 215:403-410, accessible on the world wide web at blast.ncbi.nlm.nih.gov) with the default parameters.

A vast range of functional bacteriocins can incorporate features of bacteriocins disclosed herein, thus providing for a vast degree of identity to the bacteriocins in Table 1.2. In some embodiments, a bacteriocin has at least about 50% identity, for example, at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the polypeptides of Table 1.2, including ranges between any two of the listed values, for example 70%-100%, 75%-100%, 80%-100%, 85%-100%, 90%-100%, 95%-100%, 97%-100%, 99%-100%, 70%-99%, 75%-99%, 80%-99%, 85%-99%, 90%-99%, 95%-99%, 97%-99%, 70%-95%, 75%-95%, 80%-95%, 85%-95%, 90%-95%, 70%-90%, 75%-90%, 80%-90%, and 85%-90%.

In some embodiments, a bacteriocin with identity to a bacteriocin in Table 1.2 is provided, in which the bacteriocin has been modified to remove one or more cleavage sites that are being used in a pro-polypeptide as described herein. Without being limited by theory, it is contemplated that modifying bacteriocins to remove cleavage sites (for example by making conservative substitutions, such as small non-polar to small non-polar amino acid, e.g. Leu→Ile, or small polar to small polar amino acid, e.g., Ser→Thr) can prevent the bacteriocin itself from being cleaved when the pro-polypeptide is cleaved as described herein. As such, in some embodiments a bacteriocin in a pro-polypeptide as described herein (e.g., a bacteriocin of Table 1.2, or a variant thereof) does not contain any cleavage sites that are used to separate the bacteriocins (or other elements) of the pro-polypeptide.

TABLE 1.2
Exemplary Bacteriocins

| Polypeptide | | | | Polynucleotide |
|---|---|---|---|---|
| SEQ ID NO: | Name | Class | Organism of origin | SEQ ID NO: |
| 4 | Acidocin 8912 | Unclassified | Lactobacillus acidophilus | 5 |
| 6 | Acidocin A | class | Lactobacillus acidophilus | 7 |
| | | IIA/YGNGV | | |
| 8 | Acidocin B (AcdB) | Unclassified | Lactobacillus acidophilus | 9 |
| 10 | Acidocin LF221B | Unclassified | Lactobacillus gasseri | 11 |
| | (Gassericin K7 B) | | | |
| 12 | Aureocin A53 | Unclassified | Staphylococcus aureus | 13 |
| 14 | Avicin A | class | Enterococcus avium | 15 |
| | | IIA/YGNGV | (Streptococcus avium) | |
| 16 | Bacteriocin 31 | Unclassified | Enterococcus faecalis | 17 |
| | | | (Streptococcus faecalis) | |
| 18 | Bacteriocin J46 | Unclassified | Lactococcus lactis | 19 |
| 20 | Bacteriocin T8 | class IIa | Enterococcus faecium | 21 |
| | | | (Streptococcus faecium) | |
| 22 | Boticin B | Unclassified | Clostridium botulinum | 23 |
| 24 | Bovicin HJ50 | Lantibiotic | Streptococcus equinus | 25 |
| | | | (Streptococcus bovis) | |
| 26 | Brochocin-c | Unclassified | Brochothrix campestris | 27 |
| 28 | Butyrivibriocin AR10 | Unclassified | Butyrivibrio fibrisolvens | 29 |
| 30 | Butyrivibriocin OR79 | Lantibiotic | Butyrivibrio fibrisolvens | 31 |
| 32 | Carnobacteriocin B2 | class | Carnobacterium | 33 |
| | (Carnocin CP52) | IIA/YGNGV | maltaromaticum | |
| | | | (Carnobacterium | |
| | | | piscicola) | |
| 34 | Carnobacteriocin BM1 | class | Carnobacterium | 35 |
| | (Carnobacteriocin B1) | IIA/YGNGV | maltaromaticum | |
| | | | (Carnobacterium | |
| | | | piscicola) | |
| 36 | Carnobacteriocin-A | class IIc, non | Carnobacterium | 37 |
| | (Piscicolin-61) | subgrouped | maltaromaticum | |
| | | bacteriocins | (Carnobacterium | |
| | | (problematic) | piscicola) | |
| 38 | Carnocyclin-A | Unclassified | Carnobacterium | 39 |
| | | | maltaromaticum | |
| | | | (Carnobacterium | |
| | | | piscicola) | |
| 40 | Carocin D | Unclassified | Pectobacterium | 41 |
| | | | carotovorum subsp. | |
| | | | carotovorum (Erwinia | |
| | | | carotovora subsp. | |
| | | | carotovora) | |
| 42 | Cerein 7B | Unclassified | Bacillus cereus | 43 |
| 44 | Cinnamycin | Lantibiotic | Streptoverticillium | 45 |
| | (Lanthiopeptin) | | griseoverticillatum | |
| 46 | Circularin A | Unclassified | Geobacillus kaustophilus | 47 |
| | | | (strain HTA426) | |
| 48 | Closticin 574 | Unclassified | Clostridium tyrobutyricum | 49 |
| 50 | Coagulin A | Unclassified | Bacillus coagulans | 51 |
| 52 | Colicin-10 | Unclassified | Escherichia coli | 53 |
| 54 | Colicin-E1 | Unclassified | Escherichia coli | 55 |
| 56 | Colicin-Ia | Unclassified | Escherichia coli | 57 |
| 58 | Colicin-Ib | Unclassified | Escherichia coli | 59 |
| 60 | Colicin-M | Unclassified | Escherichia coli | 61 |
| 62 | Colicin-N | Unclassified | Escherichia coli | 63 |
| 64 | Colicin-V (Microcin-V) | Unclassified | Escherichia coli | 65 |
| 66 | Columbicin A | Lantibiotic | Enterococcus columbae | 67 |
| 68 | Curvacin-A | class | Lactobacillus curvatus | 69 |
| | | IIA/YGNGV | | |
| 70 | Cypemycin | Unclassified | Streptomyces sp. | 71 |
| 72 | Cytolysin | Lantibiotic | Bacillus halodurans | 73 |
| | | | (strain ATCC BAA-125/ | |
| | | | DSM 18197/FERM 7344/ | |
| | | | JCM 9153/C-125) | |
| 74 | Divercin V41 | class | Carnobacterium | 75 |
| | | IIa/YGNGV | divergens (Lactobacillus | |
| | | | divergens) | |
| 76 | Divergicin 750 | Unclassified | Carnobacterium | 77 |
| | | | divergens (Lactobacillus | |
| | | | divergens) | |
| 78 | Divergicin A | Class IIc | Carnobacterium | 79 |
| | | | divergens (Lactobacillus | |
| | | | divergens) | |
| 80 | Durancin Q | Unclassified | Enterococcus durans | 81 |
| 82 | Durancin TW-49M | Unclassified | Enterococcus durans | 83 |
| 84 | Dysgalacticin | Unclassified | Streptococcus | 85 |
| | | | dysgalactiae subsp. | |
| | | | equisimilis (Streptococcus | |
| | | | equisimilis) | |
| 86 | Enterocin 1071A | Unclassified | Enterococcus faecalis | 87 |
| | | | (Streptococcus faecalis) | |
| 88 | Enterocin 7A | bacteriocins | Enterococcus faecalis | 89 |
| | (Enterocin L50A) | without | (Streptococcus faecalis) | |
| | | sequence | | |
| | | leader | | |
| 90 | Enterocin 7B | Unclassified | Enterococcus faecalis | 91 |
| | | | (Streptococcus faecalis) | |
| 92 | Enterocin 96 | Class II | Enterococcus faecalis | 93 |
| | | | (strain ATCC 700802/ | |
| | | | V583) | |
| 94 | Enterocin A | Class IIa, IIc | Enterococcus faecium | 95 |
| | | (problematic) | (Streptococcus faecium) | |
| 96 | Enterocin AS-48 | Unclassified | Enterococcus faecalis | 97 |
| | (BACTERIOCIN AS- | | (Streptococcus faecalis) | |
| | 48) | | | |
| 98 | Enterocin B | class IIc, non | Enterococcus faecium | 99 |
| | | subgrouped | (Streptococcus faecium) | |
| | | bacteriocins | | |
| | | (problematic) | | |
| 100 | Enterocin CRL35 | Class IIa | Enterococcus mundtii | 101 |
| | (Mundticin KS) | | | |
| 102 | Enterocin EJ97 | Unclassified | Enterococcus faecalis | 103 |
| | | | (Streptococcus faecalis) | |
| 104 | Enterocin P | Class IIa, IIb | Enterococcus faecium | 105 |
| | | and IIc | (Streptococcus faecium) | |
| | | (problematic) | | |
| 106 | Enterocin Q | Class IIc | Enterococcus faecium | 107 |
| | | | (Streptococcus faecium) | |
| 108 | Enterocin SE-K4 | Class IIa | Enterococcus faecalis | 109 |
| | | | (Streptococcus faecalis) | |
| 110 | Enterocin W alfa | Class IIb | Enterococcus faecalis | 111 |
| | | | (Streptococcus faecalis) | |
| 112 | Enterocin W beta | Class IIb | Enterococcus faecalis | 113 |
| | | | (Streptococcus faecalis) | |
| 114 | Enterocin Xalpha | Class IIb | Enterococcus faecium | 115 |
| | | | (Streptococcus faecium) | |
| 116 | Enterocin Xbeta | Class IIb | Enterococcus faecium | 117 |
| | | | (Streptococcus faecium) | |
| 118 | Enterolysin A | class III | Enterococcus faecalis | 119 |
| | | | (Streptococcus faecalis) | |
| 120 | Epicidin 280 | Lantibiotic | Staphylococcus | 121 |
| | | | epidermidis | |
| 122 | Epidermicin NI01 | Unclassified | Staphylococcus | 123 |
| | | | epidermidis | |
| 124 | Epidermin | Lantibiotic | Staphylococcus | 125 |
| | | | epidermidis | |
| 126 | Epilancin K7 | Lantibiotic | Staphylococcus | 127 |
| | | | epidermidis | |
| 128 | Gallidermin | Lantibiotic | Staphylococcus | 129 |
| | | | gallinarum | |
| 130 | Garvicin A | IId | Lactococcus garvieae | 131 |
| 132 | Garvicin ML | Unclassified | Lactococcus garvieae | 133 |
| 134 | Gassericin A | Unclassified | Lactobacillus gasseri | 135 |
| 136 | Gassericin T | Unclassified | Lactobacillus gasseri | 137 |
| | (gassericin K7 B) | | | |
| 138 | Glycocin F | Unclassified | Lactobacillus plantarum | 139 |
| 140 | Halocin H4 | Unclassified | Haloferax mediterranei | 141 |
| | | | (strain ATCC 33500/ | |
| | | | DSM 1411/JCM 8866/ | |
| | | | NBRC 14739/NCIMB | |
| | | | 2177/R-4) | |
| | | | (Halobacterium | |
| | | | mediterranei) | |
| 142 | Halocin-S8 | Unclassified | Haloarchaeon S8a | 143 |
| 144 | Helveticin-J | Unclassified | Lactobacillus helveticus | 145 |
| | | | (Lactobacillus suntoryeus) | |
| 146 | Hiracin JM79 | Class II sec- | Enterococcus hirae | 147 |
| | | dependent | | |
| 148 | Lactacin-F (lafA) | class IIB | Lactobacillus johnsonii | 149 |
| | | | (strain CNCM I-12250/ | |
| | | | La1/NCC 533) | |
| 150 | Lactacin-F (lafX) | class IIB | Lactobacillus johnsonii | 151 |
| | | | (strain CNCM I-12250/ | |
| | | | La1/NCC 533) | |
| 152 | Lacticin 3147 A1 | Lantibiotic | Lactococcus lactis subsp. | 153 |
| | | | lactis (Streptococcus | |
| | | | lactis) | |
| 154 | Lacticin 3147 A2 | Lantibiotic | Lactococcus lactis subsp. | 155 |
| | | | lactis (Streptococcus | |
| | | | lactis) | |
| 156 | Lacticin 481 | Lantibiotic | Lactococcus lactis subsp. | 157 |
| | (Lactococcin DR) | | lactis (Streptococcus | |
| | | | lactis) | |
| 158 | Lacticin Q | Unclassified | Lactococcus lactis | 159 |
| 160 | Lacticin Z | Unclassified | Lactococcus lactis | 161 |
| 162 | Lactobin-A | class IIB | Lactobacillus amylovorus | 163 |
| | (Amylovorin-L471) | | | |
| 164 | Lactocin-S | Lantibiotic | Lactobacillus sakei L45 | 165 |
| 166 | Lactococcin 972 | Unclassified | Lactococcus lactis subsp. | 167 |
| | | | lactis (Streptococcus | |
| | | | lactis) | |
| 168 | Lactococcin-A | Unclassified | Lactococcus lactis subsp. | 169 |
| | | | cremoris (Streptococcus | |
| | | | cremoris) | |
| 170 | Lactococcin-B | Unclassified | Lactococcus lactis subsp. | 171 |
| | | | cremoris (Streptococcus | |
| | | | cremoris) | |
| 172 | Lactocyclicin Q | Unclassified | Lactococcus sp. QU 12 | 173 |
| 174 | Laterosporulin | Unclassified | Brevibacillus sp. GI-9 | 175 |
| 176 | Leucocin N | Class IId | Leuconostoc | 177 |
| | | | pseudomesenteroides | |
| 178 | Leucocin Q | Class IId | Leuconostoc | 179 |
| | | | pseudomesenteroides | |
| 180 | Leucocin-A (Leucocin | class | Leuconostoc gelidum | 181 |
| | A-UAL 187) | IIA/YGNGV | | |
| 182 | Leucocin-B (Leucocin | class | Leuconostoc carnosum | 183 |
| | B-Ta11a) | IIA/YGNGV | | |
| 184 | Leucocyclicin Q | Unclassified | Leuconostoc | 185 |
| | | | mesenteroides | |
| 186 | Lichenicidin A1 | Lantibiotic (two- | Bacillus licheniformis | 187 |
| | | peptide) | (strain DSM 13/ATCC | |
| | | | 14580) | |
| 188 | Linocin M18 | Unclassified | Brevibacterium linens | 189 |
| 190 | Listeriocin 743A | Class IIa | Listeria innocua | 191 |
| 192 | Mersacidin | Lantibiotic, type | Bacillus sp. (strain HIL- | 193 |
| | | B | Y85/54728) | |
| 194 | Mesentericin Y105 | class | Leuconostoc | 195 |
| | | IIA/YGNGV | mesenteroides | |
| 196 | Michiganin-A | Lantibiotic | Clavibacter michiganensis | 197 |
| | | | subsp. michiganensis | |
| 198 | Microcin B17 | Unclassified | Escherichia coli | 199 |
| | (MccB17) | | | |
| 200 | Microcin C7 | Unclassified | Escherichia coli | 201 |
| 202 | Microcin E492 | Unclassified | Klebsiella pneumoniae | 203 |
| 204 | Microcin H47 | Unclassified | Escherichia coli | 205 |
| 206 | Microcin J25 | Unclassified | Escherichia coli | 207 |
| 208 | Microcin-24 | Unclassified | Escherichia coli | 209 |
| 210 | Mundticin KS | Unclassified | Enterococcus mundtii | 211 |
| 212 | Mundticin L | class | Enterococcus mundtii | 213 |
| | | IIA/YGNGV | | |
| 214 | Mutacin 1140 | Lantibiotic | Streptococcus mutans | 215 |
| | (Mutacin III) | | | |
| 216 | Mutacin-2 | Lantibiotic | Streptococcus mutans | 217 |
| 218 | Nisin A | Lantibiotic | Lactococcus lactis subsp. | 219 |
| | | | lactis (Streptococcus | |
| | | | lactis) | |
| 220 | Nisin F | Lantibiotic | Lactococcus lactis | 221 |
| 222 | Nisin Q | Lantibiotic | Lactococcus lactis | 223 |
| 224 | Nisin U | Lantibiotic | Streptococcus uberis | 225 |
| 226 | Nisin Z | Lantibiotic | Lactococcus lactis subsp. | 227 |
| | | | lactis (Streptococcus | |
| | | | lactis) | |
| 228 | Nukacin ISK-1 | Lantibiotic | Staphylococcus warneri | 229 |
| 230 | Paenicidin A | Lantibiotic | Paenibacillus polymyxa | 231 |
| | | | (Bacillus polymyxa) | |
| 232 | Pediocin PA-1 | class | Pediococcus acidilactici | 233 |
| | (Pediocin ACH) | IIA/YGNGV | | |
| 234 | Penocin A | class | Pediococcus pentosaceus | 235 |
| | | IIA/YGNGV | (strain ATCC 25745/183- | |
| | | | 1w) | |
| 236 | Pep5 | Lantibiotic | Staphylococcus | 237 |
| | | | epidermidis | |
| 238 | Piscicolin 126 | class | Carnobacterium | 239 |
| | | IIA/YGNGV | maltaromaticum | |
| | | | (Carnobacterium | |
| | | | piscicola) | |
| 240 | Plantaricin 1.25 β | Unclassified | Lactobacillus plantarum | 241 |
| 242 | Plantaricin 423 | class IIa | Lactobacillus plantarum | 243 |
| 244 | Plantaricin ASM1 | Unclassified | Lactobacillus plantarum | 245 |
| 246 | Plantaricin E | Unclassified | Lactobacillus plantarum | 247 |
| 248 | Plantaricin F | Class IIb | Lactobacillus plantarum | 249 |
| 250 | Plantaricin J | Class IIb | Lactobacillus plantarum | 251 |
| 252 | Plantaricin K | Unclassified | Lactobacillus plantarum | 253 |
| 254 | Plantaricin NC8 α | Unclassified | Lactobacillus plantarum | 255 |
| 256 | Plantaricin NC8 β | Unclassified | Lactobacillus plantarum | 257 |
| 258 | Plantaricin S α | Unclassified | Lactobacillus plantarum | 259 |
| 260 | Plantaricin S β | Unclassified | Lactobacillus plantarum | 261 |
| 262 | Plantaricin W α | Lantibiotic (two- | Lactobacillus plantarum | 263 |
| | | peptide) | | |
| 264 | Plantaricin W β | Lantibiotic (two- | Lactobacillus plantarum | 265 |
| | | peptide) | | |
| 266 | Plantaricin-A | Unclassified | Lactobacillus plantarum | 267 |
| | | | (strain ATCC BAA-793/ | |
| | | | NCIMB 8826/WCFS1) | |
| 268 | Propionicin SM1 | Unclassified | Propionibacterium | 269 |
| | | | jensenii | |
| 270 | Propionicin T1 | Unclassified | Propionibacterium thoenii | 271 |
| 272 | Propionicin-F | Unclassified | Propionibacterium | 273 |
| | | | freudenreichii subsp. | |
| | | | freudenreichii | |
| 274 | Pyocin S1 | Unclassified | Pseudomonas aeruginosa | 275 |
| 276 | Pyocin S2 | colicin/pyosin | Pseudomonas aeruginosa | 277 |
| | | nuclease family | (strain ATCC 15692/ | |
| | | | PAO1/1C/PRS 101/ | |
| | | | LMG 12228) | |
| 278 | Ruminococcin-A | Lantibiotic | Ruminococcus gnavus | 279 |
| 280 | Sakacin G | Class IIa | Lactobacillus sakei | 281 |
| 282 | Sakacin-A | class | Lactobacillus sakei | 283 |
| | | IIA/YGNGV | | |
| 284 | Sakacin-P (Sakacin | class | Lactobacillus sakei | 285 |
| | 674) | IIA/YGNGV | | |
| 286 | Salivaricin 9 | lantibiotic | Streptococcus salivarius | 287 |
| 288 | Salivaricin A | Lantibiotic | Streptococcus pyogenes | 289 |
| | | | serotype M28 (strain | |
| | | | MGAS6180) | |
| 290 | Salivaricin A3 | Lantibiotic | Streptococcus salivarius | 291 |
| 292 | Salivaricin-A sa | Lantibiotic | Streptococcus salivarius | 293 |
| 294 | Staphylococcin C55 | Lantibiotic (two- | Staphylococcus aureus | 295 |
| | alpha | peptide) | | |
| 296 | Staphylococcin C55 | Lantibiotic (two- | Staphylococcus aureus | 297 |
| | beta | peptide) | | |
| 298 | Streptin | lantibiotic | Streptococcus pyogenes | 299 |
| 300 | Streptococcin A-FF22 | Lantibiotic | Streptococcus pyogenes | 301 |
| 302 | Streptococcin A-M49 | Lantibiotic | Streptococcus pyogenes | 303 |
| | | | serotype M49 | |
| 304 | Sublancin 168 | Lantibiotic | Bacillus subtilis (strain | 305 |
| | | | 168) | |
| 306 | Subtilin | Lantibiotic | Bacillus subtilis | 307 |
| 308 | Subtilosin | Unclassified | Bacillus subtilis (strain | 309 |
| | | | 168) | |
| 310 | Subtilosin-A | Unclassified | Bacillus subtilis (strain | 311 |
| | | | 168) | |
| 312 | Thermophilin 1277 | Lantibiotic | Streptococcus | 313 |
| | | | thermophilus | |
| 314 | Thermophilin 13 | Unclassified | Streptococcus | 315 |
| | | | thermophilus | |
| 316 | Thermophilin A | Unclassified | Streptococcus | 317 |
| | | | thermophilus | |
| 318 | Thiocillin (Micrococcin | Unclassified | Bacillus cereus (strain | 319 |
| | P1) (Micrococcin P2) | | ATCC 14579/DSM 31) | |
| | (Thiocillin I) (Thiocillin | | | |
| | II) (Thiocillin III) | | | |
| | (Thiocillin IV) | | | |
| | (Antibiotic YM- | | | |
| | 266183) (Antibiotic | | | |
| | YM-266184) | | | |
| 320 | Thuricin CD alpha | two-peptide | Bacillus cereus 95/8201 | 321 |
| | | lantibiotic | | |
| 322 | Thuricin CD beta | two-peptide | Bacillus cereus 95/8201 | 323 |
| | | lantibiotic | | |
| 324 | Thuricin-17 | Class IId | Bacillus thuringiensis | 325 |
| 326 | Trifolitoxin | Unclassified | Rhizobium | 327 |
| | | | leguminosarum bv. trifolii | |
| 328 | Ubericin A | Class IIa | Streptococcus uberis | 329 |
| 330 | Uberolysin | Unclassified | Streptococcus uberis | 331 |
| 332 | UviB | Unclassified | Clostridium perfringens | 333 |
| 334 | Variacin | Lantibiotic, | Micrococcus varians | 335 |
| | | Type A | | |
| 336 | Zoocin A | Unclassified | Streptococcus equi | 337 |
| | | | subsp. zooepidemicus | |
| 338 | Fulvocin-C | Unclassified | Myxococcus fulvus | 339 |
| 340 | Duramycin-C | Lantibiotic | Streptomyces | 341 |
| | | | griseoluteus | |
| 342 | Duramycin | Lantibiotic B | Streptoverticillium | 343 |
| | (duramycin-B) | | griseoverticillatum | |
| | (Leucopeptin) | | | |
| 344 | Carnocin UI49 | lantibiotic | Carnobacterium sp. | 345 |
| | | | (strain UI49) | |
| 346 | Lactococcin-G α | Unclassified | Lactococcus lactis subsp. | 347 |
| | | | lactis (Streptococcus | |
| | | | lactis) | |
| 348 | Lactococcin-G β | Unclassified | Lactococcus lactis subsp. | 349 |
| | | | lactis (Streptococcus | |
| | | | lactis) | |
| 350 | Ancovenin | Lantibiotic | Streptomyces sp. (strain | 351 |
| | | | A647P-2) | |
| 352 | Actagardine | Lantibiotic | Actinoplanes liguriae | 353 |
| | (Gardimycin) | | | |
| 354 | Curvaticin FS47 | Unclassified | Lactobacillus curvatus | 355 |
| 356 | Bavaricin-MN | class | Lactobacillus sakei | 357 |
| | | IIA/YGNGV | | |
| 358 | Mutacin B-Ny266 | Lantibiotic | Streptococcus mutans | 359 |
| 360 | Mundticin | class | Enterococcus mundtii | 361 |
| | | IIA/YGNGV | | |
| 362 | Bavaricin-A | class | Lactobacillus sakei | 363 |
| | | IIA/YGNGV | | |
| 364 | Lactocin-705 | Class IIb | Lactobacillus paracasei | 365 |
| 366 | Leucocin-B | Unclassified | Leuconostoc | 367 |
| | | | mesenteroides | |
| 368 | Leucocin C | class | Leuconostoc | 369 |
| | | IIA/YGNGV | mesenteroides | |
| 370 | LCI | Unclassified | Bacillus subtilis | 371 |
| 372 | Lichenin | Unclassified | Bacillus licheniformis | 373 |
| 374 | Lactococcin MMFII | class | Lactococcus lactis subsp. | 375 |
| | | IIA/YGNGV | lactis (Streptococcus | |
| | | | lactis) | |
| 376 | Serracin-P | Phage-Tail-Like | Serratia plymuthica | 377 |
| 378 | Halocin-C8 | Unclassified | Halobacterium sp. (strain | 379 |
| | | | AS7092) | |
| 380 | Subpeptin JM4-B | Unclassified | Bacillus subtilis | 381 |
| 382 | Curvalicin-28a | Unclassified | Lactobacillus curvatus | 383 |
| 384 | Curvalicin-28b | Unclassified | Lactobacillus curvatus | 385 |
| 386 | Curvalicin-28c | Unclassified | Lactobacillus curvatus | 387 |
| 388 | Thuricin-S | Unclassified | Bacillus thuringiensis | 389 |
| | | | subsp. entomocidus | |
| 390 | Curvaticin L442 | Unclassified | Lactobacillus curvatus | 391 |
| 392 | Divergicin M35 | class | Carnobacterium | 393 |
| | | IIa/YGNGV | divergens (Lactobacillus | |
| | | | divergens) | |
| 394 | Enterocin E-760 | class IIb | Enterococcus sp. | 395 |
| 396 | Bacteriocin E50-52 | Unclassified | Enterococcus faecium | 397 |
| | | | (Streptococcus faecium) | |
| 398 | Paenibacillin | Unclassified | Paenibacillus polymyxa | 399 |
| | | | (Bacillus polymyxa) | |
| 400 | Epilancin 15x | Unclassified | Staphylococcus | 401 |
| | | | epidermidis | |
| 402 | Enterocin-HF | class IIa | Enterococcus faecium | 403 |
| | | | (Streptococcus faecium) | |
| 404 | Bacillocin 602 | Class IIa | Paenibacillus polymyxa | 405 |
| | | | (Bacillus polymyxa) | |
| 406 | Bacillocin 1580 | Class IIa | Bacillus circulans | 407 |
| 408 | Bacillocin B37 | Unclassified | Paenibacillus polymyxa | 409 |
| | | | (Bacillus polymyxa) | |
| 410 | Rhamnosin A | Unclassified | Lactobacillus rhamnosus | 411 |
| 412 | Lichenicidin A2 | Lantibiotic (two- | Bacillus licheniformis | 413 |
| | | peptide) | (strain DSM 13/ATCC | |
| | | | 14580) | |
| 414 | Plantaricin C19 | Class IIa | Lactobacillus plantarum | 415 |
| 416 | Acidocin J1132 β | Class IIb | Lactobacillus acidophilus | 417 |
| 418 | factor with anti- | Unclassified | Enterococcus faecalis | 419 |
| | Candida activity | | | |
| 420 | Ava_1098 | Unclassified | Anabaena variabilis | 421 |
| | (putative heterocyst | | ATCC 29413 | |
| | differentiation protein) | | | |
| 422 | alr2818 | Unclassified | Nostoc sp 7120 | 423 |
| | (putative heterocyst | | | |
| | differentiation protein) | | | |
| 424 | Aazo_0724 (putative | Unclassified | Nostoc azollae 0708 | 425 |
| | heterocyst | | | |
| | differentiation protein) | | | |
| 426 | AM1_4010 (putative | Unclassified | Acaryochloris marina | 427 |
| | heterocyst | | MBIC11017 | |
| | differentiation protein) | | | |
| 428 | PCC8801_3266 | Unclassified | Cyanothece PCC 8801 | 429 |
| | (putative heterocyst | | | |
| | differentiation protein) | | | |
| 430 | Cyan8802_2855 | Unclassified | Cyanothece PCC 8802 | 431 |
| | (putative heterocyst | | | |
| | differentiation protein) | | | |
| 432 | PCC7424_3517 | Unclassified | Cyanothece PCC 7424 | 433 |
| 434 | cce_2677(putative | Unclassified | Cyanothece ATCC 51142 | 435 |
| | HetP protein) | | | |
| 436 | CY0110_11572 | Unclassified | Cyanothece CCY0110 | 437 |
| | (putative heterocyst | | | |
| | differentiation protein) | | | |
| 438 | MC7420_4637 | Unclassified | Microcoleus | 439 |
| | | | chthonoplastes PCC 7420 | |
| 440 | asr1611 (putative | Unclassified | Nostoc sp 7120 | 441 |
| | DUF37 family protein) | | | |
| 442 | Ava_4222 (putative | Unclassified | Anabaena variabilis | 443 |
| | DUF37 family protein) | | ATCC 29413 | |
| 444 | N9414_07129 | Unclassified | Nodularia spumigena | 445 |
| | (putative DUF37 | | CCY9414 | |
| | family protein) | | | |
| 446 | Aazo_0083 (putative | Unclassified | Nostoc azollae 0708 | 447 |
| | DUF37 family protein) | | | |
| 448 | S7335_3409 (putative | Unclassified | Synechococcus PCC | 449 |
| | DUF37 family protein) | | 7335 | |
| 450 | P9303_21151 | Unclassified | Prochlorococcus marinus | 451 |
| | (putative DUF37 | | MIT 9303 | |
| | family protein) | | | |
| 699 | Curvalicin-28c | Unclassified | Lactobacillus curvatus | 700 |
| 701 | thruicin-S | Unclassified | Bacillus thuringiensis | 702 |
| 703 | curvaticin L442 | Unclassified | Lactobacillus curvatus | 704 |
| | | | L442 | |
| 705 | Bacteriocin divergicin | P84962 | Carnobacterium | 706 |
| | M35 | | divergens (Lactobacillus | |
| | | | divergens) | |
| 707 | Lantibiotic 107891 | P85065 | Microbispora sp. (strain | 708 |
| | | | 107891) | |
| 709 | Enterocin E-760 | P85147 | Enterococcus sp. | 710 |
| | (Bacteriocin E-760) | | | |
| 711 | Bacteriocin E50-52 | P85148 | Enterococcus faecium | 712 |
| | | | (Streptococcus faecium) | |
| 713 | Lantibiotic | P86013 | Paenibacillus polymyxa | 714 |
| | paenibacillin | | (Bacillus polymyxa) | |
| 715 | Lantibiotic epilancin | P86047 | Staphylococcus | 716 |
| | 15X | | epidermidis | |
| 717 | Enterocin-HF | P86183 | Enterococcus faecium | 718 |
| | | | (Streptococcus faecium) | |
| 719 | Bacteriocin SRCAM | P86393 | Paenibacillus polymyxa | 720 |
| | 602 | | (Bacillus polymyxa) | |
| 721 | Bacteriocin SRCAM | P86394 | Bacillus circulans | 722 |
| | 1580 | | | |
| 723 | Bacteriocin SRCAM | P86395 | Paenibacillus polymyxa | 724 |
| | 37 | | (Bacillus polymyxa) | |
| 725 | Bacteriocin rhamnosin | P86526 | Lactobacillus rhamnosus | 726 |
| | A (Fragment) | | | |
| 727 | Lantibiotic lichenicidin | P86720 | Bacillus licheniformis | 728 |
| | A2 (LchA2) (BliA2) | | (strain ATCC 14580/ | |
| | | | DSM 13/JCM 2505/ | |
| | | | NBRC 12200/NCIMB | |
| | | | 9375/NRRL NRS-1264/ | |
| | | | Gibson 46) | |
| 729 | Pyocin-S2 (EC 3.1.—.—) | | Pseudomonas aeruginosa | 730 |
| | (Killer protein) | | (strain ATCC 15692/ | |
| | | | DSM 22644/CIP 104116/ | |
| | | | JCM 14847/LMG | |
| | | | 12228/1C/PRS 101/ | |
| | | | PAO1) | |
| 731 | Plantaricin C19 | | Lactobacillus plantarum | 732 |
| | (Fragment) | | | |
| 733 | LsbB | | Lactococcus lactis subsp. | 734 |
| | | | lactis (Streptococcus | |
| | | | lactis) | |
| 735 | ACIDOCIN J1132 | | Lactobacillus acidophilus | 736 |
| | beta peptide | | | |
| | (Fragment) | | | |
| 737 | Uncharacterized | | Lactobacillus salivarius | 738 |
| | protein | | cp400 | |

As used herein, “bacteriocin polynucleotide” has its customary and ordinary meaning as understood by one of skill in the art in view of this disclosure. It refers to a polynucleotide encoding a bacteriocin. As used herein, “bacteriocin coding sequence” has its customary and ordinary meaning as understood by one of skill in the art in view of this disclosure. It refers to nucleic acid coding sequence (e.g., RNA or DNA) that encodes the polypeptide sequence of a bacteriocin. In some embodiments, the host cell comprises at least one bacteriocin.

Antimicrobial peptides are a class of peptides that confer innate immune activity to kill or arrest the growth of microbial organisms. As used herein “antimicrobial peptide” (including variations of this root term) has its customary and ordinary meaning as would be understood by one of ordinary skill in the art in view of this disclosure. Classically, antimicrobial peptides have been described as peptides produced by the innate immune systems of invertebrates and vertebrates. Thus, while bacteriocins have classically been referred to a class of microbial gene products that target microbial organisms, antimicrobial peptides have classically been referred to as a class of invertebrate and vertebrate gene products that target microbial organisms. Examples of antimicrobial peptides suitable for methods, microfluidic devices, and systems of some embodiments herein are known in the art, and can be founds, for example, at The Antimicrobial Peptide Database accessible on the world wide web at aps.unmc.edu/AP/, which is incorporated herein by reference in its entirety. Over 1000 antimicrobial peptides and variants thereof have been identified and cataloged. The Antimicrobial Peptide Database is described in Wang et al. (2016), Nucleic Acids Res. 44 (Database issue): D1087-D1093, which is incorporated herein by reference in its entirety. Examples of antimicrobial peptides include bacteriocins, antibacterial, antiviral, anti-HIV, antifungal, antiparasitic and anticancer peptides, such as Dermaseptin-B2, Abaecin, Ct-AMP1, Andropin, Aurein 1.1, Lactoferricin B, and Heliomicin. Methods, compositions, systems, and microfluidic devices of some embodiments comprise naturally-occurring antimicrobial peptides, or a nucleic acid encoding the same. Methods, compositions, systems, and microfluidic devices of some embodiments comprise non-naturally occurring antimicrobial peptides, or nucleic acids encoding the same. Methods, compositions, systems, and microfluidic devices of some embodiments include antimicrobial peptides that comprise a mutation or variation in a naturally-occurring antimicrobial peptides, or a nucleic acid encoding the same. Methods, compositions, systems, and microfluidic devices of some embodiments comprise antimicrobial peptides comprising, consisting essentially of, or consisting of non-naturally occurring peptide sequences, or nucleic acids encoding the same.

It is further contemplated that methods, systems, and microfluidic devices of some embodiments herein can be in conjunction with naturally occurring antimicrobial peptides, variants of naturally occurring antimicrobial peptides, and/or synthetic antimicrobial peptides. As such, antimicrobial peptides of methods, systems, and device of some embodiments can comprise, consist essentially of, or consist of naturally occurring antimicrobial peptides, variants of naturally occurring antimicrobial peptides, and/or synthetic antimicrobial peptides. In some embodiments, a variant antimicrobial peptide has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to a reference antimicrobial peptide (for example Dermaseptin-B2, Abaecin, Ct-AMP1, Andropin, Aurein 1.1, Lactoferricin B, or Heliomicin), including ranges between any two of the listed values, for example 70%-100%, 75%-100%, 80%-100%, 85%-100%, 90%-100%, 95%-100%, 97%-100%, 99%-100%, 70%-99%, 75%-99%, 80%-99%, 85%-99%, 90%-99%, 95%-99%, 97%-99%, 70%-95%, 75%-95%, 80%-95%, 85%-95%, 90%-95%, 70%-90%, 75%-90%, 80%-90%, and 85%-90%. Wherever bacteriocins are mentioned herein, it is expressly contemplated that any antimicrobial peptide as described herein may be substituted for one, two or more, or all of the noted bacteriocins (although it will be appreciated that in some contexts, such as a bacteriocin-immunity modulator pair, the antimicrobial peptide will have different properties and interact differently than a bacitracin). Accordingly, some embodiments of the methods and microfluidic devices described herein include bacteriocins only, some embodiments of the methods and microfluidic devices described herein include antimicrobial peptides only, and some embodiments of the methods and microfluidic devices described herein include a combination of bacteriocins and antimicrobial peptides. Accordingly, in the methods and compositions of some embodiments, a pro-polypeptide comprises an antimicrobial peptide instead of a bacteriocin. Accordingly, in the methods and compositions of some embodiments, a pro-polypeptide comprises one or more antimicrobial peptides and one or more bacteriocins.

### Bacteriocin Immunity Modulators

Exemplary bacteriocin immunity modulators are shown in Table 2. While the immunity modulators in Table 2 are naturally-occurring, the skilled artisan will appreciate that variants of the immunity modulators of Table 2, naturally-occurring immunity modulators other than the immunity modulators of Table 2, or synthetic immunity modulators can be used according to some embodiments herein. In some embodiments, a microbial cell that produced a pro-polypeptide comprising bacteriocins does not produce an immunity modulator for at least one of the bacteriocins. Without being limited by theory, it is contemplated that in some embodiments, the bacteriocins in the pro-polypeptide are inactive, and thus have little to no ability to neutralize the cell that produced them (or a cell clonally related to the cell that produced them), so that the cell does not require immunity against these bacteriocins of the pro-polypeptide.

In some embodiments, a particular immunity modulator or particular combination of immunity modulators confers immunity to a particular bacteriocin, particular class or category of bacteriocins, or particular combination of bacteriocins. Exemplary bacteriocins to which immunity modulators can confer immunity are identified in Table 2. While Table 2 identifies an “organism of origin” for exemplary immunity modulators, these immunity modulators can readily be expressed in other naturally-occurring, genetically modified, or synthetic microorganisms to provide a desired bacteriocin immunity activity in accordance with some embodiments herein. As such, as used herein “immunity modulator” has its customary and ordinary meaning as understood by one of skill in the art in view of this disclosure, and refers not only to structures expressly provided herein, but also to structure that have substantially the same effect as the “immunity modulator” structures described herein, including fully synthetic immunity modulators, and immunity modulators that provide immunity to bacteriocins that are functionally equivalent to the bacteriocins disclosed herein.

Exemplary polynucleotide sequences encoding the polypeptides of Table 2 are indicated in Table 2. The skilled artisan will readily understand that the genetic code is degenerate, and moreover, codon usage can vary based on the particular organism in which the gene product is being expressed, and as such, a particular polypeptide can be encoded by more than one polynucleotide. In some embodiments, a polynucleotide encoding a bacteriocin immunity modulator is selected based on the codon usage of the organism expressing the bacteriocin immunity modulator. In some embodiments, a polynucleotide encoding a bacteriocin immunity modulator is codon optimized based on the particular organism expressing the bacteriocin immunity modulator. A vast range of functional immunity modulators can incorporate features of immunity modulators disclosed herein, thus providing for a vast degree of identity to the immunity modulators in Table 2. In some embodiments, an immunity modulator has at least about 50% identity, for example, at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the polypeptides of Table 2, or a range of identity defined by any two of the preceding values.

TABLE 2
Exemplary bacteriocin immunity modulators

| Polypeptide | | | Polynucleotide |
|---|---|---|---|
| SEQ ID NO: | Name | Organism of origin | SEQ ID NO: |
| 452 | Microcin H47 immunity modulator | Escherichia coli | 453 |
| | Mchl | | |
| 454 | Colicin-E3 immunity modulator | Escherichia coli | 455 |
| | (Colicin-E3 chain B) (ImmE3) | | |
| | (Microcin-E3 immunity modulator) | | |
| 456 | Colicin-E1 immunity modulator | Escherichia coli | 457 |
| | (ImmE1) (Microcin-E1 immunity | | |
| | modulator) | | |
| 458 | Cloacin immunity modulator | Escherichia coli | 459 |
| 460 | Colicin-E2 immunity modulator | Escherichia coli | 461 |
| | (ImmE2) (Microcin-E2 immunity | | |
| | modulator) | | |
| 462 | Colicin-A immunity modulator | Citrobacter | 463 |
| | (Microcin-A immunity modulator) | freundii | |
| 464 | Colicin-Ia immunity modulator | Escherichia coli | 465 |
| 466 | Colicin-Ib immunity modulator | Escherichia coli | 467 |
| 468 | Colicin-N immunity modulator | Escherichia coli | 469 |
| | (Microcin-N immunity modulator) | | |
| 470 | Colicin-E8 immunity modulator | Escherichia coli | 471 |
| | (ImmE8) (Microcin-E8 immunity | | |
| | modulator) | | |
| 472 | Lactococcin-A immunity modulator | Lactococcus | 473 |
| | | lactis subsp. | |
| | | lactis | |
| | | (Streptococcus | |
| | | lactis) | |
| 474 | Lactococcin-A immunity modulator | Lactococcus | 475 |
| | | lactis subsp. | |
| | | cremoris | |
| | | (Streptococcus | |
| | | cremoris) | |
| 476 | Colicin-D immunity modulator | Escherichia coli | 477 |
| | (Microcin-D immunity modulator) | | |
| 478 | Colicin-E5 immunity modulator | Escherichia coli | 479 |
| | (ImmE5) (Microcin-E5 immunity | | |
| | modulator) | | |
| 480 | Colicin-E6 immunity modulator | Escherichia coli | 481 |
| | (ImmE6) (Microcin-E6 immunity | | |
| | modulator) | | |
| 482 | Colicin-E8 immunity modulator in | Escherichia coli | 483 |
| | ColE6 (E8Imm[E6]) | | |
| 484 | Colicin-E9 immunity modulator | Escherichia coli | 485 |
| | (ImmE9) (Microcin-E9 immunity | | |
| | modulator) | | |
| 486 | Colicin-M immunity modulator | Escherichia coli | 487 |
| | (Microcin-M immunity modulator) | | |
| 488 | Colicin-B immunity modulator | Escherichia coli | 489 |
| | (Microcin-B immunity modulator) | | |
| 490 | Colicin-V immunity modulator | Escherichia coli | 491 |
| | (Microcin-V immunity modulator) | | |
| 492 | Colicin-E1* immunity modulator | Shigella sonnei | 493 |
| | (ImmE1) (Microcin-E1* immunity | | |
| | modulator) | | |
| 494 | Colicin-E1 immunity modulator | Escherichia coli | 495 |
| | (ImmE1) (Microcin-E1 immunity | | |
| | modulator) | | |
| 496 | Probable leucocin-A immunity | Leuconostoc | 497 |
| | modulator | gelidum | |
| 498 | Lactococcin-B immunity modulator | Lactococcus | 499 |
| | | lactis subsp. | |
| | | cremoris | |
| | | (Streptococcus | |
| | | cremoris) | |
| 500 | Pediocin PA-1 immunity modulator | Pediococcus | 501 |
| | (Pediocin ACH immunity modulator) | acidilactici | |
| 502 | Putative carnobacteriocin-BM1 | Carnobacterium | 503 |
| | immunity modulator | maltaromaticum | |
| | | (Carnobacterium | |
| | | piscicola) | |
| 504 | Putative carnobacteriocin-B2 | Carnobacterium | 505 |
| | immunity modulator (Carnocin-CP52 | maltaromaticum | |
| | immunity modulator) | (Carnobacterium | |
| | | piscicola) | |
| 506 | Nisin immunity modulator | Lactococcus | 507 |
| | | lactis subsp. | |
| | | lactis | |
| | | (Streptococcus | |
| | | lactis) | |
| 508 | Trifolitoxin immunity modulator | Rhizobium | 509 |
| | | leguminosarum | |
| | | bv. trifolii | |
| 510 | Antilisterial bacteriocin subtilosin | Bacillus subtilis | 511 |
| | biosynthesis protein AlbD | (strain 168) | |
| 512 | Putative ABC transporter ATP- | Bacillus subtilis | 513 |
| | binding protein AlbC (Antilisterial | (strain 168) | |
| | bacteriocin subtilosin biosynthesis | | |
| | protein AlbC) | | |
| 514 | Antilisterial bacteriocin subtilosin | Bacillus subtilis | 515 |
| | biosynthesis protein AlbB | (strain 168) | |
| 516 | Colicin-E7 immunity modulator | Escherichia coli | 517 |
| | (ImmE7) (Microcin-E7 immunity | | |
| | modulator) | | |
| 518 | Pyocin-S1 immunity modulator | Pseudomonas | 519 |
| | | aeruginosa | |
| 520 | Pyocin-S2 immunity modulator | Pseudomonas | 521 |
| | | aeruginosa (strain | |
| | | ATCC 15692/ | |
| | | PAO1/1C/PRS | |
| | | 101/LMG | |
| | | 12228) | |
| 522 | Hiracin-JM79 immunity factor | Enterococcus | 523 |
| | | hirae | |
| 524 | Probable mesentericin-Y105 | Leuconostoc | 525 |
| | immunity modulator | mesenteroides | |
| 526 | Microcin-24 immunity modulator | Escherichia coli | 527 |
| 528 | Colicin-K immunity modulator | Escherichia coli | 529 |
| 530 | Microcin C7 self-immunity modulator | Escherichia coli | 531 |
| | MccF | | |
| 532 | Sakacin-A immunity factor | Lactobacillus | 533 |
| | | sakei | |
| 534 | Colicin-E5 immunity modulator in | Escherichia coli | 535 |
| | ColE9 (E5Imm[E91) | | |
| 536 | Antilisterial bacteriocin subtilosin | Bacillus subtilis | 537 |
| | biosynthesis protein AlbD | | |
| 538 | Microcin-J25 export ATP- | Escherichia coli | 539 |
| | binding/permease protein McjD | | |
| | (Microcin-J25 immunity modulator) | | |
| | (Microcin-J25 secretion ATP-binding | | |
| | protein McjD) | | |
| 540 | Microcin E492 immunity modulator | Klebsiella | 541 |
| | | pneumoniae | |

### Promoters

Promoters are well known in the art. A promoter can be used to drive the transcription of one or more genes. In some embodiments, a promoter drives expression of polynucleotide encoding a desired gene product as described herein. In some embodiments, a promoter drives expression of a polynucleotide encoding a pro-polypeptide comprising two or more bacteriocins as described herein. In some embodiments, a promoter drives expression of an immunity modulator polynucleotide as described herein. In some embodiments, a promoter drives expression of a polynucleotide encoding a pro-polypeptide comprising two or more bacteriocins in a microbial cell, but the microbial cell does not express immunity modulators for one or more of these bacteriocins (for example, the cell can lack a promoter driving transcription of the immunity modulator, or can lack nucleic acid encoding the immunity modulator). Some promoters can drive transcription at all times (“constitutive promoters”). Some promoters can drive transcription under only select circumstances (“conditional promoters”), for example depending on the presence or absence of an environmental condition, chemical compound, gene product, stage of the cell cycle, or the like.

The skilled artisan will appreciate that depending on the desired expression activity, an appropriate promoter can be selected, and placed in cis with a nucleic acid sequence to be expressed. Exemplary promoters with exemplary activities, and useful in some embodiments herein are provided in Tables 3.1-3.11 herein. The skilled artisan will appreciate that some promoters are compatible with particular transcriptional machinery (e.g. RNA polymerases, general transcription factors, and the like). As such, while compatible “species” are identified for some promoters described herein, it is contemplated that in some embodiments, these promoters can readily function in microorganisms other than the identified species, for example in species with compatible endogenous transcriptional machinery, genetically modified species comprising compatible transcriptional machinery, or fully synthetic microbial organisms comprising compatible transcriptional machinery.

The promoters of Tables 3.1-3.11 herein are publicly available from the Biobricks foundation. It is noted that the Biobricks foundation encourages use of these promoters in accordance with BioBrick™ Public Agreement (BPA).

It should be appreciated that any of the “coding” polynucleotides described herein (for example a bacteriocin polynucleotide, immunity polynucleotide, or nucleotide encoding a pro-polypeptide comprising two or more bacteriocins) is generally amenable to being expressed under the control of a desired promoter. In some embodiments, a single “coding” polynucleotide is under the control of a single promoter. In some embodiments, two or more “coding” polynucleotides are under the control of a single promoter, for example two, three, four, five, six, seven, eight, nine, or ten polynucleotides.

Generally, translation initiation for a particular transcript is regulated by particular sequences at or 5′ of 5′ end of the coding sequence of a transcript. For example, a coding sequence can begin with a start codon configured to pair with an initiator tRNA. While naturally-occurring translation systems typically use Met (AUG) as a start codon, it will be readily appreciated that an initiator tRNA can be engineered to bind to any desired triplet or triplets, and accordingly, triplets other than AUG can also function as start codons in certain embodiments. Additionally, sequences near the start codon can facilitate ribosomal assembly, for example a Kozak sequence ((gcc) gccRccAUGG, SEQ ID NO: 542, in which R represents “A” or “G”) or Internal Ribosome Entry Site (IRES) in typical eukaryotic translational systems, or a Shine-Delgarno sequence (GGAGGU, SEQ ID NO: 543) in typical prokaryotic translation systems. As such in some embodiments, a transcript comprising a “coding” polynucleotide sequence, for example a bacteriocin polynucleotide or immunity polynucleotide, or nucleotide encoding a pro-polypeptide comprising two or more bacteriocins, comprises an appropriate start codon and translational initiation sequence. In some embodiments, for example if two or more “coding” polynucleotide sequences are positioned in cis on a transcript, each polynucleotide sequence comprises an appropriate start codon and translational initiation sequence(s). In some embodiments, for example if two or more “coding” polynucleotide sequences are positioned in cis on a transcript, the two sequences are under control of a single translation initiation sequence, and either provide a single polypeptide that can function with both encoded polypeptides in cis.

TABLE 3.1
Exemplary Metal-Sensitive Promoters

| SEQ | | |
|---|---|---|
| ID | | |
| NO: | Name | Description |
| 544 | BBa_I721001 | Lead Promoter |
| 545 | BBa_I731004 | FecA promoter |
| 546 | BBa_I760005 | Cu-sensitive promoter |
| 547 | BBa_I765000 | Fe promoter |
| 548 | BBa_I765007 | Fe and UV promoters |
| 549 | BBa_J3902 | PrFe (PI + PII rus operon) |

TABLE 3.2
Exemplary Cell Signaling-Responsive Promoters

| SEQ | | |
|---|---|---|
| ID | | |
| NO: | Name | Description |
| 550 | BBa_I1051 | Lux cassette right promoter |
| 551 | BBa_I14015 | P(Las) TetO |
| 552 | BBa_I14016 | P(Las) CIO |
| 553 | BBa_I14017 | P(RhI) |
| 554 | BBa_I739105 | Double Promoter (LuxR/HSL, positive/cl, negative) |
| 555 | BBa_I746104 | P2 promoter in agr operon from S. aureus |
| 556 | BBa_I751501 | plux-cl hybrid promoter |
| 557 | BBa_I751502 | plux-lac hybrid promoter |
| 558 | BBa_I761011 | CinR, CinL and glucose controlled promotor |
| 559 | BBa_J06403 | RhIR promoter repressible by Cl |
| 560 | BBa_J102001 | Reverse Lux Promoter |
| 561 | BBa_J64000 | rhII promoter |
| 562 | BBa_J64010 | lasI promoter |
| 563 | BBa_J64067 | LuxR + 3OC6HSL independent R0065 |
| 564 | BBa_J64712 | LasR/LasI Inducible & RHLR/RHLI repressible Promoter |
| 565 | BBa_K091107 | pLux/cl Hybrid Promoter |
| 566 | BBa_K091117 | pLas promoter |
| 567 | BBa_K091143 | pLas/cl Hybrid Promoter |
| 568 | BBa_K091146 | pLas/Lux Hybrid Promoter |
| 569 | BBa_K091156 | pLux |
| 570 | BBa_K091157 | pLux/Las Hybrid Promoter |
| 571 | BBa_K145150 | Hybrid promoter: HSL-LuxR activated, P22 C2 repressed |
| 572 | BBa_K266000 | PAI + LasR −> LuxI (AI) |
| 573 | BBa_K266005 | PAI + LasR −> LasI & AI + LuxR --\| LasI |
| 574 | BBa_K266006 | PAI + LasR −> LasI + GFP & AI + LuxR --\| LasI + GFP |
| 575 | BBa_K266007 | Complex QS −> LuxI & LasI circuit |
| 576 | BBa_K658006 | position 3 mutated promoter lux pR-3 (luxR & HSL regulated) |
| 577 | BBa_K658007 | position 5 mutated promoter lux pR-5 (luxR & HSL regulated) |
| 578 | BBa_K658008 | position 3&5 mutated promoter lux pR-3/5 (luxR & HSL |
| | | regulated) |
| 579 | BBa_R0061 | Promoter (HSL-mediated luxR repressor) |
| 580 | BBa_R0062 | Promoter (luxR & HSL regulated -- lux pR) |
| 581 | BBa_R0063 | Promoter (luxR & HSL regulated -- lux pL) |
| 582 | BBa_R0071 | Promoter (RhIR & C4-HSL regulated) |
| 583 | BBa_R0078 | Promoter (cinR and HSL regulated) |
| 584 | BBa_R0079 | Promoter (LasR & PAI regulated) |
| 585 | BBa_R1062 | Promoter, Standard (luxR and HSL regulated -- lux pR) |

TABLE 3.3
Exemplary Constitutive E. coli σ⁷⁰ Promoters

| SEQ | | |
|---|---|---|
| ID | | |
| NO: | Name | Description |
| 586 | BBa_I14018 | P(Bla) |
| 587 | BBa_I14033 | P(Cat) |
| 588 | BBa_I14034 | P(Kat) |
| 589 | BBa_I732021 | Template for Building Primer Family Member |
| 590 | BBa_I742126 | Reverse lambda cl-regulated promoter |
| 591 | BBa_J01006 | Key Promoter absorbs 3 |
| 592 | BBa_J23100 | constitutive promoter family member |
| 593 | BBa_J23101 | constitutive promoter family member |
| 594 | BBa_J23102 | constitutive promoter family member |
| 595 | BBa_J23103 | constitutive promoter family member |
| 596 | BBa_J23104 | constitutive promoter family member |
| 597 | BBa_J23105 | constitutive promoter family member |
| 598 | BBa_J23106 | constitutive promoter family member |
| 599 | BBa_J23107 | constitutive promoter family member |
| 600 | BBa_J23108 | constitutive promoter family member |
| 601 | BBa_J23109 | constitutive promoter family member |
| 602 | BBa_J23110 | constitutive promoter family member |
| 603 | BBa_J23111 | constitutive promoter family member |
| 604 | BBa_J23112 | constitutive promoter family member |
| 605 | BBa_J23113 | constitutive promoter family member |
| 606 | BBa_J23114 | constitutive promoter family member |
| 607 | BBa_J23115 | constitutive promoter family member |
| 608 | BBa_J23116 | constitutive promoter family member |
| 609 | BBa_J23117 | constitutive promoter family member |
| 610 | BBa_J23118 | constitutive promoter family member |
| 611 | BBa_J23119 | constitutive promoter family member |
| 612 | BBa_J23150 | 1 bp mutant from J23107 |
| 613 | BBa_J23151 | 1 bp mutant from J23114 |
| 614 | BBa_J44002 | pBAD reverse |
| 615 | BBa_J48104 | NikR promoter, a protein of the ribbon helix-helix family of |
| | | trancription factors that repress expre |
| 616 | BBa_J54200 | lacq_Promoter |
| 617 | BBa_J56015 | lacIQ - promoter sequence |
| 618 | BBa_J64951 | E. Coli CreABCD phosphate sensing operon promoter |
| 619 | BBa_K088007 | GlnRS promoter |
| 620 | BBa_K119000 | Constitutive weak promoter of lacZ |
| 621 | BBa_K119001 | Mutated LacZ promoter |
| 622 | BBa_K137029 | constitutive promoter with (TA)10 between −10 and −35 |
| | | elements |
| 623 | BBa_K137030 | constitutive promoter with (TA)9 between −10 and −35 |
| | | elements |
| 624 | BBa_K137031 | constitutive promoter with (C)10 between −10 and −35 |
| | | elements |
| 625 | BBa_K137032 | constitutive promoter with (C)12 between −10 and −35 |
| | | elements |
| 626 | BBa_K137085 | optimized (TA) repeat constitutive promoter with 13 bp |
| | | between −10 and −35 elements |
| 627 | BBa_K137086 | optimized (TA) repeat constitutive promoter with 15 bp |
| | | between −10 and −35 elements |
| 628 | BBa_K137087 | optimized (TA) repeat constitutive promoter with 17 bp |
| | | between −10 and −35 elements |
| 629 | BBa_K137088 | optimized (TA) repeat constitutive promoter with 19 bp |
| | | between −10 and −35 elements |
| 630 | BBa_K137089 | optimized (TA) repeat constitutive promoter with 21 bp |
| | | between −10 and −35 elements |
| 631 | BBa_K137090 | optimized (A) repeat constitutive promoter with 17 bp |
| | | between −10 and −35 elements |
| 632 | BBa_K137091 | optimized (A) repeat constitutive promoter with 18 bp |
| | | between −10 and −35 elements |
| 633 | BBa_K256002 | J23101:GFP |
| 634 | BBa_K256018 | J23119:IFP |
| 635 | BBa_K256020 | J23119:HO1 |
| 636 | BBa_K256033 | Infrared signal reporter (J23119:1FP:J23119:HO1) |
| 637 | BBa_K292000 | Double terminator + constitutive promoter |
| 638 | BBa_K292001 | Double terminator + Constitutive promoter + Strong RBS |
| 639 | BBa_K418000 | IPTG inducible Lac promoter cassette |
| 640 | BBa_K418002 | IPTG inducible Lac promoter cassette |
| 641 | BBa_K418003 | IPTG inducible Lac promoter cassette |
| 642 | BBa_M13101 | M13K07 gene I promoter |
| 643 | BBa_M13102 | M13K07 gene II promoter |
| 644 | BBa_M13103 | M13K07 gene III promoter |
| 645 | BBa_M13104 | M13K07 gene IV promoter |
| 646 | BBa_M13105 | M13K07 gene V promoter |
| 647 | BBa_M13106 | M13K07 gene VI promoter |
| 648 | BBa_M13108 | M13K07 gene VIII promoter |
| 649 | BBa_M13110 | M13110 |
| 650 | BBa_M31519 | Modified promoter sequence of g3. |
| 651 | BBa_R1074 | Constitutive Promoter I |
| 652 | BBa_R1075 | Constitutive Promoter II |
| 653 | BBa_S03331 | --Specify Parts List-- |

TABLE 3.4
Exemplary Constitutive E. coli σ^{S} Promoters

| SEQ | | |
|---|---|---|
| ID | | |
| NO: | Name | Description |
| 654 | BBa_J45992 | Full-length stationary phase osmY promoter |
| 655 | BBa_J45993 | Minimal stationary phase osmY promoter |

TABLE 3.5
Exemplary Constitutive E. coli σ³² Promoters

| SEQ | | |
|---|---|---|
| ID | | |
| NO: | Name | Description |
| 656 | BBa_J45504 | htpG Heat Shock Promoter |

TABLE 3.6
Exemplary Constitutive B. subtilis σ^{A} Promoters

| SEQ | | |
|---|---|---|
| ID | | |
| NO: | Name | Description |
| 657 | BBa_K143012 | Promoter veg a constitutive promoter |
| | | for B. subtilis |
| 658 | BBa_K143013 | Promoter 43 a constitutive promoter |
| | | for B. subtilis |
| 659 | BBa_K780003 | Strong constitutive promoter for |
| | | Bacillus subtilis |
| 660 | BBa_K823000 | PliaG |
| 661 | BBa_K823002 | PlepA |
| 662 | BBa_K823003 | Pveg |

TABLE 3.7
Exemplary Constitutive B. subtilis σ^{B} Promoters

| SEQ | | |
|---|---|---|
| ID | | |
| NO: | Name | Description |
| 663 | BBa_K143010 | Promoter ctc for B. subtilis |
| 664 | BBa_K143011 | Promoter gsiB for B. subtilis |
| 665 | BBa_K143013 | Promoter 43 a constitutive promoter for B. subtilis |

TABLE 3.8
Exemplary Constitutive Promoters from miscellaneous prokaryotes

| SEQ | | |
|---|---|---|
| ID | | |
| NO: | Name | Description |
| 666 | a_K112706 | Pspv2 from Salmonella |
| 667 | BBa_K112707 | Pspv from Salmonella |

TABLE 3.9
Exemplary Constitutive Promoters from bacteriophage T7

| SEQ | | |
|---|---|---|
| ID | | |
| NO: | Name | Description |
| 668 | BBa_I712074 | T7 promoter (strong promoter from T7 |
| | | bacteriophage) |
| 669 | BBa_I719005 | T7 Promoter |
| 670 | BBa_J34814 | T7 Promoter |
| 671 | BBa_J64997 | T7 consensus −10 and rest |
| 672 | BBa_K113010 | overlapping T7 promoter |
| 673 | BBa_K113011 | more overlapping T7 promoter |
| 674 | BBa_K113012 | weaken overlapping T7 promoter |
| 675 | BBa_R0085 | T7 Consensus Promoter Sequence |
| 676 | BBa_R0180 | T7 RNAP promoter |
| 677 | BBa_R0181 | T7 RNAP promoter |
| 678 | BBa_R0182 | T7 RNAP promoter |
| 679 | BBa_R0183 | T7 RNAP promoter |
| 680 | BBa_Z0251 | T7 strong promoter |
| 681 | BBa_Z0252 | T7 weak binding and processivity |
| 682 | BBa_Z0253 | T7 weak binding promoter |

TABLE 3.10
Exemplary Constitutive Promoters from yeast

| SEQ | | |
|---|---|---|
| ID | | |
| NO: | Name | Description |
| 683 | BBa_I766555 | pCyc (Medium) Promoter |
| 684 | BBa_I766556 | pAdh (Strong) Promoter |
| 685 | BBa_I766557 | pSte5 (Weak) Promoter |
| 686 | BBa_J63005 | yeast ADH1 promoter |
| 687 | BBa_K105027 | cyc100 minimal promoter |
| 688 | BBa_K105028 | cyc70 minimal promoter |
| 689 | BBa_K105029 | cyc43 minimal promoter |
| 690 | BBa_K105030 | cyc28 minimal promoter |
| 691 | BBa_K105031 | cyc16 minimal promoter |
| 692 | BBa_K122000 | pPGK1 |
| 693 | BBa_K124000 | pCYC Yeast Promoter |
| 694 | BBa_K124002 | Yeast GPD (TDH3) Promoter |
| 695 | BBa_K319005 | yeast mid-length ADH1 promoter |
| 696 | BBa_M31201 | Yeast CLB1 promoter region, G2/M cell |
| | | cycle specific |

TABLE 3.11
Exemplary Constitutive Promoters from miscellaneous eukaryotes

| SEQ | | |
|---|---|---|
| ID | | |
| NO: | Name | Description |
| 697 | BBa_I712004 | CMV promoter |
| 698 | BBa_K076017 | Ubc Promoter |

The above-referenced promoters are provided by way of non-limiting example only. The skilled artisan will readily recognize that many variants of the above-referenced promoters, and many other promoters (including promoters isolated from naturally existing organisms, variations thereof, and fully synthetic promoters) can readily be used in accordance with some embodiments herein.

### Cleavage Sites

As used herein, “cleavage site” has its customary and ordinary meaning as understood by one of skill in the art in view of this disclosure. It refers to a polypeptide sequence that mediates the cleavage of a polypeptide (for example by hydrolysis of a peptide bond) to separate a single polypeptide into two or more discrete polypeptides. In some embodiments, a cleavage site comprises, consists of, or consists essentially of a consensus polypeptide sequence for cleavage by a “cleavage enzyme,” such as a peptidase. In some embodiments, the cleavage enzyme is a wild-type, a variant, or a synthetic cleavage enzyme, for example a wild-type, variant, or synthetic endopeptidase. A number of example cleavage enzymes and their corresponding cleavage sites are described herein. For reference, the cleavage sites are described with reference to formula (I), below:

Pn-P4-P3-P2-P1-∥Cleavage∥-P1′-P2′-P3′-P4′-Pm′ (I)

in which amino acid residues in a substrate undergoing cleavage are designated P1, P2, P3, P4 etc. in the N-terminal (or “upstream”) direction from the cleaved bond. Likewise, the residues in C-terminal (or “downstream”) direction of the cleavage site are designated P1′, P2′, P3′, P4′, etc.

Example cleavage enzymes and their cleavage sites useful in accordance with some embodiments herein are described in Table 4, below.

It is noted that a number of cleavage enzymes cleave at consensus sequences, and thus, a particular cleavage enzyme can cut at two or more particular polypeptide sequences that fall within the scope of a consensus sequence (or consensus sequences) for that cleavage enzyme. As such, for convenience herein, unless explicitly stated otherwise, two cleavage sites may be referred to as the “same” when they are both cut by the same cleavage enzyme under the same conditions, even if they are two different sequences (which may fall within the scope of that cleavage enzyme's consensus sequence(s)). For example, the sequences DVADL (SEQ ID NO: 739) and DVADI (SEQ ID NO: 740) may both be referred to as the “same” cleavage site for the purposes of cleavage by Caspase 2 because they both fall within the scope of consensus sequence for Caspase 2, even though these cleavage sites are not identical. On the other hand, also for convenience herein, unless explicitly stated otherwise, two cleavage sites may be referred to as “different” when they are each cut by different cleavage enzymes, but would not be cut by the same cleavage enzyme.

TABLE 4
Example Cleavage Enzymes and Cleavage Sites

| Cleavage Enzyme | Cleavage Site (with reference to Formula (I)) | | | | | |
|---|---|---|---|---|---|---|
| Name | P4 | P3 | P2 | P1 | P1′ | P2′ |
| Arg-C proteinase | — | — | — | R | — | — |
| Asp-N | — | — | — | — | D | — |
| endopeptidase | | | | | | |
| BNPS-Skatole | — | — | — | W | — | — |
| Caspase 1 | F, W, Y, | | H, A or T | D | not P, E, D, | — |
| | or L | | | | Q, K or R | |
| Caspase 2 | D | V | A | D | not P, E, D, | — |
| | | | | | Q, K or R | |
| Caspase 3 | D | M | Q | D | not P, E, D, | — |
| | | | | | Q, K or R | |
| Caspase 4 | L | E | V | D | not P, E, D, | — |
| | | | | | Q, K or R | |
| Caspase 5 | L or W | E | H | D | — | — |
| Caspase 6 | V | E | H or I | D | not P, E, D, | — |
| | | | | | Q, K or R | |
| Caspase 7 | D | E | V | D | not P, E, D, | — |
| | | | | | Q, K or R | |
| Caspase 8 | I or L | E | T | D | not P, E, D, | — |
| | | | | | Q, K or R | |
| Caspase 9 | L | E | H | D | — | — |
| Caspase 10 | I | E | A | D | — | — |
| Chymotrypsin-high | — | — | — | F or Y | not P | — |
| specificity (C-term | — | — | — | W | not M or P | — |
| to [FYW], not | — | — | — | F, L or Y | not P | — |
| before P) | — | — | — | W | not M or P | — |
| Chymotrypsin-low | — | — | — | M | Not P or Y | — |
| specificity (C-term | — | — | — | H | not D, M, | — |
| to [FYWML], not | | | | | P, or W | |
| before P | | | | | | |
| Clostripain | — | — | — | R | — | — |
| (Clostridiopeptidase | | | | | | |
| B) | | | | | | |
| CNBr | — | — | — | M | — | — |
| Enterokinase | D or E | D or E | D or E | K | — | — |
| Factor Xa | A, F, G, I, L, | D or E | G | R | — | — |
| | T, V or M | | | | | |
| Formic acid | — | — | — | D | — | — |
| Glutamyl | — | — | — | E | — | — |
| endopeptidase | | | | | | |
| GranzymeB | I | E | P | D | — | — |
| Hydroxylamine | — | — | — | N | G | — |
| Iodosobenzoic acid | — | — | — | W | — | — |
| LysC | — | — | — | K | — | — |
| Neutrophil elastase | — | — | — | A or V | — | — |
| NTCB (2-nitro-5- | — | — | — | — | C | — |
| thiocyanobenzoic | | | | | | |
| acid) | | | | | | |
| Pepsin (pH 1.3) | — | not H, K, | not P | not R | F or L | not P |
| | | or R | | | | |
| | — | not H, K, | not P | F or L | — | not P |
| | | or R | | | | |
| Pepsin (pH > 2) | — | not H, K, | not P | not R | F, L, W, | not P |
| | | or R | | | or Y | |
| | — | not H, K, | not P | F, L, W, | — | not P |
| | | or R | | or Y | | |
| Proline- | — | — | H, K or R | P | not P | — |
| endopeptidase | | | | | | |
| Proteinase K | — | — | — | A, E, F, I, L | — | — |
| | | | | T, V, W or | | |
| | | | | Y | | |
| Staphylococcal | — | — | not E | E | — | — |
| peptidase I | | | | | | |
| Thermolysin | — | — | — | not D or | A, F, I, L, | — |
| | | | | E | M or V | |
| Thrombin | A, F, G, I, L, | A, F, G, I, L, | P | R | not D or E | Not |
| | T, V or M | T, V, W or A | | | | DE |
| Trypsin (please | — | — | | K or R | not P | — |
| note the exceptions | — | — | W | K | P | — |
| in the following | — | — | M | R | P | — |
| rows) | | | | | | |
| Exceptions to the | — | — | C or D | K | D | — |
| Trypsin Consensus | — | — | C | K | H or Y | — |
| Site Noted in the | — | — | C | R | K | — |
| rows above | — | — | R | R | H or R | — |

Additional information about cleavage sites can be found on the world wide web at web.expasy.org/peptide_cutter/peptidecutter_enzymes.html, which is herein incorporated by reference in its entirety.

In some embodiments the cleavage site comprises, consists essentially of, or consists of a cleavage site of Table 4. As such, in some embodiments, the cleavage site comprises, consists essentially of, or consists of a cleavage site for Arg-C proteinase, Asp-N endopeptidase, BNPS-Skatole, Caspase 1, Caspase 2, Caspase 3, Caspase 4, Caspase 5, Caspase 6, Caspase 7, Caspase 8, Caspase 9, Caspase 10, Chymotrypsin-high specificity, Clostripain (Clostridiopeptidase B), CNBr, Enterokinase, Factor Xa, Formic acid, Glutamyl endopeptidase, GranzymeB, Hydroxylamine, lodosobenzoic acid, LysC, Neutrophil elastase, NTCB (2-nitro-5-thiocyanobenzoic acid), Pepsin (pH1.3), Pepsin (pH>2), Proline-endopeptidase, Proteinase K, Staphylococcal peptidase I, Thermolysin, Thrombin, or Trypsin. As such, in the methods of some embodiments herein, one or more of the listed enzymes is used to cleave the cleavage sites.

In some embodiments the cleavage site comprises, consists essentially of, or consists of a chemical- or pH-sensitive linker. Such linkers can undergo cleavage in the presence of a suitable chemical (in the case of chemical-sensitive linkers), or at a suitable pH (in the case of pH linkers). In some embodiments, the pH-sensitive linker undergoes enzyme-mediated cleavage when the pH is in a suitable range. For example, the pH-sensitive linkers such as the valine-citrulline (vc) dipeptide is cleaved in a pH-sensitive manner by cathepsin B at acidic pH's (e.g., in the 4.8-6 range) (See, e.g., Jain et al. Pharmaceutical Research, 32: 3526-3540, (2015)), which is hereby incorporated by reference in its entirety). In some embodiments, the pH-sensitive linker undergoes a cleavage event when the pH is in a particular range. For example, tunable pH sensitive linkers based on a phosphoraamidate backbone have also been described, which can undergo hydrolysis at a pH of less than 7.4 are described in PCT Pub. No. WO 2016028700, which is hereby incorporated by reference it its entirety). In some embodiments, the chemical-sensitive linker undergoes a cleavage event in the present of a chemical. For example, linkers comprising, consisting essentially or, consisting of disulfide bridges can be released upon the introduction of glutathione (See, e.g., Jain et al. Pharmaceutical Research, 32:3526-3540, (2015)).

### Signal Molecules

In some embodiments, pro-polypeptides comprise one or more signal molecules. As used herein, a “signal molecule” has its customary and ordinary meaning as understood by one of skill in the art in view of this disclosure. It refers to a secreted molecule that is capable of modulating, inducing, or inhibiting an activity or process in the cell that produced it, or in a different cell (a subject cell can be a microbial cell or a non-microbial cell, for example a cell of a multicellular organism such as an animal or plant). Example signal molecules include, but are not limited to, signaling peptides, quorum sensing molecules (for example, quorum sensing peptides), signal transduction receptor ligands, growth factors, hormones, and cytokines. In some embodiments, a signal molecule comprises, consists essentially of, or consists of a signaling peptides, quorum sensing molecules (for example, quorum sensing peptides), signal transduction receptor ligands, growth factors, hormones, or cytokine. In some embodiments, a signal molecule comprises, consists essentially of, or consists of a combination of two or more of signaling peptides, quorum sensing molecules (for example, quorum sensing peptides), signal transduction receptor ligands, growth factors, hormones, and cytokines, which can include combinations of two or more of the same type of molecule (for example a combination of two signaling peptides or a combination of two receptor ligands), as well as combinations of two different kinds of molecules (e.g., a combination of a cytokine and a hormone). In some embodiments, the signal molecule is for microbe-host dialog, and as such, the signal molecule is selected to target one or more cells of a host organism, for example a plant or animal. In some embodiments, the signal molecule stimulates, inhibits, increases, or decreases the production of bacteriocins and/or the growth rate of a subpopulation of a flora. In some embodiments, the signal molecule comprises, consists of, or consists essentially of a quorum sensing peptide, or a variant thereof as described herein.

Example quorum sensing peptides suitable for pro-polypeptides, methods, and/or encoded by nucleic acids of some embodiments include, but are not limited to, quorum sensing peptides such as the peptides shown in Table 5 below, including variants of these peptides, and combinations of two or more of any of these peptides. Without being limited by theory, it is contemplated that microbial cells, such as gram-positive bacteria use quorum sensing peptides to orchestrate cell-to-cell communication. A review of quorum sensing peptides can be found in Rajput et al., PLOS One DOI: 10.1371/journal.pone.0120066 Mar. 17, 2015, pp. 1-16, which is hereby incorporated by reference in its entirety. The quorum sensing peptides can induce activation of downstream response regulators and/or transcription factors in a target microbial cell. It is noted that in some embodiments, the downstream response regulators or transcription factors can be configured to activate or repress transcription of a target nucleic acid of interest. Accordingly, in some embodiments, a target microbial cell is genetically engineered to express or repress transcription (and subsequent expression) of a gene product of interest upon stimulation by a signal peptide of a pro-polypeptide.

In some embodiments, the signal molecule of the pro-polypeptide comprises, consists essentially of, or consists of a signal molecule (e.g., a quorum sensing molecule such as a quorum sensing peptide), which stimulates transcription of one or more bacteriocin polynucleotides in the target microbial cell. The target microbial cell can be genetically engineered to place one or more bacteriocin polynucleotides under the control of a transcription factor that responds to the quorum sensing molecule. In some embodiments, transcription of one or more bacteriocin polynucleotides is configured to be induced by a transcription factor (e.g., a transcriptional activator) that responds to the quorum sensing molecule. As such, signaling by the signal molecule (e.g. quorum sensing molecule) of the pro-polypeptide can stimulate the production of one or more additional bacteriocins by the target microbial cell.

In some embodiments, the target microbial cell is genetically modified to place one or more molecules of a poison-antidote system under the control of a transcription factor or response regulator that responds to a signal molecule (e.g., a quorum sensing molecule such as a quorum sensing peptide) so as to induce a suicidal response in target cell upon signaling by the signal molecule. Poison-antidote systems, which are distinct from bacteriocins, can be useful for accomplishing such a suicidal response, which in turn can be useful for containment and/or selective growth of microbial cells. Exemplary poison antidote systems are described in U.S. Pat. Nos. 5,910,438, 6,180,407, 7,176,029, and 7,183,097, each of which is hereby incorporated by reference in its entirety. In some embodiments, a poison-antidote system comprises a cytotoxic (poison) polypeptide, and a corresponding antitoxin (antidote) polypeptide in a single cell. As used herein, a “poison polynucleotide” has its customary and ordinary meaning as understood by one of skill in the art in view of this disclosure, and can refer to a polynucleotide encoding a poison polypeptide. An “antidote polynucleotide” has its customary and ordinary meaning as understood by one of skill in the art in view of this disclosure, and can refer to a polynucleotide encoding an antidote polypeptide. As such, in some embodiments, the target microbial cell is configured to induce transcription of a poison polynucleotide in response to a signal molecule such as a quorum sensing molecule, so as to induce a suicidal response in the target microbial cell. In some embodiments, the target microbial cell is configured to transcriptionally repress an antibody polynucleotide (while continuing to express a corresponding poison polypeptide) in response to a signal molecule such as a quorum sensing molecule, so as to induce a suicidal response in the target microbial cell.

In some embodiments, the quorum sensing peptides are naturally-occurring. In some embodiments, the quorum sensing peptide comprises, consists essentially of, or consists of a variant of a naturally-occurring quorum sensing peptide. In some embodiments, the quorum sensing peptide comprises, consists essentially of, or consists of a synthetic peptide. Information on quorum sensing peptides, including example sequences, can be found on the quorumpeps database, accessible on the world wide web at quorumpeps.ugent.be., which is hereby incorporated by reference in its entirety.

TABLE 5

| | | SEQ ID NO: |
|---|---|---|
| | | (if peptide |
| Name | Species | shown) |
| phrANTH3 | Bacillus anthracis | — |
| phrANTH1 | Bacillus anthracis | — |
| phrANTH2 | Bacillus anthracis | — |
| PapR5I | Bacillus cereus | — |
| PapR5IV | Bacillus cereus | — |
| PapR5II | Bacillus cereus | — |
| PapR7III | Bacillus cereus | — |
| PapR7IV | Bacillus cereus | — |
| PapR7II | Bacillus cereus | — |
| PapR5III | Bacillus cereus | — |
| PapR7I | Bacillus cereus, Bacillus thuringiensis | — |
| Phr0662 | Bacillus halodurans | — |
| phr1988 | Bacillus halodurans | — |
| ComX RO-B-2 | Bacillus mojavensis | — |
| ComX RO-H-1 | Bacillus mojavensis | — |
| ComX RO-C-2 | Bacillus mojavensis | — |
| Phr-pPL10-1, PhrPUM | Bacillus pumilus | — |
| PHRBST1 | Bacillus stearothermophilus | — |
| PhrA | Bacillus subtilis | — |
| ComX 168 | Bacillus subtilis | — |
| Mersacidin | Bacillus subtilis | — |
| PhrI | Bacillus subtilis | — |
| PhrG | Bacillus subtilis | — |
| PhrK | Bacillus subtilis | — |
| ComX RO-E-2 | Bacillus subtilis | — |
| Subtilin | Bacillus subtilis | — |
| ComX RS-B-1 | Bacillus subtilis | — |
| Phr-pLS20 | Bacillus subtilis | — |
| PhrF | Bacillus subtilis | — |
| Phr-pTA1040 | Bacillus subtilis | — |
| Phr-pPOD2000, Phr-pTA1060 | Bacillus subtilis | — |
| PhrE | Bacillus subtilis | — |
| BsEDF | Bacillus subtilis | — |
| ComX natto | Bacillus subtilis | — |
| Entianin | Bacillus subtilis | — |
| Competence and Sporulation | Bacillus subtilis, Bacillus mojavensis | — |
| Factor, CSF, PhrC | | |
| NprRB | Bacillus thuringiensis | — |
| PisN | Carnobacterium maltaromaticum | — |
| CbaX | Carnobacterium maltaromaticum | — |
| Carnobacteriocin B2, CB2, | Carnobacterium piscicola | — |
| CbnB2 | | |
| CbnS, CS | Carnobacterium piscicola, | — |
| | Carnobacterium maltaromaticum | |
| AIP, Autoinducing peptide | Clostridium acetobutylicum | — |
| PHRCACET4 | Clostridium acetobutylicum | — |
| PHRCACET1 | Clostridium acetobutylicum | — |
| AgrD2, AIP, Autoinducing | Clostridium botulinum | — |
| peptide | | |
| AgrD2, AIP, Autoinducing | Clostridium botulinum | — |
| peptide | | |
| AgrD2, AIP, Autoinducing | Clostridium botulinum | — |
| peptide | | |
| AgrD2, AIP, Autoinducing | Clostridium botulinum | — |
| peptide | | |
| AgrD1, AIP, Autoinducing | Clostridium botulinum | — |
| peptide | | |
| AgrDCp | Clostridium perfringens | — |
| WS9326A | Clostridium perfringens | — |
| WS9326B | Clostridium perfringens | — |
| Cochinmicin II/III | Clostridium perfringens | — |
| AgrD2, AIP, Autoinducing | Clostridium sporogenes | — |
| peptide | | |
| AgrD1, AIP, Autoinducing | Clostridium sporogenes | — |
| peptide | | |
| AgrD | Clostridium thermocellum | — |
| QSP1 | Cryptococcus neoformans | — |
| QSP24 | Cryptococcus neoformans | — |
| QSP2 | Cryptococcus neoformans | — |
| — | Eikenella corodens | — |
| cAM373 | Enterococcus faecalis | — |
| iCF10 | Enterococcus faecalis | — |
| iPD1 | Enterococcus faecalis | — |
| cPD1 | Enterococcus faecalis | — |
| GBAP, Gelatinase | Enterococcus faecalis | — |
| Biosynthesis-Activating | | |
| Pheromone | | |
| cAD1 | Enterococcus faecalis | — |
| iAD1 | Enterococcus faecalis | — |
| cCF10 | Enterococcus faecalis | — |
| iAM373 | Enterococcus faecalis | — |
| cOB1 | Enterococcus faecalis | — |
| EntF | Enterococcus faecium | — |
| EDF, Extracellular death | Escherichia coli | — |
| factor | | |
| LamD | Lactobacillus plantarum | — |
| PltA | Lactobacillus plantarum | — |
| Plantaricin A, PlnA | Lactobacillus plantarum | — |
| IP-TX | Lactobacillus sakei | — |
| IP-673 | Lactobacillus sakei | — |
| Orf4 | Lactobacillus sakei | — |
| Nisin A | Lactococcus lactis | — |
| AIP | Listeria monocytogenes | — |
| PaEDF-1 | Pseudomonas aeruginosa | — |
| PaEDF-2 | Pseudomonas aeruginosa | — |
| PaEDF-3 | Pseudomonas aeruginosa | — |
| AIP, Autoinducing peptide | Staphylococcus arlettae | — |
| AIP2, Autoinducing peptide 2 | Staphylococcus aureus | — |
| AIP3, Autoinducing peptide 3 | Staphylococcus aureus | — |
| AIP1, Autoinducing peptide 1 | Staphylococcus aureus | — |
| AIP4, Autoinducing peptide 4 | Staphylococcus aureus | — |
| AIP, Autoinducing peptide | Staphylococcus auricularis | — |
| AIP, Autoinducing peptide | Staphylococcus auricularis | — |
| AIP, Autoinducing peptide | Staphylococcus capitis | — |
| AIP, Autoinducing peptide | Staphylococcus capitis | — |
| AIP, Autoinducing peptide | Staphylococcus caprae | — |
| AIP, Autoinducing peptide | Staphylococcus caprae | — |
| AIP, Autoinducing peptide | Staphylococcus caprae | — |
| AIP, Autoinducing peptide | Staphylococcus carnosus | — |
| AIP, Autoinducing peptide | Staphylococcus cochnii subsp. cochnii | — |
| AIP, Autoinducing peptide | Staphylococcus cochnii subsp. | — |
| | urealyticum | |
| AIP2, Autoinducing peptide 2 | Staphylococcus epidermidis | — |
| AIP, Autoinducing peptide | Staphylococcus epidermidis | — |
| AIP, Autoinducing peptide | Staphylococcus epidermidis | — |
| AIP, Autoinducing peptide | Staphylococcus epidermidis | — |
| Pep5 | Staphylococcus epidermidis | — |
| AIP4, Autoinducing peptide 4 | Staphylococcus epidermidis | — |
| AIP1, Autoinducing peptide 1 | Staphylococcus epidermidis | — |
| AIP3, Autoinducing peptide 3 | Staphylococcus epidermidis | — |
| AIP-II | Staphylococcus epidermidis | — |
| AIP-III | Staphylococcus epidermidis | — |
| AIP, Autoinducing peptide | Staphylococcus gallinarum | — |
| AIP, Autoinducing peptide | Staphylococcus lugdunensis | — |
| AIP, Autoinducing peptide | Staphylococcus lugdunensis | — |
| AIP, Autoinducing peptide | Staphylococcus simulans | — |
| AIP, Autoinducing peptide | Staphylococcus simulans | — |
| AIP, Autoinducing peptide | Staphylococcus simulans | — |
| AIP, Autoinducing peptide | Staphylococcus warneri | — |
| AIP, Autoinducing peptide | Staphylococcus xylosus | — |
| Short Hydrophobic Peptide 3, | Streptococcus agalactiae | — |
| SHP3 | | |
| Competence Stimulating | Streptococcus crista | — |
| Peptide, CSP | | |
| SilCR | Streptococcus dysgalactiae | — |
| Competence Stimulating | Streptococcus gordonii | — |
| Peptide, CSP | | |
| Competence Stimulating | Streptococcus gordonii | — |
| Peptide, CSP | | |
| Competence Stimulating | Streptococcus milleri | — |
| Peptide, CSP | | |
| Competence Stimulating | Streptococcus mitis | — |
| Peptide, CSP | | |
| Competence Stimulating | Streptococcus mitis | — |
| Peptide, CSP | | |
| Competence Stimulating | Streptococcus mitis | — |
| Peptide, CSP | | |
| Competence Stimulating | Streptococcus mitis | — |
| Peptide, CSP | | |
| Competence Stimulating | Streptococcus mitis | — |
| Peptide, CSP | | |
| Competence Stimulating | Streptococcus mitis | — |
| Peptide, CSP | | |
| Competence Stimulating | Streptococcus mitis, Streptococcus | — |
| Peptide, CSP | pneumoniae, Streptococcus oralis | |
| 18-CSP, Competence | Streptococcus mutans | — |
| Stimulating Peptide | | |
| 21-CSP, Competence | Streptococcus mutans | — |
| Stimulating Peptide | | |
| Short Hydrophobic Peptide, | Streptococcus mutans | — |
| SHP1509 | | |
| ComS | Streptococcus mutans | — |
| Competence Stimulating | Streptococcus oralis | — |
| Peptide, CSP | | |
| Competence Stimulating | Streptococcus oralis | — |
| Peptide, CSP | | |
| Competence Stimulating | Streptococcus pneumoniae | — |
| Peptide, CSP | | |
| Competence Stimulating | Streptococcus pneumoniae | — |
| Peptide, CSP | | |
| Bacteriocin Inducing Peptide, | Streptococcus pneumoniae | — |
| BIP | | |
| Bacteriocin Inducing Peptide, | Streptococcus pneumoniae | — |
| BIP, BIP-2, BIpC | | |
| Bacteriocin Inducing Peptide, | Streptococcus pneumoniae | — |
| BIP-1, BIpC | | |
| Streptin 1 | Streptococcus pyogenes | — |
| XIP | Streptococcus pyogenes | — |
| XIP | Streptococcus pyogenes | — |
| SHP2-C10 | Streptococcus pyogenes | — |
| SHP2-C9 | Streptococcus pyogenes | — |
| SHP2-C7 | Streptococcus pyogenes | — |
| SHP3-C10 | Streptococcus pyogenes | — |
| SHP3-C9 | Streptococcus pyogenes | — |
| SHP3-C7 | Streptococcus pyogenes | — |
| Competence Stimulating | Streptococcus pyogenes, | — |
| Peptide, CSP | Streptococcus pneumoniae | |
| Competence Stimulating | Streptococcus sanguis | — |
| Peptide, CSP | | |
| STP | Streptococcus thermophilus | — |
| SHP1358(15-23) | Streptococcus thermophilus | — |
| ComS, SHP0316(18-24) | Streptococcus thermophilus | — |
| Short Hydrophobic | Streptococcus thermophilus | — |
| Peptide; SHP1299 | | |
| Competence Stimulating | Streptococcus thermophilus, | — |
| Peptide, CSP | Streptococcus constellatus, | |
| | Streptococcus anginosus | |
| Siamycin I | Streptomyces species | — |
| TM0504 | Thermotoga maritima | — |
| Synthetic RAP-binding | Synthetic (FHWWQTSPAHFS) | 741 |
| peptide, RBP | | |
| Synthetic RAP-binding | Synthetic (WPFAHWPWQYPR) | 742 |
| peptide, RBP | | |
| Synthetic AgrC ligand | Synthetic (GDSVCASYF, thiolacton | 743 |
| | linkage between C5 and F9) | |
| Synthetic AgrC ligand | Synthetic (SVCASYF, thiolacton | 744 |
| | linkage between C3 and F7) | |
| Synthetic Cry1Aa ligand | Synthetic (SKADT) | 745 |
| Synthetic Cry1Aa ligand | Synthetic (SKPAD) | 746 |
| Synthetic Fsr ligand | Synthetic (benzyloxycarbonyl- | 747 |
| | QNSAAAFAAWA, lacton linkage | |
| | between S3 and A11) | |
| Synthetic Fsr ligand | Synthetic (benzyloxycarbonyl- | 748 |
| | QNSAAAFGQWA, lacton linkage | |
| | between S3 and A11) | |
| Synthetic AgrC1, AgrC2 | Synthetic (YSTC(alpha-aminobutyric | 749 |
| | acid)FIM, thiolacton linkage between | |
| | C4 and M7) | |
| Synthetic AgrC1, AgrC2 | Synthetic (N-4-(4- | 750 |
| | benzoylphenoxy)butyryl-STCAFIM, | |
| | thiolacton linkage between C3 and | |
| | M7) | |

Cytokines are a class of signal molecules that are typically produced by cells, such as cells of the immune system, and capable of inducing a response in other cells. A number of different cytokines can be used in pro-polypeptides, methods, and/or encoded by nucleic acids of some embodiments herein. It is contemplated that a pro-polypeptide comprising a bacteriocin and a cytokine in accordance with some embodiments can be useful to induce antimicrobial activity (by the bacteriocin(s)), and a host response, for example immune cell suppression or immune cell stimulation (by the cytokine(s)). In some embodiments, the cytokine comprises, consists essentially of, or consists of a naturally-occurring cytokine, variant of a naturally occurring, or synthetic cytokine. A number of suitable cytokines can be used in pro-polypeptides, methods, compositions, cells, or encoded by nucleic acids in accordance with some embodiments herein, including, but not limited to IL-1, IL-2, IL-3, IL-4, IL-5, IL-6, IL-7, IL-8, IL-9, IL-10, IL-11, IL-12, IL-13, IL-15, IL-16, IL-17, IL-18, IL-19, IL-20, IL-21, IL-22, IL-23, IFN-α, IFN-β, IFN-γ, TNF-α, TNF-β, TGF-β1, M-CSF, G-CSF, and GM-CSF, variants of any of these, or any combination of two or more of these.

Hormones are a class of signal molecules that are typically produced by cells of multicellular organisms, and signal to other cells, frequently circulating through different tissues and/or organs of a multicellular organism. A number of different hormones can be used in pro-polypeptides, methods, and/or be encoded by nucleic acids of some embodiments herein. It is contemplated that a pro-polypeptide comprising a bacteriocin and a hormone in accordance with some embodiments can be useful to induce antimicrobial activity (by the bacteriocin(s)), along with a host response, for example cell growth or proliferation (by the hormone(s)). In some embodiments, the cytokine comprises, consists essentially of, or consists of a naturally-occurring hormone, variant of a naturally occurring hormone, or synthetic hormone. Example hormones suitable for pro-polypeptides, methods, and/or encoded by nucleic acids of some embodiments include, but are not limited to, protein and peptide hormones, for example activin and inhibin, adiponectin, adipose-derived hormones, adrenocorticotropic hormone, agouti gene, agouti signaling peptide, allatostatin, amylin, amylin family, angiotensin, ANGPTL8, asprosin, atrial natriuretic peptide, big gastrin, bovine somatotropin, bradykinin, brain-derived neurotrophic factor, calcitonin, ciliary neurotrophic factor, corticotropin-releasing hormone, crustacean neurohormone family, endothelin, enteroglucagon, erythroferrone, fellutamide, FGF15, FGF15/19, FGF19, FNDC5, follicle-stimulating hormone, gastrin, gastroinhibitory peptide, ghrelin, glucagon-like peptide-1, gonadotropin, gonadotropin release inhibitor, gonadotropin-releasing hormone, granulocyte colony-stimulating factor, growth hormone, growth hormone-releasing hormone, hepcidin, human chorionic gonadotropin, human placental lactogen, incretin, insulin, insulin analog, insulin aspart, insulin degludec, insulin glargine, insulin lispro, insulin-like growth factor, insulin-like growth factor 1, insulin-like growth factor 2, leptin, limostatin, liraglutide, little gastrin I, luteinizing hormone, melanocortin, melanocyte-stimulating hormone, alpha-melanocyte-stimulating hormone, beta-melanocyte-stimulating hormone, gamma-melanocyte-stimulating hormone, minigastrin, N-terminal prohormone of brain natriuretic peptide, nerve growth factor, neuropeptide VF precursor, neurotrophin-3, neurotrophin-4, NPH insulin, obestatin, osteocalcin, parathyroid hormone, peptide hormone, peptide YY, plasma renin activity, pramlintide, preprohormone, prolactin, relaxin, relaxin family peptide hormones, renin, salcatonin, sauvagine, secretin, secretin family, sincalide, stanniocalcin, teleost leptins, temporin, thyroid-stimulating hormone, thyrotropin-releasing hormone, urocortin, urocortin II, urocortin III, vasoactive intestinal peptide, vitellogenin, variants of any of these, or any combination of two or more of these.

### Affinity Tags

In pro-polypeptides, methods, and nucleic acids encoding pro-polypeptides of some embodiments herein, a pro-polypeptide, or component peptide thereof (e.g. a bacteriocin or signal molecule) comprises an affinity tag. Optionally, one or more cleavage sites can be positioned between the affinity tag and the rest of the pro-polypeptide to facilitate removal of the affinity tag after affinity purification. Affinity tags can be used in purification, for example by contact with a molecule that binds the affinity tag immobilized on a solid phase, such as a bead. Example affinity tags suitable for pro-polypeptides, methods, and encoding by nucleic acids of some embodiments herein can comprise, consist essentially of, or consist of His-tags, glutathione-S-transferase (GST) tags, FLAG tags, strep tags, maltose binding protein (MBP), chitin binding protein (CBP), myc tags, HA tags, NE tags, and V5 tags, variants of any of these, or any combination of two or more of these.

### Microbial Organisms

In some embodiments, genetically engineered microorganisms are provided. As used herein, genetically engineered “microbial organism,” “microorganism,” and variations of these root terms (such as pluralizations and the like), have their customary and ordinary meanings as understood by one of skill in the art in view of this disclosure. They encompass genetic modification of any naturally-occurring species or fully synthetic prokaryotic or eukaryotic unicellular organism, as well as Archae species. Thus, this expression can refer to cells of bacterial species, fungal species, and algae.

Exemplary microorganisms that can be used in accordance with embodiments herein include, but are not limited to, bacteria, yeast, and algae, for example photosynthetic microalgae. Furthermore, fully synthetic microorganism genomes can be synthesized and transplanted into single microbial cells, to produce synthetic microorganisms capable of continuous self-replication (see Gibson et al. (2010), “Creation of a Bacterial Cell Controlled by a Chemically Synthesized Genome,” Science 329:52-56, hereby incorporated by reference in its entirety). As such, in some embodiments, the microorganism is fully synthetic. A desired combination of genetic elements, including elements that regulate gene expression, and elements encoding gene products (for example bacteriocins, immunity modulators, poison, antidote, and industrially useful molecules) can be assembled on a desired chassis into a partially or fully synthetic microorganism. Description of genetically engineered microbial organisms for industrial applications can also be found in Wright, et al. (2013) “Building-in biosafety for synthetic biology” Microbiology 159:1221-1235.

A variety of bacterial species and strains can be used in accordance with embodiments herein, and genetically modified variants, or synthetic bacteria based on a “chassis” of a known species can be provided. Exemplary bacteria with industrially applicable characteristics, which can be used in accordance with embodiments herein include, but are not limited to, Bacillus species (for example Bacillus coagulans, Bacillus subtilis, and Bacillus licheniformis), Paenibacillus species, Streptomyces species, Micrococcus species, Corynebacterium species, Acetobacter species, Cyanobacteria species, Salmonella species, Rhodococcus species, Pseudomonas species, Lactobacillus species, Enterococcus species, Alcaligenes species, Klebsiella species, Paenibacillus species, Arthrobacter species, Corynebacterium species, Brevibacterium species, Thermus aquaticus, Pseudomonas stutzeri, Clostridium thermocellus, and Escherichia coli.

A variety of yeast species and strains can be used in accordance with embodiments herein, and genetically modified variants, or synthetic yeast based on a “chassis” of a known species can be provided. Exemplary yeast with industrially applicable characteristics, which can be used in accordance with embodiments herein include, but are not limited to Saccharomyces species (for example, Saccharomyces cerevisiae, Saccharomyces bayanus, Saccharomyces boulardii), Candida species (for example, Candida utilis, Candida krusei), Schizosaccharomyces species (for example Schizosaccharomyces pombe, Schizosaccharomyces japonicas), Pichia or Hansenula species (for example, Pichia pastoris or Hansenula polymorpha) species, and Brettanomyces species (for example, Brettanomyces claussenii).

A variety of algae species and strains can be used in accordance with embodiments herein, and genetically modified variants, or synthetic algae based on a “chassis” of a known species can be created. In some embodiments, the algae comprises photosynthetic microalgae. Exemplary algae species that can be useful for biofuels, and can be used in accordance with embodiments herein, include Botryococcus braunii, Chlorella species, Dunaliella tertiolecta, Gracilaria species, Pleurochrysis carterae, and Sargassum species. Additionally, many algaes can be useful for food products, fertilizer products, waste neutralization, environmental remediation, and carbohydrate manufacturing (for example, biofuels).

### Design of Pro-Polypeptides and Nucleic Acids Encoding Pro-Polypeptides

Pro-polypeptides comprising selected bacteriocins in desired ratios in accordance with some embodiments herein can be designed using a variety of approaches. For example, one option is to design a nucleic acid coding sequence comprising the bacteriocins, cleavage sites, and other features of the pro-polypeptide such as affinity tags and/or signal molecules in a single open reading frame. A polynucleotide having this sequence can then be sequenced. Additionally, computational tools are available to facilitate the design of pro-polypeptides (and nucleic acids encoding them) in accordance with some embodiments. For example, as described in Nielsen et al., “Genetic circuit design automation” Science (2016) 352: aac7341 (hereby incorporated by reference in its entirety), a computer-based design environment, Cello, permits a user to writes Verilog code that is automatically transformed into a DNA sequence. Accordingly, in some embodiments, text-based approach can readily be used to produce a nucleic acid encoding the desired elements of a pro-polypeptide, such as a particular combination and/or ratio of bacteriocins and cleavage sites separating the bacteriocins, and in a manner where bacteriocins are not cleaved by a cleavage enzyme that cleaves the cleavage sites. In some embodiments, the pro-polypeptide comprises selected bacteriocins and antimicrobial peptides in desired ratios. In some embodiments, the pro-polypeptide comprises selected antimicrobial peptides in desired ratios. In some embodiments, the pro-polypeptide comprises selected bacteriocins in desired ratios.

### Genetic Modification of Microbial Organisms for Expression of Nucleic Acids

Techniques of genetically modifying microorganisms are well known in the art. In methods and compositions of some embodiments, a microorganism is genetically modified to comprise nucleic acid sequence regulating the expression of, and encoding, bacteriocins, for example pro-polypeptides comprising bacteriocins as described herein. In methods and compositions of some embodiments herein, polynucleotides encoding pro-polypeptides can be delivered to microorganisms, and can be stably integrated into the chromosomes of these microorganisms, or can exist free of the genome, for example in a plasmid, extrachromosomal array, episome, minichromosome, or the like.

Exemplary vectors for genetic modification of microbial cells include, but are not limited to, plasmids, viruses (including bacteriophage), and transposable elements. Additionally, it will be appreciated that entire microbial genomes comprising desired sequences can be synthesized and assembled in a cell (see, e.g. Gibson et al. (2010), Science 329:52-56). As such, in some embodiments, a microbial genome (or portion thereof) is synthesized with desired features such as bacteriocin polynucleotide(s), and introduced into a microbial cell.

In some embodiments, a cassette for inserting one or more desired bacteriocin and/or immunity modulator polynucleotides into a polynucleotide sequence (for example inserting, into an expression vector, a cassette encoding a pro-polypeptide comprising bacteriocins) is provided. Exemplary cassettes include, but are not limited to, a Cre/lox cassette or FLP/FRT cassette. In some embodiments, the cassette is positioned on a plasmid, so that a plasmid with the desired polynucleotide encoding the desired pro-polypeptide can be readily introduced to the microbial cell. In some embodiments, the cassette is positioned in a desired position in the genome of the microbial cell.

In some embodiments, plasmid conjugation can be used to introduce a desired plasmid from a “donor” microbial cell to a recipient microbial cell. Goñi-Moreno, et al. (2013) Multicellular Computing Using Conjugation for Wiring. PLOS ONE 8 (6): e65986, hereby incorporated by reference in its entirety. In some embodiments, plasmid conjugation can genetically modify a recipient microbial cell by introducing a conjugation plasmid from a donor microbial cell to a recipient microbial cell. Without being limited by any particular theory, conjugation plasmids that comprise the same or functionally same set of replication genes typically cannot coexist in the same microbial cell. As such, in some embodiments, plasmid conjugation “reprograms” a recipient microbial cell by introducing a new conjugation plasmid to supplant another conjugation plasmid that was present in the recipient cell. In some embodiments, plasmid conjugation is used to engineer (or reengineer) a microbial cell with a particular nucleic acid encoding a pro-polypeptide, or combination of different nucleic acids encoding different pro-polypeptide. According to some embodiments, a variety of conjugation plasmids comprising different nucleic acids comprising a variety of different pro-polypeptides is provided. The plasmids can comprise additional genetic elements as described herein, for example promoters, translational initiation sites, and the like. In some embodiments the variety of conjugation plasmids is provided in a collection of donor cells, so that a donor cell comprising the desired plasmid can be selected for plasmid conjugation. In some embodiments, a particular combination and/or ratio of bacteriocins is selected, and an appropriate donor cell (encoding the particular pro-polypeptide) is conjugated with a microbial cell of interest to introduce a conjugation plasmid comprising that combination into a recipient cell.

### Pro-Polypeptides

As used herein, “pro-polypeptide” has its customary and ordinary meaning as understood by one of skill in the art in view of this disclosure. It refers to a precursor polypeptide comprising, consisting essentially of, or consisting of at least two component peptides in a desired ratio (e.g., at least one bacteriocin and at least one signal molecule, or two or more bacteriocins, and optionally one or more other peptides disclosed herein (for example, a signaling peptide, a quorum sensing molecule such as a quorum sensing peptide, a signal transduction receptor ligand, a growth factor, a hormones, a cytokine, or a combination of two or more of any of the listed items), and at least one cleavage site separating the component peptides of the pro-polypeptide. An example pro-polypeptide in accordance with some embodiments is schematically illustrated in FIG. 2. The component peptides can be separated from each other by cleavage sites so that, upon cleavage, the component peptides are separated from each other and present in ratios that are determined by the number of copies of each component peptide in the pro-polypeptide (See FIG. 3). In some embodiments, one or more component peptides of the pro-polypeptide are in an inactive form, and become active upon cleavage from surrounding polypeptide sequences. Without being limited by theory, it is contemplated that in some embodiments, at least some, or in some embodiments all, of the bacteriocins are inactive when they are part of the pro-polypeptide so that, advantageously, the pro-polypeptide can be produced and/or modified by microbial cells that do not require immunity to those bacteriocins that are inactive. It is expressly contemplated that the pro-polylpeptides described herein can be used in conjunction with any of the methods and kits as described herein. Furthermore, it will be appreciated that for any pro-polypeptide described herein, the polynucleotide or polynucleotides (to the extent that there are multiple possible codons) will be readily appreciated based on the skilled artisan's understanding of the genetic code.

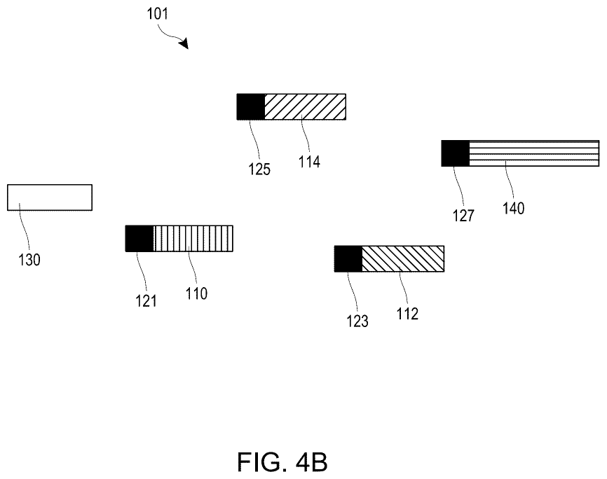

FIG. 4A is a schematic diagram illustrating a pro-polypeptide 100 in accordance with some embodiments herein. The pro-polypeptide can comprise bacteriocins 110, 112, 114. The bacteriocins can be the same or different (e.g. all of the bacteriocins can be the same, or some bacteriocins can be the same while others are different, or all of the bacteriocins can be different from each other). The pro-polypeptide can further include one or more cleavage sites 120, 122, 124, and 126 disposed between the bacteriocins, and/or other polypeptides o the pro-polypeptide. In some embodiments, the pro-polypeptide further comprise a tag 130. In some embodiments, the pro-polypeptide further comprises a signal molecule 140 such as described herein, for example a quorum sensing molecule such as a quorum sensing peptide. In some embodiments, cleavage sites 120, 122, 124, and 126 are disposed between the bacteriocins, tag, and signal molecule. In some embodiments, the tag is useful for purifying the pro-polypeptide. In some embodiments, a cleavage site (e.g., 120, 122, 124, and 126) includes two or more consensus sites for an enzyme such as an endopeptidase. It is contemplated that in some embodiments, a cleavage site contains two or more cleavage consensus sequences (which can be the same or different), at least one of which is immediately downstream of the C-terminal of an upstream peptide and/or at least one of is immediately upstream of the N-terminal of a downstream polypeptide, so that the any vestiges can of the cleavage site can essentially eliminated or entirely eliminated of that peptide. In some embodiments, a “cleavage site” includes two or more consensus sequences, at least one of which is configured to be immediately upstream or downstream of a peptide (e.g., a bacteriocin) so as to remove any vestiges of that cleavage site from the peptide upon cleavage. In some embodiments, a “cleavage site” includes two consensus sequences, one of which is configured to be immediately downstream of an upstream peptide (e.g., a bacteriocin), and the other of which is immediately upstream of a downstream peptide (e.g. a bacteriocin) so as to remove any vestiges of that cleavage site from the C-terminal portion of the upstream peptide and from the N-terminal portion of the downstream peptide upon cleavage of both cleavage sites. Optionally, additional cleavage sites are positioned between these two cleavage sites.

FIG. 4B is a schematic diagram illustrating a composition comprising bacteriocins 101 made from pro-polypeptide 100 in accordance with some embodiments herein. The composition can comprise bacteriocins 110, 112, 114. The bacteriocins can be the same or different (e.g. all of the bacteriocins can be the same, or some bacteriocins can be the same while others are different, or all of the bacteriocins can be different from each other). Optionally, some of the bacteriocins 110, 112, 114, can further comprise vestiges of cleavage sites, 121, 123, 125, 127. In some embodiments, the composition further comprises a tag 130, and/or a signal molecule 140 as described herein. Some of the bacteriocins 110, 112, 114, and/or the tag 130, and/or the signal molecule 140 can further comprise vestiges of cleavage sites, 121, 123, 125, 127. In some embodiments, following cleavage, a bacteriocin comprises no more than 0, 1, 2, 3, 4, 5, 6, 7, 8, 9, or 10, amino acids as a vestige of a cleavage site, including ranges between any two of the listed values. In some embodiments, bacteriocin does not comprise any vestiges od cleavage sites. In some embodiments, a bacteriocin comprises a vestige of a cleavage site on its N-terminal, on its C-terminal, or on its N- and C-termini. It is contemplated that in some embodiments, a signal molecule and/or tag also comprises vestiges as described herein. In some embodiments, a signal molecule and/or tag does not comprise any vestiges as described herein.

In some embodiments, a pro-polypeptide comprises, consists essentially of, or consists of at least one bacteriocin, at least one signal molecule, and one or more cleavage sites disposed between the bacteriocins and signal molecules, so that each bacteriocin or signal molecule can be separated from the rest of the pro-polypeptide by cleavage of the cleavage sites. In some embodiments, the bacteriocin(s) and signal molecule(s) are in a desired ratio.

In some embodiments, a pro-polypeptide comprises first component peptide comprising, consisting essentially of, or consisting of a bacteriocin, at least one additional component peptide (bacteriocin or signal molecule), and cleavage sites disposed between the component peptides. The pro-polypeptide can further comprise an affinity tag. In some embodiments, the affinity tag is separated from the bacteriocins (and/or other component peptides) of the pro-polypeptide by a cleavage site, so that after affinity purification of the pro-polypeptide, the affinity tag can be cleaved so as to remove it from the pro-polypeptides. In some embodiments, the pro-polypeptide comprises, consists essentially of, or consists of at least two bacteriocins, and optionally at least one signal molecule. In some embodiments, the isolated pro-polypeptide comprises three or more bacteriocins, for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bacteriocins, including ranges between any two of the listed values, for example, 3-10, 3-20, 3-30, 3-50, 5-10, 5-20, 5-30, 5-50, 7-10, 7-20, 7-30, 7-50, 10-20, 10-30, or 10-50 bacteriocins. In some embodiments, the pro-polypeptide comprises at least five bacteriocins.

It is further contemplated that the pro-polypeptide can include a signal molecule, which can facilitate communication to microbial cells, or to a host organism. Accordingly, in pro-polypeptides, methods, and nucleic acids of some embodiments, the pro-polypeptide further comprises a signal molecule. The signal molecule can be separated from other component peptides of the pro-polypeptide by a cleavage site. In some embodiments, the signal molecule comprises, consists essentially of, or consists of a quorum sensing molecule (e.g., a quorum sensing peptide), a cytokine, or a hormone as described herein. In some embodiments, a target cell is configured to produce additional gene products, for example bacteriocins, in response to the quorum sensing molecule. The target cell can be genetically engineered so that one or more bacteriocin polynucleotides are under the control of a transcription factor that responds to the quorum sensing molecule, so that, upon signaling by the quorum sensing molecule, the bacteriocin polynucleotides are transcribed, and the target cell produces additional bacteriocins.

In some embodiments, one or more cleavage sites are disposed in frame between any two adjacent component peptides (e.g., bacteriocins, signal molecules) of the pro-polypeptide. In some embodiments, one or more cleavage sites are disposed in frame between any two adjacent bacteriocins of the pro-polypeptide. It is noted that in pro-polypeptides, methods, and nucleic acids of some embodiments, component peptides (e.g., bacteriocins and/or signal molecules) or affinity tags positioned at or near the N- or C-terminus of a pro-polypeptide may be positioned near a cleavage site to separate them from the rest of the pro-polypeptide, but they are not flanked by cleavage sites on both sides. For example, for a bacteriocin on the N-terminus of a bacteriocin, there may be a cleavage site in the C-terminal direction from the bacteriocin, but no cleavage site on the N-terminal side of the bacteriocin. In some embodiments, for example, if a component peptide or affinity tag is near the N- or C-terminus of the pro-polypeptide, but there is additional N- or C-terminal sequence beyond the component peptide or affinity tag, the component peptide may comprise cleavage sites on both sides so as to facilitate removal of the additional N- or C-terminal sequence. In some embodiments, some additional N- or C-terminal sequences can remain on the component polypeptide.

It is noted that in some embodiments, the cleavage sites can comprise sequences targeted by a cleavage enzyme. In order to minimize or avoid cleavage of the bacteriocins or other component peptides (e.g., signal molecules) of the pro-polypeptide, it is contemplated that in some embodiments, the component peptides themselves do not contain cleavage sites for the cleavage enzyme(s) that target cleavage sites between the component peptides. As such, in some embodiments, the bacteriocins do not comprise cleavage sites for the cleavage enzyme or enzymes that target the cleavage sites between the bacteriocins. In some embodiments, two or more cleavage enzymes are used to target all of the cleavage sites between the component peptides of the pro-polypeptide, but the bacteriocins do not contain cleavage sites targeted by any of these cleavage enzymes. In some embodiments, at least one cleavage site is for a first cleavage enzyme, and another cleavage site is for a second cleavage enzyme different from the first cleavage enzyme, and the bacteriocins do not comprise a cleavage site for any of the first or second cleavage enzymes

In some embodiments, the signal molecule(s) do not comprise the cleavage sites for the cleavage enzyme or enzymes that target the cleavage sites between the bacteriocins and signal molecules. It is noted that as in some embodiments, cleavage is performed after affinity purifying the pro-polypeptide. Consequently, it can be acceptable for an affinity tag itself to comprise one or more cleavage sites, as the affinity tag may be dispensable by the time cleavage is performed.

In some embodiments, the cleavage sites separating the component peptides of the pro-polypeptide are all targeted by the same cleavage enzyme so that a single enzyme can be used to cleave the pro-polypeptide into separate bacteriocins (and optionally, other peptides) in the desired ratios. In some embodiments, the cleavage sites separating the component peptides of the pro-polypeptide are collectively targeted by a combination of cleavage enzyme so that the number of cleavage enzymes that is smaller than the number of cleavage sites in the pro-polypeptide. In some embodiments, the cleavage sites separating the component peptides of the pro-polypeptide are collectively targeted by one, two, three, four, five, six, seven, eight, nine, or ten cleavage enzymes. In some embodiments, the cleavage sites comprise a chemical or pH-sensitive linker. As such, in some of these embodiments, the cleavage sites separating the component peptides of the pro-polypeptide are collectively cleaved by the same chemical and/or same pH conditions.

In some embodiments, the bacteriocins themselves comprise tags, for example affinity tags, signal sequences (for secretion, internalization, nuclear localization, etc.), or stability tags. Optionally, the tags can be cleavable from the pro-polypeptides. As such, the bacteriocin can comprise a cleavage site between itself and the tag. It can be useful for the tag to remain affixed to the bacteriocin after the component peptides are cleaved from the pro-polypeptide. As such, in some embodiments, the cleavage site between the bacteriocin and the tag can be targeted by different cleavage enzymes (and/or cleaved under different chemical or pH conditions) than the cleavage sites separating the component peptides from each other.

In some embodiments, the pro-polypeptide further comprises a post-translational or co-translation modification, for example, glycosylation, acetylation, methylation, PEGYlation, SUMOylation, ubiquitination, or two or more of any of these.

In some embodiments, the pro-polypeptide comprises a quantity of two or more different component peptides (e.g., bacteriocins and/or signal molecules) in a desired ratio, or portion of a desired ratio. For example, the ratio of a first bacteriocin to a second (different) bacteriocin, or to a signal molecule can be about 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, or 9:10. In some embodiments, the pro-polypeptide comprises a quantity of two or more different bacteriocins in a desired ratio, or portion of a desired ratio. In some embodiments, the desired ratio of bacteriocins is selected to target an undesired microbial organism or population of undesired microbial organisms. In some embodiments, the desired ratio of bacteriocins is selected to balance a population of a microbiome of an animal, a human organ, a plant root, and/or soil. In some embodiments, the bacteriocins and/or signal molecules are present in a portion of a desired ratio, and the desired ratio can be achieved by adding an additional bacteriocin and/or signal molecule in a suitable ratio (for example, if the products of two different pro-polypeptides are combined, together, they can yield the desired ratio of component polypeptides such as bacteriocins and/or signal molecules).

In some embodiments, the pro-polypeptide has a length of no more than 20,000 amino acids, for example, no more than 20,000, 15,000, 10,000, 9000, 8000, 7000, 6000, 5000, 4000, 3000, 2000, 1000, 900, 800, 700, 600, 500, 400, 300, 200, or 100 amino acids, including between any two of the listed values, for example 100-20,000; 100-10,000; 100-5000; 100-2000; 100-1000; 500-20,000; 500-10,000; 500-5000; 500-2000; 500-1000; 1000-20,000; 1000-10,000; 1000-5000; or 1000-2000 amino acids. In some embodiments, the pro-polypeptide has a length of no more than 5000 amino acids. In some embodiments, the pro-polypeptide has a length of no more than 2000 amino acids.

### Nucleic Acids

Methods, kits, microbial cells, and/or nucleic acids of some embodiments herein include a nucleic acid encoding a pro-polypeptide. In some embodiments, the nucleic acid encodes a bacteriocin and a signal molecule in a single reading frame, and encodes a cleavage site coding sequences disposed between the bacteriocin and signal molecule coding sequences. In some embodiments, an isolated nucleic acid comprises two bacteriocin coding sequences in a single reading frame. The isolated nucleic acid can further comprise cleavage site coding sequences disposed between the bacteriocin coding sequences and in the single reading frame. In methods, nucleic acids, microbial cells, and/or kits of some embodiments herein, a nucleic acid encodes any pro-polypeptide described herein. In some embodiments, a kit comprises a nucleic acid composing one or more pro-polypeptides as described herein. In some embodiments, the kit further comprises a microbial cell capable of expression the pro-polypeptide.

As noted herein, for nucleic acids of some embodiments, it can be advantageous for the bacteriocins (or other polypeptides) encoded by the nucleic acids to remain intact after cleavage. As such, in some embodiments, the nucleic acid encodes cleavage sites for a cleavage enzyme, and its bacteriocin and/or signal molecule coding sequences encode bacteriocins and/or signal molecules (respectively) that do not comprise cleavage sites for the cleavage enzyme. In some embodiments, the cleavage site coding sequences of the nucleic acid encode cleavage sites for a cleavage enzyme, and the bacteriocin coding sequences encode bacteriocins that do not comprise cleavage sites for the cleavage enzyme.

In some embodiments, the nucleic acid comprises three or more bacteriocin coding sequences in the single reading frame, for example 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bacteriocin coding sequences, including ranges between any two of the listed values, for example, 3-10, 3-20, 3-30, 3-50, 5-10, 5-20, 5-30, 5-50, 7-10, 7-20, 7-30, 7-50, 10-20, 10-30, or 10-50 bacteriocin coding sequences. In some embodiments, the nucleic acid comprises at least three bacteriocin sequences in the single reading frame. In some embodiments, the nucleic acid comprises at least five bacteriocin sequences in the single reading frame. In some embodiments, at least two of the bacteriocin coding sequences encode different bacteriocins from each other.

In some embodiments, one or more cleavage site coding sequences are disposed in frame between any two adjacent bacteriocin coding sequences of the isolated nucleic acid. As noted herein, it can be advantageous for a single cleavage enzyme to be able to separate multiple, or all, of the component peptides of a pro-polypeptide. Accordingly, in some embodiments, each of the bacteriocin coding sequences (and, if present, signal molecule coding sequence(s)) are separated from each other by in-frame cleavage site coding sequences for the same enzyme. As such, upon translation of the nucleic acid, the resultant pro-polypeptide can be cleaved into its component peptides by a single enzyme.

In some embodiments, a cleavage site coding sequence encodes a cleavage site for a first cleavage enzyme, and another cleavage site coding sequence encodes another cleavage site for a different cleavage enzyme. In some embodiment, the bacteriocins encoded by the bacteriocin coding sequences do not comprise a cleavage site for any of the first or second cleavage enzymes. In some embodiment, no bacteriocins encoded by the isolated nucleic acids comprises a cleavage site for any of the cleavage enzymes that cleave the interspersed cleavage sites.

In some embodiments, all of the bacteriocin coding sequences (and, if present, signal molecule coding sequence(s)) are separated from each other by in-frame cleavage site coding sequences, which are collective cleaved by a combination of two, three, four, five, six, seven, eight, nine, or ten cleavage enzymes. As such, upon translation of the nucleic acid, that combination of cleavage enzymes can be used to cleave the pro-polypeptide into its component peptides.

In some embodiments, the cleavage site coding sequence encodes a cleavage site for a cleavage enzyme as described herein, for example in Table 4. In some embodiments the cleavage site coding sequence encodes a cleavage site that is chemically and/or pH-sensitive as described herein.

In some embodiments, the component peptide (e.g., bacteriocin and/or signal molecule) coding sequences of the isolated nucleic acid are present in a desired ratio, or portion of a desired ratio. In some embodiments, three of more bacteriocin sequences are present in a desired ratio or portion of a desired ratio. In some embodiments, the desired ratio is selected to target an undesired microbial organism or population of undesired microbial organisms. In some embodiments, the desired ratio of bacteriocins is selected to balance a population of a microbiome of an animal, a human organ, or a plant root and/or soil.

In some embodiments, the isolated nucleic acid further comprises a coding sequence for a signal molecule in the single reading frame. A cleavage site coding sequences can be disposed between the coding sequence for signal molecule and an adjacent bacteriocin coding sequence or sequences. In some embodiments, the signal molecule comprises a quorum sensing molecule, a signal transduction receptor ligand, a growth factor, a hormone, or a cytokine. In some embodiments, the signal molecule can be wild-type, mutant, or synthetic.

### Microbial Cells for Making Bacteriocins

Methods, kits, and microbial cells of some embodiments comprise a microbial cell comprising an isolated nucleic acid as described herein. Such microbial cells can be useful for producing pro-polypeptides comprising bacteriocins in desired ratios as described herein. The microbial cell can comprise a promoter as described herein. The promoter can be operably linked to the isolated nucleic acid. Thus, the microbial cell can be configured to transcribe the isolated nucleic acid and translate it into a pro-polypeptide comprising bacteriocins. As noted herein, the bacteriocins of the pro-polypeptide can be inactive while they are part of the pro-polypeptide. Accordingly in some embodiments, the isolated microbial cell does not produce a functional immunity modulator for a bacteriocin encoded by the isolated nucleic acid. For example, the microbial cell can lack a coding sequence for the immunity modulator, or if it does comprise coding sequence for the immunity modulator, the coding sequence can be transcriptionally silent (e.g. due to lack of a promoter), and/or the coding sequence can be mutated so that any immunity modulator produced by the coding sequence is nonfunctional. In some embodiments, the promoter operationally linked to the nucleic acid comprises, consists essentially of, or consists of any of the promoters of Tables 3.1-3.11.

### Methods of Making Bacteriocins

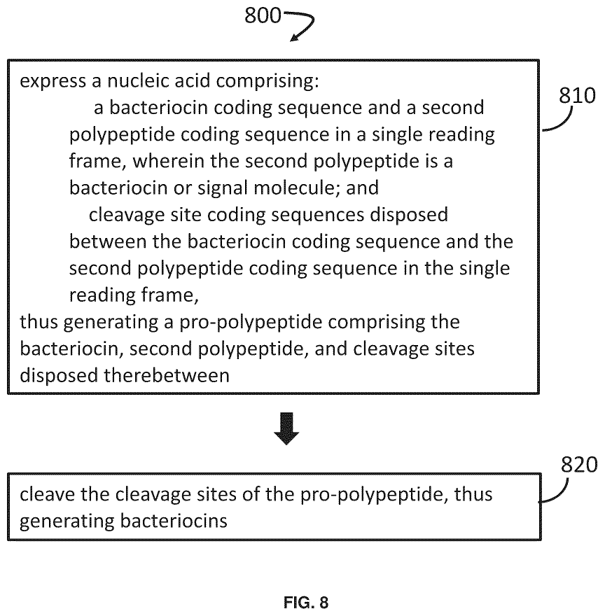

Some embodiments include methods of making bacteriocins. The methods can be useful for making compositions comprising bacteriocins (and optionally, other molecules such as signal molecules) in a desired ratio. In some embodiments, a method of making bacteriocins comprises expressing a nucleic acid. The nucleic acid can comprise two bacteriocin coding sequences in a single reading frame, or a bacteriocin and a signal molecule coding sequence in a single reading frame (see, for example, FIG. 8). The nucleic acid can further comprise cleavage site coding sequences disposed between the bacteriocin coding sequences (or between the bacteriocin and signal molecule coding sequences) and in the single reading frame. The nucleic acid can be expressed to generate a pro-polypeptide comprising the bacteriocins and cleavage sites disposed therebetween. In some embodiments, nucleic acids encoding bacteriocins (and/or signal molecules) as described herein are expressed by a gene expression system to as to produce pro-polypeptides as described herein.

In some embodiments, for example, when producing bacteriocins for a semi-controlled environment as in biopharma production, the pro-polypeptide comprises a bacteriocin coding sequence, a signal molecule coding sequence, and a cleavage site disposed between the bacteriocin and signal molecule coding sequences. It is contemplated that such a pro-polypeptide can be used to produce a composition comprising the bacteriocin and signal molecule in a desired ratio, which can be useful for inhibiting genetically drifting microbial organisms (via the bacteriocin; for example if the immunity of the target organisms is tied to their maintenance of a certain genetic state; this approach is described in additional detail in U.S. Pat. No. 9,333,227). Additionally, the signal molecule can promote the growth, proliferation, or production of a desired product by producing cells in environment (for example, genetically engineered microbial cells, or mammalian cell culture such as CHO or BHK cells).

In some embodiments, the method further comprises cleaving the cleavage site, thus separating the bacteriocins (and/or signal molecules) from each other. Upon separation of the bacteriocins (and/or signal molecules), a composition comprising bacteriocins (or combination of bacteriocins and signal molecules) is thus produced. In some embodiments, the cleavage sites are cleaved by a cleavage enzyme or combination of cleavage enzymes as described herein, for example in Table 4. In some embodiments, the cleavage sites are cleaved by exposing a peptide comprising a pH-sensitive or chemically-sensitive linker to pH or chemical conditions that induce cleavage. In some embodiments, the pro-polypeptide is stored for period of time before cleaving, for example, at least 1 day, 2 days, 3 days, 4 days, 5 days, 6 days, 1 week, 2 weeks, 3 weeks, 4 weeks, 1 month, 2 months, or 3 three months, including ranges between any two of the listed values.

In some embodiments, bacteriocins are inactive when they are part of a pro-polypeptide. Accordingly, in some embodiments, the microbial cell does not produce a functional immunity modulator for at least one of the bacteriocins, for example, the microbial cell can lack a coding sequence for the immunity modulator, or if it does comprise coding sequence for the immunity modulator, the coding sequence can be transcriptionally silent (e.g. due to lack of a promoter), and/or the coding sequence can be mutated so that any immunity modulator produced by the coding sequence is nonfunctional. In some embodiments, the microbial cell comprises E. coli or B. subtilis.

In some embodiments, at least one of the bacteriocins is inactive when it is part of the pro-polypeptide.

In some embodiments, the nucleic acid is expressed in vitro. Cell-free expression systems can be used to transcribe and/or translate nucleic acids in vitro. In some embodiments, the in vitro expression system comprises, consists of, or consists essentially of cell extracts. In some embodiments, the in vitro expression system comprises an RNA polymerase, ribosomes, tRNAs (and the corresponding amino acids), an energy source, and enzymatic cofactors. The in vitro expression system can further comprise enzymes for co- or post-translational modification, and/or cellular components that mediate protein folding such as heat shock proteins.

In some embodiments, the method further comprises isolating the pro-polypeptide prior to the cleaving. The pro-polypeptide can be isolated from the microbial cell, or from the in vitro expression system as described herein. In some embodiments, isolation comprises purifying the pro-polypeptide. In some embodiments, isolating comprises affinity purifying the pro-polypeptide. The affinity purification can be performed using an affinity tag as described herein. The nucleic acid can encode an affinity tag on the pro-polypeptide. The affinity tag can be bound by a suitable binding agent specific for the affinity tag (e.g., an anti-myc antibody for a myc tag, or GSH-coated beads for the GST, or nickel or cobalt for a His tag). The binding agent can be immobilized on a solid phase, so that, upon binding of the affinity tag, the pro-polypeptide will be immobilized on the solid phase. The solid phase can be removed from the microbial cell and/or in vitro expression system. Optionally, the solid phase can be washed. The affinity tagged pro-polypeptide can then be released from the solid phase. For example, the pro-polypeptide can be isolated by immunoprecipitation, affinity chromatography, and the like.

In some embodiments, the nucleic acid comprises at least three bacteriocin coding sequences in a single reading frame, for example at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bacteriocins, including ranges between any two of the listed values, for example, about 3-10, 3-20, 3-30, 3-50, 5-10, 5-20, 5-30, 5-50, 7-10, 7-20, 7-30, 7-50, 10-20, 10-30, or 10-50 bacteriocins. As such, the nucleic acid can produce a pro-polypeptide comprising the indicated number of bacteriocins.

In some embodiments, at least two of the bacteriocins are different from each other. In some embodiments, two or more of the bacteriocins are the same. In some embodiments, at least two of the bacteriocins are different from each other, and at least two of the bacteriocins are the same.

Upon cleavage of the pro-polypeptide, a composition comprising bacteriocins can be produced. In some embodiments, the composition comprises a desired ratio of bacteriocins. In some embodiments, at least a portion the desired ratio is achieved by a ratio of bacteriocin coding sequences in the single reading frame of the nucleic acid. Without being limited by theory, it is contemplated that in some embodiments, the nucleic acid encodes desired relative quantities of two or more different bacteriocins coding sequences in cis, the corresponding pro-polypeptide will also have these ratios of bacteriocins. For example, if the nucleic acid comprises two coding sequences for “bacteriocin A,” and three coding sequences of “bacteriocin B,” each separated by cleavage sites, the resultant pro-polypeptide will comprise a ratio of bacteriocin A to bacteriocin B of 2:3. Upon cleavage, the solution will comprise the bacteriocins in these ratios. It is noted that in some embodiments, additional proteins or polypeptides encoded by the nucleic acid are also produced in desired ratios, for example signal molecules. As such, a solution can be produced comprising bacteriocins and signal molecules in a predetermined ratio or range of ratios. In some embodiments, the predetermined ratio is achieved by a ratio of bacteriocin coding sequences in the single reading frame of the nucleic acid.

In some embodiments, the nucleic acid encodes bacteriocins (and optionally additional peptides such as signal molecules) in a portion of a desired ratio. Additional components can be added to achieve the final desired ratio. For example, a second, different pro-polypeptide can comprise bacteriocins and/or signal molecules in a second ratio. The two different polypeptides can together provide the final desired ratio of bacteriocins (and optionally other peptides, such as signal molecules) of interest. As such, in some embodiments, the desired ratio is further achieved by a second nucleic acid comprising a ratio of bacteriocin coding sequences and further comprising cleavage sites between the bacteriocin coding sequences.

A desired ratio can be selected for a number of applications. In some embodiments, a desired ratio of bacteriocins is selected to target an undesired microbial organism or population of undesired microbial organisms. For example, if an environment such as a culture medium, feedstock, fermenter, bioreactor, or microbiome contains, or is at risk of containing a population of undesired microbial organisms, a ratio of bacteriocins can be selected to target those undesired microbial organisms. In some embodiments, the desired ratio of bacteriocins is selected to balance a population of a microbiome of an animal (for example a horse, cow, sheep, pig, donkey, dog, cat, or non-human primate), a human organ (e.g., skin or a gut), or a plant root and/or soil microbiome, or to preserve a product such as a food product (human or non-human animal), pharmaceutical, or cosmetic product. In some embodiments, the desired ratio comprises a ratio between two bacteriocins in a pro-polypeptide (e.g., a first to second bacteriocin, first to third, second to third, or third to fourth, or fourth to fifth) of about 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, or 9:10, in which the first (or second, or third, or fourth) bacteriocin is different from the second (or third, or fourth, or fifth) bacteriocin. It is noted that different pairs of bacteriocins in the pro-polypeptide can have different ratios to each other. Therefore, it is contemplated that the ratios three or more bacteriocins to each other can be ascertained by the individual (pair-wise) ratios of the bacteriocins to each other. For example, a first and second bacteriocin can have a ratio of 1:2, and a second and third bacteriocin can have a ratio of 2:5, so that the ratio of the first to the second to the third bacteriocin is 1:2:5, respectively. In some embodiments, the desired ratio comprises a ratio of a first bacteriocin to a second bacteriocin to a third bacteriocin of about 1:1:2, 1:2:2, 1:1:3, 1:2:3, 1:3:3, 2:2:3, or 2:3:3.

As noted herein, it can be advantageous for the bacteriocins to remain intact following cleavage of the pro-polypeptide. As such, in some embodiments, the bacteriocins of the pro-polypeptide do not contain any of the cleavage sites that separate the bacteriocins (or optional other polypeptides such as signal molecules). Upon cleavage of the cleavage sites, the bacteriocins can remain intact. In some embodiments, the cleavage sites are for a single cleavage enzyme, and the cleavage enzyme does not cleave within the bacteriocins. In some embodiments, at least one cleavage site is for a first cleavage enzyme, and another cleavage site is for a second cleavage enzyme, and neither the first nor the second cleavage enzyme cleaves within the bacteriocins.

In some embodiments, the composition produced by the method comprises a signal molecule as described herein. The nucleic acid can encode the signal molecule in the same reading frame as the other component(s) of the pro-polypeptide (e.g., the bacteriocins). A cleavage site sequence disposed between the signal molecule and the bacteriocin coding sequence or sequences. As such, upon cleavage of the cleavage sites, the signal molecule can be separate from the bacteriocin. In some embodiments, the signal molecule comprises, consists essentially of, or consists of a quorum sensing molecule, signal transduction receptor ligand, growth factor, hormones, or cytokine, or a combination of two or more of these. In some embodiments, the signal molecule is wild-type, mutant, or synthetic.

In some embodiments, the pro-polypeptide has a length of no more than 20,000 amino acids, for example, no more than 20,000, 15,000, 10,000, 9000, 8000, 7000, 6000, 5000, 4000, 3000, 2000, 1000, 900, 800, 700, 600, 500, 400, 300, 200, or 100 amino acids, including between any two of the listed values, for example 100-20,000; 100-10,000; 100-5000; 100-2000; 100-1000; 500-20,000; 500-10,000; 500-5000; 500-2000; 500-1000; 1000-20,000; 1000-10,000; 1000-5000; or 1000-2000 amino acids. In some embodiments, the pro-polypeptide has a length of no more than 5000 amino acids. In some embodiments, the pro-polypeptide has a length of no more than 2000 amino acids.

As noted herein, the ratios of bacteriocins (and other polypeptides such as signal molecules) can be further tuned by using two or more different pro-polypeptides to obtain particular ratios and/or combinations of molecules (e.g., bacteriocins, signal molecules). In some embodiments, the method comprises expressing a second nucleic acid encoding a second pro-polypeptide comprising two bacteriocins and cleavage sites disposed between the bacteriocins. The second pro-polypeptide can be different from the first pro-polypeptide, so cleavage of the second pro-polypeptide produces a different ratio of bacteriocins and/or signal molecules than the first pro-polypeptide once it has been cleaved.

In some embodiments, the method further comprises chemically modifying the bacteriocins. Example chemical modifications include, but are not limited to glycosylation, acetylation, methylation, PEGYlation, SUMOylation, ubiquitination, or two or more of any of these. In some embodiments, the bacteriocins are chemically modified co-translationally. In some embodiments, the bacteriocins are chemically modified post-translationally. For example, the microbial cell can comprise, or can be genetically engineered to comprise enzymes for co-translational modification or post-translational modification. For example, the in vitro expression system can comprise enzymes for co-translational or post-translational modification. For example, the in vitro expression system can comprise enzymes for post-translational modification, and/or, following isolation of the pro-polypeptide from the expression system (in vitro or microbial cell), the pro-polypeptide can be contacted with enzymes that produce the desired post-translational modification. In some embodiments, bacteriocins are chemically modified after the pro-polypeptide has been cleaved.

Some embodiment include a composition comprising bacteriocins, which can be made according to methods of some embodiments herein is shown in FIG. 3. In some embodiments, the composition is produced by the cleavage of a pro-polypeptide as described herein. It is contemplated that the bacteriocins can be released by cleavage of the pro-polypeptide, for example via an endopeptidase. The result can be a mixture with the correct molecular blend according to a predetermined and/or desired result in terms of bacterial killing. In some embodiments, one strain of host cell can produce a multi-bacteriocin mixture in one fermentation. It is contemplated that there can be no toxic effect during production (and thus, no need of immunity in the host cell). Optionally, purification of the pro-polypeptide (and/or “cleaved” bacteriocins) can be performed purification via a tag. Optionally molecular adjustment between bacteriocins can be performed.

### Compositions Comprising Bacteriocins

In some embodiments, compositions comprising bacteriocins in desired ratios are provided. Optionally, the composition can further comprise additional polypeptides, for example signal molecules, in desired ratios with the bacteriocins and/or each other.

In some embodiments, a composition comprises two or more bacteriocins in a ratio selected to target a microbial cell or populations of microbial cells. Each of the bacteriocins can comprise, at its N-terminus, C-terminus, or N-terminus and C-terminus, a portion of a cleavage sequence that has been cleaved. It is contemplated that when a pro-polypeptide comprising multiple bacteriocins as described herein is cleaved at its cleavage sites, at least some of the bacteriocins will comprise the vestiges of the cleavage site at their N- and/or C-termini, depending on the cleavage site. As such, it is contemplated that not only will the bacteriocins of the composition be in desired ratios with a very high degree of accuracy, but further, the bacteriocins will be structurally distinct in that many or all of the bacteriocins will comprise N- and/or C-terminal vestiges of cleavage sites, for example partial cleavage sites. In some embodiments, the portions of cleavage sequences at the N-, C-, or N- and C-termini of the bacteriocins are for cleavage sites of the same cleavage enzyme. In some embodiments, the portions of cleavage sequences at the N-, C-, or N- and C-termini of the bacteriocins are for cleavage sites of two or more different cleavage enzymes.

In some embodiments, some or all of the bacteriocins of the composition further comprise a tag. In some embodiments, the tag is selected from the group consisting of affinity tags, a signal sequence, or a stability tag. In some embodiments, a cleavage site is disposed between the tag and the bacteriocin.

In some embodiments, the composition further comprises a signal molecule as described herein. The signal molecule can also be in the desired ratio. The signal molecule can also comprise vestiges of cleavage sites at its N- and/or C-termini.

In some embodiments, the ratio of bacteriocins in the composition is selected to target an undesired microbial organism or population of undesired microbial organisms in an environment, such as an industrial manufacturing environment, a fermenter, a food, drug, or cosmetic manufacturing environment, or a product to be preserved (e.g., a food, drug, or cosmetic product). In some embodiments, the ratio of bacteriocins is selected to balance a population of a microbiome of an animal, a human organ, or a plant root and/or soil, or to preserve a product (such as a food, drug, or cosmetic product). In some embodiments, the composition is formulated for topical or oral administration to a human subject.

### Coding Substrates

In accordance with methods and microfluidic devices and systems of some embodiments described herein, coding substrates are provided. A coding substrate can comprise a nucleic acid encoding a protein or peptide such as an antimicrobial peptide and/or bacteriocin (and, optionally, an auxiliary protein) as described herein. The coding substrates of methods and microfluidic devices of some embodiments encode antimicrobial peptides and/or bacteriocins, and can be used to produce a specified mixture of antimicrobial peptides and/or bacteriocins. The coding substrates can be discrete, and capable of being placed in fluidic isolation from each other (which may be referred to herein as “discrete” coding substrates). For example, in a method or microfluidic device of some embodiments, several discrete coding substrates may be used to produce a specified mixture of antimicrobial peptides and/or bacteriocins by way of in vitro transcription/translation, in which (i) only discrete coding substrates encoding the antimicrobial peptides and/or bacteriocins of the specified mixture are contacted and incubated with an in vitro transcription/translation solution, (ii) only coding substrates that have produced antimicrobial peptides and/or bacteriocins of the specified mixture are placed in fluidic communication with a fluidic reservoir where the specified mixture is formed, or (i) and (ii). For example, valves as described herein can control the flow of in vitro transcription/translation solution to, and/or antimicrobial peptides and/or bacteriocins from discrete coding substrates (or chambers housing the discrete coding substrates). In methods and microfluidic devices of some embodiments, different coding substrates, encoding different antimicrobial peptides and/or bacteriocins from each other may be housed in separate chambers (of a microfluidic device as described herein), each of which may be placed in fluid communication with an in vitro transcription/translation solution by a valve. As such, in methods and microfluidic devices some embodiments, the discrete coding substrates of a microfluidic device are comprised within separate chambers. In some embodiments, the discrete coding substrates comprise, consist essentially of, or consist of beads. By way of example, beads encoding the antimicrobial peptides and/or bacteriocins of the specified mixture (but not beads encoding other antimicrobial peptides and/or bacteriocins) can be placed in fluid communication with an in vitro transcription/translation solution, either mechanically, or through the opening and/or closing of valve to direct fluids to the appropriate beads. In some embodiments, different discrete coding substrates encode different antimicrobial peptides and/or bacteriocins from each other. Some embodiments comprise a system comprising a processor. The system can be configured to be placed in fluid and/or data communication with a microfluidic device as described herein. Optionally, the system comprises a pump, and/or reservoirs of reagents (e.g., in vitro transcription/translation solution) which can be placed in fluid communication with the microfluidic device. In some embodiments, the microfluidic device is formatted as a cartridge for insertion into the system. In some embodiments, the coding substrates encode bacteriocins, but not antimicrobial peptides. In some embodiments, the coding substrates encode antimicrobial peptides, but not bacteriocins. In some embodiments, the coding substrates encode a combination of antimicrobial peptides and bacteriocins.

In accordance with the methods and microfluidic devices and systems of some embodiments described herein, the coding substrates are discrete coding substrates. In some embodiments, a discrete coding substrate is comprised within a chamber. Accordingly, in some embodiments, multiple discrete coding substrates are each comprised within a separate chamber. In some embodiments, two or more discrete coding substrates are comprised within the same chamber, so that a single chamber contains two or more discrete coding substrates, and thus can produce a mixture and/or stoichiometry of antimicrobial peptides and/or bacteriocins. In some embodiments, a chamber comprises at least 1, 2, 3, 4, 5, 6, 7, 8, 9, 10, 20, 30, 40, 50, 60, 70, 80, 90, 100, 100, 1000, 5000, 10,000, 500,000, 1,000,000, 10,000,000, or 100,000,000 discrete coding substrates, including ranges between any two of the listed values, for example 1-3, 1-5, 1-10, 1-50, 2-3, 2-5, 2-10, 2-20, 2-50, 2-100, 10-50, 50-100, 50-500, 50-1000, 100-500, 100-1000, 500-1000, 1000-5000, 5000-10,000, 10,000-50,000, 50,000-100,000, 100,000-500,000, 500,000-1,000,000, 1,000,000-10,000,000, 10,000,000-100,000,000, or 100,000,000-1,000,000,000 discrete coding substrates.

In accordance with the methods and microfluidic devices and systems of some embodiments described herein, the chambers of the microfluidic device comprising the discrete coding substrates are micro-scale chambers. For example, the chambers may each have a volume of no more than 1, 5, 10, 20, 50, 100, 250 or 500 microliters, including ranges between any two of the listed values, for example, 1-5 microliters, 1-10 microliters, 1-20 microliters, 1-50 microliters, 1-100 microliters, 1-500 microliters, 5-10 microliters, 5-20 microliters, 5-50 microliters, 5-100 microliters, 5-500 microliters, 10-20 microliters, 10-50 microliters, 10-100 microliters, 10-500 microliters, 50-100 microliters, or 50-500 microliters. In some embodiments, the chambers comprise, consist essentially of, or consist of a material or product selected from the group consisting of a well, nanowell, membrane, matrix, plastic, metal, glass, polymer, polysaccharide, and paramagnetic compound, or a combination of two or more of these. In accordance with the methods and microfluidic devices of some embodiments described herein, the coding substrates comprise, consist essentially of, or consist of a material or product selected from the group consisting of a chip, bead, nanoparticle, well, nanowell, membrane, matrix, plastic, metal, glass, polymer, polysaccharide, and paramagnetic compound, or a combination of two or more of these. Some embodiments include a microfluidic device comprising separate microchambers, each comprising one or more beads, each of which comprises, consisting essentially of, or consists of a coding substrate. The beads can each comprise nucleic acids encoding antimicrobial peptides and/or bacteriocins. In some embodiments, the device includes 1, 2, 3, 4, 5, 6, 7, 8, 9, 10, 10-50, 50-100, 100-500, 500-1000 or 1000-5000 chambers, or any number therebetween.

In accordance with the methods and microfluidic devices and systems of some embodiments described herein, the coding substrates comprise nucleic acids immobilized thereon. For example, the nucleic acids can be covalently bound to the coding substrate, hybridized to the coding substrate, and/or associated with the coding substrate via one or more force, such as an electrostatic force. In some embodiments, the nucleic acid comprises DNA. In some embodiments, the nucleic acid comprises RNA.

In some embodiments, a coding substrate further encodes or comprises an auxiliary protein, such as a protein for stabilization of antimicrobial peptides and/or bacteriocins, a protein with anti-protease activity (for example a serpentin), a protein for stabilization of antimicrobial peptides and/or bacteriocins, a protein for destruction of a microbial biofilm (for example a biofilm breaker such as Dispersin B), a pheromone protein, a protein that attracts a non-pathogenic microbial organism, or a protein that enhances growth or reproduction of the non-pathogenic microbial organism in a microbiome of a subject. Without being limited by theory it is contemplated that such an auxiliary protein can enhance expression and/or activity of an antimicrobial peptide and/or bacteriocin, for example, by stabilizing the antimicrobial peptide and/or bacteriocinin a particular environment, by inhibiting a protease from a particular environment that would degrade an antimicrobial peptide and/or bacteriocin, and/or by degrading fibrils of a microbial biofilm to enhance bacterial contact by the antimicrobial peptide and/or bacteriocin. In some embodiments, a discrete coding substrate encodes an antimicrobial peptide and/or bacteriocinin addition to an auxiliary protein. In some embodiments, a first coding substrate encodes an auxiliary protein and a second coding substrate encodes an antimicrobial peptide and/or bacteriocin. In some embodiments, the auxiliary proteins are selected with the antimicrobial peptides and/or bacteriocins. For example, if a mixture of antimicrobial peptides and/or bacteriocins is for an environment comprising a particular protease, for example trypsin, a coding substrate encoding an inhibitor of that protease can be selected along with coding substrates encoding the mixture of antimicrobial peptides and/or bacteriocins. In some embodiments, a coding substrate encodes an auxiliary protein with anti-protease activity. In some embodiments, the auxiliary protein with anti-protease is a protein for stabilization of antimicrobial peptides and/or bacteriocins. In some embodiments, one or more of the discrete coding substrates encodes an auxiliary protein that attracts a non-pathogenic microbial organism, or that enhances growth or reproduction of the non-pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract. In some embodiments, the auxiliary protein is a pheromone peptide that induces an antimicrobial peptide and/or bacteriocin of a desired microbial organism such as a non-pathogenic microbial organism (although in some embodiments, a pathogenic microbial organism is a desired microbial organism). Thus, for example, in some embodiments a discrete coding substrate encodes an auxiliary protein that induces or causes a bacteria to secrete an antimicrobial peptide and/or bacteriocin that inhibits growth or reproduction of another bacteria. In some embodiments, one or more of the discrete coding substrates encodes a protein that attracts the non-pathogenic microbial organism, or that enhances growth or reproduction of the non-pathogenic microbial organism in the microbiome of a subject. In some embodiments, bacteriocins, and not antimicrobial peptides are used.

Inhibition of growth or reproduction has its customary and ordinary meaning as understood by one of skill in the art in view of this disclosure. It refers to a decrease in or arrest of proliferation of microbial organisms (or a decrease in the rate of proliferation of microbial organisms), for example, arrest of the cell cycle and/or killing of microbial organisms. Similarly, enhancement of growth or reproduction refers to an increase in proliferation or the rate of proliferation of microbial organisms. Any method known in the art may be used to detect inhibition or enhancement of growth or reproduction.

### In Vitro Transcription/Translation Solutions

In accordance with the methods, systems and microfluidic devices of some embodiments described herein, a transcription/translation solution can be used to produce an antimicrobial peptide and/or bacteriocin encoded by a coding substrate. As such, the transcription/translation solution may be useful for generating peptides, or antimicrobial peptides and/or bacteriocins, in a method or microfluidic device for producing a specified mixture of antimicrobial and/or peptides bacteriocins The term, “in vitro transcription/translation solution” encompasses an in vitro transcription solution and/or an in vitro translation solution sufficient to produce an antimicrobial peptide and/or bacteriocin from a coding substrate. By way of example, in embodiments in which the coding substrate comprises RNA transcripts encoding antimicrobial peptides and/or bacteriocins, it is contemplated that an “in vitro transcription/translation solution” comprising, consisting essentially of, or consisting of a translation solution is sufficient (since it will be understood that the RNA is already a transcript). By way of example, in embodiments in which the coding substrate comprises DNAs encoding antimicrobial peptides and/or bacteriocins, it is contemplated that an “in vitro transcription/translation solution” comprises a transcription solution (for transcribing the DNAs into RNAs) and a translation solution (for translating the RNAs into polypeptides). In some embodiments, the transcription and translation solution are together in a single solution. In some embodiments, the transcription and translation solution are in separate solutions, for example in vesicles suspended in a single solution, and/or in separate solutions that are applied sequentially. In some embodiments, components of the in vitro transcription/translation solution are lyophilized, and configured to be reconstituted into the in vitro transcription/translation solution upon the addition of water. In some embodiments, the in vitro transcription/translation solution is reconstituted by adding water to lyophilized components.

Translation solutions can be useful for translating nucleic acids in accordance with the methods, systems and/or microfluidic devices described herein. Suitable translation solutions can comprise, consist essentially of, or consist of reagents for in vitro translation (which, for convenience, may be referred to herein as “translation reagents”), and as such can be configured for in vitro translation of a transcript such as an RNA. Some embodiments include a transcription solution comprising reagents for transcription (which, for convenience, may be referred to herein as “transcription reagents”), and thus is configured for in vitro transcription and translation, for example to transcribe and translate a nucleic acid encoding an antimicrobial peptide and/or bacteriocin or other peptides as described herein. It is contemplated that in vitro transcription and translation in a single solution (such as a transcription solution further comprising a translation solution as described herein) can facilitate efficient in vitro production of antimicrobial peptides and/or bacteriocins or other peptides in accordance with methods and microfluidic devices of some embodiments. Thus, in accordance with the methods and microfluidic devices of some embodiments described herein, the in vitro transcription/translation solution comprises an in vitro transcription reagent and/or an in vitro translation reagent.

In accordance with the methods and microfluidic devices of some embodiments described herein, the translation solution comprises, consists essentially of, or consists of one or more translation reagents or in vitro translation reagents. Examples of translation reagents include, but are not limited to, a ribosome, a buffer, an amino acid, a tRNA (which may be conjugated to an amino acid), a lysate or extract such as an E. coli lysate or E. coli extract, and a cofactor or metallic ion such as Mg²⁺, or a combination of two or more of any of the listed items. In accordance with the methods, systems and kits of some embodiments described herein the translation solution further comprises a transcription solution, and thus is configured for in vitro transcription and translation. As described herein, a transcription solution further comprising a translation solution contemplates a single solution that is suitable for in vitro transcription and translation. As such, a transcription solution further comprising a translation solution encompasses a single transcription/translation solution. It will be appreciated that some components of a transcription and/or translation solution, for example ribosomes, may not be liquids, and could potentially be isolated from the transcription and/or translation solution, for example by filtration and/or centrifugation. Translation solutions of methods and microfluidic devices of some embodiments described herein (and which can be comprised by translation solutions as described herein) can comprise, consist essentially or, or consist of one or more transcription reagents. Examples of transcription reagents include an RNA polymerase, a buffer, a nucleic acid mix (for example, NTPs including ATP, GTP, CTP, and UTP), a cofactor or metallic ion such as Mg²⁺, a transcription inducer (such as a transcription factor, IPTG, or lactose), a polyadenylation enzyme, a capping enzyme, a lysate or extract such as a bacterial lysate or extract such as an E. coli lysate or E. coli extract, an SP6 polymerase, a T3 polymerase, a T7 RNA polymerase, or a mixture of two or more of any of the listed items. The transcription solution can be useful for transcribing a template, such as a candidate nucleic acid as described herein. Translation solutions of methods and microfluidic devices of some embodiments include one or more transcription reagents in combination with one or more translation reagents. In some embodiments, a microfluidic device and/or system comprises a reservoir comprising an in vitro transcription/translation solution as described herein. In some embodiments, the reservoir comprises an in vitro transcription solution and an in vitro translation solution which can be combined to produce the in vitro transcription/translation solution.

In some embodiments, the translation solution comprises a post-translational modification enzyme. Examples of post-translational modification enzymes include, but are not limited to a cleavage enzyme, a kinase, a phosphatase, a glycosyltransferase, or a mixture of any two of the listed items.

In accordance with the systems, methods and microfluidic devices of some embodiments described herein, the translation solution is provided to a coding substrate at a microliter-scale. For example, the translation solution may have a volume of 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl.

In accordance with the systems, methods and microfluidic devices of some embodiments described herein, the in vitro transcription/translation solution is lyophyilized. In some embodiments, the in vitro transcription/translation solution is configured be reconstituted in a solution such as water. As such, a microfluidic device of some embodiments can be stably stored for periods of time at ambient temperature, without substantial impact on the efficacy of the in vitro transcription/translation solution. Accordingly, it is contemplated that the microfluidic device of some embodiments is suitable for point-of-care treatment, and can be used to prepare specified mixtures of antimicrobial peptides and/or bacteriocins as needed (for example, in response to particular injuries, infections, and/or surgical procedures).

### Microfluidic Devices and Systems

A fluidic system comprising a microfluidic device can be useful for producing specified mixtures of antimicrobial peptides and/or bacteriocins in accordance with the methods and microfluidic devices and systems of some embodiments described herein. In some embodiments, a system is configured to receive a microfluidic device comprising discrete coding substrates as described herein, so that the system can direct the microfluidic device to produce a specified mixture of antimicrobial peptides and/or bacteriocins via in vitro transcription/translation. In some embodiments, the system is configured to be in fluid and/or data communication with the device. For example, disposable cartridge can comprise, consist essentially of, or consist of a microfluidic device for use in conjunction with systems as described herein. As it is contemplated that specified mixtures of antimicrobial peptides and/or bacteriocins can be customized for particular applications, for example, to interact with a particular microbiome of a particular subject, or to target a particular infection, it is contemplated that single-use disposable microfluidic devices can minimize contamination (for example from residual bacteriocins) that may interfere with the stoichiometry and composition of the specified mixture of antimicrobial peptides and/or bacteriocins, and can be useful for maintaining sterility for medical use on a subject. As such, in some embodiments, the microfluidic device is sterile. In the methods and microfluidic devices of some embodiments, the fluidic device comprises a microfluidic device. As it will be understood that a “microfluidic device” is a kind of “fluidic device,” a microfluidic device is expressly contemplated wherever a “fluidic device” is mentioned herein. Additionally, unless expressly stated otherwise, any disclosure of a fluidic device (such as a microfluidic device) herein is understood to be applicable to a microfluidic device of some embodiments, as well as methods comprising microfluidic devices as described herein.

In some embodiments, a microfluidic device for producing a specified mixture of antimicrobial peptides and/or bacteriocins is provided. In some embodiments, the device comprises discrete coding substrates, as described herein, that each encode an antimicrobial peptide and/or bacteriocin. The device can further comprise an in vitro transcription/translation solution as described herein. The device can further comprise a fluidic reservoir, one or more valves, and/or can be configured to be placed in data communication with a processor as described herein. The valves can regulate fluidic communication between the transcription/translation solution, the discrete coding substrates, and/or the fluidic reservoir. The processor can be configured to control the valves, so as to place coding substrates encoding the antimicrobial peptides and/or bacteriocins of the specified mixture in fluid communication with the in vitro transcription/translation solution so that the antimicrobial peptides and/or bacteriocins of the specified mixture are produced. The processor can be configured to control the valves (and, in some embodiments, fluidic flow) in the microfluidic device to place the antimicrobial peptides and/or bacteriocins of the mixture in fluid communication with the fluidic reservoir so that the antimicrobial peptides and/or bacteriocins of the specified mixture flow to the fluidic reservoir, thus providing the specified mixture of antimicrobial peptides and/or bacteriocins in the fluidic reservoir. Optionally, the antimicrobial peptides and/or bacteriocins of the specified mixture are actively mixed in the fluidic reservoir. In some embodiments, the antimicrobial peptides and/or bacteriocins of the specified mixture mix passively (but are not actively mixed) in the fluidic reservoir. In some embodiments, the microfluidic device comprises the processor. In some embodiments, the microfluidic device does not itself comprise the processor, but is configured to be placed in data communication with the processor. In some embodiments, the microfluidic device is for producing a specified mixture of bacteriocins, and not antimicrobial peptides. In some embodiments, the microfluidic device is for producing a specified mixture of antimicrobial peptides, and not bacteriocins. In some embodiments, the microfluidic device is for producing a specified mixture of bacteriocins and antimicrobial peptides.

Some embodiments of the methods and microfluidic devices described herein include a microfluidic device for producing a specified mixture of antimicrobial peptides and/or bacteriocins. The device can comprise: discrete coding substrates that each encode a bacteriocin and/or an antimicrobial peptide; an in vitro transcription/translation solution; a fluidic reservoir; valves each disposed on a fluidic path between a discrete coding substrate and the fluidic reservoir, each valve configured to regulate flow between the discrete coding substrate and the fluidic reservoir. The device can be configured to be placed in data communication with a processor configured to: based on the specified mixture of antimicrobial peptides and/or bacteriocins, configure the valves to place the discrete coding substrates that encode the antimicrobial peptides and/or bacteriocins of the mixture, but not other discrete coding substrates, in fluidic communication with the fluidic reservoir; permit incubation of the in vitro transcription/translation solution with the discrete coding substrates that encode the antimicrobial peptides and/or bacteriocins of the mixture, so that the antimicrobial peptides and/or bacteriocins of the specified mixture are produced; permit flow of the antimicrobial peptides and/or bacteriocins through the valves into the fluidic reservoir; and control flow of fluid in the fluidic reservoir, wherein the flow comprises movement of a fluid comprising the specified antimicrobial peptides and/or bacteriocins in the fluidic reservoir, thus producing the specified mixture of antimicrobial peptides and/or bacteriocins. In some embodiments, the microfluidic device comprises the processor. In some embodiments, the microfluidic device is for producing a specified mixture of bacteriocins, and not antimicrobial peptides. In some embodiments, the microfluidic device is for producing a specified mixture of antimicrobial peptides, and not bacteriocins. In some embodiments, the microfluidic device is for producing a specified mixture of bacteriocins and antimicrobial peptides.

In accordance with the methods and microfluidic devices of some embodiments described herein, the microfluidic device comprises discrete coding substrates but not a fluidic reservoir and/or an in vitro transcription/translation solution. As such, the microfluidic device can be placed in fluid and data communication with a system as described herein, and the system can provide the fluidic reservoir and/or the in vitro transcription/translation solution so that the specified mixture of antimicrobial peptides and/or bacteriocins can be produced. Accordingly, in some embodiments, the system comprises a fluidic reservoir and/or an in vitro transcription/translation solution as described herein. In some embodiments, the microfluidic device does not comprise the fluidic reservoir, the in vitro transcription/translation solution, and/or any other components of the system, and is separate from these components of the system until it is placed in data and/or fluid communication with the system. In some embodiments, the microfluidic device is configured to be attached to the fluidic reservoir, the in vitro transcription/translation solution, and/or another component of the system. For example, the device may comprise a cartridge that is configured to be inserted into the system to place the discrete coding substrates of the cartridge in fluid communication with an external (to the cartridge) to the fluidic reservoir, in vitro transcription/translation solution, and/or other component of the system. In some embodiments, the cartridges are consumable. For example, a cartridge may comprise discrete coding substrates, be configured to be inserted into the system described herein to produce a mixture of specified antimicrobial peptides and/or bacteriocins, and then be discarded, after which another cartridge that includes other discrete coding substrates may be inserted into the system to produce a different mixture of specified antimicrobial peptides and/or bacteriocins. In some embodiments, a cartridge is configured to receive an in vitro transcription/translation solution from the system, so that the in vitro transcription/translation solution contacts the discrete coding substrates of the cartridge that encode the bacteriocins and/or antimicrobial peptides of the specified mixture. In some embodiments, the microfluidic device is for producing a specified mixture of bacteriocins, and not antimicrobial peptides. In some embodiments, the microfluidic device is for producing a specified mixture of antimicrobial peptides, and not bacteriocins. In some embodiments, the microfluidic device is for producing a specified mixture of bacteriocins and antimicrobial peptides.

Also contemplated are embodiments in which the system and/or microfluidic device is dry. In some embodiments, the system or microfluidic device comprises a lyophilized reagent configured to be dissolved in a fluid such as water.

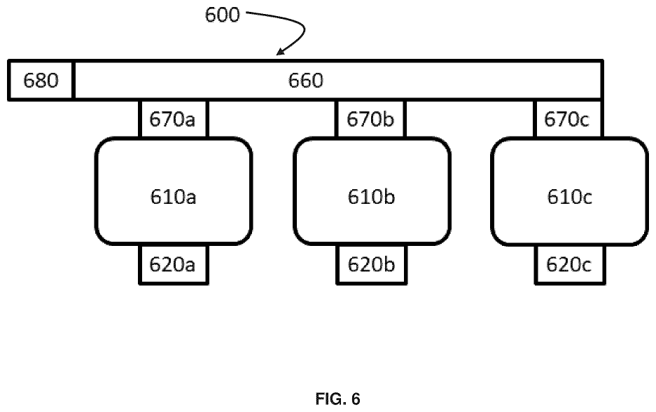

FIG. 5 is a schematic diagram depicting a microfluidic device 500 of some embodiments herein. The microfluidic device 500 can comprise discrete coding substrates 510a, 510b, 510c (while three discrete coding substrates are shown by way of example, it is contemplated that microfluidic devices of embodiments herein can comprise other quantities of discrete coding substrates, as described herein). The device can comprise a fluidic reservoir 540, which, by way of example, can receive antimicrobial peptides and/or bacteriocins produced from the discrete coding substrates 510a, 510b, 510c, and thus can contain a specified mixture of antimicrobial peptides and/or bacteriocins as described herein. Valves 520a, 520b, 520c, and/or 570a, 570b, 570c can regulate fluidic flow to and from the discrete coding substrates 510a, 510b, 510c. In some embodiments, the microfluidic device further comprises an outlet 590, which can permit flow from the fluidic reservoir 540 out of the microfluidic device. For example, the specified mixture of antimicrobial peptides and/or bacteriocins can flow through the outlet 590 to a tissue, wound, microbiome of a subject, and/or vessel as described herein. In some embodiments, a processor 595 as described herein is in data communication with the device, and controls flow through valves 520a, 520b, 520c, and/or 570a, 570b, 570c and/or 550 and/or 580. In some embodiments, the device does not itself comprise a processor 595, and is configured to placed in data communication with a processor 595 as described herein so as to controls flow through valves 520a, 520b, 520c, and/or 570a, 570b, 570c and/or 550 and/or 580. It is contemplated that in accordance with microfluidic devices and methods of some embodiments herein, the production of antimicrobial peptides and/or bacteriocins of a specified mixture can be achieved by contacting only a subset of discrete coding substrates 510a, 510b, 510c with an in vitro transcription/translation solution in the first place (so that only some discrete coding substrates are used to produce an antimicrobial peptide and/or bacteriocin), and/or by only permitting a subset of antimicrobial peptides and/or bacteriocins produced from discrete coding substrates 510a, 510b, 510c to enter the fluidic reservoir 540. It is noted that contacting only a subset of the discrete coding substrates with in vitro transcription/translation solution can enhance efficiency by avoiding the use of in vitro transcription/translation solution on coding substrates that do not encode antimicrobial peptides and/or bacteriocins of the specified mixture. In some embodiments, the microfluidic device is part of a cartridge 591 that does not comprise a reservoir of in vitro transcription/translation solution. The cartridge 591 can be configured to place the microfluidic device in data communication with a system comprising a processor 595. Optionally the cartridge can also be placed in fluid communication with the system. In some embodiments, the microfluidic device is part of a cartridge 592 that comprises a reservoir of in vitro transcription/translation solution 530. The cartridge 592 can be configured to place the microfluidic device in data communication with a system comprising a processor 595. In some embodiments, the microfluidic device comprises, consists essentially of, or consists of the microfluidic device 600 shown in FIG. 6, which includes discrete coding substrates 610a, 610b, 610c, a channel 660, and valves 620a, 620b, 620c, 670a, 670b, 670c, and/or 680, similar to the microfluidic device 500 in FIG. 5. The microfluidic device optionally does not include In some embodiments, the microfluidic device 600 can be placed in fluid communication with a system comprising a reservoir of in vitro transcription/translation solution 530 or a fluidic reservoir 540, or in data communication with a heating element 555. For example, a disposable cartridge can comprise, consist essentially of, or consist of the microfluidic device 600, and can be configured to be placed in fluid communication with a system comprising in vitro transcription/translation solution 530 and a fluidic reservoir 540. The microfluidic device 600 can also be placed in data communication with a processor 595 and/or a heating element 555 of the system. In some embodiments, the microfluidic device is for producing a specified mixture of bacteriocins, and not antimicrobial peptides. In some embodiments, the microfluidic device is for producing a specified mixture of antimicrobial peptides, and not bacteriocins. In some embodiments, the microfluidic device is for producing a specified mixture of bacteriocins and antimicrobial peptides.

In some embodiments, valves 520a, 520b, and 520c regulate flow between the discrete coding substrates 510a, 510b, 510c and the fluidic reservoir 540. Valves 570a, 570b, and 570c are optional, and/or valves 550 and 580 are optional, and/or the channel 560 is optional. In some embodiments, the reservoir of in vitro transcription/translation solution 530 can be in fluidic communication with the fluidic reservoir 540. Optionally, valve 550 is disposed between the reservoir of in vitro transcription/translation solution 530 and the fluidic reservoir 540, and regulates flow of in vitro transcription/translation solution to the fluidic reservoir 540. In some embodiments, the microfluidic device 600 of FIG. 6 is part of a cartridge.

In some embodiments, for example if only the antimicrobial peptides and/or bacteriocins of the specified mixture are permitted to enter the fluidic reservoir 540 from the coding substrates 510a, 510b, 510c, valves 570a, 570b, and 570c regulate flow between a reservoir of in vitro transcription/translation solution 530 and the discrete coding substrates 510a, 510b, 510c, and valves 520a, 520b, 520c are optional, and/or valve 550 is optional and/or valve 580 is optional. A channel 560 can connect the reservoir of in vitro transcription/translation solution 530 to the discrete coding substrates 510a, 510b, 510c, with optional intervening valve 580 between the reservoir 530 and the channel 560, and/or optional intervening valves 570a, 570b, 570c between the channel 560 and the discrete coding substrates 510a, 510b, 510c. In some embodiments, valves 570a, 570b, and 570c regulate flow between a reservoir of in vitro transcription/translation solution 530 and the discrete coding substrates 510a, 510b, 510c, and valves 520a, 520b, 520c regulate flow between the discrete coding substrates 510a, 510b, 510c and the fluidic reservoir 540. Optionally, channel 560 can connect the reservoir 530 to the discrete coding substrates 510a, 510b, 510c. Valve 580 is optional and/or valve 550 is optional.

The microfluidic device can further comprise an outlet 590. The outlet 590 can place the fluidic reservoir (and any mixture of antimicrobial peptides and/or bacteriocins therein) in fluidic communication with a tissue of a subject, a wound, and/or a microbiome of a subject as described herein. In some embodiments, the outlet 590 comprises a valve. In some embodiments, the outlet 590 can be placed in fluid communication with a vessel for storing a specified mixture of antimicrobial peptides and/or bacteriocins, for example a test tube, bag, or well.

In some embodiments, the in vitro transcription/translation solution reservoir 530 is in fluid communication with channel 560. Optional valve 580 controls from from the reservoir 530 to the channel 560. In some embodiments, valves 570a, 570b, and 570c regulate flow of transcription/translation solution from the channel 560 into discrete coding substrates 510a, 510b, 510c. By way of example, to produce a specified mixture of antimicrobial peptides and/or bacteriocins comprising antimicrobial peptides and/or bacteriocins encoded by discrete coding substrates 510a and 510b (but not 510c), valves 570a and 570b can be open, while valve 570c is closed, to permit flow of transcription/translation solution 530 into discrete coding substrates 510a and 510b, but not 510c. In some embodiments, discrete coding substrates 510a, 510b, and/or 510c are incubated with the in vitro transcription/translation solution to produce a specified mixture of antimicrobial peptides and/or bacteriocins that is released via valves 520a, 520b, and/or 520c into the fluidic reservoir 540.

In some embodiments, in vitro transcription/translation solution flows to the discrete coding substrates 510a, 510b, and/or 510c through the fluidic reservoir 540. The reservoir of in vitro transcription/translation solution 530 can be in fluid communication with the fluidic reservoir 540, optionally with flow regulated by intervening valve 550. Optionally, valves 520a, 520b, 520c regulate flow from the fluidic reservoir 540 to the discrete coding substrates 510a, 510b, and 510c. As such, the in vitro transcription/translation solution can be incubated with the appropriate discrete coding substrates to produce antimicrobial peptides and/or bacteriocins, and antimicrobial peptides and/or bacteriocins can then flow through valves 520a, 520b, and 520c to the fluidic reservoir 540. Valves 570a, 570b, 570c, the channel 560, and valve 580 can be optional. In some embodiments, discrete coding substrates 510a, 510b, and/or 510c are incubated with the in vitro transcription/translation solution to produce a specified mixture of antimicrobial peptides and/or bacteriocins that is released via valves 520a, 520b, and/or 520c into the fluidic reservoir 540. In some embodiments, the specified mixture of antimicrobial peptides and/or bacteriocins flows through the outlet 590 to a wound, tissue, microbiome of a subject, and/or vessel as described herein.

In some embodiments, valve 550 regulates flow from the in vitro transcription/translation solution reservoir 530 into fluidic reservoir 540, and valves 520a, 520b, and/or 520c regulate flow from discrete coding substrates 510a, 510b, and/or 510c to fluidic reservoir 540. Valves 570a, 570b, 570c, and 580, and channel 560 are optional. As such, in vitro transcription/translation solution can flow from the fluidic reservoir 540 to the discrete coding substrates 510a, 510b, and/or 510c, can be incubated with the discrete coding substrates 510a, 510b, and/or 510c to produce antimicrobial peptides and/or bacteriocins, and antimicrobial peptides and/or bacteriocins can flow to the fluidic reservoir 540. In some embodiments, the processor 595 regulates flow of in vitro transcription/translation solution through valves 520a, 520b, and 520c to the discrete coding substrates 510a, 510b, and/or 510c so that only discrete coding substrates encoding antimicrobial peptides and/or bacteriocins of the specified mixture receive in vitro transcription translation/solution. In some embodiments, the processor 595 permits flow of in vitro transcription translation/solution from the fluidic reservoir 540 to the discrete coding substrates 510a, 510b, and/or 510c encoding the antimicrobial peptides and/or bacteriocins of the specified mixture through valves 520a, 520b, and 520c, and after the antimicrobial peptides and/or bacteriocins have ben produced by in vitro transcription translation, the processor 595 regulates valves 520a, 520b, 520c, so that only the antimicrobial peptides and/or bacteriocins of the specified mixture to enter the fluidic reservoir 540.

It can be appreciated that in some embodiments of the fluidic device 500 depicted in FIG. 5, valves 550, 570a, 570b, 570c, 580, and channel 560, are optional. For example, some embodiments of the fluidic device include valves 580, 570a, 570b and 570c, and flow means 560, but not valve 550. In some other embodiments, the fluidic device includes valve 550, but not valves 570a, 570b, 570c, or 580, or flow means 560. In some embodiments, the fluidic device includes valves 550, 570a, 570b, 570c, and 580, and flow means 560. In some embodiments, valve 580 is included, and valves 570a, 570b, and 570c are excluded. In some embodiments, valve 580 is excluded, and valves 570a, 570b, and 570c are included. It can also be appreciated that in some embodiments of the fluidic device 500, the outlet 590 does not comprise a valve.

In some embodiments, the outlet 590 permits a wash fluid into the fluidic reservoir 540, and any of the other valves may be opened to permit flow of the wash fluid into any other fluidically connected portion of the device. In some embodiments, the wash fluid comprises a buffer or a detergent. It can be appreciated that additional valves may be present in the microfluidic device to allow the inflow or outflow of fluids in any of the fluidic portions of the device.

In some embodiments, channel 560 comprises, consists essentially of, or consists of a microchannel, tube, pipe, and a hose, and as such, can contain and/or direct flow of a fluid. In some embodiments, the channel 560 comprises material such as rubber, plastic, metal.

In accordance with the methods and microfluidic devices of some embodiments described herein, the microfluidic device is portable. For example, some embodiments of the device may be removed from a laboratory and taken into a natural environment where a scientist performs tests with various mixtures of specified antimicrobial peptides and/or bacteriocins.

In accordance with the methods and microfluidic devices of some embodiments described herein, the microfluidic device comprises an outlet 590 through which the specified mixture of antimicrobial peptides and/or bacteriocins are delivered to a wound. For example, the outlet 590 can be connected to the wound via a membrane or tube. Thus, in some embodiments, the device comprises a wound dressing or wound cleaning device.

In some embodiments, fluid is passively transferred from one component of the device to another. The microfluidic devices of some embodiments include a pump 565 that actively pumps fluid from one component of the device to another.

In accordance with the methods and microfluidic devices of some embodiments described herein, the microfluidic device comprises a heating element 555 such as a heating block. In some embodiments, the heating element is configured to heat parts of the microfluidic device, such as the fluidic reservoir 540 and/or the discrete coding substrates 510a, 510b, and/or 510c to perform incubation step. In some embodiments, the incubation step is at at least about 0° C., 4° C., 25° C., 30° C., 37° C., 38° C., 40° C., including ranges between any two of the listed values, for example 0-40° C., or 36-38° C., or 4-40° C. In some embodiments, the incubation with the in vitro transcription/translation solution at least 1, 10, 30, 60 second, or at least 2, 3, 4, 5, 6, 7, 8, 9, 10, 15, 20, 25, or 30 minutes, including ranges between any two of the listed valued, for example 1-30 seconds, 30-60 seconds, 1-2 minutes, 2-5 minutes, 5-10 minutes, 10-15 minutes, 15-30 minutes, or 30-60 minutes, 1-2 hours, 2-5 hours, 5-10 hours, 10-15 hours, 15-24 hours, 24-48 hours, or 48-72 hours. In some embodiments, the incubation with the in vitro transcription/translation solution comprises more than one temperature at different times.

In accordance with the methods and microfluidic devices of some embodiments described herein, the microfluidic device comprises a reservoir of chemical and/or phage antibiotics configured to mix with the mixture of specified antimicrobial peptides and/or bacteriocins. In some embodiments, the microfluidic device comprises an antibiotic comprising a chemical antibiotic and/or a phage. In some embodiments, the system comprises a reservoir of chemical or phage antibiotics configured to mix with the mixture of specified antimicrobial peptides and/or bacteriocins. In accordance with the methods and microfluidic devices of some embodiments described herein, the microfluidic device comprises an antibiotic comprising a chemical antibiotic and/or a phage.

### Fluidic Reservoirs

In methods and microfluidic devices of some embodiments herein, the microfluidic device comprise a fluidic reservoir 540. In some embodiments, the fluidic reservoir is or is configured to be in fluid communication with at least one discrete coding substrate (optionally, separated by a valve as described herein). In some embodiments, the fluidic reservoir is in fluid communication with discrete coding substrates or chambers housing the discrete coding substrates, as described herein. In some embodiments, the fluidic reservoir is placed in fluid communication with the discrete coding substrates (or chambers) by a channel, tubing, microfluidic tubing, membrane, mesh, opening, passageway, or two or more of these. By way of example, the channel, tubing, microfluidic tubing, membrane, mesh, opening, and/or passageway of some embodiments can comprise a material such as rubber, glass, plastic, metal, an organic compound, or two or more of these. In some embodiments, the fluidic reservoir is configured to receive antimicrobial peptides and/or bacteriocins of the specified mixture of antimicrobial peptides and/or bacteriocins. The specified antimicrobial peptides and/or bacteriocins can be mixed in the fluidic reservoir, forming the mixture, for example by passive mixing and/or by active mixing. In some embodiments, the microfluidic device comprises more than one fluidic reservoirs. In some embodiments, the fluidic reservoir has a volume of at least 1 μl, for example, at least 1, 5, 10, 100, 500, or 1000 μl, including ranges between any two of the listed values, for example, 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, 500 μl-1000 μl, 1 ml-1000 ml, 1 ml-50 ml, 1 ml-500 ml, 1 ml-900 ml, 50 ml-100 ml, 50 ml-500 ml, 50 ml-1000 ml, 100 ml-200 ml, 100 ml-500 ml, 100 ml-1000 ml, 200 ml-500 ml, 200 ml-1000 ml, 500 ml-900 ml, 500 ml-1000 ml.

### Valves

In methods and microfluidic devices of some embodiments, the microfluidic device comprise one or more valves. As used herein, “valve” encompasses mechanical valves, as well as electromagnetic fields, conditional diffusion membranes, and other devices understood in the art to permit and restrict liquid flow. In some embodiments, a valve separates the fluidic reservoir from a discrete coding substrate, and controls the flow of fluids between the discrete coding substrate and fluidic reservoir. Each valve may be disposed on a fluidic path between a discrete coding substrate and the fluidic reservoir. Each valve may be configured to regulate flow between the discrete coding substrate and the fluidic reservoir. In some embodiments, each discrete coding substrate is separated from the fluidic reservoir by a valve configured to control the flow of fluid from the discrete coding substrate to the fluidic reservoir. In some embodiments, a valve as described herein comprises a hydraulic, pneumatic, manual, solenoid, motor valve, or socket ball valve, or a combination of two or more of these. In some embodiments, a valve as described herein comprises a two-port, three-port, or four-port valve. In some embodiments, a valve as described herein comprises a microfluidic valve or microvalve, such as a solenoid microvalve, screw microvalve, pneumatic microvalve, or a combination of two or more of these. In some embodiments, a valve is binary, so that flow through the valve is either “on” or “off.” In some embodiments, a valve regulates the rate of flow though the valve.

In some embodiments, valves 520a, 520b, 520c are disposed between the fluidic reservoir 540 and the coding substrates 510a, 510b, 510c. For example, in some embodiments, valves are disposed between the fluidic reservoir and the coding substrates, and between the coding substrates and the fluidic reservoir. A single valve between the fluidic reservoir and the coding substrates may be opened to flush some or all of the coding substrates with a transcription/translation fluid, and then only valves between coding substrates of interest and the reservoir are opened after incubation, so that only antimicrobial peptides and/or bacteriocins of interest are included in a specified mixture of antimicrobial peptides and/or bacteriocins are permitted to flow into the fluidic reservoir.

In some embodiments, separate valves are disposed between the transcription/translation solution and the discrete coding substrates, so that separate valves can be opened and closed to place only discrete coding substrates of interest in contact with the transcription/translation solution, and thus produce a specified antimicrobial peptides and/or bacteriocins mixture upon incubation of the discrete coding substrates of interest with the transcription/translation solution. In view of this disclosure, the skilled artisan will appreciate that there are multiple ways for valves to regulate flow to and from the discrete coding substrates so that only antimicrobial peptides and/or bacteriocins of the specified mixture of antimicrobial peptides and/or bacteriocins are obtained in a mixture in the fluidic reservoir 540.

Smart bandages can comprise a processor, a sensor (such as a pH and/or temperature sensor), and a source of therapeutic agent, in which the processor is configured to control the administration of therapeutic agent. For example, the sensor can detect inflammation, and the processor can administer quantities of anti-inflammatory agent in response to the detected inflammation. Examples of suitable smart bandages may be found on the world wide web at www.cnet.com/news/this-smart-bandage-can-deliver-drugs-monitor-chronic-wounds, which is hereby incorporated by reference in its entirety. In some embodiments, the microfluidic device as described herein comprises a smart bandage. In some embodiments, the microfluidic device or system is configured to be placed in fluid communication with a smart bandage. For example, the port of the microfluidic device can be placed in fluid communication with a reservoir of a smart bandage, so that the smart bandage can control delivery of the specified mixture of antimicrobial peptides and/or bacteriocins to a subject. In some embodiments, the microfluidic device is comprised by a disposable cartridge that further comprises a smart bandage, and is in fluid communication with the fluidic reservoir, so that a specified mixture of antimicrobial peptides and/or bacteriocins can be delivered to a subject via the smart bandage. Optionally, the disposable cartridge comprising the smart bandage and the microfluidic device comprises an adhesive for direct application to a tissue of a subject, for example skin. As such, the microfluidic device of some embodiments is for medical use. In some embodiments, the smart bandage monitors a wound or site of inflammation and delivers a specified mixture of antimicrobial peptides and/or bacteriocins as described herein to the skin or to a wound site.

### Processors

A processor 595 can regulate valves in microfluidic devices and methods of some embodiments. The processor can be configured to, based on the specified mixture of antimicrobial peptides and/or bacteriocins, configure the valves to place the discrete coding substrates that encode the antimicrobial peptides and/or bacteriocins of the mixture, but not other discrete coding substrates, in fluidic communication with the fluidic reservoir. In some embodiments, the processor is configured to permit incubation of the in vitro transcription/translation solution with the discrete coding substrates that encode the antimicrobial peptides and/or bacteriocins of the mixture, so that the specified antimicrobial peptides and/or bacteriocins of the mixture are produced. In some embodiments, the processor is configured to permit flow of the specified antimicrobial peptides and/or bacteriocins through the valves into the fluidic reservoir. In some embodiments, the processor is configured to control flow of fluid in the fluidic reservoir. In some embodiments, the flow comprises movement of a fluid comprising the specified antimicrobial peptides and/or bacteriocins into and/or within the fluidic reservoir, thus producing the specified mixture of antimicrobial peptides and/or bacteriocins. For example, the flow can mix bacteriocins from different coding substrates once they are present in the fluidic reservoir, thus producing the specified mixture of antimicrobial peptides and/or bacteriocins. In some embodiments, the microfluidic device comprises a processor. In some embodiments, the microfluidic device does not itself comprise a processor, but is configured to be placed in data communication with the processor.

It is contemplated that a specified mixture of antimicrobial peptides and/or bacteriocins according to methods and microfluidic devices of some embodiments herein can be formed from two or more submixtures. For example, a first submixture of bacteriocins A and B in a ratio of 1:1 can be combined with the same quantity of a second submixture of bacteriocins B and C in a ratio of 1:1, thus producing a mixture of bacteriocins A, B, and C in ratios of 1:2:1. In accordance with the methods and microfluidic devices of some embodiments described herein, the specified mixture of antimicrobial peptides and/or bacteriocins comprises two or more submixtures each comprising a subset of antimicrobial peptides and/or bacteriocins, and the processor is configured to permit flow of each submixture into the fluidic reservoir, thereby producing the specified mixture of antimicrobial peptides and/or bacteriocins. In some embodiments, the specified mixture of antimicrobial peptides and/or bacteriocins comprises a sum of the submixtures of bacteriocins in a specified stoichiometry, and combination of the submixtures yields the mixture in the specified stoichiometry.

Methods for Producing a Specified Mixture of Antimicrobial Peptides and/or Bacteriocins

In some embodiments, a method for producing a specified mixture of antimicrobial peptides and/or bacteriocins is provided. The method may include producing bacteriocins of the specified mixture, but not other bacteriocins, by in vitro transcription and translation of bacteriocins encoded by discrete coding substrates, and then mixing the selected bacteriocins from the in vitro transcription and translation to produce the specified mixture of antimicrobial peptides and/or bacteriocins. In some embodiments, the method includes producing bacteriocins by in vitro transcription and translation of bacteriocins encoded by discrete coding substrates, and then mixing bacteriocins of the specified mixture, but not other bacteriocins (if any) to produce the specified mixture of antimicrobial peptides and/or bacteriocins. In some embodiments, the method for producing a specified mixture of antimicrobial peptides and/or bacteriocins is performed on a microfluidic device as described herein. In some embodiments, the method is for producing a specified mixture of bacteriocins, and not antimicrobial peptides. In some embodiments, the method is for producing a specified mixture of antimicrobial peptides, and not bacteriocins. In some embodiments, the method is for producing a specified mixture of bacteriocins and antimicrobial peptides.

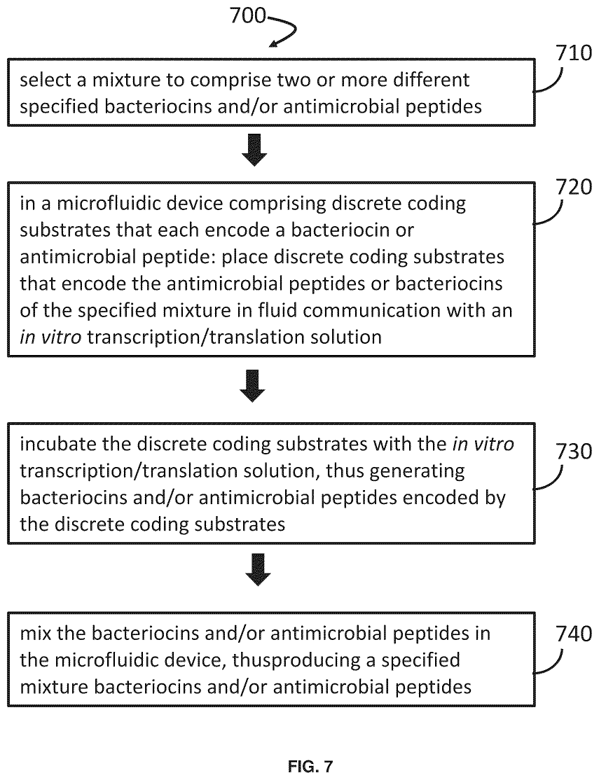

FIG. 7 is a flow diagram illustrating a method for producing a specified mixture of antimicrobial peptides and/or bacteriocins 700 of some embodiments. The method can comprise selecting the specified mixture to comprise two or more different specified bacteriocins 710; in a microfluidic device comprising discrete coding substrates that each encode an antimicrobial peptide and/or bacteriocin: placing discrete coding substrates that encode the antimicrobial peptides and/or bacteriocins of the mixture, but not other discrete coding substrates, in fluid communication with an in vitro transcription/translation solution 720; incubating the discrete coding substrates with the in vitro transcription/translation solution, thus generating antimicrobial peptides and/or bacteriocins encoded by the discrete coding substrates 730; and mixing the bacteriocins in the microfluidic device, thus producing the specified mixture of antimicrobial peptides and/or bacteriocins 740.

In some embodiments, the method of producing a specified mixture of antimicrobial peptides and/or bacteriocins further comprises producing two or more submixtures each comprising a subset of the specified mixture of antimicrobial peptides and/or bacteriocins, and combining the submixtures to produce the specified mixture of antimicrobial peptides and/or bacteriocins. For example, the method can comprise performing (a) through (d) as follows to produce a first specified submixture of antimicrobial peptides and/or bacteriocins: (a) selecting a submixture to comprise two or more different specified antimicrobial peptides and/or bacteriocins; (b) in a microfluidic device comprising discrete coding substrates that each encode an antimicrobial peptide and/or bacteriocin: placing discrete coding substrates that encode the antimicrobial peptides and/or bacteriocins of the submixture, but not other discrete coding substrates, in fluid communication with an in vitro transcription/translation solution; (c) incubating the discrete coding substrates with the in vitro transcription/translation solution, thereby generating antimicrobial peptides and/or bacteriocins encoded by the discrete coding substrates; (d) mixing the antimicrobial peptides and/or bacteriocins in the microfluidic device, thereby producing the specified submixture of antimicrobial peptides and/or bacteriocins; repeating (a) through (d) to produce a second specified submixture of antimicrobial peptides and/or bacteriocins; and combining the first specified submixture of antimicrobial peptides and/or bacteriocins the second specified submixture of antimicrobial peptides and/or bacteriocins to produce the specified mixture of antimicrobial peptides and/or bacteriocins.

In some embodiments of the method of producing a specified mixture of antimicrobial peptides and/or bacteriocins, the selecting further comprises selecting a stoichiometry of the two or more different specified antimicrobial peptides and/or bacteriocins. In some embodiments, the specified mixture of antimicrobial peptides and/or bacteriocins comprises a specified stoichiometry, and combining the submixtures results in the specified stoichiometry. For example, a first submixture of bacteriocin “A” can be combined with a second submixture of bacteriocins “A” and “B” in a ratio of 1:1, to produce a specified mixture of antimicrobial peptides and/or bacteriocins “A” and “B” in a ratio of 2:1.

In some embodiments of the method of producing a specified mixture of antimicrobial peptides and/or bacteriocins, incubating the discrete coding substrates with the in vitro transcription/translation solution comprises flowing the in vitro transcription/translation solution to each discrete coding substrate encoding a bacteriocin of the mixture. In some embodiments, the method comprises placing the discrete coding substrates that encode the antimicrobial peptides and/or bacteriocins of the mixture, but not other discrete coding substrates, in fluid communication with an in vitro transcription/translation solution, comprising (i) opening valves so as to place a source of the in vitro transcription/translation solution in fluid communication with the discrete coding substrates; (ii) closing valves so as to inhibit fluid communication between the source of the in vitro transcription/translation solution and the other discrete coding substrates, or a combination of (i) and (ii). In some embodiments, mixing the antimicrobial peptides and/or bacteriocins in the microfluidic device comprises opening a valve to place the discrete coding substrates in fluid communication with a fluidic reservoir, wherein the antimicrobial peptides and/or bacteriocins are mixed in the fluidic reservoir.

In some embodiments, the method of producing a specified mixture of antimicrobial peptides and/or bacteriocins further comprises screening the mixture of antimicrobial peptides and/or bacteriocins in situ for a desired effect. In some embodiments, the screening is for inhibition of the growth or reproduction of a pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract. For example, the pathogenic bacteria can be a rapidly evolving bacteria or a bacteria exhibiting antibiotic resistance, for example MRSA. In some embodiments, the screening is for an absence of deleterious effects of the mixture of antimicrobial peptides and/or bacteriocins on a microbiome of a subject, such as a skin, gut, ovarian follicle, lung, saliva, oral cavity, mucosa, conjunctiva, or biliary tract. In some embodiments, the screening is performed in real time. For example, the screening can be performed within 120 minutes, 60 minutes, 30 minutes, 15 minutes, 10 minutes, 5 minute, or 1 minute of the specified mixture of antimicrobial peptides and/or bacteriocins being generated. In some embodiments, the screening is for stabilization of a antimicrobial peptides and/or bacteriocins or for destruction of a microbial biofilm. In some embodiments, the screening is for enhancement of growth or reproduction of a non-pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract.

Some microbial organisms can evolve quickly. Advantageously, the methods, devices and systems described herein can be used to combat rapidly evolving microbial organisms by rapidly producing mixtures of specified bacteriocins that are tailored to an infection of a subject by the microbial organism. In some embodiments, the subject is infected with a staphylococcus such as MRSA, influenza virus, West Nile virus, or Zika virus. In some embodiments, the subject is diabetic and/or has an infection in an extremity such as a hand or foot.

Also envisioned is the use of antimicrobial peptides and/or bacteriocins in conjunction with conventional chemical antibiotics and/or phage antibiotics. In some embodiments, a method as described herein includes treatment of a microbial infection with a combination of a mixture of specified antimicrobial peptides and/or bacteriocins and a chemical antibiotic and/or a phage. Some embodiments comprise delivering the mixture of specified antimicrobial peptides and/or bacteriocins, in combination with a chemical antibiotic and/or a phage antibiotic, to a subject. Moreover, in some embodiments, a microfluidic device and/or system as described herein further comprises a conventional antibiotic, for example, an phage antibiotic or a small-molecule antibiotic such as a metabolite. In some embodiments, the microfluidic device and/or system further comprises a reservoir of antibiotic. The antibiotic can be selected from the group consisting of Amoxicillin, Amoxicillin/clavulanic acid (amoxicillin+clavulanic acid), Ampicillin, Benzathine benzylpenicillin, Benzylpenicillin, Cefalexin, Cefazolin, Cefixime, Cefotaxime, Ceftriaxone, Cloxacillin, Penicillin, Phenoxymethylpenicillin (penicillin V), Piperacillin/tazobactam, Procaine benzylpenicillin, Ceftazidimea, Meropenema, Aztreonama, Imipenem/cilastatin, Amikacin, Azithromycin [, Chloramphenicol, Ciprofloxacin, Clarithromycin, Clindamycin, Doxycycline, Erythromycin, Gentamicin, Metronidazole, Nitrofurantoin, Spectinomycin, Trimethoprim/sulfamethoxazole, Trimethoprim, Vancomycin, Clofazimine, Dapsone, Rifampicin, Ethambutol/isoniazid, Ethambutol/isoniazid/pyrazinamide/rifampicin, Ethambutol/isoniazid/rifampicin, Isoniazid, Isoniazid/pyrazinamide/rifampicin, Isoniazid/rifampicin, Pyrazinamide, Rifabutin, Rifampicin, Rifapentine, Amikacin, Bedaquiline, Capreomycin, Clofazimine, Cycloserine, Delamanid, Ethionamide, Kanamycin, Levofloxacin, Linezolid, Moxifloxacin, p-aminosalicylic acid, rifabutin, rifapentine, rifalazil, rifaximin. Streptomycin, a phage, or a combination of two or more of these antibiotics.

Some embodiments of the method of producing a specified mixture of antimicrobial peptides and/or bacteriocins further comprise delivering the specified mixture of antimicrobial peptides and/or bacteriocins to a wound via a tubing or membrane, thereby cleaning or dressing the wound. For example, the specified mixture of antimicrobial peptides and/or bacteriocins may be placed on a cut or open sore, and then the cut or sore may be closed and/or bandaged.

Some embodiments of the method of producing a specified mixture of antimicrobial peptides and/or bacteriocins include a wash of the microfluidic device. The wash can be with a wash fluid. For example, some embodiments include an incubation step with a transcription solution, followed by a wash step, followed by a separate incubation with a translation solution.

In some embodiments, the method further comprises administering the specified mixture of antimicrobial peptides and/or bacteriocins to a subject in need of treatment. For example, the subject can have an infection such as an infected wound, a surgical incision, and infection of the extremities associated with diabetes, and/or a biofilm. The specified mixture of antimicrobial peptides and/or bacteriocins can be selected to target the microbial organism(s) of the infection (or selected as a candidate to target the microbial organism(s) of the infection), and can be produced in a microfluidic device as described herein. The specified mixture of antimicrobial peptides and/or bacteriocins can be administered to the subject as described herein, for example at or near the site of infection. In some embodiments, the microfluidic device itself is directly applied to the infection (for example, as a smart bandage as described herein). In some embodiments, the method further comprises selecting the subject as having an infection in need of treatment. The method can further comprise selecting a specified mixture of antimicrobial peptides and/or bacteriocins to target the infection, and producing the specified mixture in a microfluidic device as described herein. Also contemplated are medical uses of the microfluidic device for treating, inhibiting, preventing and/or reducing the risk of an infection. In some embodiments, the method (or medical use) comprises administering the specified mixture of antimicrobial peptides and/or bacteriocins and an antibiotic as described herein (for example a small molecule antibiotic and/or a phage) to the subject.

In some embodiments, the microfluidic device is for veterinary use, for example to produce a specified mixture of antimicrobial peptides and/or bacteriocins to treat an infection of a domestic animal or a farm animal, and/or to promote growth of the animal. Advantageously, uses of the specified mixture of antimicrobial peptides and/or bacteriocins in farm animals can avoid the use of antibiotics in food production. As noted in World Health Organization recommendations, doing so can reduce the development and proliferation of antibiotic-resistant microbial o.

In some embodiments, the microfluidic device is for use in a small fermenter (e.g., a fermenter less than or equal to 100 liters, 50 liters, 40 liters, 30 liters, 20 liters, 10 liters, 5 liters, 2 liters, or 1 liters in volume, including ranges between any two of the listed values), for example to produce specified mixtures of bacteriocins to target a contaminating organism in the small fermenter.

In some embodiments, the microfluidic device is for use in sterilizing medical devices. For example, the specified mixture of antimicrobial peptides and/or bacteriocins can be selected to target contaminants, such as MRSA on the medical device, and can be applied to the medical device on-site, for example at a hospital. In some embodiments, the microfluidic device is for use in pre-treating an implant with a specified mixture of antimicrobial peptides and/or bacteriocins, so as to inhibit or prevent infection and/or biofilm formation.

In some embodiments, the microfluidic device is for use in defense against a pathogen, for example in an epidemic, or in bioterrorism defense. Specified mixtures of bacteriocins directed against the pathogen (e.g., bioterrorism agent) can be distributed to systems as described herein manually or automatically, and the specified mixtures of bacteriocins targeting the pathogen (e.g., bioterrorism agent) can be produced in microfluidic devices at or near the site of the pathogen (e.g., bioterrorism agent).

In some embodiments, the method further comprises mixing the specified mixture of antimicrobial peptides and/or bacteriocins with another reagent or antimicrobial compound to produce a final mixture. For example, the specified mixture of antimicrobial peptides and/or bacteriocins may be combined with a chemical or a natural fluid. In some embodiments, the specified mixture of antimicrobial peptides and/or bacteriocins is combined with an antibiotic drug or phage to produce the final mixture. In some embodiments, the final mixture is part of a formulation for a therapy such as, for example, an anti-pain therapy.

### Example 1

A nucleic acid is provided encoding one copy of the coding sequence of the bacteriocin Subtilin, two copies of the coding sequence of the bacteriocin Bavaricin-MN, and one copy of the coding sequence for the quorum sensing factor BsEDF all in a single reading frame. The nucleic acid further encodes cleavage sites for Caspase 2 in the same reading frame, which flank each bacteriocin coding sequence and the BsEDF coding sequence. As such, the nucleic acid encodes a pro-polypeptide that comprises one copy of Subtilin, two copies of Bavaricin-MN, and one copy of BsEDF, each flanked by Caspase 2 cleavage sites. The nucleic acid is expressed in an E. coli cell, which does not comprise coding sequence for the immunity modulators for either of Subtilosin-A or Bavaricin-MN, and thus does not produce functional immunity modulators for these bacteriocins. The pro-polypeptide is produced by the E. coli, and contains Subtilin and Bavaricin-MN in their inactive forms. The pro-polypeptide is purified by way of its His tag using a nickel-containing substrate. The purified pro-polypeptide is then cleaved using Caspase 2, producing a composition that comprises Subtilin, Bavaricin-MN, and BsEDF in a 1:2:1 ratio. The composition is added to an industrial feedstock to prevent the proliferation of undesired microbial organisms (via the bacteriocins), and to control the growth of genetically modified B. subtilis (via the BsEDF).

### Example 2

It is determined that a ratio of the bacteriocins Mundticin, Serracin-P, Thuricin-17, and Plantaricin J of 1:2:3:4 is useful for targeting a population of undesired microbial cells in animal food during storage. A nucleic acid encoding one copy of the coding sequence of Mundticin, two copies of the coding sequence of Serracin-P, three copies of the coding sequence of Thuricin-17, and four copies of the coding sequence Plantaricin J is synthesized. All of the bacteriocin coding sequences are in a single reading frame, and each bacteriocin coding sequence is separated from the other bacteriocin coding sequences by sequences encoding Granzyme B cleavage sites. The nucleic acid also encodes GST on the 3′ end of the open reading frame, and immediately downstream of a Granzyme B cleavage site coding sequence. Thus, the nucleic acid encodes a pro-peptide comprising Mundticin, Serracin-P, Thuricin-17, Plantaricin J in a 4:3:2:1 ratio, respectively, along with a C-terminal GST tag. The nucleic acid is expressed in an in vitro translation system, so as to produce the pro-polypeptide. The pro-polypeptide is purified using GSH-coated beads. The pro-polypeptide is then cleaved by GranzymeB, producing a solution comprising active Mundticin, Serracin-P, Thuricin-17, and Plantaricin J in a ratio of 1:2:3:4, respectively. The cleavage site immediately adjacent to the GST tag is also cleaved, so that none of the bacteriocins comprises GST. The composition comprising the bacteriocins in the 1:2:3:4 ratio is added to the animal food, thus targeting the population of undesired microbial cells in the animal food.

### Example 3

A microfluidic device is provided that comprises 244 chambers. Each chamber comprises a discrete coding substrate. Each discrete coding substrate is a chip, each chip comprising 10,000-20,000 DNAs. Molecules, each encoding a bacteriocin of Table 1.2. The DNA molecules in each chamber encode separate bacteriocins compared to the DNA molecules in each of the other chambers. Each of the chambers is connected by a valve to a fluidic reservoir. The fluidic reservoir is also connected by a valve to a transcription/translation solution housed in a transcription/translation solution chamber. The device includes a processor configured to open the valve connecting the fluidic reservoir to the transcription/translation solution to allow the transcription/translation solution to flow into the fluidic reservoir, close the valve connecting the fluidic reservoir to the transcription/translation solution to prevent fluid from flowing back into the transcription/translation solution chamber, open valves connecting the fluidic reservoir to chambers housing the selected coding substrates to allow transcription/translation solution to flow from the fluidic reservoir into the chambers housing the selected coding substrates, incubate selected coding substrates the at 37° C. thereby producing bacteriocins from the selected coding substrates, and open one or more valves such as the valves connecting the coding substrates to the fluidic reservoir, to release bacteriocins into the fluidic reservoir and/or a receptacle.

The device is connected electronically or wirelessly to a user input device such as a phone, touchscreen, keyboard, button, mouse, or computer. The processor selects bacteriocins based on user input entered into the user input device, or according to a pre-programmed set of instructions.

### Example 4

A device as described herein is used to make a first specified mixture of bacteriocins. The bacteriocins are applied to a transdermal patch that is applied to a subject with a burn. If the burn on the subject does not heal, the device is used to make a second specified mixture of bacteriocins different from the first specified mixture of bacteriocins. The second specified mixture of bacteriocins is then applied to a new transdermal patch that is applied to the burn. Multiple variations of specified mixtures of bacteriocins may be applied to the burn, or to burns of various subjects, depending on whether a particular formulation of bacteriocins is expected to be beneficial for the healing of the burn, or on whether a particular formulation is desired to be tested.

### Options

In addition to the items above, the following options are set forth:
1. A method of making bacteriocins, the method comprising expressing a nucleic acid comprising:
   a bacteriocin coding sequence and a second polypeptide coding sequence in a single reading frame, wherein the second polypeptide is a bacteriocin or signal molecule; and
   cleavage site coding sequences disposed between the bacteriocin coding sequence and the second polypeptide coding sequence in the single reading frame,
thereby generating a pro-polypeptide comprising the bacteriocin, second polypeptide, and cleavage sites disposed therebetween.
2. The method of option 1, further comprising cleaving the cleavage site, thereby separating the bacteriocin and second polypeptide from each other, and thereby producing a composition comprising the bacteriocin and the second polypeptide.
3. The method of any one of options 1 or 2, wherein the second polypeptide is the bacteriocin.
4. The method of any one of options 1 or 2, wherein the second polypeptide is the signal molecule.
5. The method of any one of options 1-4, wherein the expressing is performed by a microbial cell that does not produce a functional immunity modulator for at least one of the bacteriocins.
6. The method of option 5, wherein the microbial cell does not produce a functional immunity modulator for any of the bacteriocins.
7. The method of any one of options 1-4, wherein the expressing is performed in vitro.
8. The method of any one of options 1-7, wherein at least one of the bacteriocins is inactive when it is part of the pro-polypeptide.
9. The method of any one of options 2-8, further comprising isolating the pro-polypeptide prior to the cleaving.
10. The method of option 9, wherein said isolating comprises affinity purifying the pro-polypeptide, wherein the affinity purification comprises binding an affinity tag encoded by the nucleic acid.
11. The method of any one of options 1-8, wherein the nucleic acid comprises three bacteriocin coding sequences in the single reading frame.
12. The method of any one of options 1-3 or 5-11, wherein at least two of the bacteriocins are different from each other.
13. The method of any one of options 2-12, wherein the composition comprises a desired ratio of bacteriocins, or a desired ratio of signal molecules and bacteriocins.
14. The method of option 13, wherein at least a portion the desired ratio is achieved by a ratio of bacteriocin coding sequences, or bacteriocin and signal molecule coding sequences in the single reading frame of the nucleic acid.
15. The method of any one of options 12-14, wherein the desired ratio is further achieved by a second nucleic acid comprising a ratio of bacteriocins coding sequences and further comprising cleavage sites between the bacteriocin coding sequences.
16. The method of option 15, wherein the desired ratio is achieved by a ratio of bacteriocin coding sequences in the single reading frame of the nucleic acid.
17. The method of any one of options 12-16, wherein the desired ratio of bacteriocins is selected to target an undesired microbial organism or population of undesired microbial organisms, and/or wherein the desired ratio of bacteriocins is selected to balance a population of a microbiome of an animal, a human organ, a plant root, or soil.
18. The method of any one of options 12-16, wherein the desired ratio of bacteriocins and signal molecules is selected to control genetic drift of a target microbial cell, and stimulate growth or production of a producing cell.
19. The method of any one of options 10-18, wherein the desired ratio comprises a ratio of a first bacteriocin to a second bacteriocin of 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, or 9:10, wherein the first bacteriocin is different from the second bacteriocin.
20. The method of any one of options 1-18, wherein the cleavage sites are for a wild-type, variant, or synthetic cleavage enzyme, such as an endopeptidase.
21. The method of any one of options 1-20, wherein the cleavage sites are for a cleavage enzyme selected from the group consisting of: Arg-C proteinase, Asp-N endopeptidase, BNPS-Skatole, Caspase 1, Caspase 2, Caspase 3, Caspase 4, Caspase 5, Caspase 6, Caspase 7, Caspase 8, Caspase 9, Caspase 10, Chymotrypsin-high specificity, Clostripain (Clostridiopeptidase B), CNBr, Enterokinase, Factor Xa, Formic acid, Glutamyl endopeptidase, GranzymeB, Hydroxylamine, lodosobenzoic acid, LysC, Neutrophil elastase, NTCB (2-nitro-5-thiocyanobenzoic acid), Pepsin (pH1.3), Pepsin (pH>2), Proline-endopeptidase, Proteinase K, Staphylococcal peptidase I, Thermolysin, Thrombin, or Trypsin.
22. The method of any one of options 1-21, wherein the cleavage sites are for a single cleavage enzyme, and wherein the cleavage enzyme does not cleave within the bacteriocins.
23 The method of any one of options 1-22, wherein at least one cleavage site is for a first cleavage enzyme, and another cleavage site is for a second cleavage enzyme, and wherein neither the first nor the second cleavage enzyme cleaves within the bacteriocins.
24. The method of any one of options 1-23, wherein the composition further comprises a signal molecule, and wherein the nucleotide further comprises:
   a coding sequence for a signal molecule in the single reading frame; and
   a cleavage site sequence disposed between the signal molecule and a bacteriocin coding sequence.
25. The method of any one of options 1-2 or 4-24, wherein the signal molecule is selected from the group consisting of: quorum sensing molecules, signal transduction receptor ligands, growth factors, hormones, and cytokines, and wherein the signal molecule can be wild-type, mutant, or synthetic.
26. The method of any one of options 1-25, wherein the pro-polypeptide has a length of no more than about 2000 amino acids.
27. The method of any one of options 1-26, further comprising expressing a second nucleic acid encoding a second pro-polypeptide comprising two bacteriocins and cleavage sites disposed therebetween, wherein the second pro-polypeptide is different from the first pro-polypeptide.
28. The method of any one of options 2-19 or 24-27, wherein cleaving the pro-polypeptide comprises physical treatment of a peptide linker comprised by the cleavage site, wherein the peptide linker is chemical-sensitive or pH sensitive.
29. The method of any one of options 1-28, further comprising chemically modifying the bacteriocins.
30. The method of option 29, wherein the bacteriocins are chemically modified co-translationally.
31. The method of any one of options 2-30, further comprising chemically modifying the bacteriocins following the cleaving.
32. An isolated nucleic acid comprising:
   a bacteriocin coding sequence and a second polypeptide coding sequence in a single reading frame, wherein the second polypeptide is a bacteriocin or a signal molecule; and
   cleavage site coding sequences disposed between the bacteriocin coding sequences and in the single reading frame.
33. The isolated nucleic acid of option 32, wherein the second polypeptide is the bacteriocin.
34. The isolated nucleic acid of option 32, wherein the second polypeptide is the signal molecule.
35. The isolated nucleic acid of any one of options 32-34, wherein the cleavage site coding sequences encode cleavage sites for a cleavage enzyme, and wherein the bacteriocin coding sequences do not comprise cleavage sites for the cleavage enzyme.
35. The isolated nucleic acid of any one of options 32-35, wherein the nucleic acid comprises three bacteriocin coding sequences in the single reading frame.
36. The isolated nucleic acid of any one of options 32-35, wherein the nucleic acid comprises at least 5, 10, 15, or 20 bacteriocin sequences in the single reading frame.
37. The isolated nucleic acid of any one of options 32-36, wherein a cleavage site coding sequence is disposed in frame between any two adjacent bacteriocin and/or signal molecule coding sequences.
38. The isolated nucleic acid of any one of options 32-36, wherein at least two of the bacteriocin sequences encode different bacteriocins from each other.
39. The isolated nucleic acid of option 35, wherein the three bacteriocin sequences are present in a desired ratio or portion of a desired ratio.
40. The isolated nucleic acid of option 39, wherein the desired ratio is selected to target an undesired microbial organism or population of undesired microbial organisms.
41. The isolated nucleic acid of option 39 wherein the desired ratio of bacteriocins is selected to balance a population of a microbiome of an animal, a human organ, a plant root, or soil.
42. The isolated nucleic acid of any one of options 32-41, wherein the cleavage sites are for a wild-type, variant, or synthetic cleavage enzyme, such as an endopeptidase.
43. The isolated nucleic acid of any one of options 32-42, wherein the cleavage sites are for a cleavage enzyme selected from the group consisting of: Arg-C proteinase, Asp-N endopeptidase, BNPS-Skatole, Caspase 1, Caspase 2, Caspase 3, Caspase 4, Caspase 5, Caspase 6, Caspase 7, Caspase 8, Caspase 9, Caspase 10, Chymotrypsin-high specificity, Clostripain (Clostridiopeptidase B), CNBr, Enterokinase, Factor Xa, Formic acid, Glutamyl endopeptidase, GranzymeB, Hydroxylamine, lodosobenzoic acid, LysC, Neutrophil elastase, NTCB (2-nitro-5-thiocyanobenzoic acid), Pepsin (pH1.3), Pepsin (pH>2), Proline-endopeptidase, Proteinase K, Staphylococcal peptidase I, Thermolysin, Thrombin, or Trypsin.
44. The isolated nucleic acid of any one of options 42-43, wherein a first cleavage site coding sequence encodes a first cleavage site is for a first cleavage enzyme, and wherein a second cleavage site coding sequence encodes a cleavage site for a second cleavage enzyme that is different from the first cleavage enzyme, and wherein the bacteriocins do not comprise a cleavage site for any of the first or second cleavage enzyme.
45. The isolated nucleic acid of any one of options 32-41, wherein the cleavage sites comprise a pH- or chemically-sensitive linker.
46. The isolated nucleic acid of any one of options 32, 33, or 35-44, wherein the isolated nucleic acid further comprises a coding sequence for a signal molecule in the single reading frame, wherein a cleavage site coding sequences is disposed between the coding sequence for signal molecule and an adjacent bacteriocin coding sequence.
47. The isolated nucleic acid of any one of options 32 or 24-46, wherein the signal molecule is selected from the group consisting of: quorum sensing molecules, signal transduction receptor ligands, growth factors, hormones, and cytokines, and wherein the signal molecule can be wild-type, mutant, or synthetic.
48. A microbial cell, comprising a promoter operably linked to the isolated nucleic acid of any one of options 32-47, wherein the isolated microbial cell does not produce a functional immunity modulator for a bacteriocin encoded by the isolated nucleic acid.
49. The microbial cell of option 48, wherein the cell does not produce a functional immunity modulator for any of the bacteriocins encoded by the isolated nucleic acid.
50. An isolated pro-polypeptide comprising:
   two bacteriocins, and/or a bacteriocin and a signal molecule;
   cleavage sites disposed between the bacteriocins and/or the bacteriocin and the signal molecule; and
   an affinity tag.
51. The isolated pro-polypeptide of option 50, wherein the pro-polypeptide comprises the two bacteriocins.
52. The isolated pro-polypeptide of option 50, wherein the pro-polypeptide comprises the bacteriocin and the signal molecule.
53. The isolated pro-polypeptide of any one of options 50-52, wherein the pro-polypeptide comprises three bacteriocins.
54. The isolated pro-polypeptide of any one of options 50-52, wherein the pro-polypeptide comprises at least 5, 10, 15, or 20 bacteriocins.
55. The isolated pro-polypeptide of any one of options 50, 51, 53, or 54, wherein the pro-polypeptide comprises a signal molecule.
56. The isolated pro-polypeptide of any one of options 50-55, wherein the cleavage sites are for a cleavage enzyme, and wherein the bacteriocin coding sequences do not comprise cleavage sites for the cleavage enzyme.
57. The isolated pro-polypeptide of any one of options 50-55, wherein a cleavage site is for a first cleavage enzyme, and wherein another cleavage site is for a second cleavage enzyme different from the first cleavage enzyme, and wherein the bacteriocins do not comprise a cleavage site for any of the first or second cleavage enzymes.
58. The isolated pro-polypeptide of any one of options 50-57, further comprising a co-translational or post-translational modification.
59. A composition comprising two more bacteriocins in a ratio selected to target a microbial cell or populations of microbial cells, wherein each of the bacteriocins comprises, at its N-terminus, C-terminus, or N-terminus and C-terminus, a portion of a cleavage sequence that has been cleaved, wherein the portions of cleavage sequences at the N-, C-, or N- and C-termini of the bacteriocins are for cleavage sites of the same or different cleavage enzyme.
60. The composition of option 59, wherein at least some of the bacteriocins further comprise a tag.
61. The composition of option 60, wherein the tag is selected from the group consisting of affinity tags, a signal sequence, or a stability tag.
62. The composition of any one of options 59-61, further comprising a signal molecule in a desired ratio with the bacteriocins, wherein the signal molecule comprises, at its N-terminus, C-terminus, or N-terminus and C-terminus, a portion of a cleavage sequence that has been cleaved, wherein the portions of cleavage sequences at the N-, C-, or N- and C-termini of the signal molecule are for cleavage sites of the same or different cleavage enzymes
63. The composition of any one of options 59-62, wherein the ratio of bacteriocins is selected to target an undesired microbial organism or population of undesired microbial organisms.
64. The composition of any one of options 59-63, wherein the ratio of bacteriocins is selected to balance a population of a microbiome of an animal, a human organ, a plant root, or soil.
65. The composition of option 64, wherein the composition is formulated for topical or oral administration to a human subject.
66. The composition of option 64 or 65, wherein the composition is formulated for use in balancing a population of a microbiome of an animal, a human organ, a plant root, or soil.
67. A method for producing a specified mixture of bacteriocins and/or antimicrobial peptides, the method comprising:
   selecting the mixture to comprise two or more different bacteriocins and/or antimicrobial peptides;
   in a microfluidic device comprising discrete coding substrates that each encode a bacteriocin or antimicrobial peptide: placing discrete coding substrates that encode the antimicrobial peptides or bacteriocins of the specified mixture in fluid communication with an in vitro transcription/translation solution;
   incubating the discrete coding substrates with the in vitro transcription/translation solution, thereby generating antimicrobial peptides and/or bacteriocins encoded by the discrete coding substrates; and mixing the antimicrobial peptides and/or bacteriocins in the microfluidic device, thereby producing the specified mixture of antimicrobial peptides and/or bacteriocins.
68. The method of option 67, further comprising:
   producing two or more submixtures each comprising a subset of the specified mixture of antimicrobial peptides and/or bacteriocins; and
   combining the submixtures to produce the specified mixture of antimicrobial peptides and/or bacteriocins.
69. The method of option 67 or 68, wherein selecting further comprises selecting a stoichiometry of the two or more different antimicrobial peptides and/or bacteriocins of the specified mixture.
70. The method of any one of options 68-69, wherein the specified mixture of antimicrobial peptides and/or bacteriocins comprises a specified stoichiometry, and wherein combining the submixtures results in the specified stoichiometry.
71. The method of any one of options 67-70, wherein the discrete coding substrates are comprised within separate chambers.
72. The method of any one of options 67-71, wherein the discrete coding substrates comprise nucleic acids immobilized thereon.
73. The method of any one of options 67-72, wherein discrete coding substrate encoding antimicrobial peptides and/or bacteriocins of the specified mixture, but not other discrete coding substrates, are placed in fluidic communication with the in vitro transcription/translation solution.
74. The method of any one of options 67-73, wherein incubating the discrete coding substrates with the in vitro transcription/translation solution comprises flowing the in vitro transcription/translation solution into each chamber.
75. The method of any one of options 67-74, wherein the in vitro transcription/translation solution comprises an in vitro transcription reagent and/or an in vitro translation reagent.
76. The method of any one of options 67-75, wherein placing the discrete coding substrates that encode the antimicrobial peptides and/or bacteriocins of the specified mixture in fluid communication with an in vitro transcription/translation solution comprises (i) opening valves so as to place a source of the in vitro transcription/translation solution in fluid communication with the discrete coding substrates; (ii) closing valves so as to inhibit fluid communication between the source of the in vitro transcription/translation solution and the other discrete coding substrates, or a combination of (i) and (ii).
77. The method of any one of options 67-76, wherein mixing the antimicrobial peptides and/or bacteriocins in the microfluidic device comprises opening a valve to place the discrete coding substrates in fluid communication with a fluidic reservoir, wherein the antimicrobial peptides and/or bacteriocins are mixed in the fluidic reservoir.
78. The method of any one of options 67-77, further comprising screening the mixture of antimicrobial peptides and/or bacteriocins in situ for a desired effect.
79. The method of option 78, wherein the screening is for inhibition of the growth or reproduction of a pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract.
80. The method of option 78, wherein the screening is for an absence of deleterious effects of the mixture of antimicrobial peptides and/or bacteriocins on a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract.
81. The method of option 78, wherein said screening is performed in real time.
82. The method of option 78, the screening is for stabilization of an antimicrobial peptide and/or bacteriocin or for destruction of a microbial biofilm.
83. The method of option 82, wherein one or more of the discrete coding substrates encodes an auxiliary protein with anti-protease activity.
84. The method of option 78, wherein the screening is for enhancement of growth or reproduction of a non-pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract.
85. The method of option 84, wherein one or more of the discrete coding substrates encodes an auxiliary protein that attracts the non-pathogenic microbial organism, or that enhances growth or reproduction of the non-pathogenic microbial organism in the microbiome of a subject.
86. The method of any one of options 67-85, further comprising delivering the specified mixture of antimicrobial peptides and/or bacteriocins to a wound via a tubing or membrane, thereby cleaning or dressing the wound.
87. A microfluidic device for producing a specified mixture of antimicrobial peptides and/or bacteriocins, the device comprising:
discrete coding substrates that each encode an antimicrobial peptide and/or bacteriocin;
an in vitro transcription/translation solution;
a fluidic reservoir; and
valves each disposed on a fluidic path between a discrete coding substrate and the fluidic reservoir, each valve configured to regulate flow between the discrete coding substrate and the fluidic reservoir, wherein the device is configured to be placed in data communication with a processor configured to:
   based on the specified mixture of antimicrobial peptides and/or bacteriocins, configure the valves to place the discrete coding substrates that encode the antimicrobial peptides and/or bacteriocins of the mixture, but not other discrete coding substrates, in fluidic communication with the fluidic reservoir;
   permit incubation of the in vitro transcription/translation solution with the discrete coding substrates that encode the antimicrobial peptides and/or bacteriocins of the specified mixture, whereby the antimicrobial peptides and/or bacteriocins of the specified mixture are produced;
   permit flow of the antimicrobial peptides and/or bacteriocins through the valves into the fluidic reservoir; and
   control flow of fluid in the fluidic reservoir, wherein the flow comprises movement of the antimicrobial peptides and/or bacteriocins in the fluidic reservoir, thereby producing the specified mixture of antimicrobial peptides and/or bacteriocins in the fluidic reservoir.
88. The microfluidic device of option 87, wherein:
   the specified mixture of antimicrobial peptides and/or bacteriocins comprises two or more submixtures each comprising a subset of antimicrobial peptides and/or bacteriocins; and
   the processor is configured to permit flow of each submixture into the fluidic reservoir, thereby producing the specified mixture of antimicrobial peptides and/or bacteriocins.
89. The microfluidic device of option 88, wherein the specified mixture of antimicrobial peptides and/or bacteriocins comprises a sum of the subsets of antimicrobial peptides and/or bacteriocins in a specified stoichiometry, and wherein combination of the submixtures yields the specified stoichiometry.
90. The microfluidic device of any one of options 87-89, wherein the discrete coding substrates are comprised within separate chambers.
91. The microfluidic device of any one of options 87-90, wherein the discrete coding substrates comprise nucleic acids immobilized thereon.
92. The microfluidic device of option 91, wherein the discrete coding substrates comprise a material or product selected from the group consisting of a chip, bead, nanoparticle, well, membrane, matrix, plastic, metal, glass, polymer, polysaccharide, and paramagnetic compound.
93. The microfluidic device of any one of options 87-92, wherein the in vitro transcription/translation solution comprises an in vitro transcription reagent and/or an in vitro translation reagent.
94. The microfluidic device of any one of options 87-93, wherein the device is portable.
95. The microfluidic device of any one of options 87-94, wherein one or more of the discrete coding substrates encodes an auxiliary protein comprising a protein for stabilization of antimicrobial peptides and/or bacteriocins, a protein with anti-protease activity, or a protein for destruction of a microbial biofilm.
96. The microfluidic device of any one of options 87-95, wherein one or more of the discrete coding substrates encodes an auxiliary protein that attracts a non-pathogenic microbial organism, or that enhances growth or reproduction of the non-pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract.
97. The microfluidic device of any one of options 87-96, wherein the fluidic reservoir is configured to be placed in fluid communication with a tissue of a subject.
98. The microfluidic device of any one of option 97, wherein the tissue comprises a microbiome, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract.
99. The microfluidic device of any one of options 87-98, wherein the fluidic reservoir is configured to be placed in fluid communication with a wound of a subject.
100. The microfluidic device of any one of options 97-99, further comprising a fluidic passage such as a tube or membrane through which the fluidic reservoir is in fluid communication with the microbiome or wound, through which the specified mixture of antimicrobial peptides and/or bacteriocins is capable of being delivered to the microbiome or wound.
101. The microfluidic device of any one of options 87-100, wherein each discrete coding substrate encodes a different antimicrobial peptide and/or bacteriocin.
102. The microfluidic device of any one of options 87-102, wherein a discrete coding substrate comprises the isolated nucleic acid of any of options 32-49.
103. The microfluidic device of any one of options 87-102, wherein a discrete coding substrate encodes the isolated pro-polypeptide of any one of options 50-58.
104. The microfluidic device of any one of options 87-103, further comprising the processor.
105. A system comprising:
the microfluidic device of any one of options 87-104, and
a processor configured to:
   based on the specified mixture of antimicrobial peptides and/or bacteriocins, configure the valves to place the discrete coding substrates that encode the antimicrobial peptides and/or bacteriocins of the mixture, but not other discrete coding substrates, in fluidic communication with the fluidic reservoir;
   permit incubation of the in vitro transcription/translation solution with the discrete coding substrates that encode the antimicrobial peptides and/or bacteriocins of the specified mixture, whereby the antimicrobial peptides and/or bacteriocins of the specified mixture are produced;
   permit flow of the antimicrobial peptides and/or bacteriocins through the valves into the fluidic reservoir; and
   control flow of fluid in the fluidic reservoir, wherein the flow comprises movement of the antimicrobial peptides and/or bacteriocins in the fluidic reservoir, thereby producing the specified mixture of antimicrobial peptides and/or bacteriocins in the fluidic reservoir.
106. The system of option 105, wherein the microfluidic device is comprised by a cartridge (or is a cartridge), the system comprising a coupling for placing the cartridge in data communication with the processor.
107. The system of any one of options 105-106 further comprising a reservoir of in vitro transcription/translation solution.
108. A microfluidic device for producing a specified mixture of antimicrobial peptides and/or bacteriocins, the device comprising:
discrete coding substrates that each encode an antimicrobial peptide and/or bacteriocin;
valves each disposed on a fluidic path connected to a discrete coding substrate, each valve configured to regulate flow to or from the discrete coding substrate, and wherein the device is configured to be placed in fluid communication with a fluidic reservoir or an in vitro transcription/translation solution.
109. The microfluidic device of option 108 further comprising a fluidic reservoir or an in vitro transcription/translation solution.
110. The method of option 78, wherein the desired effect comprises antimicrobial activity.
111. The method of option 110, further comprising screening 1, 2, 3, 4, 5, 6, 7, 8, 9, or 10 different mixtures against a microbial infection.
112. The method of any one of options 67-86 further comprising delivering the mixture of specified antimicrobial peptides and/or bacteriocins, in combination with a chemical antibiotic and/or a phage antibiotic, to a subject.
113. The method of any one of options 67-86 wherein the in vitro transcription/translation solution is lyophyilized, and further comprising adding water to the in vitro transcription/translation solution.
114. The microfluidic device of any one of claims 87-104, further comprising a reservoir of chemical or phage antibiotics configured to mix with the mixture of specified antimicrobial peptides and/or bacteriocins.
115. The microfluidic device of any one of options 87-104, further comprising an antibiotic comprising a chemical antibiotic and/or a phage.
116. The microfluidic device of any one of options 87-104, wherein the in vitro transcription/translation solution is lyophyilized.
117. The system of any one of options 105-107 further comprising a reservoir of chemical or phage antibiotics configured to mix with the mixture of specified antimicrobial peptides and/or bacteriocins.
118. The system of any one of options 105-107 further comprising an antibiotic comprising a chemical antibiotic and/or a phage.
119. The system of option 107 wherein the in vitro transcription/translation solution is lyophyilized.
120. The method of any one of options 67-86 and 110-113, wherein the method is for making a specified mixture of antimicrobial peptides that does not comprise bacteriocins.
121. The method of any one of options 67-86 and 110-113, wherein the method is for making a specified mixture of bacteriocins that does not comprise antimicrobial peptides.
122. The method of any one of options 67-86 and 110-113, wherein the method is for making a specified mixture of bacteriocins and antimicrobial peptides.
123. The microfluidic device of any one of options 87-104, 108, 109, and 114-116, wherein the device is for producing a specified mixture of bacteriocins that does not comprise antimicrobial peptides.
124. The microfluidic device of any one of options 87-104, 108, 109, and 114-116, wherein the device is for producing a specified mixture of antimicrobial peptides that does not comprise bacteriocins.
125. The microfluidic device of any one of options 87-104, 108, 109, and 114-116, wherein the device is for producing a specified mixture of antimicrobial peptides and bacteriocins.
126. The system of any one of options 105-107 and 117-119, wherein the system is for producing a specified mixture of bacteriocins that does not comprise antimicrobial peptides.
127. The system of any one of options 105-107 and 117-119, wherein the system is for producing a specified mixture of antimicrobial peptides that does not comprise bacteriocins.
128. The system of any one of options 105-107 and 117-119, wherein the system is for producing a specified mixture of antimicrobial peptides and bacteriocins.

In at least some of the embodiments described herein, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

What is claimed is:

1. A method of making bacteriocins, the method comprising expressing a nucleic acid comprising:
a bacteriocin coding sequence and a second polypeptide coding sequence in a single reading frame, wherein the second polypeptide is a bacteriocin or signal molecule; and
cleavage site coding sequences disposed between the bacteriocin coding sequence and the second polypeptide coding sequence in the single reading frame,
thereby generating a pro-polypeptide comprising the bacteriocin, second polypeptide, and cleavage sites disposed therebetween.

2. The method of claim 1, further comprising cleaving the cleavage site, thereby separating the bacteriocin and second polypeptide from each other, and thereby producing a composition comprising the bacteriocin and the second polypeptide.

3. The method of claim 1, wherein the expressing is performed by a microbial cell that does not produce a functional immunity modulator for at least one of the bacteriocins.

4. The method of claim 1, wherein at least one of the bacteriocins is inactive when it is part of the pro-polypeptide.

5. The method of any one of claims 1-4, wherein the composition comprises a desired ratio of bacteriocins, or a desired ratio of signal molecules and bacteriocins.

6. The method of claim 5, wherein at least a portion the desired ratio is achieved by a ratio of bacteriocin coding sequences, or bacteriocin and signal molecule coding sequences in the single reading frame of the nucleic acid.

7. The method of claim 5, wherein the desired ratio of bacteriocins is selected to target an undesired microbial organism or population of undesired microbial organisms, and/or
wherein the desired ratio of bacteriocins is selected to balance a population of a microbiome of an animal, a human organ, a plant root, or soil.

8. An isolated nucleic acid comprising:
a bacteriocin coding sequence and a second polypeptide coding sequence in a single reading frame, wherein the second polypeptide is a bacteriocin or a signal molecule; and
cleavage site coding sequences disposed between the bacteriocin coding sequences and in the single reading frame.

9. The isolated nucleic acid of claim 8, wherein the cleavage site coding sequences encode cleavage sites for a cleavage enzyme, and wherein the bacteriocin coding sequences do not comprise cleavage sites for the cleavage enzyme.

10. A microbial cell, comprising a promoter operably linked to the isolated nucleic acid of any one of claims 8-9, wherein the microbial cell does not produce a functional immunity modulator for a bacteriocin encoded by the isolated nucleic acid.

11. An isolated pro-polypeptide comprising:
two bacteriocins, and/or a bacteriocin and a signal molecule;
cleavage sites disposed between the bacteriocins and/or the bacteriocin and the signal molecule; and
an affinity tag.

12. The isolated pro-polypeptide of claim 11, wherein the cleavage sites are for a cleavage enzyme, and wherein the bacteriocin coding sequences do not comprise cleavage sites for the cleavage enzyme.

13. A composition comprising two more bacteriocins in a ratio selected to target a microbial cell or populations of microbial cells,
wherein each of the bacteriocins comprises, at its N-terminus, C-terminus, or N-terminus and C-terminus, a portion of a cleavage sequence that has been cleaved, wherein the portions of cleavage sequences at the N-, C-, or N- and C-termini of the bacteriocins are for cleavage sites of the same or different cleavage enzyme.

14. The composition of claim 13, wherein at least some of the bacteriocins further comprise a tag.

15. The composition of claim 14, wherein the tag is selected from the group consisting of affinity tags, a signal sequence, or a stability tag.

16. A method for producing a specified mixture of bacteriocins and/or antimicrobial peptides, the method comprising:
selecting the mixture to comprise two or more different bacteriocins and/or antimicrobial peptides;
in a microfluidic device comprising discrete coding substrates that each encode a bacteriocin or antimicrobial peptide: placing discrete coding substrates that encode the antimicrobial peptides or bacteriocins of the specified mixture in fluid communication with an in vitro transcription/translation solution;
incubating the discrete coding substrates with the in vitro transcription/translation solution, thereby generating antimicrobial peptides and/or bacteriocins encoded by the discrete coding substrates; and
mixing the antimicrobial peptides and/or bacteriocins in the microfluidic device, thereby producing the specified mixture of antimicrobial peptides and/or bacteriocins.

17. The method of claim 16, further comprising:
producing two or more submixtures each comprising a subset of the specified mixture of antimicrobial peptides and/or bacteriocins; and
combining the submixtures to produce the specified mixture of antimicrobial peptides and/or bacteriocins.

18. The method of claim 16, wherein selecting further comprises selecting a stoichiometry of the two or more different antimicrobial peptides and/or bacteriocins of the specified mixture.

19. The method of claim 16, wherein the specified mixture of antimicrobial peptides and/or bacteriocins comprises a specified stoichiometry, and wherein combining the submixtures results in the specified stoichiometry.

20. The method of any one of claims 16-19, wherein the discrete coding substrates are comprised within separate chambers.

21. The method of any one of claims 16-19, wherein discrete coding substrate encoding antimicrobial peptides and/or bacteriocins of the specified mixture, but not other discrete coding substrates, are placed in fluidic communication with the in vitro transcription/translation solution.

22. The method of any one of claims 16-19, further comprising screening the mixture of antimicrobial peptides and/or bacteriocins in situ for a desired effect.

23. The method of claim 22, wherein the screening is for enhancement of growth or reproduction of a non-pathogenic microbial organism in a microbiome of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract.

24. A microfluidic device for producing a specified mixture of antimicrobial peptides and/or bacteriocins, the device comprising:
discrete coding substrates that each encode an antimicrobial peptide and/or bacteriocin;
an in vitro transcription/translation solution;
a fluidic reservoir; and
valves each disposed on a fluidic path between a discrete coding substrate and the fluidic reservoir, each valve configured to regulate flow between the discrete coding substrate and the fluidic reservoir, wherein the device is configured to be placed in data communication with a processor configured to:
based on the specified mixture of antimicrobial peptides and/or bacteriocins, configure the valves to place the discrete coding substrates that encode the antimicrobial peptides and/or bacteriocins of the mixture, but not other discrete coding substrates, in fluidic communication with the fluidic reservoir;
permit incubation of the in vitro transcription/translation solution with the discrete coding substrates that encode the antimicrobial peptides and/or bacteriocins of the specified mixture, whereby the antimicrobial peptides and/or bacteriocins of the specified mixture are produced;
permit flow of the antimicrobial peptides and/or bacteriocins through the valves into the fluidic reservoir; and
control flow of fluid in the fluidic reservoir, wherein the flow comprises movement of the antimicrobial peptides and/or bacteriocins in the fluidic reservoir, thereby producing the specified mixture of antimicrobial peptides and/or bacteriocins in the fluidic reservoir.

25. The microfluidic device of claim 24, wherein:
the specified mixture of antimicrobial peptides and/or bacteriocins comprises two or more submixtures each comprising a subset of antimicrobial peptides and/or bacteriocins; and
the processor is configured to permit flow of each submixture into the fluidic reservoir, thereby producing the specified mixture of antimicrobial peptides and/or bacteriocins.

26. The microfluidic device of claim 24, wherein the specified mixture of antimicrobial peptides and/or bacteriocins comprises a sum of the subsets of antimicrobial peptides and/or bacteriocins in a specified stoichiometry, and wherein combination of the submixtures yields the specified stoichiometry.

27. The microfluidic device of any one of claims 24-26, wherein the discrete coding substrates are comprised within separate chambers.

28. The microfluidic device of any one of claims 24-26, wherein the fluidic reservoir is configured to be placed in fluid communication with a tissue of a subject.

29. The microfluidic device of any one of claims 24-26, further comprising the processor.

30. A system comprising:
the microfluidic device of any one of claims 24-26, and
a processor configured to:
based on the specified mixture of antimicrobial peptides and/or bacteriocins, configure the valves to place the discrete coding substrates that encode the antimicrobial peptides and/or bacteriocins of the mixture, but not other discrete coding substrates, in fluidic communication with the fluidic reservoir;
permit incubation of the in vitro transcription/translation solution with the discrete coding substrates that encode the antimicrobial peptides and/or bacteriocins of the specified mixture, whereby the antimicrobial peptides and/or bacteriocins of the specified mixture are produced;
permit flow of the antimicrobial peptides and/or bacteriocins through the valves into the fluidic reservoir; and
control flow of fluid in the fluidic reservoir, wherein the flow comprises movement of the antimicrobial peptides and/or bacteriocins in the fluidic reservoir, thereby producing the specified mixture of antimicrobial peptides and/or bacteriocins in the fluidic reservoir.

31. The system of claim 30, wherein the microfluidic device is comprised by a cartridge, the system comprising a coupling for placing the cartridge in data communication with the processor.

32. The system of claim 30 wherein the in vitro transcription/translation solution is lyophyilized.

33. A microfluidic device for producing a specified mixture of antimicrobial peptides and/or bacteriocins, the device comprising:
discrete coding substrates that each encode an antimicrobial peptide and/or bacteriocin;
valves each disposed on a fluidic path connected to a discrete coding substrate, each valve configured to regulate flow to or from the discrete coding substrate, and wherein the device is configured to be placed in fluid communication with a fluidic reservoir of an in vitro transcription/translation solution.

34. The microfluidic device of claim 33, further comprising a reservoir of chemical or phage antibiotics configured to mix with the mixture of specified antimicrobial peptides and/or bacteriocins.


SEQUENCE LISTING (750 sequences)

SEQ ID NO: 1 - protein, 40 aa, Enterococcus faecalis
WLPPAGLLGRCGRWFRPWLLWLQSGAQYKWLGNLFGLGPK

SEQ ID NO: 2 - protein, 5 aa, synthetic construct
  REGION 1..5: note = N-terminal motif of class terminal IIa
  VARIANT 3: note = Xaa = any amino acid
YGXGV

SEQ ID NO: 3 - protein, 135 aa, synthetic construct
  REGION 1..135: note = Hybrid bacteriocin Ent35-MccV
GKYYGNGVSCNKKGCSVDWGRAIGIIGNNSAANLATGGAAGWKSGGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 4 - protein, 46 aa, Lactobacillus acidophilus
MISSHQKTLTDKELALISGGKTHYPTNAWKSLWKGFWESLRYTDGF

SEQ ID NO: 5 - DNA, 141 bp, Lactobacillus acidophilus
  source 1..141: mol_type = genomic DNA
atgatttcatctcatcaaaaaacgttaactgataaagaattagcattaatttctggggggaaaacgcactacccgactaatgcatggaaaagtctttggaaaggtttctgggaaagccttcgttatactgacggtttttag

SEQ ID NO: 6 - protein, 81 aa, Lactobacillus acidophilus
MISMISSHQKTLTDKELALISGGKTYYGTNGVHCTKKSLWGKVRLKNVIPGTLCRKQSLPIKQDLKILLGWATGAFGKTFH

SEQ ID NO: 7 - DNA, 246 bp, Lactobacillus acidophilus
  source 1..246: mol_type = genomic DNA
atgatttcaatgatttcatctcatcaaaaaacgttaactgataaagaattagcattaatttctggggggaaaacgtactatggtactaatggtgtgcattgtactaaaaagagtctttggggtaaagtacgcttaaaaaacgtgattcctggaactctttgtcgtaagcaatcgttgccgatcaaacaggatttaaaaattttactgggctgggctacaggtgcttttggcaagacatttcattaa

SEQ ID NO: 8 - protein, 60 aa, Lactobacillus acidophilus
MDKKTKILFEVLYIICIIGPQFILFVTAKNNMYQLVGSFVGIVWFSYIFWYIFFKQHKKM

SEQ ID NO: 9 - DNA, 183 bp, Lactobacillus acidophilus
  source 1..183: mol_type = genomic DNA
atggataagaaaacaaaaatattatttgaagtattatacatcatctgtataataggccctcaatttatattatttgtgactgcaaaaaacaatatgtatcagttggtgggttcgtttgttggaatagtatggttttcgtatattttttggtatatttttttcaaacaacataaaaaaatgtag

SEQ ID NO: 10 - protein, 65 aa, Lactobacillus gasseri
MALKTLEKHELRNVMGGNKWGNAVIGAATGATRGVSWCRGFGPWGMTACALGGAAIGGYLGYKSN

SEQ ID NO: 11 - DNA, 198 bp, Lactobacillus gasseri
  source 1..198: mol_type = genomic DNA
atggctttaaaaacattagaaaaacatgaattaagaaatgtaatgggtggaaacaagtgggggaatgctgtaataggagctgctacgggagctactcgcggagtaagttggtgcagaggattcggaccatggggaatgactgcctgtgcgttaggaggtgctgcaattggaggatatctgggatataagagtaattaa

SEQ ID NO: 12 - protein, 51 aa, Staphylococcus aureus [sequence omitted]

SEQ ID NO: 13 - DNA, 156 bp, Staphylococcus aureus
  source 1..156: mol_type = genomic DNA
atgagttggttaaattttttaaaatacatcgctaaatatggcaaaaaagcggtatctgctgcttggaagtacaaaggtaaagtattagaatggcttaatgttggtcctactcttgaatgggtatggcaaaaattaaagaaaattgctggattataa

SEQ ID NO: 14 - protein, 61 aa, Enterococcus avium
MTRSKKLNLREMKNVVGGTYYGNGVSCNKKGCSVDWGKAISIIGNNSAANLATGGAAGWKS

SEQ ID NO: 15 - DNA, 186 bp, Enterococcus avium
  source 1..186: mol_type = genomic DNA
atgacaagatcaaaaaaattaaatttacgcgaaatgaagaatgttgttggtggtacctactatggaaatggtgtatcttgtaacaagaaaggctgttcagttgactggggcaaagccatcagtattataggaaataattccgcagcaaacttagcaactggtggtgctgctggttggaagtcataa

SEQ ID NO: 16 - protein, 67 aa, Enterococcus faecalis
MKKKLVICGIIGIGFTALGTNVEAATYYGNGLYCNKQKCWVDWNKASREIGKIIVNGWVQHGPWAPR

SEQ ID NO: 17 - DNA, 204 bp, Enterococcus faecalis
  source 1..204: mol_type = genomic DNA
atgaaaaagaaattagttatttgtggcattattgggattggttttacagcattaggaacaaatgtagaagctgctacgtattacggaaatggtttatattgtaataagcaaaaatgttgggtagactggaataaagcttcaagggaaattggaaaaattattgttaatggttgggtacaacatggcccttgggctcctagatag

SEQ ID NO: 18 - protein, 51 aa, Lactococcus lactis
MKEQNSFNLLQEVTESELDLILGAKGGSGVIHTISHEVIYNSWNFVFTCCS

SEQ ID NO: 19 - DNA, 156 bp, Lactococcus lactis
  source 1..156: mol_type = genomic DNA
atgaaagaacaaaactcttttaatcttcttcaagaagtgacagaaagtgaattggaccttattttaggtgcaaaaggcggcagtggagttattcatacaatttctcatgaagtaatatataatagctggaactttgtatttacttgctgctcttaa

SEQ ID NO: 20 - protein, 74 aa, Enterococcus faecium
MKKKVLKHCVILGILGTCLAGIGTGIKVDAATYYGNGLYCNKEKCWVDWNQAKGEIGKIIVNGWVNHGPWAPRR

SEQ ID NO: 21 - DNA, 225 bp, Enterococcus faecium
  source 1..225: mol_type = genomic DNA
atgaaaaagaaagtattaaaacattgtgttattctaggaatattaggaacttgtctagctggcatcggtacaggaataaaagttgatgcagctacttactatggaaatggtctttattgtaacaaagaaaaatgttgggtagattggaatcaagctaaaggagaaattggaaaaattattgttaatggttgggttaatcatggtccatgggcacctagaaggtag

SEQ ID NO: 22 - protein, 50 aa, Clostridium botulinum
MQKPEIISADLGLCAVNEFVALAAIPGGAATFAVCQMPNLDEIVSNAAYV

SEQ ID NO: 23 - DNA, 153 bp, Clostridium botulinum
  source 1..153: mol_type = genomic DNA
atgcaaaaaccagaaattattagtgctgatttagggctttgtgcagttaatgaatttgtagctcttgctgccattcctggtggtgctgctacatttgcagtatgccaaatgccaaacttggatgagattgttagtaatgcagcatatgtttaa

SEQ ID NO: 24 - protein, 58 aa, Streptococcus equinus
MMNATENQIFVETVSDQELEMLIGGADRGWIKTLTKDCPNVISSICAGTIITACKNCA

SEQ ID NO: 25 - DNA, 177 bp, Streptococcus equinus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcttgtaaaaattgtgcttaa

SEQ ID NO: 26 - protein, 77 aa, Brochothrix campestris
MHKVKKLNNQELQQIVGGYSSKDCLKDIGKGIGAGTVAGAAGGGLAAGLGAIPGAFVGAHFGVIGGSAACIGGLLGN

SEQ ID NO: 27 - DNA, 234 bp, Brochothrix campestris
  source 1..234: mol_type = genomic DNA
atgcacaaggtaaaaaaattaaacaatcaagagttacaacagatcgtgggaggttacagttcaaaagattgtctaaaagatattggtaaaggaattggtgctggtacagtagctggggcagccggcggtggcctagctgcaggattaggtgctatcccaggagcattcgttggagcacattttggagtaatcggcggatctgccgcatgcattggtggattattaggtaactag

SEQ ID NO: 28 - protein, 80 aa, Butyrivibrio fibrisolvens
MSKKQIMSNCISIALLIALIPNIYFIADKMGIQLAPAWYQDIVNWVSAGGTLTTGFAIIVGVTVPAWIAEAAAAFGIASA

SEQ ID NO: 29 - DNA, 243 bp, Butyrivibrio fibrisolvens
  source 1..243: mol_type = genomic DNA
atgagtaaaaaacaaattatgagtaactgtatatcaattgcattattaatagcactaattcctaatatctattttattgcagataaaatgggaattcagttagcacctgcttggtatcaagatattgtgaattgggtatctgctggtggaacacttactactggttttgcgattattgtaggagttacagtaccggcatggatagcagaagcagctgcagcttttggtatagcttcagcatga

SEQ ID NO: 30 - protein, 48 aa, Butyrivibrio fibrisolvens
MNKELNALTNPIDEKELEQILGGGNGVIKTISHECHMNTWQFIFTCCS

SEQ ID NO: 31 - DNA, 147 bp, Butyrivibrio fibrisolvens
  source 1..147: mol_type = genomic DNA
atgaacaaagaacttaatgcacttacaaatcctattgacgagaaggagcttgagcagatcctcggtggtggcaatggtgtcatcaagacaatcagccacgagtgccacatgaacacatggcagttcattttcacatgttgctcttaa

SEQ ID NO: 32 - protein, 66 aa, Carnobacterium maltaromaticum
MNSVKELNVKEMKQLHGGVNYGNGVSCSKTKCSVNWGQAFQERYTAGINSFVSGVASGAGSIGRRP

SEQ ID NO: 33 - DNA, 201 bp, Carnobacterium maltaromaticum
  source 1..201: mol_type = genomic DNA
atgaatagcgtaaaagaattaaacgtgaaagaaatgaaacaattacacggtggagtaaattatggtaatggtgtttcttgcagtaaaacaaaatgttcagttaactggggacaagcctttcaagaaagatacacagctggaattaactcatttgtaagtggagtcgcttctggggcaggatccattggtaggagaccgtaa

SEQ ID NO: 34 - protein, 61 aa, Carnobacterium maltaromaticum
MKSVKELNKKEMQQINGGAISYGNGVYCNKEKCWVNKAENKQAITGIVIGGWASSLAGMGH

SEQ ID NO: 35 - DNA, 186 bp, Carnobacterium maltaromaticum
  source 1..186: mol_type = genomic DNA
atgaaaagcgttaaagaactaaataaaaaagaaatgcaacaaattaatggtggagctatctcttatggcaatggtgtttattgtaacaaagagaaatgttgggtaaacaaggcagaaaacaaacaagctattactggaatagttatcggtggatgggcttctagtttagcaggaatgggacattaa

SEQ ID NO: 36 - protein, 71 aa, Carnobacterium maltaromaticum
MNNVKELSIKEMQQVTGGDQMSDGVNYGKGSSLSKGGAKCGLGIVGGLATIPSGPLGWLAGAAGVINSCMK

SEQ ID NO: 37 - DNA, 216 bp, Carnobacterium maltaromaticum
  source 1..216: mol_type = genomic DNA
atgaataatgtaaaagagttaagtattaaagaaatgcaacaagttactggtggagaccaaatgtcagatggtgtaaattatggaaaaggctctagcttatcaaaaggtggtgccaaatgtggtttagggatcgtcggcggattagctactatcccttcaggtcctttaggctggttagccggagcagcaggtgtaattaatagctgtatgaaataa

SEQ ID NO: 38 - protein, 64 aa, Carnobacterium maltaromaticum
MLYELVAYGIAQGTAEKVVSLINAGLTVGSIISILGGVTVGLSGVFTAVKAAIAKQGIKKAIQL

SEQ ID NO: 39 - DNA, 195 bp, Carnobacterium maltaromaticum
  source 1..195: mol_type = genomic DNA
atgttatatgaattagttgcatatggtatcgcacaaggtacagctgaaaaggttgtaagtctaattaacgcaggtttaacagtagggtctattatttcaattttgggtggggtcacagtcggtttatcaggtgtcttcacagcagttaaagcagcaattgctaaacaaggaataaaaaaagcaattcaattataa

SEQ ID NO: 40 - protein, 836 aa, Pectobacterium carotovorum
  source 1..836: note = subsp. carotovorum
MIKYRLYAPNDGDTMTVSGGGGWVSNDDRKGGNDRDNGKGGSAVDFSKNPEKQAIVNPYLAIAIPMPVYPLYGKLGFTINTTAIETELANVRAAINTKLATLSAVIGRSLPVVGRVFGVTAAGMWPSSTAPSSLDSIYNQAHQQALAQLAAQQGVLNKGYNVTAMPAGFVSSLPVSEIKSLPTAPASLLAQSVINTELSQRQLALTQPTTNAPVANIPVVKAEKTAMPGVYSAKIIAGEPAFQIKVDNTKPALAQNPPKVKDDIQVSSFLSSPVADTHHAFIDFGSDHEPVYVSLSKIVTAEEEKKQVEEAKRREQEWLLRHPITAAERKLTEIRQVISFAQQLKESSVATISEKTKTVAVYQEQVNTAAKNRDNFYNQNRGLLSAGITGGPGYPIYLALWQTMNNFHQAYFRANNALEQESHVLNLARSDLAKAEQLLAENNRLQVETERTLAEEKEIKRNRVNVSTFGTVQTQLSKLLSDFYAVTSLSQSVPSGALASFSYNPQGMIGSGKIVGKDVDVLFSIPVKDIPGYKSPINLDDLAKKNGSLDLPIRLAFSDENGERVLRAFKADSLRIPSSVRGVAGSYDKNTGIFSAEIDGVSSRLVLENPAFPPTGNVGNTGNTAPDYKALLNTGVDVKPVDKITVTVTPVADPVDIDDYIIWLPTASGSGVEPIYVVFNSNPYGGTEKGKYSKRYYNPDKAGGPILELDWKNVKIDHAGVDNVKLHTGRFKASVENKVMIERLENILNGQITATDTDKRFYTHELRELNRYRNLGIKDGEVPSSIQEESAVWNDTHTATLEDYKINEKEQPLYTDAALQAAYEQELKDALGGKHG

SEQ ID NO: 41 - DNA, 2511 bp, Pectobacterium carotovorum
  source 1..2511: mol_type = genomic DNA; note = subsp. carotovorum
atgattaaataccgtttatatgctccaaatgatggagacaccatgacagtgagtggtggtggtggttgggtttcaaacgatgatcgcaaaggtggtaatgacagggacaatggcaaaggtggttctgccgttgattttagtaaaaatccagaaaagcaggctatcgttaatccctatttggcaatcgcgataccgatgccggtctaccctctttatggaaagctagggttcacaataaatacgacggcaattgagactgaactcgcaaatgtcagagcagcaattaacactaaacttgcaacactcagtgcagtgattggcagatcacttccggtcgttgggcgggtatttggtgttactgccgccggaatgtggccttctagtaccgctcccagtagtctcgattctatatacaatcaagcacatcagcaggctttagcccagttagctgctcaacagggagtattaaataaagggtataacgttacagcaatgcctgcaggtttcgtcagcagtttgcctgttagtgaaatcaaatcattgccaacagctcccgccagtttactggcacaaagtgtgattaataccgaactttcccagcgtcaactggctcttactcagcccacgacgaatgcaccagtcgcgaatattcccgtagttaaagcagagaaaacagcaatgccaggtgtgtattcagcgaaaattattgctggtgagcctgcattccaaatcaaggtcgataataccaaacctgctttggcacagaatccgccgaaagtaaaagatgatattcaggtatcttctttcctttcctcgccagtagctgatacgcaccatgcatttattgattttggcagcgatcatgaaccggtatacgtgtctctttcaaagatcgtgacagccgaggaggagaaaaaacaggttgaagaggccaagcgccgtgagcaggagtggttgttgcgtcatccaattacagctgcggagcgaaaattaactgaaatccgccaagtgatctcttttgctcaacagctaaaagaaagctctgtcgcaaccatttcagaaaaaactaaaactgttgcggtttaccaagaacaggtgaataccgctgcaaaaaatcgcgacaatttttataatcaaaatagaggtctgttaagtgcgggtataactgggggaccgggatatcctatttatcttgctttatggcaaacgatgaataactttcatcaggcttatttcagagcaaataatgcattggaacaagagagtcatgttctgaacctggctcgttctgatctggctaaggctgagcaattgcttgctgagaataatcgacttcaggttgaaacggagcgaacgcttgccgaagaaaaagagataaaacgcaacagggttaatgtatcaacatttggcacagtgcaaactcaacttagtaaattgctgtcagatttttatgctgttacatcactttcccaaagtgttccttcgggggcattagcctctttttcatataatccacaagggatgattggcagcggtaagattgttgggaaggatgtcgatgttttattttccatcccagtaaaagatattccgggatataaatctcctattaacttggacgatttagccaagaaaaatggaagtctggatcttcccattcgtctggcattttctgatgagaatggagaaagggttcttcgggcattcaaagcggatagtctgcgaatcccttcgagtgtcagaggtgtagcgggcagttatgacaaaaatacgggtatttttagtgcagaaattgatggtgtttcatctcgccttgtactggaaaacccagcgtttcctccgaccggaaatgtcggtaatacgggtaatactgcacctgactataaagcattactgaatactggtgttgatgttaaacctgttgataaaatcacagttacggtaacaccagttgctgatccagtggatattgatgactatataatctggttgccaactgcgtctggttctggcgtggaacccatttatgtcgtgtttaacagtaatccgtatggtgggacggaaaaaggaaaatatagcaaacgttattataatccagataaggcaggcggtccgatcttggagctggattggaaaaacgttaagattgaccatgcaggtgtggacaatgttaaattacacacagggcgtttcaaagcgtcggttgaaaacaaagtgatgattgaacgtttggaaaacatactgaatggtcaaatcacggccacggatactgacaagcgattctatacgcatgaattaagagagttaaaccgctacagaaatttaggcatcaaagacggtgaagtgcctagtagcattcaagaagaaagcgctgtttggaacgacacacacacagcgacgcttgaagactacaaaattaatgagaaagagcaaccgttgtacactgatgctgctttgcaggcagcctacgaacaggaactcaaagacgcattaggagggaaacatggctaa

SEQ ID NO: 42 - protein, 74 aa, Bacillus cereus
MENLQMLTEEELMEIEGGGWWNSWGKCVAGTIGGAGTGGLGGAAAGSAVPVIGTGIGGAIGGVSGGLTGAATFC

SEQ ID NO: 43 - DNA, 225 bp, Bacillus cereus
  source 1..225: mol_type = genomic DNA
atggaaaacttacaaatgttaactgaagaagaattaatggaaattgaaggtggaggctggtggaatagctggggtaaatgtgttgctggaactatcggtggagctggaactggtggtttaggtggagctgctgcaggttcagctgttccggttattggtactggtattggtggcgctattggtggagttagcggtggccttacaggtgcagctactttttgctaa

SEQ ID NO: 44 - protein, 78 aa, Streptoverticillium griseoverticillatum
MTASILQQSVVDADFRAALLENPAAFGASAAALPTPVEAQDQASLDFWTKDIAATEAFACRQSCSFGPFTFVCDGNTK

SEQ ID NO: 45 - DNA, 237 bp, Streptoverticillium griseoverticillatum
  source 1..237: mol_type = genomic DNA
atgaccgcttccattcttcagcagtccgtcgtggacgccgacttccgcgcggcgctgcttgagaaccccgccgccttcggcgcttccgccgcggccctgcccacgcccgtcgaggcccaggaccaggcgtcccttgacttctggaccaaggacatcgccgccacggaagccttcgcctgccgccagagctgcagcttcggcccgttcaccttcgtgtgcgacggcaacaccaagtaa

SEQ ID NO: 46 - protein, 76 aa, Geobacillus kaustophilus
MSLLALVAGTLGVSQSIATTVVSIVLTGSTLISIILGITAILSGGVDAILEIGWSAFVATVKKIVAERGKAAAIAW

SEQ ID NO: 47 - DNA, 231 bp, Geobacillus kaustophilus
  source 1..231: mol_type = genomic DNA
atgagtttgctggcgcttgttgccgggacgctcggcgtgtcacagtcaatcgcgacgacggttgtttcgattgtgttgaccggctccactctcatttctattattcttgggatcaccgctattttgtcaggtggagtcgacgccattttggaaattgggtggtcagcttttgtcgcgacggtgaaaaaaatagtggcggaacgaggaaaagcggcagcgattgcatggtaa

SEQ ID NO: 48 - protein, 309 aa, Clostridium tyrobutyricum
MRKVFLRSIISTLVMCAFVSSSFSVNADESKPNDEKIINNIENVTTTKDIVKSNKNNIVYLDEGVMSIPLSGRKPIAIKDDNNKEDLTVTLPIKNTGDISKISSNGTILYKNNSSNSSNIALQPKNDGFKALININDKLANKEYEFTFNLPKNSKLISAATYLGKEYDTKEVFVVDKNNIITSIISPAWAKDANGHNVSTYYKIVSNNKLVQVVEFTENTAFPVVADPNWTKIGKCAGSIAWAIGSGLFGGAKLIKIKKYIAELGGLQKAAKLLVGATTWEEKLHAGGYALINLAAELTGVAGIQANCF

SEQ ID NO: 49 - DNA, 930 bp, Clostridium tyrobutyricum
  source 1..930: mol_type = genomic DNA
ttgagaaaagtatttttaagatcaataatttcaacattagttatgtgtgcatttgtttcaagcagcttttcagtaaatgcggatgaaagcaaaccaaatgatgaaaaaataattaataacatagaaaacgttactactactaaagatattgtaaaaagtaataaaaataatattgtatatttagatgaaggtgtaatgagtattccattgtctgggagaaaacccattgctattaaagatgataataataaagaagatttaactgttacattacctattaagaatactggagatatatctaaaattagtagtaatggtactattctgtataaaaataatagtagtaattcatctaatatagctttacaacctaaaaatgatggatttaaggctttaataaatattaatgataagttagctaataaagaatatgaatttacatttaatttacccaaaaacagtaaattaattagtgctgccacatatttgggtaaagaatatgatacaaaagaagtatttgtagtagacaaaaataatataattacgagtattattagtccagcttgggctaaagatgcaaatggacataatgtttctacttattataagatagtatcgaataataaattagtacaagttgttgaattcacagaaaatactgcattcccggtggtagctgatcctaattggactaaaattgggaaatgcgctgggtcaatagcatgggctataggttctggcctttttggtggagcaaagctaattaaaataaaaaaatatatagcagagcttggaggacttcaaaaagcagctaaattattagttggtgcaaccacttgggaagaaaaattacacgcaggcggttatgcattaattaacttagctgctgagctaacaggtgtagcaggtatacaagcaaattgtttttaa

SEQ ID NO: 50 - protein, 62 aa, Bacillus coagulans
MKKIEKLTEKEMANIIGGKYYGNGVTCGKHSCSVDWGKATTCIINNGAMAWATGGHQGTHKC

SEQ ID NO: 51 - DNA, 189 bp, Bacillus coagulans
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacctgcataatcaataatggagctatggcatgggctactggtggacatcaaggtactcataaatgctag

SEQ ID NO: 52 - protein, 490 aa, Escherichia coli
MDKVTDNSPDVESTESTEGSFPTVGVDTGDTITATLATGTENVGGGGGAFGGASESSAAIHATAKWSTAQLKKHQAEQAARAAAAEAALAKAKSQRDALTQRLKDIVNDALRANAARSPSVTDLAHANNMAMQAEAERLRLAKAEQKAREEAEAAEKALREAERQRDEIARQQAETAHLLAMAEAAEAEKNRQDSLDEEHRAVEVAEKKLAEAKAELAKAESDVQSKQAIVSRVAGELENAQKSVDVKVTGFPGWRDVQKKLERQLQDKKNEYSSVTNALNSAVSIRDAKKTEVQNAEIKLKEAKDALEKSQVKDSVDTMVGFYQYITEQYGEKYSRIAQDLAEKAKGSKFNSVDEALAAFEKYKNVLDKKFSKVDRDDIFNALESITYDEWAKHLEKISRALKVTGYLSFGYDVWDGTLKGLKTGDWKPLFVTLEKSAVDFGVAKIVALMFSFIVGAPLGFWGIAIITGIVSSYIGDDELNKLNELLGI

SEQ ID NO: 53 - DNA, 1473 bp, Escherichia coli
  source 1..1473: mol_type = genomic DNA
atggataaagtcactgataattctccagatgtggagagcacagaatctactgaggggtcattcccaactgttggggttgatactggcgatacgattacagcgacgcttgcaactggaactgaaaatgttggtggaggcggtggagcatttggtggggccagtgaaagttctgctgcgatacatgcaaccgctaaatggtctaccgcgcagttgaaaaaacatcaggctgaacaggctgcccgtgctgctgcggctgaggcagcattggcaaaagcgaaatctcagcgtgatgccctgactcaacgtctcaaggatattgttaatgacgctttacgtgctaatgccgctcgtagtccatcagtaactgaccttgctcatgccaataatatggcaatgcaggcagaggctgagcgtttgcgccttgcgaaggcagagcaaaaagcccgtgaagaagctgaagcagcagaaaaagcgctccgggaagcagaacgccaacgtgatgagattgcccgccaacaggctgaaaccgcgcatttgttagcaatggcggaggcagcagaggctgagaaaaatcgacaggattctcttgatgaagagcatcgggctgtggaagtggcagagaagaagctggctgaggctaaagctgaactggcgaaggccgaaagcgatgtacagagtaagcaagcgattgtttccagagttgcaggggagcttgaaaacgctcaaaaaagtgttgatgtgaaggttaccggatttcctggatggcgtgatgttcagaaaaaactggagagacaattgcaggataagaagaatgaatattcgtcagtgacgaatgctcttaattctgctgttagcattagagatgctaaaaaaacagaagttcagaatgctgagataaaattaaaagaagctaaggatgctcttgagaagagtcaggtaaaagactctgttgatactatggttgggttttatcaatatataaccgaacaatatggggaaaaatattccagaatagctcaggatttagctgaaaaggcgaagggtagtaaatttaatagtgttgatgaagcacttgctgcatttgaaaagtataaaaatgtactggataagaaattcagtaaggttgatagggatgatatttttaatgctttagagtctattacttatgatgagtgggccaagcatctagaaaagatctctagggctcttaaggttactggatatttgtctttcgggtatgatgtatgggatggtaccctaaagggattaaaaacaggagactggaagcctttatttgtcactctggagaagagcgcggtagatttcggcgtggcaaaaattgtggcattaatgtttagttttattgttggtgcgcctcttggcttctggggaattgcaattatcacaggtattgtttcttcttacataggggatgatgagttgaacaagcttaatgaattactaggtatttaa

SEQ ID NO: 54 - protein, 522 aa, Escherichia coli
METAVAYYKDGVPYDDKGQVIITLLNGTPDGSGSGGGGGKGGSKSESSAAIHATAKWSTAQLKKTQAEQAARAKAAAEAQAKAKANRDALTQRLKDIVNEALRHNASRTPSATELAHANNAAMQAEDERLRLAKAEEKARKEAEAAEKAFQEAEQRRKEIEREKAETERQLKLAEAEEKRLAALSEEAKAVEIAQKKLSAAQSEVVKMDGEIKTLNSRLSSSIHARDAEMKTLAGKRNELAQASAKYKELDELVKKLSPRANDPLQNRPFFEATRRRVGAGKIREEKQKQVTASETRINRINADITQIQKAISQVSNNRNAGIARVHEAEENLKKAQNNLLNSQIKDAVDATVSFYQTLTEKYGEKYSKMAQELADKSKGKKIGNVNEALAAFEKYKDVLNKKFSKADRDAIFNALASVKYDDWAKHLDQFAKYLKITGHVSFGYDVVSDILKIKDTGDWKPLFLTLEKKAADAGVSYVVALLFSLLAGTTLGIWGIAIVTGILCSYIDKNKLNTINEVLGI

SEQ ID NO: 55 - DNA, 1569 bp, Escherichia coli
  source 1..1569: mol_type = genomic DNA
atggaaaccgcggtagcgtactataaagatggtgttccttatgatgataagggacaggtaattattactcttttgaatggtactcctgacgggagtggctctggcggcggaggtggaaaaggaggcagtaaaagtgaaagttctgcagctattcatgcaactgctaaatggtctactgctcaattaaagaaaacacaggcagagcaggctgcccgggcaaaagctgcagcggaagcacaggcgaaagcaaaggcaaacagggatgcgctgactcagcgcctgaaggatatcgtgaatgaggctcttcgtcacaatgcctcacgtacgccttcagcaacagagcttgctcatgctaataatgcagctatgcaggcggaagacgagcgtttgcgccttgcgaaagcagaagaaaaagcccgtaaagaagcggaagcagcagaaaaggcttttcaggaagcagaacaacgacgtaaagagattgaacgggagaaggctgaaacagaacgccagttgaaactggctgaagctgaagagaaacgactggctgcattgagtgaagaagctaaagctgttgagatcgcccaaaaaaaactttctgctgcacaatctgaagtggtgaaaatggatggagagattaagactctcaattctcgtttaagctccagtatccatgcccgtgatgcagaaatgaaaacgctcgctggaaaacgaaatgaactggctcaggcatccgctaaatataaagaactggatgagctggtcaaaaaactatcaccaagagccaatgatccgcttcagaaccgtcctttttttgaagcaaccagacgacgggttggggccggtaagattagagaagaaaaacaaaaacaggtaacagcatcagaaacacgtattaaccggataaatgctgatataactcagatccagaaggctatttctcaggtcagtaataatcgtaatgccggtatcgctcgtgttcatgaagctgaagaaaatttgaaaaaagcacagaataatctccttaattcacagattaaggatgctgttgatgcaacagttagcttttatcaaacgctgactgaaaaatatggtgaaaaatattcgaaaatggcacaggaacttgctgataagtctaaaggtaagaaaatcggcaatgtgaatgaagctctcgctgcttttgaaaaatacaaggatgttttaaataagaaattcagcaaagccgatcgtgatgctatttttaatgcgttggcatcggtgaagtatgatgactgggctaaacatttagatcagtttgccaagtacttgaagattacggggcatgtttcttttggatatgatgtggtatctgatatcctaaaaattaaggatacaggtgactggaagccactatttcttacattagagaagaaagctgcagatgcaggggtgagttatgttgttgctttactttttagcttgcttgctggaactacattaggtatttggggtattgctattgttacaggaattctatgctcctatattgataagaataaacttaatactataaatgaggtgttagggatttaa

SEQ ID NO: 56 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 57 - DNA, 1881 bp, Escherichia coli
  source 1..1881: mol_type = genomic DNA
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatgattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatccgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 58 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLKTARNALTRAEQQLTQQKNTPDGKTIVSPEKFPGRSSTNHSIVVSGDPRFAGTIKITTSAVIDNRANLNYLLTHSGLDYKRNILNDRNPVVTEDVEGDKKIYNAEVAEWDKLRQRLLDARNKITSAESAINSARNNVSARTNEQKHANDALNALLKEKENIRSQLADINQKIAEEKRKRDEINMVKDAIKLTSDFYRTIYDEFGKQASELAKELASVSQGKQIKSVDDALNAFDKFRNNLNKKYNIQDRMAISKALEAINQVHMAENFKLFSKAFGFTGKVIERYDVAVELQKAVKTDNWRPFFVKLESLAAGRAASAVTAWAFSVMLGTPVGILGFAIIMAAVSALVNDKFIEQVNKLIGI

SEQ ID NO: 59 - DNA, 1881 bp, Escherichia coli
  source 1..1881: mol_type = genomic DNA
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgaacgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatctga

SEQ ID NO: 60 - protein, 271 aa, Escherichia coli
METLTVHAPSPSTNLPSYGNGAFSLSAPHVPGAGPLLVQVVYSFFQSPNMCLQALTQLEDYIKKHGASNPLTLQIISTNIGYFCNADRNLVLHPGISVYDAYHFAKPAPSQYDYRSMNMKQMSGNVTTPIVALAHYLWGNGAERSVNIANIGLKISPMKINQIKDIIKSGVVGTFPVSTKFTHATGDYNVITGAYLGNITLKTEGTLTISANGSWTYNGVVRSYDDKYDFNASTHRGIIGESLTRLGAMFSGKEYQILLPGEIHIKESGKR

SEQ ID NO: 61 - DNA, 816 bp, Escherichia coli
  source 1..816: mol_type = genomic DNA
atggaaaccttaactgttcatgcaccatcaccatcaactaacttaccaagttatggcaatggtgcattttctctttcagcaccacatgtgcctggtgctggccctcttttagtccaggttgtttatagttttttccagagtccaaacatgtgtcttcaggctttaactcaacttgaggattacatcaaaaaacatggggccagcaaccctctcacattgcagatcatatcgacaaatattggttacttctgtaacgccgaccgaaatctggttcttcaccctggaataagcgtttatgacgcttaccacttcgcaaaaccagcgccaagtcaatatgactatcgctcaatgaatatgaaacaaatgagcggtaatgtcactacaccaattgtggcgcttgctcactatttatggggtaatggcgctgaaaggagcgttaatatcgccaacattggtcttaaaatttcccctatgaaaattaatcagataaaagacattataaaatctggtgtagtaggcacattccctgtttctacaaagttcacacatgccactggtgattataatgttattaccggtgcatatcttggtaatatcacactgaaaacagaaggtactttaactatctctgccaatggctcctggacttacaatggcgttgttcgttcatatgatgataaatacgattttaacgccagcactcaccgtggcattatcggagagtcgctcacaaggctcggggcgatgttttctggtaaagagtaccagatactgcttcctggtgaaattcacattaaagaaagtggtaagcgataa

SEQ ID NO: 62 - protein, 387 aa, Escherichia coli
MGSNGADNAHNNAFGGGKNPGIGNTSGAGSNGSASSNRGNSNGWSWSNKPHKNDGFHSDGSYHITFHGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGVVAGVAISLFGAVLSFLPISGLAVTALGVIGIMTISYLSSFIDANRVSNINNIISSVIR

SEQ ID NO: 63 - DNA, 51 bp, Escherichia coli
  source 1..51: mol_type = genomic DNA
gcaaatcgagtttcgaatataaataacattatatctagtgttattcgatga

SEQ ID NO: 64 - protein, 103 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 65 - DNA, 312 bp, Escherichia coli
  source 1..312: mol_type = genomic DNA
atgagaactctgactctaaatgaattagattctgtttctggtggtgcttcagggcgtgatattgcgatggctataggaacactatccggacaatttgttgcaggaggaattggagcagctgctgggggtgtggctggaggtgcaatatatgactatgcatccactcacaaacctaatcctgcaatgtctccatccggtttaggaggaacaattaagcaaaaacccgaagggataccttcagaagcatggaactatgctgcgggaagattgtgtaattggagtccaaataatcttagtgatgtttgtttataa

SEQ ID NO: 66 - protein, 58 aa, Enterococcus columbae
MMNATENQIFVETVSDQELEMLIGGAGRGWIKTLTKDCPNVISSICAGTIITACKNCA

SEQ ID NO: 67 - DNA, 177 bp, Enterococcus columbae
  source 1..177: mol_type = genomic DNA
atgatgaatgctactgaaaaccaaatttttgttgagactgtgagtgaccaagaattagaaatgttaattggtggtgcaggtcgtggatggattaagactttaacaaaagattgtccaaatgtgatttcttcaatttgtgcaggtacaattattacagcttgtaaaaattgtgcttaa

SEQ ID NO: 68 - protein, 59 aa, Lactobacillus curvatus
MNNVKELSMTELQTITGGARSYGNGVYCNNKKCWVNRGEATQSIIGGMISGWASGLAGM

SEQ ID NO: 69 - DNA, 180 bp, Lactobacillus curvatus
  source 1..180: mol_type = genomic DNA
atgaataatgtaaaagaattaagtatgacagaattacaaacaattaccggcggtgctagatcatatggcaacggtgtttactgtaataataaaaaatgttgggtaaatcggggtgaagcaacgcaaagtattattggtggtatgattagcggctgggctagtggtttagctggaatgtaa

SEQ ID NO: 70 - protein, 64 aa, Streptomyces sp.
MRSEMTLTSTNSAEALAAQDFANTVLSAAAPGFHADCETPAMATPATPTVAQFVIQGSTICLVC

SEQ ID NO: 71 - DNA, 195 bp, Streptomyces sp.
  source 1..195: mol_type = genomic DNA
gtgcgatctgagatgactcttacgagcacgaattccgctgaggctctggcggcgcaggactttgcgaacaccgttctcagcgcggcggccccgggcttccacgcggactgcgagacgccggccatggccaccccggccacgccgaccgtcgcccagttcgtgatccagggcagcacgatctgcctggtctgctga

SEQ ID NO: 72 - protein, 65 aa, Bacillus halodurans
MVNSKDLRNPEFRKAQGLQFVDEVNEKELSSLAGSGDVHAQTTWPCATVGVSVALCPTTKCTSQC

SEQ ID NO: 73 - DNA, 198 bp, Bacillus halodurans
  source 1..198: mol_type = genomic DNA
atggtaaattcaaaagatttgcgtaatcctgaattccgcaaagcccaaggtctacaattcgttgacgaggtgaacgagaaggaactttcgtctctagctggttcaggagatgtgcatgcacaaacaacttggccttgcgctacagttggtgtctccgtagccttgtgcccaactacaaagtgtacaagccagtgctaa

SEQ ID NO: 74 - protein, 66 aa, Carnobacterium divergens
MKNLKEGSYTAVNTDELKSINGGTKYYGNGVYCNSKKCWVDWGQASGCIGQTVVGGWLGGAIPGKC

SEQ ID NO: 75 - DNA, 201 bp, Carnobacterium divergens
  source 1..201: mol_type = genomic DNA
atgaaaaacttaaaagaaggttcatacactgctgttaatactgatgaattaaaaagtatcaatggtggaacaaaatattatgggaatggcgtttattgcaattctaaaaaatgttgggtagattggggacaagcttcaggttgtatcggtcaaactgttgttggcggatggctaggcggagctataccaggtaaatgctaa

SEQ ID NO: 76 - protein, 63 aa, Carnobacterium divergens
MIKREKNRTISSLGYEEISNHKLQEIQGGKGILGKLGVVQAGVDFVSGVWAGIKQSAKDHPNA

SEQ ID NO: 77 - DNA, 192 bp, Carnobacterium divergens
  source 1..192: mol_type = genomic DNA
atgattaaaagagaaaagaacagaacaatttcttcccttggttatgaagaaatttctaatcataaattgcaagaaatacaaggtggaaaaggaattcttggtaaactaggagtagtacaggcaggagtggattttgtatcaggagtgtgggctggaataaaacagtctgccaaagatcatcctaatgcgtaa

SEQ ID NO: 78 - protein, 75 aa, Carnobacterium divergens
MKKQILKGLVIVVCLSGATFFSTPQQASAAAPKITQKQKNCVNGQLGGMLAGALGGPGGVVLGGIGGAIAGGCFN

SEQ ID NO: 79 - DNA, 228 bp, Carnobacterium divergens
  source 1..228: mol_type = genomic DNA
atgaaaaaacaaattttaaaagggttggttatagttgtttgtttatctggggcaacatttttctcaacaccacaacaagcttctgctgctgcaccgaaaattactcaaaaacaaaaaaattgtgttaatggacaattaggtggaatgcttgctggagctttgggtggacctggcggagttgtgttaggtggtataggtggtgcaatagcaggaggttgttttaattaa

SEQ ID NO: 80 - protein, 72 aa, Enterococcus durans
MQTIKELNTMELQEIIGGENDHRMPYELNRPNNLSKGGAKCAAGILGAGLGAVGGGPGGFISAGISAVLGCM

SEQ ID NO: 81 - DNA, 219 bp, Enterococcus durans
  source 1..219: mol_type = genomic DNA
atgcaaacgatcaaagaattgaacacgatggaattacaagaaataattggaggtgaaaatgaccatcggatgccttacgaattgaaccgtccaaataatttatccaaaggtggggctaagtgtgctgctggaatacttggcgctggactaggcgcagtaggcggtggacctggcggatttattagtgccggaatcagtgctgttcttggttgtatgtaa

SEQ ID NO: 82 - protein, 72 aa, Enterococcus durans
MQTIKELNTMELQKIIGGENDHRMPYELNRPNNLSKGGAKCAAGILGAGLGAVGGGPGGFISAGISAVLGCM

SEQ ID NO: 83 - DNA, 219 bp, Enterococcus durans
  source 1..219: mol_type = genomic DNA
atgcaaacgatcaaagaattgaacacgatggaattacaaaaaataattggaggtgaaaatgaccatcggatgccttacgaattgaaccgtccaaataatttatccaaaggtggagctaagtgcgctgccggaatacttggtgctggattaggcgcagtaggcggtggacctggcggatttattagtgccggaatcagtgctgttcttggttgtatgtaa

SEQ ID NO: 84 - protein, 220 aa, Streptococcus dysgalactiae
  source 1..220: note = subsp. equisimilis
MKKLKRLVISLVTSLLVISSTVPALVYANETNNFAETQKEITTNSEATLTNEDYTKLTSEVKTIYTNLIQYDQTKNKFYVDEDKTEQYYNYDDESIKGVYLMKDSLNDELNNNNSSNYSEIINQKISEIDYVLQGNDINNLIPSNTRVKRSADFSWIQRCLEEAWGYAISLVTLKGIINLFKAGKFEAAAAKLASATAGRIAGMAALFAFVATCGATTVS

SEQ ID NO: 85 - DNA, 663 bp, Streptococcus dysgalactiae
  source 1..663: mol_type = genomic DNA; note = subsp. equisimilis
atgaaaaaattaaaacgtcttgttatctctcttgttacttcattactagtaatttcaagtacagttccagcacttgtttacgctaatgaaacaaataactttgcagaaactcaaaaagaaattacaacaaattcagaagcaacattaaccaatgaagactacactaaattaacttccgaagtaaaaacaatttatacaaatctgattcaatacgaccaaacaaaaaacaaattttacgtcgatgaagacaaaactgaacaatattataactacgatgatgaaagtataaaaggggtttatctcatgaaagatagtttgaacgatgagttaaacaataataactcttcaaactattctgaaataattaatcaaaaaatctctgaaattgactatgtccttcaaggaaacgatataaataatttaattcctagcaataccagagtaaaaagatcagcagatttttcttggattcaaagatgtctagaagaagcatggggatatgctattagtctagttactctaaaaggaataatcaatctatttaaagcaggaaaatttgaagctgctgctgctaaattagcttctgctacagcaggtagaatcgctggaatggctgccttatttgctttcgtagcaacttgcggtgcgacaactgtatcataa

SEQ ID NO: 86 - protein, 57 aa, Enterococcus faecalis
MKQYKVLNEKEMKKPIGGESVFSKIGNAVGPAAYWILKGLGNMSDVNQADRINRKKH

SEQ ID NO: 87 - DNA, 174 bp, Enterococcus faecalis
  source 1..174: mol_type = genomic DNA
atgaagcaatataaagtattgaatgaaaaagaaatgaaaaaacctattgggggagagtcggtttttagtaaaataggtaatgctgtaggtccagctgcttattggattttaaaaggattaggtaatatgagtgatgtaaaccaagctgatagaattaatagaaagaaacattaa

SEQ ID NO: 88 - protein, 44 aa, Enterococcus faecalis
MGAIAKLVAKFGWPIVKKYYKQIMQFIGEGWAINKIIDWIKKHI

SEQ ID NO: 89 - DNA, 135 bp, Enterococcus faecalis
  source 1..135: mol_type = genomic DNA
atgggagcaatcgcaaaattagtagcaaagtttggatggccaattgttaaaaagtattacaaacaaattatgcaatttattggagaaggatgggcaattaacaaaattattgattggatcaaaaaacatatttaa

SEQ ID NO: 90 - protein, 43 aa, Enterococcus faecalis
MGAIAKLVAKFGWPFIKKFYKQIMQFIGQGWTIDQIEKWLKRH

SEQ ID NO: 91 - DNA, 132 bp, Enterococcus faecalis
  source 1..132: mol_type = genomic DNA
atgggagcaatcgcaaaattagtagcaaagtttggatggccatttattaaaaaattctacaaacaaattatgcagtttatcggacaaggatggacaatagatcaaattgaaaaatggttaaaaagacattga

SEQ ID NO: 92 - protein, 74 aa, Enterococcus faecalis
MLNKKLLENGVVNAVTIDELDAQFGGMSKRDCNLMKACCAGQAVTYAIHSLLNRLGGDSSDPAGCNDIVRKYCK

SEQ ID NO: 93 - DNA, 225 bp, Enterococcus faecalis
  source 1..225: mol_type = genomic DNA
atgttaaataaaaaattattagaaaatggtgtagtaaatgctgtaacaattgatgaacttgatgctcaatttggtggaatgagcaaacgtgattgtaacttgatgaaggcgtgttgtgctggacaagcagtaacatatgctattcatagtcttttaaatcgattaggtggagactctagtgatccagctggttgtaatgatattgtaagaaaatattgtaaataa

SEQ ID NO: 94 - protein, 65 aa, Enterococcus faecium
MKHLKILSIKETQLIYGGTTHSGKYYGNGVYCTKNKCTVDWAKATTCIAGMSIGGFLGGAIPGKC

SEQ ID NO: 95 - DNA, 195 bp, Enterococcus faecium
  source 1..195: mol_type = genomic DNA
atgaaacatttaaaaattttgtctattaaagagacacaacttatctatgggggtaccactcatagtggaaaatattatggaaatggagtgtattgcactaaaaataaatgtacggtcgattgggccaaggcaactacttgtattgcaggaatgtctataggtggttttttaggtggagcaattccagggaagtgc

SEQ ID NO: 96 - protein, 105 aa, Enterococcus faecalis
MVKENKFSKIFILMALSFLGLALFSASLQFLPIAHMAKEFGIPAAVAGTVLNVVEAGGWVTTIVSILTAVGSGGLSLLAAAGRESIKAYLKKEIKKKGKRAVIAW

SEQ ID NO: 97 - DNA, 318 bp, Enterococcus faecalis
  source 1..318: mol_type = genomic DNA
atggttaaagaaaataaattttctaagatttttattttaatggctttgagttttttggggttagccttgtttagtgcaagtcttcagtttttgcccattgcacatatggctaaagagttcggtataccagcagcagttgcaggaactgtgcttaatgtagttgaagctggtggatgggtcactactattgtatcaattcttactgctgtaggtagcggaggtctttctttactcgctgcagcaggaagagagtcaattaaagcataccttaagaaagaaattaagaaaaaaggaaaaagagcagttattgcttggtaa

SEQ ID NO: 98 - protein, 71 aa, Enterococcus faecium
MQNVKELSTKEMKQIIGGENDHRMPNELNRPNNLSKGGAKCGAAIAGGLFGIPKGPLAWAAGLANVYSKCN

SEQ ID NO: 99 - DNA, 216 bp, Enterococcus faecium
  source 1..216: mol_type = genomic DNA
atgcaaaatgtaaaagaattaagtacgaaagagatgaaacaaattatcggtggagaaaatgatcacagaatgcctaatgagttaaatagacctaacaacttatctaaaggtggagcaaaatgtggtgctgcaattgctgggggattatttggaatcccaaaaggaccactagcatgggctgctgggttagcaaatgtatactctaaatgcaactaa

SEQ ID NO: 100 - protein, 58 aa, Enterococcus mundtii
MKKLTSKEMAQVVGGKYYGNGVSCNKKGCSVDWGKAIGIIGNNSAANLATGGAAGWKS

SEQ ID NO: 101 - DNA, 177 bp, Enterococcus mundtii
  source 1..177: mol_type = genomic DNA
ttgaagaaattaacatcaaaagaaatggcacaagtagtaggtggaaaatactacggtaatggagtctcatgtaataaaaaagggtgcagtgttgattggggaaaagctattggcattattggaaataattctgctgcgaatttagctactggtggagcagctggttggaaaagttaa

SEQ ID NO: 102 - protein, 44 aa, Enterococcus faecalis
MLAKIKAMIKKFPNPYTLAAKLTTYEINWYKQQYGRYPWERPVA

SEQ ID NO: 103 - DNA, 135 bp, Enterococcus faecalis
  source 1..135: mol_type = genomic DNA
atgttagcaaaaattaaagcgatgattaagaagtttccgaacccttatactttagcagctaagctaacgacttacgaaattaattggtataaacaacaatacggtcgttatccttgggagcgccctgtagcataa

SEQ ID NO: 104 - protein, 71 aa, Enterococcus faecium
MRKKLFSLALIGIFGLVVTNFGTKVDAATRSYGNGVYCNNSKCWVNWGEAKENIAGIVISGWASGLAGMGH

SEQ ID NO: 105 - DNA, 216 bp, Enterococcus faecium
  source 1..216: mol_type = genomic DNA
atgagaaaaaaattatttagtttagctcttattggaatatttgggttagttgtgacaaattttggtacaaaagttgatgcagctacgcgttcatatggtaatggtgtttattgtaataatagtaaatgctgggttaactggggagaagctaaagagaatattgcaggaatcgttattagtggctgggcttctggtttggcaggtatgggacattaa

SEQ ID NO: 106 - protein, 34 aa, Enterococcus faecium
MNFLKNGIAKWMTGAELQAYKKKYGCLPWEKISC

SEQ ID NO: 107 - DNA, 105 bp, Enterococcus faecium
  source 1..105: mol_type = genomic DNA
atgaattttcttaaaaatggtatcgcaaaatggatgaccggtgctgaattgcaagcgtataaaaagaaatatggatgcttgccatgggaaaaaatttcttgttaa

SEQ ID NO: 108 - protein, 76 aa, Enterococcus faecalis
MKKKLVKGLVICGMIGIGFTALGTNVEAATYYGNGVYCNKQKCWVDWSRARSEIIDRGVKAYVNGFTKVLGGIGGR

SEQ ID NO: 109 - DNA, 231 bp, Enterococcus faecalis
  source 1..231: mol_type = genomic DNA
atgaaaaagaaattagttaaaggcttagttatttgtggcatgattgggattggttttacagcattaggaacaaatgtagaagccgccacgtattacggaaatggtgtctattgcaataagcaaaaatgttgggtagattggagtagagcacgttctgaaattatagacagaggcgtaaaagcatacgtcaatggatttacgaaagtgttaggtggtataggtggaagataa

SEQ ID NO: 110 - protein, 60 aa, Enterococcus faecalis
MKKEELVGMAKEDFLNVICENDNKLENSGAKCPWWNLSCHLGNDGKICTYSHECTAGCNA

SEQ ID NO: 111 - DNA, 183 bp, Enterococcus faecalis
  source 1..183: mol_type = genomic DNA
atgaaaaaagaagaattagtaggaatggctaaggaagactttttaaatgttatttgtgaaaatgacaacaaactagaaaatagtggagcaaaatgtccttggtggaatctttcttgtcatttaggcaatgatggtaaaatttgcacttattcacatgaatgtaccgcaggttgtaatgcataa

SEQ ID NO: 112 - protein, 61 aa, Enterococcus faecalis
MTELNKRLQLKRDVSTENSLKKISNTDETHGGVTTSIPCTVMVSAAVCPTLVCSNKCGGRG

SEQ ID NO: 113 - DNA, 186 bp, Enterococcus faecalis
  source 1..186: mol_type = genomic DNA
atgactgaacttaacaaaagattacaattaaaaagagatgtttcaacagaaaatagtttgaaaaaaatttctaatactgatgaaacacatgggggagttactacatcaattccatgtacagtaatggttagtgcggcagtatgtcctacccttgtttgctcgaataaatgtggcggtagaggctag

SEQ ID NO: 114 - protein, 58 aa, Enterococcus faecium
MQNVKEVSVKEMKQIIGGSNDSLWYGVGQFMGKQANCITNHPVKHMIIPGYCLSKILG

SEQ ID NO: 115 - DNA, 177 bp, Enterococcus faecium
  source 1..177: mol_type = genomic DNA
atgcaaaatgtaaaagaagtttctgtaaaagagatgaaacaaattatcggtggttctaatgatagtctttggtatggtgtaggacaatttatgggtaaacaagcaaactgtataacaaaccatcctgttaaacacatgataattcctggatattgtttatcgaaaattttagggtaa

SEQ ID NO: 116 - protein, 55 aa, Enterococcus faecium
MKKYNELSKKELLQIQGGIAPIIVAGLGYLVKDAWDHSDQIISGFKKGWNGGRRK

SEQ ID NO: 117 - DNA, 168 bp, Enterococcus faecium
  source 1..168: mol_type = genomic DNA
atgaaaaaatataatgagttatctaaaaaagaacttctacagattcaaggaggaatagcacctattatagttgctggccttggctatttagtaaaagatgcatgggatcactcagatcaaataatctcaggatttaaaaaaggttggaatggtggacgtagaaaataa

SEQ ID NO: 118 - protein, 343 aa, Enterococcus faecalis
MKNILLSILGVLSIVVSLAFSSYSVNAASNEWSWPLGKPYAGRYEEGQQFGNTAFNRGGTYFHDGFDFGSAIYGNGSVYAVHDGKILYAGWDPVGGGSLGAFIVLQAGNTNVIYQEFSRNVGDIKVSTGQTVKKGQLIGKFTSSHLHLGMTKKEWRSAHSSWNKDDGTWFNPIPILQGGSTPTPPNPGPKNFTTNVRYGLRVLGGSWLPEVTNFNNTNDGFAGYPNRQHDMLYIKVDKGQMKYRVHTAQSGWLPWVSKGDKSDTVNGAAGMPGQAIDGVQLNYITPKGEKLSQAYYRSQTTKRSGWLKVSADNGSIPGLDSYAGIFGEPLDRLQIGISQSNPF

SEQ ID NO: 119 - DNA, 1032 bp, Enterococcus faecalis
  source 1..1032: mol_type = genomic DNA
atgaaaaatattttactttctattctaggggtattatctatcgttgtttctttggcgttttcttcttattctgtcaacgcagcttctaatgagtggtcgtggccactgggcaaaccatatgcgggaagatatgaagaaggacaacaattcgggaacactgcatttaaccgaggaggtacttatttccatgatgggtttgactttggttctgctatttatggaaatggcagtgtgtatgctgtgcatgatggtaaaattttatatgctggttgggatcctgtaggtggaggctcattaggtgcatttattgtactacaagcgggaaacacaaatgtgatttatcaagaatttagccgaaatgttggagatattaaagttagcactggacaaactgttaaaaaaggacagctgataggaaagtttacttctagtcatttacatttaggaatgacaaaaaaagaatggcgttctgctcattcttcttggaataaagatgatggcacttggtttaacccaattcctatacttcaaggaggatctacgcctacgcctccaaatccaggaccaaaaaatttcacaacaaatgttcgttacggattgcgggtcctcggaggttcatggttaccagaagtaaccaactttaacaataccaatgatggtttcgcaggttaccctaatcgtcaacatgatatgctttatataaaggtagataaagggcaaatgaaatatcgtgttcacacggctcaaagtggatggttgccttgggtaagtaaaggggataagagcgatacagtaaatggagcggcaggtatgcctggacaagcaattgatggtgttcagctaaactatataactcctaagggagaaaaattatcacaggcttactatcgttcacaaactacgaaacgatcaggctggttaaaagtaagtgcagataatggttctattcctggactagacagttatgcaggaatctttggagaaccgttggatcgcttgcaaataggtatttcacagtcaaatccattttaa

SEQ ID NO: 120 - protein, 56 aa, Staphylococcus epidermidis
MENKKDLFDLEIKKDNMENNNELEAQSLGPAIKATRQVCPKATRFVTVSCKKSDCQ

SEQ ID NO: 121 - DNA, 171 bp, Staphylococcus epidermidis
  source 1..171: mol_type = genomic DNA
atggaaaacaaaaaagatttatttgatttagaaatcaaaaaagataatatggaaaataataatgaattagaagctcaatctcttggtcctgcaattaaggcaactagacaggtatgtcctaaagcaacacgttttgttacagtttcttgtaaaaaaagtgattgtcaatag

SEQ ID NO: 122 - protein, 51 aa, Staphylococcus epidermidis
MAAFMKLIQFLATKGQKYVSLAWKHKGTILKWINAGQSFEWIYKQIKKLWA

SEQ ID NO: 123 - DNA, 156 bp, Staphylococcus epidermidis
  source 1..156: mol_type = genomic DNA
atggcagcatttatgaagttaattcagttcttagcaactaaaggtcaaaagtatgtttcacttgcatggaaacataaaggtactattttaaaatggattaacgccggtcaaagttttgaatggatttataaacaaatcaaaaaattatgggcataa

SEQ ID NO: 124 - protein, 52 aa, Staphylococcus epidermidis
MEAVKEKNDLFNLDVKVNAKESNDSGAEPRIASKFICTPGCAKTGSFNSYCC

SEQ ID NO: 125 - DNA, 159 bp, Staphylococcus epidermidis
  source 1..159: mol_type = genomic DNA
atggaagcagtaaaagaaaaaaatgatctttttaatcttgatgttaaagttaatgcaaaagaatctaacgattcaggagctgaaccaagaattgctagtaaatttatatgtactcctggatgtgcaaaaacaggtagttttaacagttattgttgttaa

SEQ ID NO: 126 - protein, 55 aa, Staphylococcus epidermidis
MNNSLFDLNLNKGVETQKSDLSPQSASVLKTSIKVSKKYCKGVTLTCGCNITGGK

SEQ ID NO: 127 - DNA, 168 bp, Staphylococcus epidermidis
  source 1..168: mol_type = genomic DNA
atgaataactcattattcgatttaaacctaaacaaaggtgtagaaactcaaaagagtgatttaagtccgcaatctgctagtgtcttgaagacttctattaaagtatctaaaaaatattgtaaaggtgttactttaacatgcggttgcaatattactggtggtaaataa

SEQ ID NO: 128 - protein, 52 aa, Staphylococcus gallinarum
MEAVKEKNELFDLDVKVNAKESNDSGAEPRIASKFLCTPGCAKTGSFNSYCC

SEQ ID NO: 129 - DNA, 159 bp, Staphylococcus gallinarum
  source 1..159: mol_type = genomic DNA
atggaagcagtaaaagagaaaaatgaactttttgatcttgacgttaaagtaaatgcaaaagagtctaatgattcaggcgcagaaccacgaattgctagtaaatttttatgtactcctggatgtgccaaaacaggtagcttcaatagctactgttgttaa

SEQ ID NO: 130 - protein, 63 aa, Lactococcus garvieae
MENNNYTVLSDEELQKIDGGIGGALGNALNGLGTWANMMNGGGFVNQWQVYANKGKINQYRPY

SEQ ID NO: 131 - DNA, 192 bp, Lactococcus garvieae
  source 1..192: mol_type = genomic DNA
atggaaaacaacaattacacagtactttcagatgaagaactacaaaaaattgatggtggaatcggcggggctcttggtaatgctctcaacggattaggtacctgggcaaacatgatgaacggtggaggatttgttaatcagtggcaagtttatgctaataaaggaaaaataaatcaataccgtccgtattaa

SEQ ID NO: 132 - protein, 63 aa, Lactococcus garvieae
MFDLVATGMAAGVAKTIVNAVSAGMDIATALSLFSGAFTAAGGIMALIKKYAQKKLWKQLIAA

SEQ ID NO: 133 - DNA, 192 bp, Lactococcus garvieae
  source 1..192: mol_type = genomic DNA
atgtttgatttagtcgcgactggaatggctgcaggtgtagcaaaaactattgttaatgccgttagtgctggtatggatattgccactgctttatcattgttctcaggagcttttactgcagctgggggaattatggcactcattaaaaaatatgctcaaaagaaattatggaaacagcttattgctgcataa

SEQ ID NO: 134 - protein, 91 aa, Lactobacillus gasseri
MVTKYGRNLGLNKVELFAIWAVLVVALLLTTANIYWIADQFGIHLATGTARKLLDAMASGASLGTAFAAILGVTLPAWALAAAGALGATAA

SEQ ID NO: 135 - DNA, 276 bp, Lactobacillus gasseri
  source 1..276: mol_type = genomic DNA
atggttactaagtacggacgtaatttaggtttgaacaaggtagagttgtttgcaatttgggcggttttagtagttgctcttttattgaccacagcgaacatttattggattgctgatcaattcgggattcatttagcgactggaacagcccgtaagttattagatgcaatggcttctggtgcctcattgggaactgcctttgctgctattttgggcgtgacattacctgcatgggctttggcagctgcaggagcattgggagcgactgcagcctag

SEQ ID NO: 136 - protein, 75 aa, Lactobacillus gasseri
MKNFNTLSFETLANIVGGRNNWAANIGGVGGATVAGWALGNAVCGPACGFVGAHYVPIAWAGVTAATGGFGKIRK

SEQ ID NO: 137 - DNA, 228 bp, Lactobacillus gasseri
  source 1..228: mol_type = genomic DNA
atgaaaaattttaatacattatcatttgaaacattggctaacatagttggtgggagaaataattgggctgctaatataggtggagtaggtggagcgacagtcgctggatgggctcttggaaatgcagtttgcggtcctgcttgtggctttgttggagcacactatgttccaatagcatgggctggcgtaacggcagctactggtggattcggaaagataagaaagtag

SEQ ID NO: 138 - protein, 64 aa, Lactobacillus plantarum
MSKLVKTLTISEISKAQNNGGKPAWCWYTLAMCGAGYDSGTCDYMYSHCFGIKHHSSGSSSYHC

SEQ ID NO: 139 - DNA, 195 bp, Lactobacillus plantarum
  source 1..195: mol_type = genomic DNA
atgagtaaattggttaagacacttactataagtgaaatttctaaggctcaaaacaacggtggaaaacctgcatggtgttggtatactttagcaatgtgtggtgctggttatgattcgggaacctgtgattatatgtattcgcattgttttggtataaagcatcatagtagtggtagtagcagttatcattgttag

SEQ ID NO: 140 - protein, 359 aa, Haloferax mediterranei
MSKDRDGRRTSRRGTLKKIGGFSLGALSFGAVGRTQAATGSSVTTADIAPPGPNGDPKSVQIDDKYTGAEMYGEGDFRVGLGTDLTMYPPVYRESLGNGSGGWEFDFTVCGSTACRFVDSNGDVKEDDKAKEMWWQEINFNDINQDLYSRNDSDWVGSTPADTQPEFDYTEFALARDGVTLALTALNPAMGSLALGATYFLSDMVNWIASQHEDDSSLKRKWDYDGLSGPLYADSSTYLLARDEMTSNSYESFTIDNIAVAFPEFPVRTKYYVTFTAPDDPSTQSISTLEEEGIYRVPATEVAAARPPGSRRSKSAADEMVYVADPKKFIEVEPVKNPSIPDRIYEEIEQKKKQRSRKQ

SEQ ID NO: 141 - DNA, 1080 bp, Haloferax mediterranei
  source 1..1080: mol_type = genomic DNA
atgtcgaaagacagagatgggagaaggacaagtcggcgaggcacgttaaagaaaatcggcggtttcagtctcggagcgcttagtttcggggcagtcggacgaactcaagcggcgaccggctcatcggttacgaccgctgatatcgcacctcccggaccgaacggagacccgaagagtgttcagatagatgataaatacaccggagccgagatgtacggcgagggtgacttcagagtcggtctcggaactgacctgacgatgtatccgcccgtgtaccgtgagagtcttggaaatggaagcgggggttgggaattcgacttcaccgtttgtgggtccactgcctgtcgatttgtggacagtaacggtgacgtcaaagaggacgacaaggcgaaagaaatgtggtggcaggaaattaacttcaacgacataaatcaggatttatacagtcggaacgattccgactgggtcgggtcgacccctgccgatacccaaccggagttcgattacaccgactttgcgctcgctcgggacggagtgacgctcgctctcacggcactcaaccccgcaatggggagtcttgcactcggtgccacgtacttcctcagcgacatggtgaactggattgcgagccagcacgaagacgacagttcgctcaagagaaaatgggattacgacgggctaagtgggccgttgtacgccgattcgtcgacgtacctactggcacgcgacgagatgacttcgaactcgtacgaatcattcacgatcgataacatcgccgttgccttcccagagttccccgtccggaccaagtactacgtcacattcactgcgccggatgacccgtcaacgcagtcgatatctacgctcgaagaggagggaatctaccgagtgcccgctacggaagtggctgcggccagaccaccggggtcccgacgttccaaatcggcagccgacgagatggtgtacgttgccgatccgaagaagttcatagaggtcgagccggtgaagaacccaagtatcccggaccgaatctacgaggagatagagcaaaaaaagaaacaacggagtaggaaacagtag

SEQ ID NO: 142 - protein, 311 aa, Haloarchaeon sp.
  source 1..311: note = S8a
MSDKDSINRRNVLRKIGGIGVASAVGFSGLASGESLSDDEKQDVIDTIYKSQRVEQIKKKFGGVNIEPKKVQSVTTNQSGDLVTAKLSVSDGDLVYSSVKDTTVIVQFDRSASEIGESWPKNTEAFIKSTSSGVDLLRTATDEEIKDVTEGVNTSEIESADAVNIFIDPESQTYYMEKYDFNNKVLEMFELATGGTSSGKISPTREDQNHEYNVREHKVFNSEKQNIQLQSDCNINSNTAADVILCFNQVGSCALCSPTLVGGPVPTVACLLVVCFGTPNAVSAILEEVDNSCFNLIKDVISCWDEWTSFW

SEQ ID NO: 143 - DNA, 936 bp, Haloarchaeon sp.
  source 1..936: mol_type = genomic DNA; note = S8a
atgtcggataaagacagcattaacagaagaaatgtattaagaaaaattggcggtatcggtgtggcttcagctgtcggattttctggtttggcaagcggggaaagtcttagcgatgatgagaaacaagatgttattgacacaatttacaaatcacaaagagttgaacagataaagaaaaagttcggaggagtgaatattgagccgaaaaaggttcaatctgtaacgaccaatcagagcggagatcttgttacggcgaagctgtcggttagtgatggggatttggtatattcgagtgtcaaagatacaactgtaatagttcagttcgatagatcggcttctgaaattggtgaaagttggcccaagaatactgaggcattcatcaaatcgacgtcctctggggtcgatcttctacgtacagcaactgatgaagaaataaaggacgttactgagggagtcaacacatctgaaattgaatctgcggatgctgttaacatatttattgatcctgaatcacagacatactatatggagaaatatgactttaataataaggtacttgagatgtttgaattagcgacaggtgggacaagtagtggtaaaatctcccccacacgtgaagaccagaatcacgaatataatgttagggaacataaagtatttaactcagaaaaacagaatatacaacttcagagtgactgtaatataaacagtaacaccgctgctgatgttattctatgcttcaaccaggttggttcttgtgcactctgctccccgactttagtcggaggtccagtccctacagttgcatgtctcttagtcgtctgtttcggcactccaaatgctgtgtccgcgatacttgaagaagtcgataattcttgctttaacttgatcaaggatgtaatttcgtgttgggatgaatggactagcttctggtga

SEQ ID NO: 144 - protein, 333 aa, Lactobacillus helveticus
MKHLNETTNVRILSQFDMDTGYQAVVQKGNVGSKYVYGLQLRKGATTILRGYRGSKINNPILELSGQAGGHTQTWEFAGDRKDINGEERAGQWFIGVKPSKIEGSKIIWAKQIARVDLRNQMGPHYSNTDFPRLSYLNRAGSNPFAGNKMTHAEAAVSPDYTKFLIATVENNCIGHFTIYNLDTINEKLDEKGNSEDVNLETVKYEDSFIIDNLYGDDNNSIVNSIQGYDLDNDGNIYISSQKAPDFDGSYYAHHKQIVKIPYYARSKESEDQWRAVNLSEFGGLDIPGKHSEVESIQIIGENHCYLTVAYHSKNKAGENKTTLNEIYELSWN

SEQ ID NO: 145 - DNA, 1002 bp, Lactobacillus helveticus
  source 1..1002: mol_type = genomic DNA
atgaagcatttaaatgaaacaactaatgttagaattttaagtcaatttgatatggatactggctatcaagcagtagttcaaaaaggcaatgtaggttcaaaatatgtatatggattacaacttcgcaaaggtgctactactatcttgcgtggttaccgtggaagtaaaattaataaccctattcttgaattatctggtcaagcaggtggtcacacacagacatgggaatttgctggtgatcgtaaagacattaatggtgaagaaagagcaggtcaatggtttataggtgttaaaccatcgaaaattgaaggaagcaaaattatttgggcaaagcaaattgcaagagttgatcttagaaatcaaatgggacctcattattcaaatactgactttcctcgattatcctacttgaatcgcgccggttctaatccatttgctggtaataagatgacgcatgccgaagccgcagtatcacctgattatactaagtttttaattgctactgttgaaaataactgtattggtcattttactatatacaatttagatacaattaatgaaaaacttgatgaaaagggaaatagtgaagatgttaatctcgaaactgttaaatacgaagatagttttatcattgataatttatatggtgatgataataattctattgtaaattcaattcaagggtatgatttggataatgatggaaatatttatatttccagtcaaaaagcgccagattttgatggctcttattatgcacatcataagcagattgttaagattccatattatgctcggtctaaagaaagcgaagaccaatggagagctgtaaatttaagcgaattcggtggcttggatattccaggtaaacatagtgaagttgaaagcatccaaattattggtgagaatcattgttacttaactgttgcatatcattctaaaaataaagcgggtgaaaataaaactactttgaatgagatttatgaattatcttggaattag

SEQ ID NO: 146 - protein, 74 aa, Enterococcus hirae; identical to SEQ ID NO: 20

SEQ ID NO: 147 - DNA, 225 bp, Enterococcus hirae; identical to SEQ ID NO: 21
  source 1..225: mol_type = genomic DNA

SEQ ID NO: 148 - protein, 75 aa, Lactobacillus johnsonii
MKQFNYLSHKDLAVVVGGRNNWQTNVGGAVGSAMIGATVGGTICGPACAVAGAHYLPILWTAVTAATGGFGKIRK

SEQ ID NO: 149 - DNA, 228 bp, Lactobacillus johnsonii
  source 1..228: mol_type = genomic DNA
atgaaacaatttaattatttatcacataaagatttagcagtcgttgttggtggaagaaataattggcaaacaaatgtgggaggagcagtgggatcagctatgattggggctacagttggtggtacaatttgtggacctgcatgtgctgtagctggtgcccattatcttcctattttatggacagcggttacagctgcaacaggtggttttggcaagataagaaagtag

SEQ ID NO: 150 - protein, 62 aa, Lactobacillus johnsonii
MKLNDKELSKIVGGNRWGDTVLSAASGAGTGIKACKSFGPWGMAICGVGGAAIGGYFGYTHN

SEQ ID NO: 151 - DNA, 189 bp, Lactobacillus johnsonii
  source 1..189: mol_type = genomic DNA
atgaaattaaatgacaaagaattatcaaagattgttggtggaaatcgatggggagatactgttttatcagctgctagtggcgcaggaactggtattaaagcatgtaaaagttttggcccatggggaatggcaatttgtggtgtaggaggtgcagcaataggaggttattttggctatactcataattaa

SEQ ID NO: 152 - protein, 59 aa, Lactococcus lactis
  source 1..59: note = subsp. lactis
MNKNEIETQPVTWLEEVSDQNFDEDVFGACSTNTFSLSDYWGNNGAWCTLTHECMAWCK

SEQ ID NO: 153 - DNA, 180 bp, Lactococcus lactis
  source 1..180: mol_type = genomic DNA; note = subsp. lactis
atgaacaaaaatgaaattgaaacacaaccagttacatggttggaagaagtatctgatcaaaattttgatgaagatgtatttggtgcgtgtagtactaacacattctcgctcagtgattactggggaaataacggggcttggtgtacactcactcatgaatgtatggcttggtgtaaataa

SEQ ID NO: 154 - protein, 65 aa, Lactococcus lactis
  source 1..65: note = subsp. lactis
MKEKNMKKNDTIELQLGKYLEDDMIELAEGDESHGGTTPATPAISILSAYISTNTCPTTKCTRAC

SEQ ID NO: 155 - DNA, 198 bp, Lactococcus lactis
  source 1..198: mol_type = genomic DNA; note = subsp. lactis
atgaaagaaaaaaatatgaaaaagaatgacactattgaattacaattgggaaaataccttgaagatgatatgattgaattagctgaaggggatgagtctcatggaggaacaacaccagcaactcctgcaatctctattctcagtgcatatattagtaccaatacttgtccaacaacaaaatgtacacgtgcttgttaa

SEQ ID NO: 156 - protein, 51 aa, Lactococcus lactis
  source 1..51: note = subsp. lactis
MKEQNSFNLLQEVTESELDLILGAKGGSGVIHTISHECNMNSWQFVFTCCS

SEQ ID NO: 157 - DNA, 156 bp, Lactococcus lactis
  source 1..156: mol_type = genomic DNA; note = subsp. lactis
atgaaagaacaaaactcttttaatcttcttcaagaagtgacagaaagtgaattggaccttattttaggtgcaaaaggcggcagtggagttattcatacaatttctcatgaatgtaatatgaatagctggcaatttgtatttacttgctgctcttaa

SEQ ID NO: 158 - protein, 53 aa, Lactococcus lactis
  source 1..53: note = subsp. lactis
MAGFLKVVQLLAKYGSKAVQWAWANKGKILDWLNAGQAIDWVVSKIKQILGIK

SEQ ID NO: 159 - DNA, 162 bp, Lactococcus lactis
  source 1..162: mol_type = genomic DNA; note = subsp. lactis
atggcagggtttttaaaagtagttcaattactagctaaatatggttctaaagctgtacaatgggcttgggcaaacaagggtaagattttagattggcttaatgcaggtcaggctattgattgggtagtttcgaaaattaagcaaattttaggtattaagtaa

SEQ ID NO: 160 - protein, 53 aa, Lactococcus lactis
  source 1..53: note = subsp. lactis
MAGFLKVVQILAKYGSKAVQWAWANKGKILDWINAGQAIDWVVEKIKQILGIK

SEQ ID NO: 161 - DNA, 162 bp, Lactococcus lactis
  source 1..162: mol_type = genomic DNA; note = subsp. lactis
atggcagggtttttaaaagtagtccaaattttggctaagtatggttctaaagccgtacaatgggcatgggcaaataaaggaaaaatcttagattggattaatgcaggtcaagctattgactgggtagttgaaaagattaagcaaattttgggtattaaataa

SEQ ID NO: 162 - protein, 65 aa, Lactobacillus amylovorus
MKQLNSEQLQNIIGGNRWTNAYSAALGCAVPGVKYGKKLGGVWGAVIGGVGGAAVCGLAGYVRKG

SEQ ID NO: 163 - DNA, 198 bp, Lactobacillus amylovorus
  source 1..198: mol_type = genomic DNA
atgaaacaattgaattcagaacaattacaaaatattatcggtggaaatagatggactaatgcatacagcgcagctttgggatgcgctgtccctggagttaaatatggaaaaaaacttggtggcgtatggggtgctgtaattggtggcgtaggcggtgcagcagtctgtggcttggcgggttatgttcgtaaaggctaa

SEQ ID NO: 164 - protein, 68 aa, Lactobacillus sakei
  source 1..68: note = L45
MKTEKKVLDELSLHASAKMGARDVESSMNADSTPVLASVAVSMELLPTASVLYSDVAGCFKYSAKHHC

SEQ ID NO: 165 - DNA, 207 bp, Lactobacillus sakei
  source 1..207: mol_type = genomic DNA; note = L45
atgaaaacagaaaaaaaggttttagatgaactgagcttacacgcttctgcaaaaatgggagcacgtgatgttgaatccagcatgaatgcagactcaacaccagttttagcatcagtcgctgtatccatggaattattgccaactgcgtctgttctttattcggatgttgcaggttgcttcaaatattctgcaaaacatcattgttag

SEQ ID NO: 166 - protein, 91 aa, Lactococcus lactis
  source 1..91: note = subsp. lactis
MKTKSLVLALSAVTLFSAGGIVAQAEGTWQHGYGVSSAYSNYHHGSKTHSATVVNNNTGRQGKDTQRAGVWAKATVGRNLTEKASFYYNFW

SEQ ID NO: 167 - DNA, 276 bp, Lactococcus lactis
  source 1..276: mol_type = genomic DNA; note = subsp. lactis
atgaaaaccaagtctctcgtattggcattatctgcggttacgttattctctgccggaggaattgtagctcaagctgaaggaacatggcaacatggatatggtgttagttcggcatattcaaattatcatcatggtagcaaaactcattcagccacagttgtaaataataatactggccgacaaggtaaggatacacaacgtgccggtgtttgggcaaaagctactgttggacgtaacttaactgaaaaagcttcattttattataacttttggtaa

SEQ ID NO: 168 - protein, 75 aa, Lactococcus lactis
  source 1..75: note = subsp. cremoris
MKNQLNFNIVSDEELSEANGGKLTFIQSTAAGDLYYNTNTHKYVYQQTQNAFGAAANTIVNGWMGGAAGGFGLHH

SEQ ID NO: 169 - DNA, 228 bp, Lactococcus lactis
  source 1..228: mol_type = genomic DNA; note = subsp. cremoris
atgaaaaatcaattaaattttaatattgtttcagatgaagaactttcagaagctaacggaggaaaattaacatttattcaatcgacagcggctggagatttatattacaatactaatacacacaaatatgtttaccaacaaactcaaaacgcttttggggctgctgctaataccattgttaatggatggatgggtggcgctgctggaggtttcgggttgcaccattga

SEQ ID NO: 170 - protein, 68 aa, Lactococcus lactis
  source 1..68: note = subsp. cremoris
MKNQLNFNIVSDEELAEVNGGSLQYVMSAGPYTWYKDTRTGKTICKQTIDTASYTFGVMAEGWGKTFH

SEQ ID NO: 171 - DNA, 207 bp, Lactococcus lactis
  source 1..207: mol_type = genomic DNA; note = subsp. cremoris
atgaaaaatcaattaaattttaatattgtttctgatgaagaacttgcagaagttaatggaggaagcttgcagtatgttatgagtgctggaccatatacttggtataaagatactagaacaggaaaaacaatatgtaaacagacaattgacacagcaagttatacatttggtgtaatggcagaaggatggggaaaaacattccactaa

SEQ ID NO: 172 - protein, 63 aa, Lactococcus sp.
  source 1..63: note = QU 12
MKLIDHLGAPRWAVDTILGAIAVGNLASWVLALVPGPGWAVKAGLATAAAIVKHQGKAAAAAW

SEQ ID NO: 173 - DNA, 192 bp, Lactococcus sp.
  source 1..192: mol_type = genomic DNA; note = QU 12
atgaaattaattgatcatttaggtgctccaagatgggccgttgatactattttaggtgcaatcgcagttgggaacttagcaagttgggttctagcgcttgtccctggtccagggtgggcagtaaaagctggtttagcaactgctgctgccatcgttaaacatcaaggtaaagctgccgctgctgcttggtaa

SEQ ID NO: 174 - protein, 50 aa, Brevibacillus sp.
  source 1..50: note = GI-9
MACQCPDAISGWTHTDYQCHGLENKMYRHVYAICMNGTQVYCRTEWGSSC

SEQ ID NO: 175 - DNA, 153 bp, Brevibacillus sp.
  source 1..153: mol_type = genomic DNA; note = GI-9
atggcttgccaatgtccagatgcgatctcaggttggacgcatacagattaccagtgtcacggtttggagaataaaatgtatagacatgtttatgcaatttgcatgaacggtactcaagtatattgcagaacagagtggggtagcagctgctag

SEQ ID NO: 176 - protein, 57 aa, Leuconostoc pseudomesenteroides
MNKEYNSISNFKKITNKDLQNINGGFIGRAIGDFVYFGAKGLRESGKLLNYYYKHKH

SEQ ID NO: 177 - DNA, 174 bp, Leuconostoc pseudomesenteroides
  source 1..174: mol_type = genomic DNA
atgaataaagaatataatagcattagcaattttaaaaaaattactaataaagacttgcaaaacataaatggtggatttattggtagggcaataggtgactttgtgtactttggagcgaagggactaagagaatctggtaaactacttaattattactataagcataagcattga

SEQ ID NO: 178 - protein, 53 aa, Leuconostoc pseudomesenteroides
MKNQLMSFEVISEKELSTVQGGKGLGKLIGIDWLLGQAKDAVKQYKKDYKRWH

SEQ ID NO: 179 - DNA, 162 bp, Leuconostoc pseudomesenteroides
  source 1..162: mol_type = genomic DNA
atgaaaaatcagttaatgtctttcgaagtgatatcagaaaaagaattgtccacggtacaaggtggcaaaggcttaggtaaactcataggaattgattggcttttgggtcaagctaaggacgctgttaaacagtacaagaaggattacaaacgttggcactaa

SEQ ID NO: 180 - protein, 61 aa, Leuconostoc gelidum
MMNMKPTESYEQLDNSALEQVVGGKYYGNGVHCTKSGCSVNWGEAFSAGVHRLANGGNGFW

SEQ ID NO: 181 - DNA, 186 bp, Leuconostoc gelidum
  source 1..186: mol_type = genomic DNA
atgatgaacatgaaacctacggaaagctatgagcaattggataatagtgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 182 - protein, 61 aa, Leuconostoc carnosum
MNNMKSADNYQQLDNNALEQVVGGKYYGNGVHCTKSGCSVNWGEAFSAGVHRLANGGNGFW

SEQ ID NO: 183 - DNA, 186 bp, Leuconostoc carnosum
  source 1..186: mol_type = genomic DNA
atgaataacatgaaatctgcggataattatcagcaattggataataatgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 184 - protein, 63 aa, Leuconostoc mesenteroides
MFLVNQLGISKSLANTILGAIAVGNLASWLLALVPGPGWATKAALATAETIVKHEGKAAAIAW

SEQ ID NO: 185 - DNA, 192 bp, Leuconostoc mesenteroides
  source 1..192: mol_type = genomic DNA
atgttcttggtaaatcagttagggatttcaaaatcgttagctaatactattcttggtgcaattgctgttggtaatttggccagttggttattagctttggttcctggtccgggttgggcaacaaaagcagcacttgcgacagctgaaacaattgtgaagcatgaaggaaaagcagctgctattgcgtggtaa

SEQ ID NO: 186 - protein, 74 aa, Bacillus licheniformis
MSKKEMILSWKNPMYRTESSYHPAGNILKELQEEEQHSIAGGTITLSTCAILSKPLGNNGYLCTVTKECMPSCN

SEQ ID NO: 187 - DNA, 225 bp, Bacillus licheniformis
  source 1..225: mol_type = genomic DNA
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 188 - protein, 266 aa, Brevibacterium linens
MNNLYRELAPIPGPAWAEIEEEARRTFKRNIAGRRIVDVAGPTGFETSAVTTGHIRDVQSETSGLQVKQRIVQEYIELRTPFTVTRQAIDDVARGSGDSDWQPVKDAATTIAMAEDRAILHGLDAAGIGGIVPGSSNAAVAIPDAVEDFADAVAQALSVLRTVGVDGPYSLLLSSAEYTKVSESTDHGYPIREHLSRQLGAGEIIWAPALEGALLVSTRGGDYELHLGQDLSIGYYSHDSETVELYLQETFGFLALTDESSVPLSL

SEQ ID NO: 189 - DNA, 801 bp, Brevibacterium linens
  source 1..801: mol_type = genomic DNA
gtgaataacctctatcgcgagcttgcccccatccccggcccggcctgggcggagatcgaggaggaggctcgacggacattcaaacgcaatatcgccggccgccggatcgtcgatgtcgcagggcccacgggcttcgagacctccgcggtgaccactggccacatccgagacgtccagtcggagacgagcggactgcaggttaagcagcgcatcgtgcaggaatacatcgagctgcggaccccattcaccgtgactcggcaggccatcgatgacgtggcccgcgggtccggtgactcggactggcagcccgtcaaggatgcggccacgacgatcgcgatggctgaagatcgggccattctccacgggctcgatgcggccgggatcggcggaatcgttcccggcagctcgaatgccgcagtggccatccccgacgccgtcgaggacttcgcggacgccgtcgcccaggcgctgagtgtgctgcgcacggtgggagtcgacgggccctacagcctgttgctctcctccgcggagtacaccaaggtctccgagtccaccgaccacggctacccgatccgcgagcacctctcccggcagctcggcgccggagagatcatctgggcgcccgcgctcgaaggggcgctgctcgtctccacgcgcgggggtgactacgagctccacctcggccaggacctgtcgatcggttactacagccacgacagcgagaccgtcgaactctatctgcaggagaccttcggattcctcgcgctgaccgacgaatccagtgtgcctttgagcctctga

SEQ ID NO: 190 - protein, 71 aa, Listeria innocua
MKKAALKFIIVIAILGFSFSFFSIQSEAKSYGNGVQCNKKKCWVDWGSAISTIGNNSAANWATGGAAGWKS

SEQ ID NO: 191 - DNA, 216 bp, Listeria innocua
  source 1..216: mol_type = genomic DNA
ttgaagaaggcagcgttaaaatttattattgttattgctattctaggtttcagtttttctttctttagcatacaatctgaagctaaatcttatggaaatggagttcagtgtaataagaaaaaatgttgggtagattggggtagtgctataagtactattggaaataattctgcagcgaattgggctacaggtggagcagctggttggaaaagctga

SEQ ID NO: 192 - protein, 68 aa, Bacillus sp.
MSQEAIIRSWKDPFSRENSTQNPAGNPFSELKEAQMDKLVGAGDMEAACTFTLPGGGGVCTLTSECIC

SEQ ID NO: 193 - DNA, 207 bp, Bacillus sp.
  source 1..207: mol_type = genomic DNA
atgagtcaagaagctatcattcgttcatggaaagatcctttttcccgtgaaaattctacacaaaatccagctggtaacccattcagtgagctgaaagaagcacaaatggataagttagtaggtgcgggagacatggaagcagcatgtacttttacattgcctggtggcggcggtgtttgtactctaacttctgaatgtatttgttaa

SEQ ID NO: 194 - protein, 61 aa, Leuconostoc mesenteroides
MTNMKSVEAYQQLDNQNLKKVVGGKYYGNGVHCTKSGCSVNWGEAASAGIHRLANGGNGFW

SEQ ID NO: 195 - DNA, 186 bp, Leuconostoc mesenteroides
  source 1..186: mol_type = genomic DNA
atgacgaatatgaagtctgtggaagcatatcagcaattagataaccagaatctcaagaaagttgttggtggaaagtattatgggaatggtgttcactgtacaaaaagtggatgctctgttaactggggagaagctgcctcagctggcatacatcgtttggccaatggtggaaatggattttggtaa

SEQ ID NO: 196 - protein, 68 aa, Clavibacter michiganensis
  source 1..68: note = subsp. michiganensis
MNDILETETPVMVSPRWDMLLDAGEDTSPSVQTQIDAEFRRVVSPYMSSSGWLCTLTIECGTIICACR

SEQ ID NO: 197 - DNA, 207 bp, Clavibacter michiganensis
  source 1..207: mol_type = genomic DNA; note = subsp. michiganensis
atgaacgacatcctcgagacggagacccccgtcatggtcagcccccggtgggacatgctgctcgacgcgggcgaggacaccagcccgtccgtccagacccagatcgacgcggagttccgtcgcgtcgtgagcccgtacatgtccagcagcggctggctctgcacgctcaccatcgaatgtggcaccatcatctgcgcgtgtcgctga

SEQ ID NO: 198 - protein, 69 aa, Escherichia coli
MELKASEFGVVLSVDALKLSRQSPLGVGIGGGGGGGGGGSCGGQGGGCGGCSNGCSGGNGGSGGSGSHI

SEQ ID NO: 199 - DNA, 207 bp, Escherichia coli
  source 1..207: mol_type = genomic DNA
atggaattaaaagcgagtgaatttggtgtagttttgtccgttgatgctcttaaattatcacgccagtctccattaggtgttggcattggtggtggtggcggcggcggcggcggcggtagctgcggtggtcaaggtggcggttgtggtggttgcagcaacggttgtagtggtggaaacggtggcagcggcggaagtggttcacatatc

SEQ ID NO: 200 - protein, 7 aa, Escherichia coli
MRTGNAN

SEQ ID NO: 201 - DNA, 24 bp, Escherichia coli
  source 1..24: mol_type = genomic DNA
atgcgtactggtaatgcaaactaa

SEQ ID NO: 202 - protein, 99 aa, Klebsiella pneumoniae
MREISQKDLNLAFGAGETDPNTQLLNDLGNNMAWGAALGAPGGLGSAALGAAGGALQTVGQGLIDHGPVNVPIPVLIGPSWNGSGSGYNSATSSSGSGS

SEQ ID NO: 203 - DNA, 300 bp, Klebsiella pneumoniae
  source 1..300: mol_type = genomic DNA
atgagagaaattagtcaaaaggacttaaatcttgcttttggtgcaggagagaccgatccaaatactcaacttctaaacgaccttggaaataatatggcatggggtgctgctcttggcgctcctggcggattaggatcagcagctttgggggccgcgggaggtgcattacaaactgtagggcaaggattaattgaccatggtcctgtaaatgtccccatccctgtactcatcgggccaagctggaatggtagcggtagtggttataacagcgcaacatccagttccggtagtggtagttaa

SEQ ID NO: 204 - protein, 75 aa, Escherichia coli
MREITESQLRYISGAGGAPATSANAAGAAAIVGALAGIPGGPLGVVVGAVSAGLTTAIGSTVGSGSASSSAGGGS

SEQ ID NO: 205 - DNA, 228 bp, Escherichia coli
  source 1..228: mol_type = genomic DNA
atgcgagaaataacagaatcacagttaagatatatttccggggcgggaggtgcgccagcgacttcagctaatgccgcaggtgctgcagctattgttggagctctcgccggaatacctggtggtccacttggggttgtagttggagccgtatctgccggtttgacaacagcaattggctcgaccgtgggaagtggtagtgccagttcttctgctggtggcggtagctaa

SEQ ID NO: 206 - protein, 58 aa, Escherichia coli
MIKHFHFNKLSSGKKNNVPSPAKGVIQIKKSASQLTKGGAGHVPEYFVGIGTPISFYG

SEQ ID NO: 207 - DNA, 177 bp, Escherichia coli
  source 1..177: mol_type = genomic DNA
atgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctga

SEQ ID NO: 208 - protein, 90 aa, Escherichia coli
MYMRELDREELNCVGGAGDPLADPNSQIVRQIMSNAAWGPPLVPERFRGMAVGAAGGVTQTVLQGAAAHMPVNVPIPKVPMGPSWNGSKG

SEQ ID NO: 209 - DNA, 273 bp, Escherichia coli
  source 1..273: mol_type = genomic DNA
atgtatatgagagagttagatagagaggaattaaattgcgttggtggggctggagatccgcttgcagatcctaattcccaaattgtaagacagataatgtctaatgcggcatggggcccgcctttggtgccagagcggtttaggggaatggctgttggagccgcaggtggggttacgcagacagttcttcaaggagcagcagctcatatgccggttaatgtccctatacctaaagttccgatgggaccctcatggaacggaagtaaaggataa

SEQ ID NO: 210 - protein, 50 aa, Enterococcus mundtii
MSQVVGGKYYGNGVSCNKKGCSVDWGKAIGIIGNNSAANLATGGAAGWKS

SEQ ID NO: 211 - DNA, 153 bp, Enterococcus mundtii
  source 1..153: mol_type = genomic DNA
atgtcacaggtagtaggtggaaaatactacggtaatggagtctcatgtaataaaaaagggtgcagtgttgattggggaaaagcgattggcattattggaaataattctgctgcgaatttagctactggtggagcagctggttggaaaagttaa

SEQ ID NO: 212 - protein, 58 aa, Enterococcus mundtii
MKKLTSKEMAQVVGGKYYGNGLSCNKKGCSVDWGKAIGIIGNNSAANLATGGAAGWKS

SEQ ID NO: 213 - DNA, 177 bp, Enterococcus mundtii
  source 1..177: mol_type = genomic DNA
ttgaagaaattaacatcaaaagaaatggcacaagtagtaggtgggaaatactacggtaatggattatcatgtaataaaaaagggtgcagtgttgattggggaaaagctattggcattattggaaataattctgctgcgaatttagctactggtggagcagctggttggaaaagttaa

SEQ ID NO: 214 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 215 - DNA, 192 bp, Streptococcus mutans
  source 1..192: mol_type = genomic DNA
atgtcaaacacacaattattagaagtccttggtactgaaacttttgatgttcaagaagatctctttgcttttgatacaacagatactactattgtggcaagcaacgacgatccagatactcgtttcaaaagttggagcctttgtacgcctggttgtgcaaggacaggtagtttcaatagttactgttgctga

SEQ ID NO: 216 - protein, 53 aa, Streptococcus mutans
MNKLNSNAVVSLNEVSDSELDTILGGNRWWQGVVPTVSYECRMNSWQHVFTCC

SEQ ID NO: 217 - DNA, 162 bp, Streptococcus mutans
  source 1..162: mol_type = genomic DNA
atgaacaagttaaacagtaacgcagtagtttctttgaatgaagtttcagattctgaattggatactattttgggtggtaatcgttggtggcaaggtgttgtgccaacggtctcatatgagtgtcgcatgaattcatggcaacatgttttcacttgctgttaa

SEQ ID NO: 218 - protein, 57 aa, Lactococcus lactis
  source 1..57: note = subsp. lactis
MSTKDFNLDLVSVSKKDSGASPRITSISLCTPGCKTGALMGCNMKTATCHCSIHVSK

SEQ ID NO: 219 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 220 - protein, 57 aa, Lactococcus lactis
MSTKDFNLDLVSVSKKDSGASPRITSISLCTPGCKTGALMGCNMKTATCNCSVHVSK

SEQ ID NO: 221 - DNA, 171 bp, Lactococcus lactis
  source 1..171: mol_type = genomic DNA
atgagtacaaaagatttcaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagcgttcacgtaagcaaa

SEQ ID NO: 222 - protein, 57 aa, Lactococcus lactis
MSTKDFNLDLVSVSKTDSGASTRITSISLCTPGCKTGVLMGCNLKTATCNCSVHVSK

SEQ ID NO: 223 - DNA, 174 bp, Lactococcus lactis
  source 1..174: mol_type = genomic DNA
atgagtacaaaagatttcaacttagatttggtatctgtttcaaaaacagattctggcgcttcaacacgtattaccagcatttcgctttgtacaccaggttgtaaaacaggtgttctgatgggatgtaacctgaaaacagcaacttgtaattgtagcgttcacgtaagcaaataa

SEQ ID NO: 224 - protein, 55 aa, Streptococcus uberis
MNNEDFNLDLIKISKENNSGASPRITSKSLCTPGCKTGILMTCPLKTATCGCHFG

SEQ ID NO: 225 - DNA, 168 bp, Streptococcus uberis
  source 1..168: mol_type = genomic DNA
atgaacaatgaagattttaatttggatctcatcaaaatctcaaaggaaaacaactcaggagcttcacctcgaataactagtaaatcattatgtactcctggatgtaagacgggtattttgatgacttgtccactaaaaactgcaacctgtggttgtcattttggataa

SEQ ID NO: 226 - protein, 57 aa, Lactococcus lactis
  source 1..57: note = subsp. lactis
MSTKDFNLDLVSVSKKDSGASPRITSISLCTPGCKTGALMGCNMKTATCNCSIHVSK

SEQ ID NO: 227 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagtattcacgtaagcaaataa

SEQ ID NO: 228 - protein, 57 aa, Staphylococcus warneri
MENSKVMKDIEVANLLEEVQEDELNEVLGAKKKSGVIPTVSHDCHMNSFQFVFTCCS

SEQ ID NO: 229 - DNA, 174 bp, Staphylococcus warneri
  source 1..174: mol_type = genomic DNA
atggaaaattctaaagttatgaaggacattgaagtagcaaatttattagaagaggttcaagaagatgaattgaatgaagtcttaggagctaagaaaaagtcaggagtaatcccaactgtgtcacacgattgccatatgaattctttccaatttgtatttacttgttgttcataa

SEQ ID NO: 230 - protein, 58 aa, Paenibacillus polymyxa
MAENLFDLDIQVNKSQGSVEPQVLSIVACSSGCGSGKTAASCVETCGNRCFTNVGSLC

SEQ ID NO: 231 - DNA, 177 bp, Paenibacillus polymyxa
  source 1..177: mol_type = genomic DNA
atggctgaaaacttatttgatctggacattcaagtaaacaaatctcaaggttctgtagagcctcaggttctgagcattgttgcatgttctagcggatgtggtagcggtaaaacagctgccagttgtgttgaaacttgtggcaaccggtgctttactaacgttggttcactctgctaa

SEQ ID NO: 232 - protein, 62 aa, Pediococcus acidilactici
MKKIEKLTEKEMANIIGGKYYGNGVTCGKHSCSVDWGKATTCIINNGAMAWATGGHQGNHKC

SEQ ID NO: 233 - DNA, 189 bp, Pediococcus acidilactici
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacttgcataatcaataatggagctatggcatgggctactggtggacatcaaggtaatcataaatgctag

SEQ ID NO: 234 - protein, 60 aa, Pediococcus pentosaceus
MTEIKVLNDKELKNVVGGKYYGNGVHCGKKTCYVDWGQATASIGKIIVNGWTQHGPWAHR

SEQ ID NO: 235 - DNA, 183 bp, Pediococcus pentosaceus
  source 1..183: mol_type = genomic DNA
atgactgaaattaaagtactaaacgataaggaactaaaaaatgtcgtaggaggaaagtattacggtaacggagtgcattgtggtaaaaagacttgctatgtggactggggacaagctacagctagcattggaaaaattatagtgaacggatggacacaacacgggccttgggcacatagataa

SEQ ID NO: 236 - protein, 60 aa, Staphylococcus epidermidis
MKNNKNLFDLEIKKETSQNTDELEPQTAGPAIRASVKQCQKTLKATRLFTVSCKGKNGCK

SEQ ID NO: 237 - DNA, 183 bp, Staphylococcus epidermidis
  source 1..183: mol_type = genomic DNA
atgaaaaataacaaaaatttatttgatttagaaattaaaaaagaaacaagtcaaaacactgatgaacttgaacctcaaactgctggaccagcgattagagcttctgtgaaacaatgtcagaaaactttgaaagctacgcgtttatttacagtgtcttgcaaaggaaaaaacggatgtaaatag

SEQ ID NO: 238 - protein, 62 aa, Carnobacterium maltaromaticum
MKTVKELSVKEMQLTTGGKYYGNGVSCNKNGCTVDWSKAIGIIGNNAAANLTTGGAAGWNKG

SEQ ID NO: 239 - DNA, 189 bp, Carnobacterium maltaromaticum
  source 1..189: mol_type = genomic DNA
atgaaaactgttaaagaacttagcgttaaagaaatgcaactaactacaggaggtaagtattacggaaatggcgtttcctgtaataaaaatggttgtactgtagattggagcaaagctattgggattataggaaacaatgcagcagcaaatttgactacaggtggagccgctggttggaacaaaggataa

SEQ ID NO: 240 - protein, 69 aa, Lactobacillus plantarum
  VARIANT 40: note = Xaa = any amino acid
MYKELTVDELALIDGGKKKKKKVACTWGNAATAAASGAVXGILGGPTGALAGAIWGVSQCASNNLHGMH

SEQ ID NO: 241 - DNA, 210 bp, Lactobacillus plantarum
  misc_feature 119: note = n = a, c, t or g
  source 1..210: mol_type = genomic DNA
atgtataaagaattaacagttgatgaattagcattgattgatggaggaaaaaagaagaagaaaaaagtagcttgtacttggggaaatgcagcaacagccgctgcttctggtgcagttangggtattcttggtgggcctactggtgcactggctggagctatctggggcgtttcacaatgcgcgtctaacaacttacacggcatgcactaa

SEQ ID NO: 242 - protein, 56 aa, Lactobacillus plantarum
MMKKIEKLTEKEMANIIGGKYYGNGVTCGKHSCSVNWGQAFSCSVSHLANFGHGKC

SEQ ID NO: 243 - DNA, 171 bp, Lactobacillus plantarum
  source 1..171: mol_type = genomic DNA
atgatgaaaaaaattgaaaaattaactgaaaaagaaatggccaatatcattggtggtaaatactatggtaatggggttacttgtggtaaacattcctgctctgttaactggggccaagcattttcttgtagtgtgtcacatttagctaacttcggtcatggaaagtgctaa

SEQ ID NO: 244 - protein, 64 aa, Lactobacillus plantarum
MSKLVKTLTVDEISKIQTNGGKPAWCWYTLAMCGAGYDSGTCDYMYSHCFGVKHSSGGGGSYHC

SEQ ID NO: 245 - DNA, 195 bp, Lactobacillus plantarum
  source 1..195: mol_type = genomic DNA
atgagtaaactagttaaaacattaactgtcgatgaaatctctaagattcaaaccaatggtggaaaacctgcatggtgttggtacacattggcaatgtgcggtgctggttatgattcaggcacttgtgattatatgtattcacactgctttggtgtaaaacactctagcggtggtggcggtagctaccattgttag

SEQ ID NO: 246 - protein, 56 aa, Lactobacillus plantarum
MLQFEKLQYSRLPQKKLAKISGGFNRGGYNFGKSVRHVVDAIGSVAGIRGILKSIR

SEQ ID NO: 247 - DNA, 171 bp, Lactobacillus plantarum
  source 1..171: mol_type = genomic DNA
atgctacagtttgagaaattacaatattccaggttgccgcaaaaaaagcttgccaaaatatctggtggttttaatcggggcggttataactttggtaaaagtgttcgacatgttgttgatgcaattggttcagttgcaggcattcgtggtattttgaaaagtattcgttaa

SEQ ID NO: 248 - protein, 52 aa, Lactobacillus plantarum
MKKFLVLRDRELNAISGGVFHAYSARGVRNNYKSAVGPADWVISAVRGFIHG

SEQ ID NO: 249 - DNA, 159 bp, Lactobacillus plantarum
  source 1..159: mol_type = genomic DNA
atgaaaaaatttctagttttgcgtgaccgtgaattaaatgctatttcaggtggcgttttccatgcctatagcgcgcgtggcgttcggaataattataaaagtgctgttgggcctgccgattgggtcattagcgctgtccgaggattcatccacggatag

SEQ ID NO: 250 - protein, 55 aa, Lactobacillus plantarum
MTVNKMIKDLDVVDAFAPISNNKLNGVVGGGAWKNFWSSLRKGFYDGEAGRAIRR

SEQ ID NO: 251 - DNA, 168 bp, Lactobacillus plantarum
  source 1..168: mol_type = genomic DNA
atgactgtgaacaaaatgattaaggatttggatgtagtagatgcatttgcacctatttctaataataagttgaacggggttgttgggggaggcgcttggaaaaatttctggtctagtttaagaaaaggattttatgatggcgaagctggcagagcaatccgtcgttaa

SEQ ID NO: 252 - protein, 57 aa, Lactobacillus plantarum
MKIKLTVLNEFEELTADAEKNISGGRRSRKNGIGYAIGYAFGAVERAVLGGSRDYNK

SEQ ID NO: 253 - DNA, 174 bp, Lactobacillus plantarum
  source 1..174: mol_type = genomic DNA
atgaaaattaaattaactgttttaaatgaatttgaagaattaactgctgacgctgaaaagaatatttctggtggccgtcggagtcgtaaaaatggaattggatacgctattggttatgcgtttggcgcggttgaacgggccgtgcttggtggttcaagggattataataagtga

SEQ ID NO: 254 - protein, 47 aa, Lactobacillus plantarum
MDKFEKISTSNLEKISGGDLTTKLWSSWGYYLGKKARWNLKHPYVQF

SEQ ID NO: 255 - DNA, 141 bp, Lactobacillus plantarum
  source 1..141: mol_type = genomic DNA
atggataaatttgaaaaaattagtacatctaacctagaaaagatctctggcggtgatttaacaaccaagttatggagctcttggggatattatcttggcaagaaagcacgttggaatttaaagcacccatatgttcaattt

SEQ ID NO: 256 - protein, 55 aa, Lactobacillus plantarum
MNNLNKFSTLGKSSLSQIEGGSVPTSVYTLGIKILWSAYKHRKTIEKSFNKGFYH

SEQ ID NO: 257 - DNA, 168 bp, Lactobacillus plantarum
  source 1..168: mol_type = genomic DNA
atgaataacttgaataaattttctactctaggcaagagtagcttgtctcaaattgagggcggatcagtcccaacttcagtatatacgcttggaattaaaattctatggtctgcgtataagcatcgcaaaacgattgaaaaaagttttaataaaggcttttatcattaa

SEQ ID NO: 258 - protein, 55 aa, Lactobacillus plantarum
MNNALSFEQQFTDFSTLSDSELESVEGGRNKLAYNMGHYAGKATIFGLAAWALLA

SEQ ID NO: 259 - DNA, 168 bp, Lactobacillus plantarum
  source 1..168: mol_type = genomic DNA
atgaataacgcattaagttttgaacaacaatttacagacttcagcaccttatcggactctgaattagaatccgttgagggtggccgaaataagcttgcatataatatggggcattacgctggtaaggcaaccatttttggacttgcagcatgggcactccttgcatga

SEQ ID NO: 260 - protein, 47 aa, Lactobacillus plantarum
MDKIIKFQGISDDQLNAVIGGKKKKQSWYAAAGDAIVSFGEGFLNAW

SEQ ID NO: 261 - DNA, 144 bp, Lactobacillus plantarum
  source 1..144: mol_type = genomic DNA
atggataagattattaagtttcaagggatttctgatgatcaattaaatgctgttatcggtgggaaaaagaaaaaacaatcttggtacgcagcagctggtgatgcaatcgttagttttggtgaaggatttttaaatgcttggtaa

SEQ ID NO: 262 - protein, 59 aa, Lactobacillus plantarum
MKISKIEAQARKDFFKKIDTNSNLLNVNGAKCKWWNISCDLGNNGHVCTLSHECQVSCN

SEQ ID NO: 263 - DNA, 180 bp, Lactobacillus plantarum
  source 1..180: mol_type = genomic DNA
atgaaaatttctaagattgaagctcaggctcgtaaagatttttttaaaaaaatcgatactaactcgaacttattaaatgtaaatggtgccaaatgcaagtggtggaatatttcgtgtgatttaggaaataatggccatgtttgtaccttgtcacatgaatgccaagtatcttgtaactaa

SEQ ID NO: 264 - protein, 67 aa, Lactobacillus plantarum
MTKTSRRKNAIANYLEPVDEKSINESFGAGDPEARSGIPCTIGAAVAASIAVCPTTKCSKRCGKRKK

SEQ ID NO: 265 - DNA, 204 bp, Lactobacillus plantarum
  source 1..204: mol_type = genomic DNA
atgactaaaactagtcgtcgtaagaatgctattgctaattatttagaaccagtcgacgaaaaaagtattaatgaatcttttggggctggggatccggaagcaagatccggaattccatgtacaatcggcgcagctgtcgcagcatcaattgcagtttgtccaactactaagtgtagtaaacgttgtggcaagcgtaagaaataa

SEQ ID NO: 266 - protein, 48 aa, Lactobacillus plantarum
MKIQIKGMKQLSNKEMQKIVGGKSSAYSLQMGATAIKQVKKLFKKWGW

SEQ ID NO: 267 - DNA, 147 bp, Lactobacillus plantarum
  source 1..147: mol_type = genomic DNA
atgaaaattcaaattaaaggtatgaagcaacttagtaataaggaaatgcaaaaaatagtaggtggaaagagtagtgcgtattctttgcagatgggggcaactgcaattaaacaggtaaagaaactgtttaaaaaatggggatggtaa

SEQ ID NO: 268 - protein, 207 aa, Propionibacterium jensenii
MNKTHKMATLVIAAILAAGMTAPTAYADSPGNTRITASEQSVLTQILGHKPTQTEYNRYVETYGSVPTEADINAYIEASESEGSSSQTAAHDDSTSPGTSTEIYTQAAPARFSMFFLSGTWITRSGVVSLSLKPRKGGIGNEGDERTWKTVYDKFHNAGQWTRYKNNGVDASMKKQYMCHFKYGMVKTPWNLEPHKKAADVSPVKCN

SEQ ID NO: 269 - DNA, 624 bp, Propionibacterium jensenii
  source 1..624: mol_type = genomic DNA
atgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatggaatctggagccccacaagaaggctgcagacgtcagtccagtcaagtgcaactag

SEQ ID NO: 270 - protein, 96 aa, Propionibacterium thoenii
MKKTLLRSGTIALATAAAFGASLAAAPSAMAVPGGCTYTRSNRDVIGTCKTGSGQFRIRLDCNNAPDKTSVWAKPKVMVSVHCLVGQPRSISFETK

SEQ ID NO: 271 - DNA, 291 bp, Propionibacterium thoenii
  source 1..291: mol_type = genomic DNA
atgaagaagaccctcctgcgaagtggaacgatcgcactggcgaccgcggctgcatttggcgcatcattggcagccgccccatctgccatggccgttcctggtggttgcacgtacacaagaagcaatcgcgacgtcatcggtacctgcaagactggaagcggccagttccgaatccgacttgactgcaacaacgctccagacaaaacttcagtctgggccaagcccaaggtaatggtgtcggttcactgtcttgttggtcaaccgaggtccatctcgttcgagaccaagtga

SEQ ID NO: 272 - protein, 255 aa, Propionibacterium freudenreichii
  source 1..255: note = subsp. freudenreii
MNTKAVNLKSENTTKLVSYLTENQLDEFIRRIRIDGALVEEVSQNAKQALDNTGLNGWINTDCDEGLLSDFISKIASARWIPLAESIRPAVTDRDKYRVSCWFYQGMNIAIYANIGGVANIIGYTEAAVATLLGAVVAVAPVVPGTPTPPKDKSSQYKEVPLAVRLSETYHEEGVRGLFDELNYSESRMISTLRRASTDGVLINSWNDGQDTILLKKYNFQDLQLTVRSRIVGNQTIIEECKITDGRKTLSDETV

SEQ ID NO: 273 - DNA, 768 bp, Propionibacterium freudenreichii
  source 1..768: mol_type = genomic DNA; note = subsp. freudenreii
atgaataccaaagctgtaaatctgaagtcagaaaacacgactaagttggtgagctaccttacggaaaatcaattggatgagtttattagaaggattcgcattgatggcgctcttgtggaagaggtcagtcaaaatgctaagcaggccttagataatactgggctcaatggctggataaatactgattgcgatgaaggccttctctctgatttcatttcaaagatagcaagtgctagatggattccattagctgagtcaattcgacctgcggtgactgacagggataagtatcgagtaagttgctggttctaccaggggatgaatatagcaatttacgcaaatatcggtggcgtggccaatattatcggctatacggaggccgcagtcgcaacactccttggtgcagttgtggcggtagctcctgtggtccctggaactccaacccctccaaaggacaagagttcgcaatataaggaggttccccttgccgttcgtctttccgaaacataccacgaagagggagtacgaggtctattcgacgagctgaactactccgagagccgtatgatctctactctaaggcgagcatcaaccgatggagtcctaattaattcttggaacgatgggcaggatacaattctgcttaagaagtacaatttccaagacttgcaactgactgtcaggagccgcattgttgggaatcaaacaataattgaagaatgcaaaatcactgatggtagaaaaactctttcagacgagactgtgtag

SEQ ID NO: 274 - protein, 618 aa, Pseudomonas aeruginosa
MARPIADLIHFNSTTVTASGDVYYGPGGGTGIGPIARPIEHGLDSSTENGWQEFESYADVGVDPRRYVPLQVKEKRREIELQFRDAEKKLEASVQAELDKADAALGPAKNLAPLDVINRSLTIVGNALQQKNQKLLLNQKKITSLGAKNFLTRTAEEIGEQAVREGNINGPEAYMRFLDREMEGLTAAYNVKLFTEAISSLQIRMNTLTAAKASIEAAAANKAREQAAAEAKRKAEEQARQQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRVADGGGVYNMGNLVAVTPKRHIEIHKGGK

SEQ ID NO: 275 - DNA, 1857 bp, Pseudomonas aeruginosa
  source 1..1857: mol_type = genomic DNA
atggcacgacccattgctgaccttatccacttcaactctacaactgtcacggcaagcggagacgtttattacggccctgggggaggtaccggcattggccccattgccagacctatagagcacggcttggattcgtccactgaaaatggctggcaagagtttgaaagttatgctgatgtgggcgttgaccccagacgctatgttcctcttcaggttaaagaaaaacgcagggagatcgagcttcagttccgagatgccgagaaaaaacttgaggcgtcggtacaagccgagctggataaggctgatgccgctcttggtccggcaaagaatcttgcaccattggacgtcatcaaccgcagtctgaccatcgttggaaacgccctccagcaaaagaatcaaaaactactgctgaatcagaagaagattaccagcctgggtgcaaagaatttccttacccgtacggcggaagagatcggtgaacaagcggtgcgagaaggcaatattaacgggcctgaagcctatatgcgcttcctcgacagggaaatggaaggtctcacggcagcttataacgtaaaactcttcaccgaagcgatcagtagtctccagatccgcatgaatacgttgaccgccgccaaagcaagtattgaggcggccgcagcaaacaaggcgcgtgaacaagcagcggctgaggccaaacgcaaagccgaagagcaggcccgccagcaagcggcgataagagctgccaatacctatgccatgccggccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgagtagcagatggaggcggcgtttacaatatggggaaccttgttgcagtaacgccaaaacgtcatatagaaatccacaagggagggaagtga

SEQ ID NO: 276 - protein, 689 aa, Pseudomonas aeruginosa
MAVNDYEPGSMVITHVQGGGRDIIQYIPARSSYGTPPFVPPGPSPYVGTGMQEYRKLRSTLDKSHSELKKNLKNETLKEVDELKSEAGLPGKAVSANDIRDEKSIVDALMDAKAKSLKAIEDRPANLYTASDFPQKSESMYQSQLLASRKFYGEFLDRHMSELAKAYSADIYKAQIAILKQTSQELENKARSLEAEAQRAAAEVEADYKARKANVEKKVQSELDQAGNALPQLTNPTPEQWLERATQLVTQAIANKKKLQTANNALIAKAPNALEKQKATYNADLLVDEIASLQARLDKLNAETARRKEIARQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 277 - DNA, 2070 bp, Pseudomonas aeruginosa
  source 1..2070: mol_type = genomic DNA
atggctgtcaatgattacgaacctggttcgatggttattacacatgtgcagggtggtgggcgtgacataatccagtatattcctgctcgatcaagctacggtactccaccatttgtcccaccaggaccaagtccgtatgtcggtactggaatgcaggagtacaggaagctaagaagtacgcttgataagtcccattcagaactcaagaaaaacctgaaaaatgaaaccctgaaggaggttgatgaactcaagagtgaagcggggttgccaggtaaagcggtcagtgccaatgacatccgcgatgaaaagagtatcgttgatgcactcatggatgccaaagcaaaatcgctaaaggccattgaggatcgcccggccaatctttatacggcttcagactttcctcagaagtcagagtcgatgtaccagagtcagttgctggccagccgaaaattctatggagagttcctggatcgccatatgagtgagctggccaaagcgtacagcgccgatatctataaggcgcaaatcgctatcttgaaacaaacgtctcaagagctggagaataaagcccggtcattggaagcagaagcccagcgagccgctgctgaggtggaggcggactacaaggccaggaaggcaaatgtcgagaaaaaagtgcagtccgagcttgaccaggctgggaatgctttgcctcaactgaccaatccaacgccagagcagtggcttgaacgcgctactcaactggttacgcaggcgatcgccaataagaagaaattgcagactgcaaacaatgccttgattgccaaggcacccaatgcactggagaaacaaaaggcaacctacaacgccgatctcctagtggatgaaatcgccagcctgcaagcacggctggacaagctgaacgccgaaacggcaaggcgcaaggaaatcgctcgtcaagcggcgatcagggctgccaatacttatgccatgccagccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgaatagcagatggaggcggcgtttacaatatggggaaccttgttgcagtaacgccaaaacgtcatatagaaatccacaagggagggaagtga

SEQ ID NO: 278 - protein, 47 aa, Ruminococcus gnavus
MRNDVLTLTNPMEEKELEQILGGGNGVLKTISHECNMNTWQFLFTCC

SEQ ID NO: 279 - DNA, 144 bp, Ruminococcus gnavus
  source 1..144: mol_type = genomic DNA
atgagaaatgacgtattaacattaacaaacccaatggaagagaacgaactggagcagatcttaggtggtggcaatggtgtgttaaaaacgattagccacgaatgcaatatgaacacatggcagttcctgtttacttgttgctaa

SEQ ID NO: 280 - protein, 55 aa, Lactobacillus sakei
MKNAKSLTIQEMKSITGGKYYGNGVSCNSHGCSVNWGQAWTCGVNHLANGGHGVC

SEQ ID NO: 281 - DNA, 168 bp, Lactobacillus sakei
  source 1..168: mol_type = genomic DNA
atgaaaaacgcaaaaagcctaacaattcaagaaatgaaatctattacaggtggtaaatactatggtaatggcgttagctgtaactctcacggctgttcagtaaattgggggcaagcatggacttgtggagtaaaccatctagctaatggcggtcatggagtttgttaa

SEQ ID NO: 282 - protein, 59 aa, Lactobacillus sakei; identical to SEQ ID NO: 68

SEQ ID NO: 283 - DNA, 180 bp, Lactobacillus sakei; identical to SEQ ID NO: 69
  source 1..180: mol_type = genomic DNA

SEQ ID NO: 284 - protein, 61 aa, Lactobacillus sakei
MEKFIELSLKEVTAITGGKYYGNGVHCGKHSCTVDWGTAIGNIGNNAAANWATGGNAGWNK

SEQ ID NO: 285 - DNA, 186 bp, Lactobacillus sakei
  source 1..186: mol_type = genomic DNA
atggaaaagtttattgaattatctttaaaagaagtaacagcaattacaggtggaaaatattatggtaacggtgtacactgtggaaaacattcatgtaccgtagactggggaacagctattggaaatatcggaaataatgcagctgcaaactgggccacaggcggaaacgctggctggaataaataa

SEQ ID NO: 286 - protein, 56 aa, Streptococcus salivarius
MKSTNNQSIAEIAAVNSLQEVSMEELDQIIGAGNGVVLTLTHECNLATWTKKLKCC

SEQ ID NO: 287 - DNA, 171 bp, Streptococcus salivarius
  source 1..171: mol_type = genomic DNA
atgaaatcaacaaataatcaaagtatcgcagaaattgcagcagtaaactcactacaagaagtaagtatggaggaactagaccaaattattggtgccggaaacggagtggttcttactcttactcatgaatgtaacctagcaacttggacaaaaaaactaaaatgttgctaa

SEQ ID NO: 288 - protein, 51 aa, Streptococcus pyogenes
  source 1..51: note = serotype M28
MSFMKNSKDILTNAIEEVSEKELMEVAGGKKGSGWFATITDDCPNSVFVCC

SEQ ID NO: 289 - DNA, 156 bp, Streptococcus pyogenes
  source 1..156: mol_type = genomic DNA; note = serotype M28
atgagttttatgaaaaattcaaaggatattttgactaatgctatcgaagaagtttctgaaaaagaacttatggaagtagctggtggtaaaaaaggttccggttggtttgcaactattactgatgactgtccgaactcagtattcgtttgttgttaa

SEQ ID NO: 290 - protein, 48 aa, Streptococcus salivarius
MKNSKDVLNNAIEEVSEKELMEVAGGKKGPGWIATITDDCPNSIFVCC

SEQ ID NO: 291 - DNA, 147 bp, Streptococcus salivarius
  source 1..147: mol_type = genomic DNA
atgaaaaactcaaaagatgttttgaacaatgctatcgaagaggtttctgaaaaagaacttatggaagtagctggtggtaaaaaaggtccaggttggattgcaactattactgatgactgtccaaactcaatattcgtttgttgttaa

SEQ ID NO: 292 - protein, 48 aa, Streptococcus salivarius
MKNSKDILNNAIEEVSEKELMEVAGGKRGSGWIATITDDCPNSVFVCC

SEQ ID NO: 293 - DNA, 147 bp, Streptococcus salivarius
  source 1..147: mol_type = genomic DNA
atgaaaaactcaaaagatattttgaacaatgctatcgaagaagtttctgaaaaagaacttatggaagtagctggtggtaaaagaggttcaggttggattgcaactattactgatgactgtccaaactcagtattcgtttgttgttaa

SEQ ID NO: 294 - protein, 62 aa, Staphylococcus aureus
MKSSFLEKDIEEQVTWFEEVSEQEFDDDIFGACSTNTFSLSDYWGNKGNWCTATHECMSWCK

SEQ ID NO: 295 - DNA, 189 bp, Staphylococcus aureus
  source 1..189: mol_type = genomic DNA
atgaaaagttcttttttagaaaaagatatagaagaacaagtgacatggttcgaggaagtttcagaacaagaatttgacgatgatatttttggagcttgtagtacaaacactttttctttgagtgactattggggtaataaaggaaattggtgtactgctactcacgaatgtatgtcttggtgtaaataa

SEQ ID NO: 296 - protein, 67 aa, Staphylococcus aureus
MKNELGKFLEENELELGKFSESDMLEITDDEVYAAGTPLALLGGAATGVIGYISNQTCPTTACTRAC

SEQ ID NO: 297 - DNA, 204 bp, Staphylococcus aureus
  source 1..204: mol_type = genomic DNA
atgaaaaatgaattaggtaagtttttagaagaaaacgaattagagttaggtaaattttcagaatcagacatgctagaaattactgatgatgaagtatatgcagctggaacacctttagccttattgggtggagctgccaccggggtgataggttatatttctaaccaaacatgtccaacaactgcttgtacacgcgcttgctag

SEQ ID NO: 298 - protein, 46 aa, Streptococcus pyogenes
MNNTIKDFDLDLKTNKKDTATPYVGSRYLCTPGSCWKLVCFTTTVK

SEQ ID NO: 299 - DNA, 141 bp, Streptococcus pyogenes
  source 1..141: mol_type = genomic DNA
atgaataacacaattaaagactttgatctcgatttgaaaacaaataaaaaagacactgctacaccttatgttggtagccgttacctatgtacccctggttcttgttggaaattagtttgctttacaacaactgttaaataa

SEQ ID NO: 300 - protein, 51 aa, Streptococcus pyogenes
MEKNNEVINSIQEVSLEELDQIIGAGKNGVFKTISHECHLNTWAFLATCCS

SEQ ID NO: 301 - DNA, 156 bp, Streptococcus pyogenes
  source 1..156: mol_type = genomic DNA
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgttcataa

SEQ ID NO: 302 - protein, 51 aa, Streptococcus pyogenes
  source 1..51: note = serotype M49
MTKEHEIINSIQEVSLEELDQIIGAGKNGVFKTISHECHLNTWAFLATCCS

SEQ ID NO: 303 - DNA, 156 bp, Streptococcus pyogenes
  source 1..156: mol_type = genomic DNA; note = serotype M49
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgctcataa

SEQ ID NO: 304 - protein, 56 aa, Bacillus subtilis
MEKLFKEVKLEELENQKGSGLGKAQCAALWLQCASGGTIGCGGGAVACQNYRQFCR

SEQ ID NO: 305 - DNA, 171 bp, Bacillus subtilis
  source 1..171: mol_type = genomic DNA
atggaaaagctatttaaagaagttaaactagaggaactcgaaaaccaaaaaggtagtggattaggaaaagctcagtgtgctgcgttgtggctacaatgtgctagtggcggtacaattggttgtggtggcggagctgttgcttgtcaaaactatcgtcaattctgcagataa

SEQ ID NO: 306 - protein, 56 aa, Bacillus subtilis
MSKFDDFDLDVVKVSKQDSKITPQWKSESLCTPGCVTGALQTCFLQTLTCNCKISK

SEQ ID NO: 307 - DNA, 171 bp, Bacillus subtilis
  source 1..171: mol_type = genomic DNA
atgtcaaagttcgatgatttcgatttggatgttgtgaaagtctctaaacaagactcaaaaatcactccgcaatggaaaagtgaatcactttgtacaccaggatgtgtaactggtgcattgcaaacttgcttccttcaaacactaacttgtaactgcaaaatctctaaataa

SEQ ID NO: 308 - protein, 50 aa, Bacillus subtilis
MKLPVQQVYSVYGGKDLPKGHSHSTMPFLSKLQFLTKIYLLDIHTQPFFI

SEQ ID NO: 309 - DNA, 153 bp, Bacillus subtilis
  source 1..153: mol_type = genomic DNA
ttgaaattgccggtgcaacaggtctattcggtctatgggggtaaggatctcccaaaagggcatagtcattctactatgccctttttaagtaaattacaatttttaactaaaatctacctcttggatatacatacacaaccgtttttcatttga

SEQ ID NO: 310 - protein, 43 aa, Bacillus subtilis
MKKAVIVENKGCATCSIGAACLVDGPIPDFEIAGATGLFGLWG

SEQ ID NO: 311 - DNA, 132 bp, Bacillus subtilis
  source 1..132: mol_type = genomic DNA
atgaaaaaagctgtcattgtagaaaacaaaggttgtgcaacatgctcgatcggagccgcttgtctagtggacggtcctatccctgattttgaaattgccggtgcaacaggtctattcggtctatgggggtaa

SEQ ID NO: 312 - protein, 58 aa, Streptococcus thermophilus; identical to SEQ ID NO: 24

SEQ ID NO: 313 - DNA, 177 bp, Streptococcus thermophilus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcctgtaaaaattgtgcttaa

SEQ ID NO: 314 - protein, 64 aa, Streptococcus thermophilus
MKQYNGFEVLHELDLANVTGGQINWGSVVGHCIGGAIIGGAFSGGAAAGVGCLVGSGKAIINGL

SEQ ID NO: 315 - DNA, 195 bp, Streptococcus thermophilus
  source 1..195: mol_type = genomic DNA
atgaagcagtataatggttttgaggttctacatgaacttgacttagcaaatgtaactggcggtcaaattaattggggatcagttgtaggacactgtataggtggagctattatcggaggtgcattttcaggaggtgcagcggctggagtaggatgccttgttgggagcggaaaggcaatcataaatggattataa

SEQ ID NO: 316 - protein, 85 aa, Streptococcus thermophilus
MNTITICKFDVLDAELLSTVEGGYSGKDCLKDMGGYALAGAGSGALWGAPAGGVGALPGAFVGAHVGAIAGGFACMGGMIGNKFN

SEQ ID NO: 317 - DNA, 258 bp, Streptococcus thermophilus
  source 1..258: mol_type = genomic DNA
atgaatacaataactatttgtaaatttgatgttttagatgctgaacttctttcgacagttgagggtggatactctggtaaggattgtttaaaagacatgggaggatatgcattggcaggagctggaagtggagctctgtggggagctccagcaggaggtgttggagcacttccaggtgcatttgtcggagctcatgttggggcaattgcaggaggctttgcatgtatgggtggaatgattggtaataagtttaactaa

SEQ ID NO: 318 - protein, 52 aa, Bacillus cereus
MSEIKKALNTLEIEDFDAIEMVDVDAMPENEALEIMGASCTTCVCTCSCCTT

SEQ ID NO: 319 - DNA, 159 bp, Bacillus cereus
  source 1..159: mol_type = genomic DNA
atgagtgaaattaaaaaagcattaaatacgcttgaaattgaagattttgatgcaattgaaatggttgatgttgatgctatgccagaaaacgaagcgcttgaaattatgggagcgtcatgtacgacatgcgtatgtacatgcagttgttgtacaacttga

SEQ ID NO: 320 - protein, 47 aa, Bacillus cereus
MEVMNNALITKVDEEIGGNAACVIGCIGSCVISEGIGSLVGTAFTLG

SEQ ID NO: 321 - DNA, 144 bp, Bacillus cereus
  source 1..144: mol_type = genomic DNA
atggaagttatgaacaatgctttaattacaaaagtagatgaggagattggaggaaacgctgcttgtgtaattggttgtattggcagttgcgtaattagtgaaggaattggttcacttgtaggaacagcatttactttaggttaa

SEQ ID NO: 322 - protein, 49 aa, Bacillus cereus
MEVLNKQNVNIIPESEEVGGWVACVGACGTVCLASGGVGTEFAAASYFL

SEQ ID NO: 323 - DNA, 150 bp, Bacillus cereus
  source 1..150: mol_type = genomic DNA
atggaagttttaaacaaacaaaatgtaaatattattccagaatctgaagaagtaggtggatgggtagcatgtgttggagcatgtggtacagtatgtcttgctagtggtggtgttggaacagagtttgcagctgcatcttatttcctataa

SEQ ID NO: 324 - protein, 40 aa, Bacillus thuringiensis
METPVVQPRDWTCWSCLVCAACSVELLNLVTAATGASTAS

SEQ ID NO: 325 - DNA, 123 bp, Bacillus thuringiensis
  source 1..123: mol_type = genomic DNA
atggaaacaccagtagtacaaccaagggattggacttgttggagttgcttagtatgtgcagcatgttctgtggaattattaaatttagttactgcggcaacaggggctagtactgcaagctaa

SEQ ID NO: 326 - protein, 42 aa, Rhizobium leguminosarum
  source 1..42: note = bv. trifolii
MDNKVAKNVEVKKGSIKATFKAAVLKSKTKVDIGGSRQGCVA

SEQ ID NO: 327 - DNA, 129 bp, Rhizobium leguminosarum
  source 1..129: mol_type = genomic DNA; note = bv. trifolii
atggataacaaggttgcgaagaatgtcgaagtgaagaagggctccatcaaggcgaccttcaaggctgctgttctgaagtcgaagacgaaggtcgacatcggaggtagccgtcagggctgcgtcgcttaa

SEQ ID NO: 328 - protein, 70 aa, Streptococcus uberis
MNTIEKFENIKLFSLKKIIGGKTVNYGNGLYCNQKKCWVNWSETATTIVNNSIMNGLTGGNAGWHSGGRA

SEQ ID NO: 329 - DNA, 213 bp, Streptococcus uberis
  source 1..213: mol_type = genomic DNA
atgaatacaattgaaaaatttgaaaatattaaacttttttcactaaagaaaattatcggtggcaaaactgtaaattatggtaatggcctttattgtaaccaaaaaaaatgctgggtaaactggtcagaaactgctacaacaatagtaaataattccatcatgaacgggctcacaggtggtaatgcgggttggcactcaggcgggagagcataa

SEQ ID NO: 330 - protein, 76 aa, Streptococcus uberis
MDILLELAGYTGIASGTAKKVVDAIDKGAAAFVIISIISTVISAGALGAVSASADFIILTVKNYISRNLKAQAVIW

SEQ ID NO: 331 - DNA, 231 bp, Streptococcus uberis
  source 1..231: mol_type = genomic DNA
atggacattttattagaactcgcaggatatactgggatagcctcaggtactgcaaaaaaagttgttgatgccattgataaaggagctgcagcctttgttattatttcaattatctcaacagtaattagtgcgggagcattgggagcagtttcagcctcagctgattttattattttaactgtaaaaaattacattagtagaaatttaaaagcacaagctgtcatttggtaa

SEQ ID NO: 332 - protein, 64 aa, Clostridium perfringens
MDSELFKLMATQGAFAILFSYLLFYVLKENSKREDKYQNIIEELTELLPKIKEDVEDIKEKLNK

SEQ ID NO: 333 - DNA, 195 bp, Clostridium perfringens
  source 1..195: mol_type = genomic DNA
atggatagtgaattatttaagttaatggcaacacaaggagcctttgcaatattattttcgtatttattgttttatgttttaaaagagaatagtaaaagagaagataagtatcaaaatataatagaggagcttacagaattattgccaaaaataaaagaagatgtagaagatataaaagaaaaacttaataaatag

SEQ ID NO: 334 - protein, 47 aa, Micrococcus varians
MTNAFQALDEVTDAELDAILGGGSGVIPTISHECHMNSFQFVFTCCS

SEQ ID NO: 335 - DNA, 144 bp, Micrococcus varians
  source 1..144: mol_type = genomic DNA
atgacgaacgcatttcaggcactggacgaagtcacggacgccgagctcgacgccatccttggcgggggcagtggtgttattcccacgatcagccacgagtgccacatgaactccttccagttcgtgttcacctgctgctcctga

SEQ ID NO: 336 - protein, 285 aa, Streptococcus equi
  source 1..285: note = subsp. zooepidemicus
MKRIFFAFLSLCLFIFGTQTVSAATYTRPLDTGNITTGFNGYPGHVGVDYAVPVGTPVRAVANGTVKFAGNGANHPWMLWMAGNCVLIQHADGMHTGYAHLSKISVSTDSTVKQGQIIGYTGATGQVTGPHLHFEMLPANPNWQNGFSGRIDPTGYIANAPVFNGTTPTEPTTPTTNLKIYKVDDLQKINGIWQVRNNILVPTDFTWVDNGIAADDVIEVTSNGTRTSDQVLQKGGYFVINPNNVKSVGTPMKGSGGLSWAQVNFTTGGNVWLNTTSKDNLLYGK

SEQ ID NO: 337 - DNA, 858 bp, Streptococcus equi
  source 1..858: mol_type = genomic DNA; note = subsp. zooepidemicus
atgaaacgtatattttttgctttcttaagtttatgcttatttatattcggaacacaaacggtatctgcagctacttatactcggccattagatacgggaaatatcactacagggtttaacggataccctggtcatgttggagtcgattatgcagtacccgttggaactccggttagagcagttgcaaatggtacagtcaaatttgcaggtaatggggctaatcacccatggatgctttggatggctggaaactgtgttctaattcaacatgctgacgggatgcatactggatatgcacacttatcaaaaatttcagttagcacagatagtacagttaaacaaggacaaatcataggttatactggtgccaccggccaagttaccggtccacatttgcattttgaaatgttgccagcaaatcctaactggcaaaatggtttttctggaagaatagatccaaccggatacatcgctaatgcccctgtatttaatggaacaacacctacagaacctactactcctacaacaaatttaaaaatctataaagttgatgatttacaaaaaattaatggtatttggcaagtaagaaataacatacttgtaccaactgatttcacatgggttgataatggaattgcagcagatgatgtaattgaagtaactagcaatggaacaagaacctctgaccaagttcttcaaaaaggtggttattttgtcatcaatcctaataatgttaaaagtgttggaactccgatgaaaggtagtggtggtctatcttgggctcaagtaaactttacaacaggtggaaatgtctggttaaatactactagcaaagacaacttactttacggaaaataa

SEQ ID NO: 338 - protein, 45 aa, Myxococcus fulvus
ANCSCSTASDYCPILTFCTTGTACSYTPTGCGTGWVYCACNGNFY

SEQ ID NO: 339 - DNA, 135 bp, synthetic construct
  misc_feature 1..135: note = Polynucleotide encoding the polypeptide of SEQ IDNO: 338
  source 1..135: mol_type = other DNA
gcgaactgcagctgcagcaccgcgagcgattattgcccgattctgaccttttgcaccaccggcaccgcgtgcagctataccccgaccggctgcggcaccggctgggtgtattgcgcgtgcaacggcaacttttat

SEQ ID NO: 340 - protein, 19 aa, Streptomyces griseoluteus
CANSCSYGPLTWSCDGNTK

SEQ ID NO: 341 - DNA, 57 bp, synthetic construct
  misc_feature 1..57: note = Polynucleotide encoding the polypeptide of SEQ IDNO: 340
  source 1..57: mol_type = other DNA
tgcgcgaacagctgcagctatggcccgctgacctggagctgcgatggcaacaccaaa

SEQ ID NO: 342 - protein, 19 aa, Streptoverticillium griseoverticillatum
CKQSCSFGPFTFVCDGNTK

SEQ ID NO: 343 - DNA, 57 bp, synthetic construct
  misc_feature 1..57: note = Polynucleotide encoding the polypeptide of SEQ IDNO: 342
  source 1..57: mol_type = other DNA
tgcaaacagagctgcagctttggcccgtttacctttgtgtgcgatggcaacaccaaa

SEQ ID NO: 344 - protein, 7 aa, Carnobacterium sp.
GSEIQPR

SEQ ID NO: 345 - DNA, 21 bp, synthetic construct
  misc_feature 1..21: note = Polynucleotide encoding the polypeptide of SEQ IDNO: 344
  source 1..21: mol_type = other DNA
ggcagcgaaattcagccgcgc

SEQ ID NO: 346 - protein, 39 aa, Lactococcus lactis
  source 1..39: note = subsp. lactis
GTWDDIGQGIGRVAYWVGKAMGNMSDVNQASRINRKKKH

SEQ ID NO: 347 - DNA, 117 bp, synthetic construct
  misc_feature 1..117: note = Polynucleotide encoding the polypeptide of SEQ IDNO: 346
  source 1..117: mol_type = other DNA
ggcacctgggatgatattggccagggcattggccgcgtggcgtattgggtgggcaaagcgatgggcaacatgagcgatgtgaaccaggcgagccgcattaaccgcaaaaaaaaacat

SEQ ID NO: 348 - protein, 35 aa, Lactococcus lactis
  source 1..35: note = subsp. lactis
KKWGWLAWVDPAYEFIKGFGKGAIKEGNKDKWKNI

SEQ ID NO: 349 - DNA, 105 bp, synthetic construct
  misc_feature 1..105: note = Polynucleotide encoding the polypeptide of SEQ IDNO: 348
  source 1..105: mol_type = other DNA
aaaaaatggggctggctggcgtgggtggatccggcgtatgaatttattaaaggctttggcaaaggcgcgattaaagaaggcaacaaagataaatggaaaaacatt

SEQ ID NO: 350 - protein, 19 aa, Streptomyces sp.
CVQSCSFGPLTWSCDGNTK

SEQ ID NO: 351 - DNA, 57 bp, synthetic construct
  misc_feature 1..57: note = Polynucleotide encoding the polypeptide of SEQ IDNO: 350
  source 1..57: mol_type = other DNA
tgcgtgcagagctgcagctttggcccgctgacctggagctgcgatggcaacaccaaa

SEQ ID NO: 352 - protein, 19 aa, Actinoplanes liguriae
SSGWVCTLTIECGTVICAC

SEQ ID NO: 353 - DNA, 57 bp, synthetic construct
  misc_feature 1..57: note = Polynucleotide encoding the polypeptide of SEQ IDNO: 352
  source 1..57: mol_type = other DNA
agcagcggctgggtgtgcaccctgaccattgaatgcggcaccgtgatttgcgcgtgc

SEQ ID NO: 354 - protein, 38 aa, Lactobacillus curvatus
  VARIANT 34: note = Xaa = any amino acid
  VARIANT 37: note = Xaa = any amino acid
YTAKQCLQAIGSCGIAGTGAGAAGGPAGAFVGAXVVXI

SEQ ID NO: 355 - DNA, 114 bp, synthetic construct
  misc_feature 1..114: note = Polynucleotide encoding the polypeptide of SEQ IDNO: 354
  misc_feature 100..111: note = n = A,T,C or G
  misc_feature 100..102: note = nnn = a codon other than a stop codon
  misc_feature 109..111: note = nnn = a codon other than a stop codon
  source 1..114: mol_type = other DNA
tataccgcgaaacagtgcctgcaggcgattggcagctgcggcattgcgggcaccggcgcgggcgcggcgggcggcccggcgggcgcgtttgtgggcgcgnnngtggtgnnnatt

SEQ ID NO: 356 - protein, 42 aa, Lactobacillus sakei
  VARIANT 32: note = Xaa = any amino acid
TKYYGNGVYCNSKKCWVDWGQAAGGIGQTVVXGWLGGAIPGK

SEQ ID NO: 357 - DNA, 126 bp, synthetic construct
  misc_feature 1..126: note = Polynucleotide encoding the polypeptide of SEQ IDNO: 356
  misc_feature 94..96: note = nnn = any amino acid-coding triplet
  source 1..126: mol_type = other DNA
accaaatattatggcaacggcgtgtattgcaacagcaaaaaatgctgggtggattggggccaggcggcgggcggcattggccagaccgtggtgnnnggctggctgggcggcgcgattccgggcaaa

SEQ ID NO: 358 - protein, 22 aa, Streptococcus mutans
FKSWSFCTPGCAKTGSFNSYCC

SEQ ID NO: 359 - DNA, 132 bp, synthetic construct
  misc_feature 1..132: note = Polynucleotide encoding the polypeptide of SEQ IDNO: 358
  source 1..132: mol_type = other DNA
tttaaaagctggagcttttgcaccccgggctgcgcgaaaaccggcagctttaacagctattgctgctttaaaagctggagcttttgcaccccgggctgcgcgaaaaccggcagctttaacagctattgctgc

SEQ ID NO: 360 - protein, 43 aa, Enterococcus mundtii
KYYGNGVSCNKKGCSVDWGKAIGIIGNNSAANLATGGAAGWSK

SEQ ID NO: 361 - DNA, 129 bp, synthetic construct
  misc_feature 1..129: note = Polynucleotide encoding the polypeptide of SEQ IDNO: 360
  source 1..129: mol_type = other DNA
aaatattatggcaacggcgtgagctgcaacaaaaaaggctgcagcgtggattggggcaaagcgattggcattattggcaacaacagcgcggcgaacctggcgaccggcggcgcggcgggctggagcaaa

SEQ ID NO: 362 - protein, 41 aa, Lactobacillus sakei
  VARIANT 9..37: note = X= any amino acid
KYYGNGVHXGKHSXTVDWGTAIGNIGNNAAANXATGXNAGG

SEQ ID NO: 363 - DNA, 123 bp, synthetic construct
  misc_feature 1..123: note = Polynucleotide encoding the polypeptide of SEQ IDNO: 362
  misc_feature 25..27: note = nnn = any amino acid-coding triplet
  misc_feature 40..42: note = nnn = any amino acid-coding triplet
  misc_feature 97..99: note = nnn = any amino acid-coding triplet
  misc_feature 109..111: note = nnn = any amino acid-coding triplet
  source 1..123: mol_type = other DNA
aaatattatggcaacggcgtgcatnnnggcaaacatagcnnnaccgtggattggggcaccgcgattggcaacattggcaacaacgcggcggcgaacnnngcgaccggcnnnaacgcgggcggc

SEQ ID NO: 364 - protein, 31 aa, Lactobacillus paracasei
GMSGYIQGIPDFLKGYLHGISAANKHKKGRL

SEQ ID NO: 365 - DNA, 93 bp, synthetic construct
  misc_feature 1..93: note = Polynucleotide encoding the polypeptide of SEQ IDNO: 364
  source 1..93: mol_type = other DNA
ggcatgagcggctatattcagggcattccggattttctgaaaggctatctgcatggcattagcgcggcgaacaaacataaaaaaggccgcctg

SEQ ID NO: 366 - protein, 31 aa, Leuconostoc mesenteroides
KGKGFWSWASKATSWLTGPQQPGSPLLKKHR

SEQ ID NO: 367 - DNA, 93 bp, synthetic construct
  misc_feature 1..93: note = Polynucleotide encoding the polypeptide of SEQ IDNO: 366
  source 1..93: mol_type = other DNA
aaaggcaaaggcttttggagctgggcgagcaaagcgaccagctggctgaccggcccgcagcagccgggcagcccgctgctgaaaaaacatcgc

SEQ ID NO: 368 - protein, 43 aa, Leuconostoc mesenteroides
KNYGNGVHCTKKGCSVDWGYAWTNIANNSVMNGLTGGNAGWHN

SEQ ID NO: 369 - DNA, 129 bp, synthetic construct
  misc_feature 1..129: note = Polynucleotide encoding the polypeptide of SEQ IDNO: 368
  source 1..129: mol_type = other DNA
aaaaactatggcaacggcgtgcattgcaccaaaaaaggctgcagcgtggattggggctatgcgtggaccaacattgcgaacaacagcgtgatgaacggcctgaccggcggcaacgcgggctggcataac

SEQ ID NO: 370 - protein, 47 aa, Bacillus subtilis
AIKLVQSPNGNFAASFVLDGTKWIFKSKYYDSSKGYWVGIYEVWDRK

SEQ ID NO: 371 - DNA, 141 bp, synthetic construct
  misc_feature 1..141: note = Polynucleotide encoding the polypeptide of SEQ IDNO: 370
  source 1..141: mol_type = other DNA
gcgattaaactggtgcagagcccgaacggcaactttgcggcgagctttgtgctggatggcaccaaatggatttttaaaagcaaatattatgatagcagcaaaggctattgggtgggcatttatgaagtgtgggatcgcaaa

SEQ ID NO: 372 - protein, 12 aa, Bacillus licheniformis
  VARIANT 7: note = Xaa = any amino acid
ISLEICXIFHDN

SEQ ID NO: 373 - DNA, 36 bp, synthetic construct
  misc_feature 1..36: note = Polynucleotide encoding the polypeptide of SEQ IDNO: 372
  misc_feature 19..21: note = nnn = any amino acid-coding triplet
  source 1..36: mol_type = other DNA
attagcctggaaatttgcnnnatttttcatgataac

SEQ ID NO: 374 - protein, 37 aa, Lactococcus lactis
  source 1..37: note = subsp. lactis
TSYGNGVHCNKSKCWIDVSELETYKAGTVSNPKDILW

SEQ ID NO: 375 - DNA, 111 bp, synthetic construct
  misc_feature 1..111: note = Polynucleotide encoding the polypeptide of SEQ IDNO: 374
  source 1..111: mol_type = other DNA
accagctatggcaacggcgtgcattgcaacaaaagcaaatgctggattgatgtgagcgaactggaaacctataaagcgggcaccgtgagcaacccgaaagatattctgtgg

SEQ ID NO: 376 - protein, 9 aa, Serratia plymuthica
DYHHGVRVL

SEQ ID NO: 377 - DNA, 27 bp, synthetic construct
  misc_feature 1..27: note = Polynucleotide encoding the polypeptide of SEQ IDNO: 376
  source 1..27: mol_type = other DNA
gattatcatcatggcgtgcgcgtgctg

SEQ ID NO: 378 - protein, 15 aa, Halobacterium sp.
DIDITGCSACKYAAG

SEQ ID NO: 379 - DNA, 45 bp, synthetic construct
  misc_feature 1..45: note = Polynucleotide encoding the polypeptide of SEQ IDNO: 378
  source 1..45: mol_type = other DNA
gatattgatattaccggctgcagcgcgtgcaaatatgcggcgggc

SEQ ID NO: 380 - protein, 12 aa, Bacillus subtilis
  VARIANT 1..6: note = X= any amino acid
XXKEIXHIFHDN

SEQ ID NO: 381 - DNA, 36 bp, synthetic construct
  misc_feature 1..36: note = Polynucleotide encoding the polypeptide of SEQ IDNO: 380
  misc_feature 1..3: note = nnn = any amino acid-coding triplet
  misc_feature 4..6: note = nnn = any amino acid-coding triplet
  misc_feature 16..18: note = nnn = any amino acid-coding triplet
  source 1..36: mol_type = other DNA
nnnnnnaaagaaattnnncatatttttcatgataac

SEQ ID NO: 382 - protein, 12 aa, Lactobacillus curvatus
TPVVNPPFLQQT

SEQ ID NO: 383 - DNA, 36 bp, synthetic construct
  misc_feature 1..36: note = Polynucleotide encoding the polypeptide of SEQ IDNO: 382
  source 1..36: mol_type = other DNA
accccggtggtgaacccgccgtttctgcagcagacc

SEQ ID NO: 384 - protein, 10 aa, Lactobacillus curvatus
  VARIANT 10: note = Xaa = any amino acid
VAPFPEQFLX

SEQ ID NO: 385 - DNA, 30 bp, synthetic construct
  misc_feature 1..30: note = Polynucleotide encoding the polypeptide of SEQ IDNO: 384
  misc_feature 28..30: note = nnn = any amino acid-coding triplet
  source 1..30: mol_type = other DNA
gtggcgccgtttccggaacagtttctgnnn

SEQ ID NO: 386 - protein, 9 aa, Lactobacillus curvatus
NIPQLTPTP

SEQ ID NO: 387 - DNA, 27 bp, synthetic construct
  misc_feature 1..27: note = Polynucleotide encoding the polypeptide of SEQ IDNO: 386
  source 1..27: mol_type = other DNA
aacattccgcagctgaccccgaccccg

SEQ ID NO: 388 - protein, 18 aa, Bacillus thuringiensis
  VARIANT 4..10: note = X= any amino acid
  source 1..18: note = subsp. entomocidus
DWTXWSXLVXAACSVELL

SEQ ID NO: 389 - DNA, 54 bp, synthetic construct
  misc_feature 1..54: note = Polynucleotide encoding the polypeptide of SEQ IDNO: 388
  misc_feature 10..12: note = nnn = any amino acid-coding triplet
  misc_feature 19..21: note = nnn = any amino acid-coding triplet
  misc_feature 28..30: note = nnn = any amino acid-coding triplet
  source 1..54: mol_type = other DNA
gattggaccnnntggagcnnnctggtgnnngcggcgtgcagcgtggaactgctg

SEQ ID NO: 390 - protein, 30 aa, Lactobacillus curvatus
AYPGNGVHCGKYSCTVDKQTAIGNIGNNAA

SEQ ID NO: 391 - DNA, 90 bp, synthetic construct
  misc_feature 1..90: note = Polynucleotide encoding the polypeptide of SEQ IDNO: 390
  source 1..90: mol_type = other DNA
gcgtatccgggcaacggcgtgcattgcggcaaatatagctgcaccgtggataaacagaccgcgattggcaacattggcaacaacgcggcg

SEQ ID NO: 392 - protein, 43 aa, Carnobacterium divergens
TKYYGNGVYCNSKKCWVDWGTAQGCIDVVIGQLGGGIPGKGKC

SEQ ID NO: 393 - DNA, 129 bp, synthetic construct
  misc_feature 1..129: note = Polynucleotide encoding the polypeptide of SEQ IDNO: 392
  source 1..129: mol_type = other DNA
accaaatattatggcaacggcgtgtattgcaacagcaaaaaatgctgggtggattggggcaccgcgcagggctgcattgatgtggtgattggccagctgggcggcggcattccgggcaaaggcaaatgc

SEQ ID NO: 394 - protein, 62 aa, Enterococcus sp.
NRWYCNSAAGGVGGAAVCGLAGYVGEAKENIAGEVRKGWGMAGGFTHNKACKSFPGSGWASG

SEQ ID NO: 395 - DNA, 186 bp, synthetic construct
  misc_feature 1..186: note = Polynucleotide encoding the polypeptide of SEQ IDNO: 394
  source 1..186: mol_type = other DNA
aaccgctggtattgcaacagcgcggcgggcggcgtgggcggcgcggcggtgtgcggcctggcgggctatgtgggcgaagcgaaagaaaacattgcgggcgaagtgcgcaaaggctggggcatggcgggcggctttacccataacaaagcgtgcaaaagctttccgggcagcggctgggcgagcggc

SEQ ID NO: 396 - protein, 39 aa, Enterococcus faecium
TTKNYGNGVCNSVNWCQCGNVWASCNLATGCAAWLCKLA

SEQ ID NO: 397 - DNA, 117 bp, synthetic construct
  misc_feature 1..117: note = Polynucleotide encoding the polypeptide of SEQ IDNO: 396
  source 1..117: mol_type = other DNA
accaccaaaaactatggcaacggcgtgtgcaacagcgtgaactggtgccagtgcggcaacgtgtgggcgagctgcaacctggcgaccggctgcgcggcgtggctgtgcaaactggcg

SEQ ID NO: 398 - protein, 30 aa, Paenibacillus polymyxa
ASIIKTTIKVSKAVCKTLTCICTGSCSNCK

SEQ ID NO: 399 - DNA, 90 bp, synthetic construct
  misc_feature 1..90: note = Polynucleotide encoding the polypeptide of SEQ IDNO: 398
  source 1..90: mol_type = other DNA
gcgagcattattaaaaccaccattaaagtgagcaaagcggtgtgcaaaaccctgacctgcatttgcaccggcagctgcagcaactgcaaa

SEQ ID NO: 400 - protein, 31 aa, Staphylococcus epidermidis
SASIVKTTIKASKKLCRGFTLTCGCHFTGKK

SEQ ID NO: 401 - DNA, 93 bp, synthetic construct
  misc_feature 1..93: note = Polynucleotide encoding the polypeptide of SEQ IDNO: 400
  source 1..93: mol_type = other DNA
agcgcgagcattgtgaaaaccaccattaaagcgagcaaaaaactgtgccgcggctttaccctgacctgcggctgccattttaccggcaaaaaa

SEQ ID NO: 402 - protein, 43 aa, Enterococcus faecium
KYYGNGVSCNKKGCSVDWGKAIGIIGNNAAANLTTGGKAAWAC

SEQ ID NO: 403 - DNA, 129 bp, synthetic construct
  misc_feature 1..129: note = Polynucleotide encoding the polypeptide of SEQ IDNO: 402
  source 1..129: mol_type = other DNA
aaatattatggcaacggcgtgagctgcaacaaaaaaggctgcagcgtggattggggcaaagcgattggcattattggcaacaacgcggcggcgaacctgaccaccggcggcaaagcggcgtgggcgtgc

SEQ ID NO: 404 - protein, 39 aa, Paenibacillus polymyxa
ATYYGNGLYCNKQKHYTWVDWNKASREIGKITVNGWVQH

SEQ ID NO: 405 - DNA, 117 bp, synthetic construct
  misc_feature 1..117: note = Polynucleotide encoding the polypeptide of SEQ IDNO: 404
  source 1..117: mol_type = other DNA
gcgacctattatggcaacggcctgtattgcaacaaacagaaacattatacctgggtggattggaacaaagcgagccgcgaaattggcaaaattaccgtgaacggctgggtgcagcat

SEQ ID NO: 406 - protein, 35 aa, Bacillus circulans
VNYGNGVSCSKTKCSVNWGIITHQAFRVTSGVASG

SEQ ID NO: 407 - DNA, 105 bp, synthetic construct
  misc_feature 1..105: note = Polynucleotide encoding the polypeptide of SEQ IDNO: 406
  source 1..105: mol_type = other DNA
gtgaactatggcaacggcgtgagctgcagcaaaaccaaatgcagcgtgaactggggcattattacccatcaggcgtttcgcgtgaccagcggcgtggcgagcggc

SEQ ID NO: 408 - protein, 30 aa, Paenibacillus polymyxa
FVYGNGVTSILVQAQFLVNGQRRFFYTPDK

SEQ ID NO: 409 - DNA, 90 bp, synthetic construct
  misc_feature 1..90: note = Polynucleotide encoding the polypeptide of SEQ IDNO: 408
  source 1..90: mol_type = other DNA
tttgtgtatggcaacggcgtgaccagcattctggtgcaggcgcagtttctggtgaacggccagcgccgctttttttataccccggataaa

SEQ ID NO: 410 - protein, 13 aa, Lactobacillus rhamnosus
AVPAVRKTNETLD

SEQ ID NO: 411 - DNA, 39 bp, synthetic construct
  misc_feature 1..39: note = Polynucleotide encoding the polypeptide of SEQ IDNO: 410
  source 1..39: mol_type = other DNA
gcggtgccggcggtgcgcaaaaccaacgaaaccctggat

SEQ ID NO: 412 - protein, 69 aa, Bacillus licheniformis
MKNSAAREAFKGANHPAGMVSEEELKALVGGNDVNPETTPATTSSWTCITAGVTVSASLCPTTKCTSRC

SEQ ID NO: 413 - DNA, 207 bp, synthetic construct
  misc_feature 1..207: note = Polynucleotide encoding the polypeptide of SEQ IDNO: 412
  source 1..207: mol_type = other DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgcccgaccaccaaatgcaccagccgctgc

SEQ ID NO: 414 - protein, 36 aa, Lactobacillus plantarum
KYYGNGLSCSKKGCTVNWGQAFSCGVNRVATAGHGK

SEQ ID NO: 415 - DNA, 108 bp, synthetic construct
  misc_feature 1..108: note = Polynucleotide encoding the polypeptide of SEQ IDNO: 414
  source 1..108: mol_type = other DNA
aaatattatggcaacggcctgagctgcagcaaaaaaggctgcaccgtgaactggggccaggcgtttagctgcggcgtgaaccgcgtggcgaccgcgggccatggcaaa

SEQ ID NO: 416 - protein, 24 aa, Lactobacillus acidophilus
GNPKVAHCASQIGRSTAWGAVSGA

SEQ ID NO: 417 - DNA, 72 bp, synthetic construct
  misc_feature 1..72: note = Polynucleotide encoding the polypeptide of SEQ IDNO: 416
  source 1..72: mol_type = other DNA
ggcaacccgaaagtggcgcattgcgcgagccagattggccgcagcaccgcgtggggcgcggtgagcggcgcg

SEQ ID NO: 418 - protein, 40 aa, Enterococcus faecalis; identical to SEQ ID NO: 1

SEQ ID NO: 419 - DNA, 120 bp, synthetic construct
  misc_feature 1..120: note = Polynucleotide encoding the polypeptide of SEQ IDNO: 418
  source 1..120: mol_type = other DNA
tggctgccgccggcgggcctgctgggccgctgcggccgctggtttcgcccgtggctgctgtggctgcagagcggcgcgcagtataaatggctgggcaacctgtttggcctgggcccgaaa

SEQ ID NO: 420 - protein, 40 aa, Anabaena variabilis
NLDQWLTEQVHEFQDMYLEPQAISNQDITFKLSDLDFIHN

SEQ ID NO: 421 - DNA, 124 bp, Anabaena variabilis
  source 1..124: mol_type = genomic DNA
taatttagatcagtggttaacagaacaagttcatgagtttcaagatatgtacttggaaccacaagcaatatccaatcaagacattaccttcaaactatctgacctagattttattcataattga

SEQ ID NO: 422 - protein, 40 aa, Nostoc sp.; identical to SEQ ID NO: 420

SEQ ID NO: 423 - DNA, 123 bp, Nostoc sp.
  source 1..123: mol_type = genomic DNA
aatttagatcaatggttaacagaacaagttcatgagtttcaagatatgtacttggaaccacaagcaatatccaatcaagacattaccttcaaactgtcagacctagattttattcataattga

SEQ ID NO: 424 - protein, 7 aa, Nostoc azollae
HREKKSA

SEQ ID NO: 425 - DNA, 24 bp, Nostoc azollae
  source 1..24: mol_type = genomic DNA
cacagagagaaaaaatcagcatag

SEQ ID NO: 426 - protein, 23 aa, Acaryochloris marina
TSNNWLAKNYLSMWNKKSSNPNL

SEQ ID NO: 427 - DNA, 72 bp, Acaryochloris marina
  source 1..72: mol_type = genomic DNA
acaagcaataactggctagccaaaaactatctttctatgtggaataaaaagagcagtaatccaaacctttag

SEQ ID NO: 428 - protein, 6 aa, Cyanothece sp.
FRYFWW

SEQ ID NO: 429 - DNA, 21 bp, Cyanothece sp.
  source 1..21: mol_type = genomic DNA
tttagatatttttggtggtaa

SEQ ID NO: 430 - protein, 6 aa, Cyanothece sp.
FRYFWW

SEQ ID NO: 431 - DNA, 21 bp, Cyanothece sp.
  source 1..21: mol_type = genomic DNA
tttagatatttttggtggtaa

SEQ ID NO: 432 - protein, 9 aa, Cyanothece sp.
CGEKWRIFS

SEQ ID NO: 433 - DNA, 27 bp, Cyanothece sp.
  source 1..27: mol_type = genomic DNA
tgtggagaaaaatggagaatttttagc

SEQ ID NO: 434 - protein, 8 aa, Cyanothece sp.
FRLQLWQF

SEQ ID NO: 435 - DNA, 24 bp, Cyanothece sp.
  source 1..24: mol_type = genomic DNA
tttcgcttacaactgtggcaattt

SEQ ID NO: 436 - protein, 16 aa, Cyanothece sp.
LGCNQSSIWSIFFWNH

SEQ ID NO: 437 - DNA, 51 bp, Cyanothece sp.
  source 1..51: mol_type = genomic DNA
ctaggatgtaaccagagcagtatctggtcaatttttttctggaatcattaa

SEQ ID NO: 438 - protein, 40 aa, Microcoleus chthonoplastes
YNLQGLPAIESEDCIPDSVAPSDDWFSGVSSLFNRLTGLG

SEQ ID NO: 439 - DNA, 123 bp, Microcoleus chthonoplastes
  source 1..123: mol_type = genomic DNA
tataacctacaggggttgccagcaattgagtcagaagactgtatcccagattctgtagcgccttcggatgattggttttcaggcgtatcgtctctgtttaaccgcttgactgggttgggttag

SEQ ID NO: 440 - protein, 37 aa, Nostoc sp.
WMAIRRILRCHPFHPGGYDPVPELGEHCCHHDSGNKG

SEQ ID NO: 441 - DNA, 114 bp, Nostoc sp.
  source 1..114: mol_type = genomic DNA
tggatggcgattcgccgcattttgcgttgtcatccattccacccagggggttatgatcctgtaccagagttgggtgagcattgttgtcatcatgatagcgggaataaggggtga

SEQ ID NO: 442 - protein, 35 aa, Anabaena variabilis
WMGIRRILRCHPFHPGGYDPVPEVGEHCCHHDSGK

SEQ ID NO: 443 - DNA, 108 bp, Anabaena variabilis
  source 1..108: mol_type = genomic DNA
tggatggggattcgccgcattttgcgttgtcatccattccacccaggcggttatgatcctgtaccagaggtgggtgagcattgttgtcatcatgatagcgggaagtag

SEQ ID NO: 444 - protein, 48 aa, Nodularia spumigena
WMATRRILRCHPFHPGGYDPVPEVKHNCCDQHLSDSGKQTTEDHHKGS

SEQ ID NO: 445 - DNA, 147 bp, Nodularia spumigena
  source 1..147: mol_type = genomic DNA
tggatggcgactcggcggattttgcgttgtcatcccttccatcctggtggatatgatccagttccagaggtaaaacacaattgctgcgatcagcatctgtccgattctgggaaacagaccacagaagaccatcacaaaggctcgtag

SEQ ID NO: 446 - protein, 34 aa, Nostoc azollae
WMATLRILRCHPFHPGGYDPVPGLAEKSCCDHHD

SEQ ID NO: 447 - DNA, 105 bp, Nostoc azollae
  source 1..105: mol_type = genomic DNA
tggatggcaactttgcggattttacgctgtcatcctttccatcctggtggttatgatcctgtaccaggactagcggaaaaatcctgttgtgaccatcatgattga

SEQ ID NO: 448 - protein, 28 aa, Synechococcus sp
WLTAKRFCRCHPLHPGGYDPVPEKKSVL

SEQ ID NO: 449 - DNA, 87 bp, Synechococcus sp
  source 1..87: mol_type = genomic DNA
tggctaacagccaagcgcttttgtcgctgtcatccgcttcatcctggcgggtatgatccggtaccggagaagaaatcggtactctaa

SEQ ID NO: 450 - protein, 23 aa, Prochlorococcus marinus
WLTLRRLSRCHPFTPCGCDPVPD

SEQ ID NO: 451 - DNA, 72 bp, Prochlorococcus marinus
  source 1..72: mol_type = genomic DNA
tggctcaccctgcggcgcctgtctcgttgccatccttttaccccctgtggttgcgacccggtgcctgattaa

SEQ ID NO: 452 - protein, 69 aa, Escherichia coli
MSYKKLYQLTAIFSLPLTILLVSLSSLRIVGEGNSYVDVFLSFIIFLGFIELIHGIRKILVWSGWKNGS

SEQ ID NO: 453 - DNA, 210 bp, Escherichia coli
  source 1..210: mol_type = genomic DNA
atgagttataaaaaactgtaccaattgacggctatatttagtttacctcttactatcttattggtttcactttcatcccttcggattgttggcgaagggaattcttatgttgacgtttttctaagctttataatatttcttggttttattgagctgattcatgggattcgaaagattttggtctggtcaggctggaaaaacggaagttaa

SEQ ID NO: 454 - protein, 85 aa, Escherichia coli
MGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 455 - DNA, 258 bp, Escherichia coli
  source 1..258: mol_type = genomic DNA
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtga

SEQ ID NO: 456 - protein, 113 aa, Escherichia coli
MSLRYYIKNILFGLYCTLIYIYLITKNSEGYYFLVSDKMLYAIVISTILCPYSKYAIEYIAFNFIKKDFFERRKNLNNAPVAKLNLFMLYNLLCLVLAIPFGLLGLFISIKNN

SEQ ID NO: 457 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagggtattatttccttgtgtcagataagatgctatatgcaatagtgataagcactattctatgtccatattcaaaatatgctattgaatacatagcttttaacttcataaagaaagattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 458 - protein, 85 aa, Escherichia coli
MGLKLHIHWFDKKTEEFKGGEYSKDFGDDGSVIESLGMPLKDNINNGWFDVEKPWVSILQPHFKNVIDISKFDYFVSFVYRDGNW

SEQ ID NO: 459 - DNA, 258 bp, Escherichia coli
  source 1..258: mol_type = genomic DNA
atggggcttaaattacatattcattggtttgataagaaaaccgaagagtttaaaggcggtgaatactcaaaagacttcggtgatgatggttctgtcattgaaagtctggggatgcctttaaaggataatattaataatggttggtttgatgttgaaaaaccatgggtttcgatattacagccacactttaaaaatgtaatcgatattagtaaatttgattactttgtatcctttgtttaccgggatggtaactggtaa

SEQ ID NO: 460 - protein, 86 aa, Escherichia coli
MELKHSISDYTEAEFLEFVKKICRAEGATEEDDNKLVREFERLTEHPDGSDLIYYPRDDREDSPEGIVKEIKEWRAANGKSGFKQG

SEQ ID NO: 461 - DNA, 261 bp, Escherichia coli
  source 1..261: mol_type = genomic DNA
atggaactgaaacatagtattagtgattataccgaggctgaatttctggagtttgtaaaaaaaatatgtagagctgaaggtgctactgaagaggatgacaataaattagtgagagagtttgagcgattaactgagcacccagatggttcagatctgatttattatcctcgcgatgacagggaagatagtcctgaagggattgtcaaggaaattaaagaatggcgagctgctaacggtaagtcaggatttaaacagggctga

SEQ ID NO: 462 - protein, 178 aa, Citrobacter freundii
MMNEHSIDTDNRKANNALYLFIIIGLIPLLCIFVVYYKTPDALLLRKIATSTENLPSITSSYNPLMTKVMDIYCKTAPFLALILYILTFKIRKLINNTDRNTVLRSCLLSPLVYAAIVYLFCFRNFELTTAGRPVRLMATNDATLLLFYIGLYSIIFFTTYITLFTPVTAFKLLKKRQ

SEQ ID NO: 463 - DNA, 537 bp, Citrobacter freundii
  source 1..537: mol_type = genomic DNA
atgatgaatgaacactcaatagatacggacaacagaaaggccaataacgcattgtatttatttataataatcggattaataccattattgtgcatttttgttgtttactacaaaacgccagacgctttacttttacgtaaaattgctacaagcactgagaatctcccgtcaataacatcctcctacaacccattaatgacaaaggttatggatatttattgtaaaacagcgcctttccttgccttaatactatacatcctaacctttaaaatcagaaaattaatcaacaacaccgacaggaacactgtacttagatcttgtttattaagtccattggtctatgcagcaattgtttatctattctgcttccgaaattttgagttaacaacagccggaaggcctgtcagattaatggccaccaatgacgcaacactattgttattttatattggtctgtactcaataattttctttacaacctatatcacgctattcacaccagtcactgcatttaaattattaaaaaaaaggcagtaa

SEQ ID NO: 464 - protein, 111 aa, Escherichia coli
MNRKYYFNNMWWGWVTGGYMLYMSWDYEFKYRLLFWCISLCGMVLYPVAKWYIEDTALKFTRPDFWNSGFFADTPGKMGLLAVYTGTVFILSLPLSMIYILSVIIKRLSVR

SEQ ID NO: 465 - DNA, 336 bp, Escherichia coli
  source 1..336: mol_type = genomic DNA
atgaacagaaaatattattttaataatatgtggtggggatgggtgacggggggatatatgctgtatatgtcatgggattatgagtttaaatacagattactgttctggtgtatttctctctgcggaatggttttgtatccggttgcaaaatggtatattgaagatacagctctaaaatttacccggcctgatttctggaacagcggtttttttgctgatacacctggaaaaatggggttgcttgcggtttatacgggtactgttttcatattatctcttccgttaagtatgatatatattctttctgttattataaaaaggctgtctgtaagatag

SEQ ID NO: 466 - protein, 115 aa, Escherichia coli
MKLDISVKYLLKSLIPILIILTVFYLGWKDNQENARMFYAFIGCIISAITFPFSMRIIQKMVIRFTGKEFWQKDFFTNPVGGSLTAIFELFCFVISVPVVAIYLIFILCKALSGK

SEQ ID NO: 467 - DNA, 348 bp, Escherichia coli
  source 1..348: mol_type = genomic DNA
atgaaactggatatatctgtaaagtatttactgaaaagcctgataccaatcctcattattcttacagttttttatctgggatggaaagataaccaggaaaatgcaagaatgttttatgcgttcatcggatgcattatcagtgccattacttttcctttttcaatgaggataatacagaaaatggtaataaggtttacagggaaagaattctggcaaaaagacttctttacaaatccagttggcggaagcttaactgcaatatttgaattattctgtttcgttatatcagttcctgtggttgccatttacttaatttttatactctgcaaagccctttcaggaaaatga

SEQ ID NO: 468 - protein, 131 aa, Escherichia coli
MHNTLLEKIIAYLSLPGFHSLNNPPLSEAFNLYVHTAPLAATSLFIFTHKELELKPKSSPLRALKILTPFTILYISMIYCFLLTDTELTLSSKTFVLIVKKRSVFVFFLYNTIYWDIYIHIFVLLVPYRNI

SEQ ID NO: 469 - DNA, 396 bp, Escherichia coli
  source 1..396: mol_type = genomic DNA
atgcacaatacactcctcgaaaaaatcatcgcatacctatccctaccaggatttcattcattaaacaacccgcccctaagcgaagcattcaatctctatgttcatacagcccctttagctgcaaccagcttattcatattcacacacaaagaattagagttaaaaccaaagtcgtcacctctgcgggcactaaagatattaactcctttcactattctttatatatccatgatatactgtttcttgctaactgacacagaactaaccttgtcatcaaaaacatttgtattaatagtcaaaaaacgatctgtttttgtcttttttctatataacactatatattgggatatatatattcacatatttgtacttttggttccttataggaacatataa

SEQ ID NO: 470 - protein, 85 aa, Escherichia coli
MELKNSISDYTETEFKKIIEDIINCEGDEKKQDDNLEHFISVTEHPSGSDLIYYPEGNNDGSPEAVIKEIKEWRAANGKSGFKQG

SEQ ID NO: 471 - DNA, 258 bp, Escherichia coli
  source 1..258: mol_type = genomic DNA
atggaactgaaaaacagcattagtgattacactgaaactgaattcaaaaaaattattgaagacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 472 - protein, 98 aa, Lactococcus lactis
  source 1..98: note = subsp. lactis
MKKKQIEFENELRSMLATALEKDISQEERNALNIAEKALDNSEYLPKIILNLRKALTPLAINRTLNHDLSELYKFITSSKASNKNLGGGLIMSWGRLF

SEQ ID NO: 473 - DNA, 297 bp, Lactococcus lactis
  source 1..297: mol_type = genomic DNA; note = subsp. lactis
atgaaaaaaaaacaaatagaatttgaaaacgagctaagaagtatgttggctaccgcccttgaaaaagacattagtcaagaggaaagaaatgctctgaatattgcagaaaaggcgcttgacaattctgaatatttaccaaaaattattttaaacctcagaaaagccctaactccattagctataaatcgaacacttaaccatgatttatctgaactgtataaattcattacaagttccaaagcatcaaacaaaaatttaggtggtggtttaattatgtcgtggggacgactattctaa

SEQ ID NO: 474 - protein, 98 aa, Lactococcus lactis; identical to SEQ ID NO: 472
  source 1..98: note = subsp. cremoris

SEQ ID NO: 475 - DNA, 297 bp, Lactococcus lactis; identical to SEQ ID NO: 473
  source 1..297: mol_type = genomic DNA; note = subsp. cremoris

SEQ ID NO: 476 - protein, 87 aa, Escherichia coli
MNKMAMIDLAKLFLASKITAIEFSERICVERRRLYGVKDLSPNILNCGEELFMAAERFEPDADRANYEIDDNGLKVEVRSILEKFKL

SEQ ID NO: 477 - DNA, 249 bp, Escherichia coli
  source 1..249: mol_type = genomic DNA
atgatcgatttggcgaaattatttttagcttcgaaaattacagtgattgagttttcagagcgaatttgtgttgaacggagaagattgtatggtgttaaggatttgtctccgaatatattaaattgtggggaagagttgtctatggctgctgagcgatttgagcctgatgcagatagggctaattatgaaattgatgataatggacttaaggtcgaggtccgatctatcttggaaaaacttaaatcataa

SEQ ID NO: 478 - protein, 83 aa, Escherichia coli
MKLSPKAAIEVCNEAAKKGLWILGIDGGHWLNPGFRIDSSASWTYDMPEEYKSKIPENNRLAIENIKDDIENGYTAFIITLKM

SEQ ID NO: 479 - DNA, 243 bp, Escherichia coli
  source 1..243: mol_type = genomic DNA
atgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggagaatacaaatcaaaaatccctgaaaataatagattggctattgaaaatattaaagatgatattgagaatggatacactgctttcattatcacgttaa

SEQ ID NO: 480 - protein, 85 aa, Escherichia coli
MGLKLHINWFDKRTEEFKGGEYSKDFGDDGSVIERLGMPFKDNINNGWFDVIAEWVPLLQPYFNHQIDISDNEYFVSFDYRDGDW

SEQ ID NO: 481 - DNA, 258 bp, Escherichia coli
  source 1..258: mol_type = genomic DNA
atggggcttaaattacatattaattggtttgataagacgaccgaggaatttaaaggtggtgagtattcaaaagattttggagatgatggctcggtcattgaacgtcttggaatgcctttaaaagataatatcaataatggttggtttgatgttatagctgaatgggtacctttgctacaaccatactttaatcatcaaattgatatttccgataatgagtattttgtttcgtttgattatcgtgatggtgattggtga

SEQ ID NO: 482 - protein, 85 aa, Escherichia coli
MELKKSIGDYTETEFKKIIENIINCEGDEKKQDDNLEHFISVTEHPSGSDLIYYPEGNNDGSPEAVIKEIKEWRAANGKSGFKQG

SEQ ID NO: 483 - DNA, 258 bp, Escherichia coli
  source 1..258: mol_type = genomic DNA
gtggagctaaagaaaagtattggtgattacactgaaaccgaattcaaaaaaattattgaaaacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 484 - protein, 86 aa, Escherichia coli [sequence omitted]

SEQ ID NO: 485 - DNA, 261 bp, Escherichia coli
  source 1..261: mol_type = genomic DNA
atggaactgaagcatagcattagtgattatacagaagctgaatttttacaacttgtaacaacaatttgtaatgcgaacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaa

SEQ ID NO: 486 - protein, 141 aa, Escherichia coli
MLTLYGYIRNVFLYRMNDRSCGDFMKVISMKFIFILTIIALAAVFFWSEDKGPACYQVSDEQARTFVKNDYLQRMKRWDNDVQLLGTEIPKITWEKIERSLTDVEDEKTLLVPFKAEGPDGKRMYYGMYHCEEGYVEYAND

SEQ ID NO: 487 - DNA, 354 bp, Escherichia coli
  source 1..354: mol_type = genomic DNA
atgaaagtaattagcatgaaatttatttttattttaacgattattgctcttgctgctgtttttttctggtctgaagataaaggtccggcatgctatcaggtcagcgatgaacaggccagaacgtttgtaaaaaatgattacctgcaaagaatgaaacgctgggacaacgatgtacaacttcttggtacagaaatcccgaaaattacatgggaaaagattgagagaagtttaacagatgttgaagatgaaaaaacacttcttgtcccatttaaagctgaaggcccggacggtaagagaatgtattatggcatgtaccattgtgaggagggatatgttgaatatgcgaatgactaa

SEQ ID NO: 488 - protein, 175 aa, Escherichia coli
MTSNKDKNKKANEILYAFSIIGIIPLMAILILRINDPYSQVLYYLYNKVAFLPSITSLHDPVMTTLMSNYNKTAPVMGILVFLCTYKTREIIKPVTRKLVVQSCFWGPVFYAILIYITLFYNLELTTAGGFFKLLSHNVITLFILYCSIYFTVLTMTYAILLMPLLVIKYFKGRQ

SEQ ID NO: 489 - DNA, 528 bp, Escherichia coli
  source 1..528: mol_type = genomic DNA
atgaccagcaataaagataagaacaagaaagcaaacgaaatattatatgcattttccataatcgggattattccattaatggctatattaatacttcgaataaatgatccatattctcaagtgctgtactacttatataataaggtggcatttctcccttctattacatcattgcatgatcccgtcatgacaacacttatgtcaaactacaacaagacagcgccagttatgggtattctcgtttttctttgcacatataagacaagagaaatcataaagccagtaacaagaaaacttgttgtgcaatcctgtttctgggggcccgttttttatgccattctgatttatatcacactgttctataatctggaactaacaacagcaggtggtttttttaaattattatctcataatgtcatcactctgtttattttatattgctccatttactttactgttttaaccatgacatatgcgattttactgatgccattacttgtcattaaatattttaaagggaggcagtaa

SEQ ID NO: 490 - protein, 78 aa, Escherichia coli
MDRKRTKLELLFAFIINATAIYIALAIYDCVFRGKDFLSMHTFCFSALMSAICYFVGDNYYSISDKIKRRSYENSDSK

SEQ ID NO: 491 - DNA, 237 bp, Escherichia coli
  source 1..237: mol_type = genomic DNA
atggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatga

SEQ ID NO: 492 - protein, 113 aa, Shigella sonnei
MSLRYYIKNILFGLYCALIYIYLITKNNEGYYFLASDKMLYAIVISTILCPYSKYAIEHIFFKFIKKDFFRKRKNLNKCPRGKIKPYLCVYNLLCLVLAIPFGLLGLVYINKE

SEQ ID NO: 493 - DNA, 342 bp, Shigella sonnei
  source 1..342: mol_type = genomic DNA
atgagtttaagatactacataaaaaatattttgtttggcctatactgcgcacttatatatatataccttataacaaaaaacaacgaagggtattatttcctagcgtcagataagatgctatacgcaatagtgataagcactattctatgcccatattcaaaatatgctattgaacacatattttttaagttcataaagaaagattttttcagaaaaagaaaaaacctaaataaatgcccccgtggcaaaattaaaccgtatttatgcgtatacaatctactttgtttggtcctagcaatcccatttggattgctaggacttgtttatatcaataaagaataa

SEQ ID NO: 494 - protein, 113 aa, Escherichia coli
MSLRYYIKNILFGLYCTLIYIYLITKNSEEYYFLVTDKMLYAIVISTILCPYSKYAIEHIAFNFIKKHFFERRKNLNNAPVAKLNLFMLYNLLCLVLAIPFGLLGLFISIKNN

SEQ ID NO: 495 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagagtattatttccttgtgacagataagatgctatatgcaatagtgataagcactattctatgtccatattcaaaatatgctattgaacacatagcttttaacttcataaagaaacattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 496 - protein, 113 aa, Leuconostoc gelidum
MRKNNILLDDAKIYTNKLYLLLIDRKDDAGYGDICDVLFQVSKKLDSTKNVEALINRLVNYIRITASTNRIKFSKDEEAVIIELGVIGQKAGLNGQYMADFSDKSQFYSIFER

SEQ ID NO: 497 - DNA, 342 bp, Leuconostoc gelidum
  source 1..342: mol_type = genomic DNA
ttgagaaaaaataacattttattggacgatgctaaaatatacacgaacaaactctatttgctattaatcgatagaaaagatgacgctgggtatggagatatttgtgatgttttgtttcaggtatccaaaaaattagatagcacaaaaaatgtagaagcattgattaaccgattggtcaattatatacgaattaccgcttcaacaaacagaattaagttttcaaaagatgaagaggctgtaattatagaacttggtgtaattggtcagaaggctggattaaacggccaatacatggctgatttttctgacaaatctcagttttatagtatctttgaaagataa

SEQ ID NO: 498 - protein, 91 aa, Lactococcus lactis
  source 1..91: note = subsp. cremoris
MKKKVDTEKQITSWASDLASKNETKVQEKLILSSYIQDIENHVYFPKAMISLEKKLRDQNNICALSKEVNQFYFKVVEVNQRKSWMVGLIV

SEQ ID NO: 499 - DNA, 276 bp, Lactococcus lactis
  source 1..276: mol_type = genomic DNA; note = subsp. cremoris
atgaaaaaaaaagttgatacagaaaaacaaattacttcttgggcatctgacttagcttccaaaaatgaaacaaaggttcaagaaaaattaatactgtcttcttatattcaggacatcgaaaaccatgtttactttccaaaagcaatgatttctttagaaaaaaaattacgagaccaaaataatatttgcgctttatcaaaagaagtcaatcagttttattttaaagttgttgaagtaaatcaaagaaaatcctggatggtaggtttgatagtttaa

SEQ ID NO: 500 - protein, 112 aa, Pediococcus acidilactici
MNKTKSEHIKQQALDLFTRLQFLLQKHDTIEPYQYVLDILETGISKTKHNQQTPERQARVVYNKIASQALVDKLHFTAEENKVLAAINELAHSQKGWGEFNMLDTTNTWPSQ

SEQ ID NO: 501 - DNA, 339 bp, Pediococcus acidilactici
  source 1..339: mol_type = genomic DNA
atgaataagactaagtcggaacatattaaacaacaagctttggacttatttactaggctacagtttttactacagaagcacgatactatcgaaccttaccagtacgttttagatattctggagactggtatcagtaaaactaaacataaccagcaaacgcctgaacgacaagctcgtgtagtctacaacaagattgccagccaagcgttagtagataagttacattttactgccgaagaaaacaaagttctagcagccatcaatgaattggcgcattctcaaaaagggtggggcgagtttaacatgctagatactaccaatacgtggcctagccaatag

SEQ ID NO: 502 - protein, 88 aa, Carnobacterium maltaromaticum
MIKDEKINKIYALVKSALDNTDVKNDKKLSLLLMRIQETSINGELFYDYKKELQPAISMYSIQHNFRVPDDLVKLLALVQTPKAWSGF

SEQ ID NO: 503 - DNA, 267 bp, Carnobacterium maltaromaticum
  source 1..267: mol_type = genomic DNA
atgataaaagatgaaaaaataaataaaatctatgctttagttaagagcgcacttgataatacggatgttaagaatgataaaaaactttctttacttcttatgagaatacaagaaacatcaattaatggagaactattttacgattataaaaaagaattacagccagctattagtatgtactctattcaacataactttcgggttcctgacgatctagtaaaactgttagcattagttcaaacacctaaagcttggtcagggttttaa

SEQ ID NO: 504 - protein, 111 aa, Carnobacterium maltaromaticum
MDIKSQTLYLNLSEAYKDPEVKANEFLSKLVVQCAGKLTASNSENSYIEVISLLSRGISSYYLSHKRIIPSSMLTIYTQIQKDIKNGNIDTEKLRKYEIAKGLMSVPYIYF

SEQ ID NO: 505 - DNA, 336 bp, Carnobacterium maltaromaticum
  source 1..336: mol_type = genomic DNA
atggatataaagtctcaaacattatatttgaatctaagcgaggcatataaagaccctgaagtaaaagctaatgaattcttatcaaaattagttgtacaatgtgctgggaaattaacagcttcaaacagtgagaacagttatattgaagtaatatcattgctatctaggggtatttctagttattatttatcccataaacgtataattccttcaagtatgttaactatatatactcaaatacaaaaggatataaaaaacgggaatattgacaccgaaaaattaaggaaatatgagatagcaaaaggattaatgtccgttccttatatatatttctaa

SEQ ID NO: 506 - protein, 245 aa, Lactococcus lactis
  source 1..245: note = subsp. lactis
MRRYLILIVALIGITGLSGCYQTSHKKVRFDEGSYTNFIYDNKSYFVTDKEIPQENVNNSKVKFYKLLIVDMKSEKLLSSSNKNSVTLVLNNIYEASDKSLCMGINDRYYKILPESDKGAVKALRLQNFDVTSDISDDNFVIDKNDSRKIDYMGNIYSISDTTVSDEELGEYQDVLAEVRVFDSVSGKSIPRSEWGRIDKDGSNSKQSRTEWDYGEIHSIRGKSLTEAFAVEINDDFKLATKVGN

SEQ ID NO: 507 - DNA, 738 bp, Lactococcus lactis
  source 1..738: mol_type = genomic DNA; note = subsp. lactis
atgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactag

SEQ ID NO: 508 - protein, 261 aa, Rhizobium leguminosarum
  source 1..261: note = bv. trifolii
MNDEICLTGGGRTTVTRRGGVVYREGGPWSSTVISLLRHLEASGFAEAPSVVGTGFDERGRETLSFIEGEFVHPGPWSEEAFPQFGMMLRRLHDATASFKPPENSMWRDWFGRNLGEGQHVIGHCDTGPWNIVCRSGLPVGLIDWEVAGPVRADIELAQACWLNAQLYDDDIAERVGLGSVTMRAHQVRLLLDGYGLSRKQRGGFVDKLITFAVHDAAEQAKEAAVTPESNDAEPLWAIAWRTRSASWMLHHRQTLEAALA

SEQ ID NO: 509 - DNA, 786 bp, Rhizobium leguminosarum
  source 1..786: mol_type = genomic DNA; note = bv. trifolii
atgaatgatgagatttgcctgacaggtggcggacgaacgactgtcacgcggcgcggcggagtcgtgtatcgcgaaggcggcccgtggtcatcaaccgtcatttcgctcctgcggcatctggaagcctctggcttcgctgaagctccttccgttgtcggcaccggtttcgatgagcgcggccgggagacattatcgtttatcgagggtgagtttgttcacccaggcccttggtcggaggaggcttttccgcaatttggaatgatgttgcggcgactgcacgatgccaccgcctcgttcaaacctcccgaaaactcgatgtggcgcgattggttcgggcgtaacctcggtgagggtcaacacgtaataggacactgcgacacaggcccatggaacattgtttgccggtcaggattgcctgtcgggttgatagattgggaggtggctgggcctgtcagggcggatatcgaattggcccaggcttgttggctgaatgcccagctctacgatgacgacattgcggagagggtcggattaggctctgtgaccatgagagcgcatcaagttcgcctgctgcttgacggctatggtctgtctcggaagcaacgcggcggcttcgtcgacaagctaatcacgttcgcagttcacgatgcggccgagcaggcgaaagaggcggctgtcacgccagagtcgaacgatgcggaaccgctatgggcaattgcctggcgcactagaagtgcctcctggatgctccatcatcggcaaacactggaagcagcgctggcatag

SEQ ID NO: 510 - protein, 436 aa, Bacillus subtilis
MNNIIPIMSLLFKQLYSRQGKKDAIRIAAGLVILAVFEIGLIRQAGIDESVLRKTYIILALLLMNTYMVFLSVTSQWKESYMKLSCLLPISSRSFWLAQSVVLFVDTCLRRTLFFFILPLFLFGNGTLSGAQTLFWLGRFSFFTVYSIIFGVVLSNHFVKKKNLMFLLHAAIFACVCISAALMPAATIPLCAVHILWAVVIDFPVFLQAPPQQGKMHSFMRRSEFSFYKREWNRFISSKAMLLNYAVMAVFSGFFSFQMMNTGIFNQQVIYIVISALLLICSPIALLYSIEKNDRMLLITLPIKRKTMFWAKYRFYSGLLAGGFLLVVMIVGFISGRSISVLTFLQCIELLLAGAYIRLTADEKRPSFSWQTEQQLWSGFSKYRSYLFCLPLFLAILAGTAVSLAVIPIAGLVIVYYLQKQDGGFFDTSKRERLGS

SEQ ID NO: 511 - DNA, 1000 bp, Bacillus subtilis
  source 1..1000: mol_type = genomic DNA
atgaataacataatccctatcatgtctttgctgttcaaacagctttacagccggcaagggaaaaaggacgccatccgcattgccgcaggccttgtcattctggccgtgtttgaaatcgggctgatccgccaggccggcattgatgaatcggtgttgcgcaaaacgtatatcatactcgcgcttcttttgatgaacacatatatggtgtttctttccgtgacatcacaatggaaggaatcttatatgaagctgagctgcctgctgccgatttcttcacggagcttttggctcgcccagagtgtcgttttgtttgtcgatacctgtttgagaagaactttattcttttttattttaccgctgttcttatttggaaacggaacgctgtcaggggcgcaaacattgttttggctcggcaggttttcgttttttaccgtttactccattattttcggagttgtgctaagcaaccacttcgtcaaaaagaagaacttgatgtttctgctgcatgcggcgatattcgcctgtgtatgtatcagcgccgctttgatgccggccgccacgattccgctttgcgcggttcatatcctgtgggcggtggtcattgactttcctgtctttctgcaggcgcctccgcagcagggcaagatgcattcatttatgcggcgatctgaattttcgttttacaaaagagaatggaaccgatttatctcttctaaagcgatgctgttaaattacgcggtaatggcggtattcagcggcttcttttcgttccagatgatgaacaccggcatcttcaatcagcaagtgatttatatcgtgatttccgcgcttttgctcatctgctcgccgatcgcccttttgtattcgattgaaaaaaatgaccggatgctgctcatcacgcttccgatcaagcgaaaaacgatgttttgggcgaaatatcgcttttattcaggcctattggcaggcggatttctccttgtcgtgatgattgtgggtttca

SEQ ID NO: 512 - protein, 239 aa, Bacillus subtilis
MSILDIHDVSVWYERDNVILEQVDLHLEKGAVYGLLGVNGAGKTTLINTLTGVNRNFSGRFTLCGIEAEAGMPQKTSDQLKTHRYFAADYPLLFTEITAKDYVSFVHSLYQKDFSEQQFASLAEAFHFSKYINRRISELSLGNRQKVVLMTGLLLRAPLFILDEPLVGLDVESIEVFYQKMREYCEAGGTILFSSHLLDVVQRFCDYAAILHNKQIQKVIPIGEETDLRREFFEVIGHE

SEQ ID NO: 513 - DNA, 716 bp, Bacillus subtilis
  source 1..716: mol_type = genomic DNA
gcattttggatatacacgatgtatccgtttggtatgaacgggacaacgtcatcttagagcacgtggacttacacttagaaaaaggcgccgtttacggattgcttggggtaaacggtgccggcaaaacaacactgatcaatacgctgacaggagtgaaccgcaattacagcgggggctttacgctgtgcggcattgaagctgaggccggcatgccgcagaaaacatcagatcaactgaagattcaccgttacttcgccgctgattatccgctgctgtttacagaaattacggcgaaggactatgtgtctttcgtccattcgctttatcaaaaggatttttcagagcgacagtttgccagtttggctgaggcctttcatttttcaaaatacatcaacaggagaatctcggagctgtccttggggaacaggcaaaaggttgtgttgatgacaggattattgctgcgggctcccctgtttattttggatgagccgctcgtcggtttggatgtggaatcaatagaggtcttttatcagaaaatgcgggagtactgtgaggaaggcggaaccattttgttttcttcccatctgctcgatgtcgtgcagagattttgtgattttgcggccattctgcacaacaaacagatccaaaaggtcattccgattggggaggagaccgatctgcggcgggaattttttgaggttatcggccatgaataa

SEQ ID NO: 514 - protein, 53 aa, Bacillus subtilis
MSPAQRRILLYILSFIFVIGAVVYFVKSDYLFTLIFIAIAILFGMRARKADSR

SEQ ID NO: 515 - DNA, 162 bp, Bacillus subtilis
  source 1..162: mol_type = genomic DNA
ttgtcaccagcacaaagaagaattttactgtatatcctttcatttatctttgtcatcggcgcagtcgtctattttgtcaaaagcgattatctgtttacgctgattttcattgccattgccattctgttcgggatgcgcgcgcggaaggctgactcgcgatga

SEQ ID NO: 516 - protein, 87 aa, Escherichia coli
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 517 - DNA, 264 bp, Escherichia coli
  source 1..264: mol_type = genomic DNA
atggaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggctga

SEQ ID NO: 518 - protein, 87 aa, Pseudomonas aeruginosa
MKSKISEYTEKEFLEFVEDIYTNNKKKFPTEESHIQAVLEFKKLTEHPSGSDLLYYPNENREDSPAGVVKEVKEWRASKGLPGFKAG

SEQ ID NO: 519 - DNA, 264 bp, Pseudomonas aeruginosa
  source 1..264: mol_type = genomic DNA
atgaagtccaagatttccgaatatacggaaaaagagtttcttgagtttgttgaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 520 - protein, 87 aa, Pseudomonas aeruginosa
MKSKISEYTEKEFLEFVKDIYTNNKKKFPTEESHIQAVLEFKKLTEHPSGSDLLYYPNENREDSPAGVVKEVKEWRASKGLPGFKAG

SEQ ID NO: 521 - DNA, 264 bp, Pseudomonas aeruginosa
  source 1..264: mol_type = genomic DNA
atgaagtccaagatttccgaatatacggaaaaagagtttcttgagtttgttaaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 522 - protein, 95 aa, Enterococcus hirae
MDFTKEEKLLNAISKVYNEATIDDYPDLKEKLFLYSKEISEGKSVGEVSMKLSSFLGRYILKHKFGLPKSLIELQEIVSKESQVYRGWASIGIWS

SEQ ID NO: 523 - DNA, 288 bp, Enterococcus hirae
  source 1..288: mol_type = genomic DNA
atggattttactaaagaagaaaaacttttaaatgcaattagtaaagtatacaatgaagcaactatagatgactatcctgacttaaaagaaaagctctttctttattctaaagaaatcagtgagggaaaaagtgttggtgaagttagtatgaaattaagtagttttcttggaagatatattttaaaacataaatttggattacctaaatctttaatagaattacaagaaattgttagtaaggaatctcaagtatatagaggatgggcttctattggtatttggagttaa

SEQ ID NO: 524 - protein, 113 aa, Leuconostoc mesenteroides
MKKKYRYLEDSKNYTSTLYSLLVDNVDKPGYSDICDVLLQVSKKLDNTQSVEALINRLVNYIRITASTYKIIFSKKEEELIIKLGVIGQKAGLNGQYMADFSDKSQFYSVFDQ

SEQ ID NO: 525 - DNA, 342 bp, Leuconostoc mesenteroides
  source 1..342: mol_type = genomic DNA
ttgaaaaaaaagtatcggtatttagaagatagcaaaaattacactagtacactctattctctgttagttgataatgttgacaaacctggatactcagatatttgcgatgttttgcttcaagtttctaagaagttggataatactcaaagtgttgaagcgctaattaatcgattggttaattatattcgtattactgcttcaacatacaaaattattttttcaaaaaaagaagaggaattgattataaaacttggtgttattggacaaaaagctggacttaatggtcagtatatggctgatttttcagacaagtctcagttttacagcgttttcgatcagtaa

SEQ ID NO: 526 - protein, 93 aa, Escherichia coli
MSFLNFAFSPVFFSIMACYFIVWRNKRNEFVCNRLLSIIIISFLICFIYPWLNYKIEVKYYIFEQFYLFCFLSSLVAVVINLIVYFILYRRCI

SEQ ID NO: 527 - DNA, 282 bp, Escherichia coli
  source 1..282: mol_type = genomic DNA
atgagttttcttaattttgcattttctcctgtattcttctccattatggcgtgttatttcattgtatggagaaataaacgaaacgaatttgtctgcaatagattgctatcaattataataatatcttttttgatatgcttcatatatccatggctaaattacaaaatcgaagttaaatattatatatttgaacagttttatcttttttgttttttatcgtcactcgtggctgttgtaataaacctaattgtatactttatattatacaggagatgtatatga

SEQ ID NO: 528 - protein, 96 aa, Escherichia coli
MHLKYYLHNLPESLIPWILILIFNDNDNTPLLFIFISSIHVLLYPYSKLTISRYIKENTKLKKEPWYLCKLSALFYLLMAIPVGLPSFIYYTLKRN

SEQ ID NO: 529 - DNA, 291 bp, Escherichia coli
  source 1..291: mol_type = genomic DNA
atgcatttaaaatactacctacataatttacctgaatcacttataccatggattcttattttaatatttaacgacaatgataacactcctttgttatttatatttatatcatcaatacatgtattgctatatccatactctaaattaaccatatctagatatatcaaagaaaatacaaagttaaaaaaagaaccctggtacttatgcaagttatctgcattgttttatttattaatggcaatcccagtaggattgccaagtttcatatattacactctaaagagaaattaa

SEQ ID NO: 530 - protein, 344 aa, Escherichia coli
MMIQSHPLLAAPLAVGDTIGFFSSSAPATVTAKNRFFRGVEFLQRKGFKLVSGKLTGKTDFYRSGTIKERAQEFNELVYNPDITCIMSTIGGDNSNSLLPFLDYDAIIANPKIIIGYSDTTALLAGIYAKTGLITFYGPALIPSFGEHPPLVDITYESFIKILTRKQSGIYTYTLPEKWSDESINWNENKILRPKKLYKNNCAFYGSGKVEGRVIGGNLNTLTGIWGSEWMPEILNGDILFIEDSRKSIATIERLFSMLKLNRVFDKVSAIILGKHELFDCAGSKRRPYEVLTEVLDGKQIPVLDGFDCSHTHPMLTLPLGVKLAIDFDNKNISITEQYLSTEK

SEQ ID NO: 531 - DNA, 1000 bp, Escherichia coli
  source 1..1000: mol_type = genomic DNA
atgatgatacaatctcatccactactggccgctcccctggcagtaggagatacaattggtttcttttcatcatctgctccggcaacagttactgcaaaaaatcgtttttttcggggagttgagtttcttcagagaaagggatttaagctggtatcagggaagcttaccggtaaaacagatttttatcgttcaggtactattaaagaaagagctcaagaatttaatgagttagtctacaatcctgatattacctgtataatgtcaacgatcggtggagataacagtaattcactactaccgtttctggactatgatgctatcattgcaaaccccaaaattatcataggttactcagatacaactgctttattagcaggaatatatgcaaaaacagggttaataacattctatggaccagctcttattccttcgtttggtgaacatccacctcttgtggatataacatatgaatcatttattaaaatactaacaagaaaacaatcaggaatatatacctacacattacctgaaaagtggagtgatgagagcataaactggaatgaaaacaagatattaaggcctaagaagctatataaaaacaactgtgccttttatggttccggaaaagttgaggggcgtgtaattggaggaaatctaaatactttgacaggtatatgggggagtgaatggatgcctgaaattcttaatggagatatattgtttattgaggacagtcggaaaagcattgcaacaattgaacgattattctctatgctaaagcttaatcgcgtgtttgataaagttagtgcaataatactcgggaaacatgagctttttgattgtgcaggaagtaaacgcagaccatatgaagtattaacagaggtattagatgggaaacagattcctgtactggatggatttgattgttcacatacacatccaatgctaactcttccacttggtgtaaaattagctattgactttgacaacaaaaatatat

SEQ ID NO: 532 - protein, 90 aa, Lactobacillus sakei
MKADYKKINSILTYTSTALKNPKIIKDKDLVVLLTIIQEEAKQNRIFYDYKRKFRPAVTRFTIDNNFEIPDCLVKLLSAVETPKAWSGFS

SEQ ID NO: 533 - DNA, 268 bp, Lactobacillus sakei
  source 1..268: mol_type = genomic DNA
ggcagattataaaaaaataaattcaatactaacttacacatctactgctttaaaaaaccctaaaattataaaagataaagatttagtagtccttctaactattattcaagaagaagccaaacaaaatagaatcttttatgattataaaagaaaatttcgtccagcggttactcgctttacaattgataataattttgagattcctgattgtttggttaaactactgtcagctgttgaaacacctaaggcgtggtctggatttagttag

SEQ ID NO: 534 - protein, 83 aa, Escherichia coli
MKLSPKAAIEVCNEAAKKGLWILGIDGGHWLNPGFRIDSSASWTYDMPEEYKSKTPENNRLAIENIKDDIENGYTAFIITLKM

SEQ ID NO: 535 - DNA, 251 bp, Escherichia coli
  source 1..251: mol_type = genomic DNA
tgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggaggaatacaaatcaaaaacccctgaaaataatagattggctattgaaaatattaaagatgatattgagaatggatacactgctttcattatcacgttaaagatgtaa

SEQ ID NO: 536 - protein, 436 aa, Bacillus subtilis
MNNIFPIMSLLFKQLYSRQGKKDAIRIAAGLVILAVFEIGLIRQAGIDESVLGKTYIILALLLMNTYMVFLSVTSQWKESYMKLSCLLPISSRSFWLAQSVVLFVDTCLRRTLFFFILPLFLFGNGTLSGAQTLFWLGRFSFFTVYSILFGVMLSNHFVKKKNSMFLLHAAVFAFVCLSAAFMPAVTIPLCAVHMLWAVIIDFPVFLQAPPHQSKMHFFMRRSEFSFYKREWNRFISSKAMLLNYVVMAAFSGFFSFQMMNTGIFNQQVIYIVISALLLICSPIALLYSIEKNDRMLLITLPIKRRTMFWAKYRFYSGLLAGGFLLVAIIVGFISGRPISALTFVQCMELLLAGAFIRLTADEKRPSFGWQTEQQLWSGFSKYRSYLFCLPLFLATLAGTAVSLAVIPIAALIIVYYLQKQDGGFFDTSKRERIGS

SEQ ID NO: 537 - DNA, 1000 bp, Bacillus subtilis
  source 1..1000: mol_type = genomic DNA
ttggggaggagaccgatctgcggcgggaattttttgaggttatcggccatgaataacatattccccatcatgtcgttgctgttcaaacagctgtacagccggcaagggaaaaaggacgctatccgcattgctgcagggcttgtgattctcgccgtgtttgaaatcgggctgatccgacaagccggcattgacgaatcggtgttgggaaaaacgtatatcatattggcgcttctcttaatgaacacgtatatggtgtttctttccgtgacatcacaatggaaggaatcttatatgaagctgagctgtctgctgccgatttcatcacggagcttttggctcgcccagagtgtcgttctgtttgtcgatacctgtttgagaagaacgttattcttttttattttaccgctgttcttatttggaaacggaacgctgtcaggggcgcaaacattgttttggcttggcagattttcgttttttaccgtttactcgattctattcggagttatgctaagcaaccatttcgtcaaaaagaagaactcgatgtttctgctgcatgcggcggtattcgcctttgtatgcctcagtgccgcttttatgccggccgtcacgatcccgctatgcgcggttcacatgctatgggcggtgatcattgactttccggtctttctgcaggcgcctccgcatcagagcaagatgcatttttttatgcggcgatctgaattttcgttttacaaaagagaatggaaccgatttatttcttctaaagcgatgctgttaaattacgtggtgatggcggcgttcagcggattcttttcgttccagatgatgaacactggcatcttcaatcagcaagtgatttatattgtgatttccgctctattgctgatttgctcgccgatcgcccttttgtactctattgaaaaaaacgatcgcatgctgctcatcacgcttccaattaaaagaagaacgatgttttgggcgaaatatcgcttttattcag

SEQ ID NO: 538 - protein, 580 aa, Escherichia coli
MERKQKNSLFNYIYSLMDVRGKFLFFSMLFITSLSSIIISISPLILAKITDLLSGSLSNFSYEYLVLLACLYMFCVISNKASVFLFMILQSSLRINMQKKMSLKYLRELYNENITNLSKNNAGYTTQSLNQASNDIYILVRNVSQNILSPVIQLISTIVVVLSTKDWFSAGVFFLYILVFVIFNTRLTGSLASLRKHSMDITLNSYSLLSDTVDNMIAAKKNNALRLISERYEDALTQENNAQKKYWLLSSKVLLLNSLLAVILFGSVFIYNILGVLNGVVSIGHFIMITSYIILLSTPVENIGALLSEIRQSMSSLAGFIQRHAENKATSPSIPFLNMERKLNLSIRELSFSYSDDKKILNSVSLDLFTGKMYSLTGPSGSGKSTLVKIISGYYKNYFGDIYLNDISLRNISDEDLNDAIYYLTQDDYIFMDTLRFNLRLANYDASENEIFKVLKLANLSVVNNEPVSLDTHLINRGNNYSGGQKQRISLARLFLRKPAIIIIDEATSALDYINESEILSSIRTHFPDALIINISHRINLLECSDCVYVLNEGNIVASGHFRDLMVSNEYISGLASVTE

SEQ ID NO: 539 - DNA, 1000 bp, Escherichia coli
  source 1..1000: mol_type = genomic DNA
atggaaagaaaacagaaaaactcattatttaattatatttattcattaatggatgtaagaggtaaatttttattcttttccatgttattcattacatcattatcatcgataatcatatctatttcaccattgattcttgcaaagattacagatttactgtctggctcattgtcaaattttagttatgaatatctggttttacttgcctgtttatacatgttttgcgttatatctaataaagcaagtgtttttttatttatgatactgcaaagtagtctacgtattaacatgcagaaaaaaatgtcgctaaagtatttgagagaattgtataacgaaaatataactaacttgagtaaaaataatgctggatatacaacgcaaagtcttaaccaggcttcaaatgacatttatattcttgtgagaaatgtttcccagaatatcctgtcacctgttatacaacttatttccactattgttgttgttttatctacgaaggactggttttctgccggtgtgttttttctctatattctggtatttgtaatttttaataccagactgactggcagtttagcgtctctcagaaaacacagcatggatatcactcttaactcttatagtctgttatctgatactgttgataacatgatagcagctaaaaagaataatgcattaagacttatttctgaacgttatgaagatgctctcactcaggaaaacaatgctcagaaaaaatactggttactcagttctaaagttcttttattgaactctttacttgctgtaatattatttggttctgtattcatatataatattttaggtgtgctgaatggtgtagttagtatcggccacttcattatgattacatcatatatcattcttctttcaacgccagtggaaaatataggggcattgctaagtgagatcaggcagtcaatgtctagcctggcaggttttattcaacgtcatgccgagaataaagccacatctccttcaa

SEQ ID NO: 540 - protein, 95 aa, Klebsiella pneumoniae
MTLLSFGFSPVFFSVMAFCIISRSKFYPQRTRNKVIVLILLTFFICFLYPLTKVYLVGSYGIFDKFYLFCFISTLIAIAINVVILTINGAKNERN

SEQ ID NO: 541 - DNA, 288 bp, Klebsiella pneumoniae
  source 1..288: mol_type = genomic DNA
atgacattactttcatttggattttctcctgttttcttttcagtcatggcgttctgtatcatttcacgtagtaaattctatccgcagagaacgcgaaacaaagttattgttctgattttactaactttttttatttgttttttatatccattaacaaaagtgtatctggtgggaagttacggtatatttgacaaattctacctcttttgctttatttctacgttaattgcaatagcaattaacgtagtgatacttacaataaatggagctaagaatgagagaaattag

SEQ ID NO: 542 - RNA, 13 nt, synthetic construct
  misc_feature 1..13: note = Kozak sequence
  source 1..13: mol_type = other RNA
gccgccrccatgg

SEQ ID NO: 543 - skipped

SEQ ID NO: 544 - DNA, 30 bp, synthetic construct
  misc_feature 1..30: note = Lead promoter
  source 1..30: mol_type = other DNA
gaaaaccttgtcaatgaagagcgatctatg

SEQ ID NO: 545 - DNA, 30 bp, synthetic construct
  misc_feature 1..30: note = FecA promoter
  source 1..30: mol_type = other DNA
ttctcgttcgactcatagctgaacacaaca

SEQ ID NO: 546 - DNA, 16 bp, synthetic construct
  misc_feature 1..16: note = Cu-sensitive promoter
  source 1..16: mol_type = other DNA
atgacaaaattgtcat

SEQ ID NO: 547 - DNA, 30 bp, synthetic construct
  misc_feature 1..30: note = Fe promoter
  source 1..30: mol_type = other DNA
accaatgctgggaacggccagggcacctaa

SEQ ID NO: 548 - DNA, 30 bp, synthetic construct
  misc_feature 1..30: note = Fe and UV promoters
  source 1..30: mol_type = other DNA
ctgaaagcgcataccgctatggagggggtt

SEQ ID NO: 549 - DNA, 30 bp, synthetic construct
  misc_feature 1..30: note = PrFe (PI + PII rus operon)
  source 1..30: mol_type = other DNA
tagatatgcctgaaagcgcataccgctatg

SEQ ID NO: 550 - DNA, 30 bp, synthetic construct
  misc_feature 1..30: note = Lux cassette right promoter
  source 1..30: mol_type = other DNA
tgttatagtcgaatacctctggcggtgata

SEQ ID NO: 551 - DNA, 30 bp, synthetic construct
  misc_feature 1..30: note = P(Las) TetO
  source 1..30: mol_type = other DNA
ttttggtacactccctatcagtgatagaga

SEQ ID NO: 552 - DNA, 30 bp, synthetic construct
  misc_feature 1..30: note = P(Las) CIO
  source 1..30: mol_type = other DNA
ctttttggtacactacctctggcggtgata

SEQ ID NO: 553 - DNA, 30 bp, synthetic construct
  misc_feature 1..30: note = P(Rhl)
  source 1..30: mol_type = other DNA
tacgcaagaaaatggtttgttatagtcgaa

SEQ ID NO: 554 - DNA, 30 bp, synthetic construct
  misc_feature 1..30: note = Double Promoter (LuxR/HSL, positive / cI,negative)
  source 1..30: mol_type = other DNA
cgtgcgtgttgataacaccgtgcgtgttga

SEQ ID NO: 555 - DNA, 30 bp, synthetic construct
  misc_feature 1..30: note = P2 promoter in agr operon from S. aureus
  source 1..30: mol_type = other DNA
agattgtactaaatcgtataatgacagtga

SEQ ID NO: 556 - DNA, 30 bp, synthetic construct
  misc_feature 1..30: note = plux-cI hybrid promoter
  source 1..30: mol_type = other DNA
gtgttgatgcttttatcaccgccagtggta

SEQ ID NO: 557 - DNA, 30 bp, synthetic construct
  misc_feature 1..30: note = plux-lac hybrid promoter
  source 1..30: mol_type = other DNA
agtgtgtggaattgtgagcggataacaatt

SEQ ID NO: 558 - DNA, 30 bp, synthetic construct
  misc_feature 1..30: note = CinR, CinL and glucose controlled promotor
  source 1..30: mol_type = other DNA
acatcttaaaagttttagtatcatattcgt

SEQ ID NO: 559 - DNA, 30 bp, synthetic construct; identical to SEQ ID NO: 553
  misc_feature 1..30: note = RhIR promoter repressible by CI
  source 1..30: mol_type = other DNA

SEQ ID NO: 560 - DNA, 30 bp, synthetic construct
  misc_feature 1..30: note = Reverse Lux Promoter
  source 1..30: mol_type = other DNA
tcttgcgtaaacctgtacgatcctacaggt

SEQ ID NO: 561 - DNA, 30 bp, synthetic construct
  misc_feature 1..30: note = rhlI promoter
  source 1..30: mol_type = other DNA
atcctcctttagtcttccccctcatgtgtg

SEQ ID NO: 562 - DNA, 30 bp, synthetic construct
  misc_feature 1..30: note = lasI promoter
  source 1..30: mol_type = other DNA
taaaattatgaaatttgcataaattcttca

SEQ ID NO: 563 - DNA, 30 bp, synthetic construct
  misc_feature 1..30: note = LuxR+3OC6HSL independent R0065
  source 1..30: mol_type = other DNA
gtgttgactattttacctctggcggtgata

SEQ ID NO: 564 - DNA, 30 bp, synthetic construct
  misc_feature 1..30: note = LasR/LasI Inducible & RHLR/RHLI repressiblePromoter
  source 1..30: mol_type = other DNA
gaaatctggcagtttttggtacacgaaagc

SEQ ID NO: 565 - DNA, 30 bp, synthetic construct
  misc_feature 1..30: note = pLux/cI Hybrid Promoter
  source 1..30: mol_type = other DNA
acaccgtgcgtgttgatatagtcgaataaa

SEQ ID NO: 566 - DNA, 30 bp, synthetic construct
  misc_feature 1..30: note = pLas promoter
  source 1..30: mol_type = other DNA
aaaattatgaaatttgtataaattcttcag

SEQ ID NO: 567 - DNA, 30 bp, synthetic construct
  misc_feature 1..30: note = pLas/cI Hybrid Promoter
  source 1..30: mol_type = other DNA
ggttctttttggtacctctggcggtgataa

SEQ ID NO: 568 - DNA, 30 bp, synthetic construct
  misc_feature 1..30: note = pLas/Lux Hybrid Promoter
  source 1..30: mol_type = other DNA
tgtaggatcgtacaggtataaattcttcag

SEQ ID NO: 569 - DNA, 30 bp, synthetic construct
  misc_feature 1..30: note = pLux
  source 1..30: mol_type = other DNA
caagaaaatggtttgttatagtcgaataaa

SEQ ID NO: 570 - DNA, 30 bp, synthetic construct
  misc_feature 1..30: note = pLux/Las Hybrid Promoter
  source 1..30: mol_type = other DNA
ctatctcatttgctagtatagtcgaataaa

SEQ ID NO: 571 - DNA, 30 bp, synthetic construct
  misc_feature 1..30: note = Hybrid promoter: HSL-LuxR activated, P22 C2repressed
  source 1..30: mol_type = other DNA
tagtttataatttaagtgttctttaatttc

SEQ ID NO: 572 - DNA, 30 bp, synthetic construct
  misc_feature 1..30: note = PAI+LasR -> LuxI (AI)
  source 1..30: mol_type = other DNA
caccttcgggtgggcctttctgcgtttata

SEQ ID NO: 573 - DNA, 30 bp, synthetic construct
  misc_feature 1..30: note = PAI+LasR -> LasI & AI+LuxR --| LasI
  source 1..30: mol_type = other DNA
aataactctgatagtgctagtgtagatctc

SEQ ID NO: 574 - DNA, 30 bp, synthetic construct; identical to SEQ ID NO: 572
  misc_feature 1..30: note = PAI+LasR -> LasI+GFP & AI+LuxR --| LasI+GFP
  source 1..30: mol_type = other DNA

SEQ ID NO: 575 - DNA, 30 bp, synthetic construct; identical to SEQ ID NO: 572
  misc_feature 1..30: note = Complex QS -> LuxI & LasI circuit
  source 1..30: mol_type = other DNA

SEQ ID NO: 576 - DNA, 30 bp, synthetic construct; identical to SEQ ID NO: 569
  misc_feature 1..30: note = position 3 mutated promoter lux pR-3 (luxR & HSLregulated)
  source 1..30: mol_type = other DNA

SEQ ID NO: 577 - DNA, 30 bp, synthetic construct; identical to SEQ ID NO: 569
  misc_feature 1..30: note = position 5 mutated promoter lux pR-5 (luxR & HSLregulated)
  source 1..30: mol_type = other DNA

SEQ ID NO: 578 - DNA, 30 bp, synthetic construct; identical to SEQ ID NO: 569
  misc_feature 1..30: note = position 3&5 mutated promoter lux pR-3/5 (luxR &HSL regulated)
  source 1..30: mol_type = other DNA

SEQ ID NO: 579 - DNA, 30 bp, synthetic construct
  misc_feature 1..30: note = Promoter (HSL-mediated luxR repressor)
  source 1..30: mol_type = other DNA
ttgacacctgtaggatcgtacaggtataat

SEQ ID NO: 580 - DNA, 30 bp, synthetic construct; identical to SEQ ID NO: 569
  misc_feature 1..30: note = Promoter (luxR & HSL regulated -- lux pR)
  source 1..30: mol_type = other DNA

SEQ ID NO: 581 - DNA, 30 bp, synthetic construct
  misc_feature 1..30: note = Promoter (luxR & HSL regulated -- lux pL)
  source 1..30: mol_type = other DNA
cacgcaaaacttgcgacaaacaataggtaa

SEQ ID NO: 582 - DNA, 30 bp, synthetic construct
  misc_feature 1..30: note = Promoter (RhlR & C4-HSL regulated)
  source 1..30: mol_type = other DNA
gttagctttcgaattggctaaaaagtgttc

SEQ ID NO: 583 - DNA, 30 bp, synthetic construct
  misc_feature 1..30: note = Promoter (cinR and HSL regulated)
  source 1..30: mol_type = other DNA
ccattctgctttccacgaacttgaaaacgc

SEQ ID NO: 584 - DNA, 30 bp, synthetic construct
  misc_feature 1..30: note = Promoter (LasR & PAI regulated)
  source 1..30: mol_type = other DNA
ggccgcgggttctttttggtacacgaaagc

SEQ ID NO: 585 - DNA, 30 bp, synthetic construct
  misc_feature 1..30: note = Promoter, Standard (luxR and HSL regulated -- luxpR)
  source 1..30: mol_type = other DNA
aagaaaatggtttgttgatactcgaataaa

SEQ ID NO: 586 - DNA, 30 bp, synthetic construct
  misc_feature 1..30: note = P(Bla)
  source 1..30: mol_type = other DNA
gtttatacataggcgagtactctgttatgg

SEQ ID NO: 587 - DNA, 30 bp, synthetic construct
  misc_feature 1..30: note = P(Cat)
  source 1..30: mol_type = other DNA
agaggttccaactttcaccataatgaaaca

SEQ ID NO: 588 - DNA, 30 bp, synthetic construct
  misc_feature 1..30: note = P(Kat)
  source 1..30: mol_type = other DNA
taaacaactaacggacaattctacctaaca

SEQ ID NO: 589 - DNA, 30 bp, synthetic construct
  misc_feature 1..30: note = Template for Building Primer Family Member
  source 1..30: mol_type = other DNA
acatcaagccaaattaaacaggattaacac

SEQ ID NO: 590 - DNA, 30 bp, synthetic construct
  misc_feature 1..30: note = Reverse lambda cI-regulated promoter
  source 1..30: mol_type = other DNA
gaggtaaaatagtcaacacgcacggtgtta

SEQ ID NO: 591 - DNA, 30 bp, synthetic construct
  misc_feature 1..30: note = Key Promoter absorbs 3
  source 1..30: mol_type = other DNA
caggccggaataactccctataatgcgcca

SEQ ID NO: 592 - DNA, 30 bp, synthetic construct
  misc_feature 1..30: note = constitutive promoter family member
  source 1..30: mol_type = other DNA
ggctagctcagtcctaggtacagtgctagc

SEQ ID NO: 593 - DNA, 30 bp, synthetic construct
  misc_feature 1..30: note = constitutive promoter family member
  source 1..30: mol_type = other DNA
agctagctcagtcctaggtattatgctagc

SEQ ID NO: 594 - DNA, 30 bp, synthetic construct
  misc_feature 1..30: note = constitutive promoter family member
  source 1..30: mol_type = other DNA
agctagctcagtcctaggtactgtgctagc

SEQ ID NO: 595 - DNA, 30 bp, synthetic construct
  misc_feature 1..30: note = constitutive promoter family member
  source 1..30: mol_type = other DNA
agctagctcagtcctagggattatgctagc

SEQ ID NO: 596 - DNA, 30 bp, synthetic construct
  misc_feature 1..30: note = constitutive promoter family member
  source 1..30: mol_type = other DNA
agctagctcagtcctaggtattgtgctagc

SEQ ID NO: 597 - DNA, 30 bp, synthetic construct
  misc_feature 1..30: note = constitutive promoter family member
  source 1..30: mol_type = other DNA
ggctagctcagtcctaggtactatgctagc

SEQ ID NO: 598 - DNA, 30 bp, synthetic construct
  misc_feature 1..30: note = constitutive promoter family member
  source 1..30: mol_type = other DNA
ggctagctcagtcctaggtatagtgctagc

SEQ ID NO: 599 - DNA, 30 bp, synthetic construct
  misc_feature 1..30: note = constitutive promoter family member
  source 1..30: mol_type = other DNA
ggctagctcagccctaggtattatgctagc

SEQ ID NO: 600 - DNA, 30 bp, synthetic construct
  misc_feature 1..30: note = constitutive promoter family member
  source 1..30: mol_type = other DNA
agctagctcagtcctaggtataatgctagc

SEQ ID NO: 601 - DNA, 30 bp, synthetic construct
  misc_feature 1..30: note = constitutive promoter family member
  source 1..30: mol_type = other DNA
agctagctcagtcctagggactgtgctagc

SEQ ID NO: 602 - DNA, 30 bp, synthetic construct
  misc_feature 1..30: note = constitutive promoter family member
  source 1..30: mol_type = other DNA
ggctagctcagtcctaggtacaatgctagc

SEQ ID NO: 603 - DNA, 30 bp, synthetic construct; identical to SEQ ID NO: 598
  misc_feature 1..30: note = constitutive promoter family member
  source 1..30: mol_type = other DNA

SEQ ID NO: 604 - DNA, 30 bp, synthetic construct; identical to SEQ ID NO: 595
  misc_feature 1..30: note = constitutive promoter family member
  source 1..30: mol_type = other DNA

SEQ ID NO: 605 - DNA, 30 bp, synthetic construct
  misc_feature 1..30: note = constitutive promoter family member
  source 1..30: mol_type = other DNA
ggctagctcagtcctagggattatgctagc

SEQ ID NO: 606 - DNA, 30 bp, synthetic construct; identical to SEQ ID NO: 602
  misc_feature 1..30: note = constitutive promoter family member
  source 1..30: mol_type = other DNA

SEQ ID NO: 607 - DNA, 30 bp, synthetic construct
  misc_feature 1..30: note = constitutive promoter family member
  source 1..30: mol_type = other DNA
agctagctcagcccttggtacaatgctagc

SEQ ID NO: 608 - DNA, 30 bp, synthetic construct
  misc_feature 1..30: note = constitutive promoter family member
  source 1..30: mol_type = other DNA
agctagctcagtcctagggactatgctagc

SEQ ID NO: 609 - DNA, 30 bp, synthetic construct
  misc_feature 1..30: note = constitutive promoter family member
  source 1..30: mol_type = other DNA
agctagctcagtcctagggattgtgctagc

SEQ ID NO: 610 - DNA, 30 bp, synthetic construct
  misc_feature 1..30: note = constitutive promoter family member
  source 1..30: mol_type = other DNA
ggctagctcagtcctaggtattgtgctagc

SEQ ID NO: 611 - DNA, 30 bp, synthetic construct; identical to SEQ ID NO: 600
  misc_feature 1..30: note = constitutive promoter family member
  source 1..30: mol_type = other DNA

SEQ ID NO: 612 - DNA, 30 bp, synthetic construct
  misc_feature 1..30: note = 1bp mutant from J23107
  source 1..30: mol_type = other DNA
ggctagctcagtcctaggtattatgctagc

SEQ ID NO: 613 - DNA, 30 bp, synthetic construct; identical to SEQ ID NO: 602
  misc_feature 1..30: note = 1bp mutant from J23114
  source 1..30: mol_type = other DNA

SEQ ID NO: 614 - DNA, 30 bp, synthetic construct
  misc_feature 1..30: note = pBAD reverse
  source 1..30: mol_type = other DNA
aaagtgtgacgccgtgcaaataatcaatgt

SEQ ID NO: 615 - DNA, 30 bp, synthetic construct
  misc_feature 1..30: note = NikR promoter, a protein of the ribbon helix-helixfamily of trancription factors that repress expre
  source 1..30: mol_type = other DNA
gacgaatacttaaaatcgtcatacttattt

SEQ ID NO: 616 - DNA, 30 bp, synthetic construct
  misc_feature 1..30: note = lacq_Promoter
  source 1..30: mol_type = other DNA
aaacctttcgcggtatggcatgatagcgcc

SEQ ID NO: 617 - DNA, 30 bp, synthetic construct
  misc_feature 1..30: note = lacIQ - promoter sequence
  source 1..30: mol_type = other DNA
tgatagcgcccggaagagagtcaattcagg

SEQ ID NO: 618 - DNA, 30 bp, synthetic construct
  misc_feature 1..30: note = E. Coli CreABCD phosphate sensing operon promoter
  source 1..30: mol_type = other DNA
ttatttaccgtgacgaactaattgctcgtg

SEQ ID NO: 619 - DNA, 30 bp, synthetic construct
  misc_feature 1..30: note = GlnRS promoter
  source 1..30: mol_type = other DNA
catacgccgttatacgttgtttacgctttg

SEQ ID NO: 620 - DNA, 30 bp, synthetic construct
  misc_feature 1..30: note = Constitutive weak promoter of lacZ
  source 1..30: mol_type = other DNA
ttatgcttccggctcgtatgttgtgtggac

SEQ ID NO: 621 - DNA, 30 bp, synthetic construct
  misc_feature 1..30: note = Mutated LacZ promoter
  source 1..30: mol_type = other DNA
ttatgcttccggctcgtatggtgtgtggac

SEQ ID NO: 622 - DNA, 30 bp, synthetic construct
  misc_feature 1..30: note = constitutive promoter with (TA)10 between -10 and-35 elements
  source 1..30: mol_type = other DNA
atatatatatatatataatggaagcgtttt

SEQ ID NO: 623 - DNA, 30 bp, synthetic construct; identical to SEQ ID NO: 622
  misc_feature 1..30: note = constitutive promoter with (TA)9 between -10 and-35 elements
  source 1..30: mol_type = other DNA

SEQ ID NO: 624 - DNA, 30 bp, synthetic construct
  misc_feature 1..30: note = constitutive promoter with (C)10 between -10 and-35 elements
  source 1..30: mol_type = other DNA
ccccgaaagcttaagaatataattgtaagc

SEQ ID NO: 625 - DNA, 30 bp, synthetic construct; identical to SEQ ID NO: 624
  misc_feature 1..30: note = constitutive promoter with (C)12 between -10 and-35 elements
  source 1..30: mol_type = other DNA

SEQ ID NO: 626 - DNA, 30 bp, synthetic construct
  misc_feature 1..30: note = optimized (TA) repeat constitutive promoter with13 bp between -10 and -35 elements
  source 1..30: mol_type = other DNA
tgacaatatatatatatatataatgctagc

SEQ ID NO: 627 - DNA, 30 bp, synthetic construct
  misc_feature 1..30: note = optimized (TA) repeat constitutive promoter with15 bp between -10 and -35 elements
  source 1..30: mol_type = other DNA
acaatatatatatatatatataatgctagc

SEQ ID NO: 628 - DNA, 30 bp, synthetic construct
  misc_feature 1..30: note = optimized (TA) repeat constitutive promoter with17 bp between -10 and -35 elements
  source 1..30: mol_type = other DNA
aatatatatatatatatatataatgctagc

SEQ ID NO: 629 - DNA, 30 bp, synthetic construct
  misc_feature 1..30: note = optimized (TA) repeat constitutive promoter with19 bp between -10 and -35 elements
  source 1..30: mol_type = other DNA
tatatatatatatatatatataatgctagc

SEQ ID NO: 630 - DNA, 30 bp, synthetic construct; identical to SEQ ID NO: 629
  misc_feature 1..30: note = optimized (TA) repeat constitutive promoter with21 bp between -10 and -35 elements
  source 1..30: mol_type = other DNA

SEQ ID NO: 631 - DNA, 30 bp, synthetic construct
  misc_feature 1..30: note = optimized (A) repeat constitutive promoter with 17bp between -10 and -35 elements
  source 1..30: mol_type = other DNA
aaaaaaaaaaaaaaaaaatataatgctagc

SEQ ID NO: 632 - DNA, 30 bp, synthetic construct; identical to SEQ ID NO: 631
  misc_feature 1..30: note = optimized (A) repeat constitutive promoter with 18bp between -10 and -35 elements
  source 1..30: mol_type = other DNA

SEQ ID NO: 633 - DNA, 30 bp, synthetic construct; identical to SEQ ID NO: 572
  misc_feature 1..30: note = J23101:GFP
  source 1..30: mol_type = other DNA

SEQ ID NO: 634 - DNA, 30 bp, synthetic construct; identical to SEQ ID NO: 572
  misc_feature 1..30: note = J23119:IFP
  source 1..30: mol_type = other DNA

SEQ ID NO: 635 - DNA, 30 bp, synthetic construct; identical to SEQ ID NO: 572
  misc_feature 1..30: note = J23119:HO1
  source 1..30: mol_type = other DNA

SEQ ID NO: 636 - DNA, 30 bp, synthetic construct; identical to SEQ ID NO: 572
  misc_feature 1..30: note = Infrared signal reporter (J23119:IFP:J23119:HO1)
  source 1..30: mol_type = other DNA

SEQ ID NO: 637 - DNA, 30 bp, synthetic construct; identical to SEQ ID NO: 592
  misc_feature 1..30: note = Double terminator + constitutive promoter
  source 1..30: mol_type = other DNA

SEQ ID NO: 638 - DNA, 30 bp, synthetic construct
  misc_feature 1..30: note = Double terminator + Constitutive promoter + StrongRBS
  source 1..30: mol_type = other DNA
tgctagctactagagattaaagaggagaaa

SEQ ID NO: 639 - DNA, 30 bp, synthetic construct
  misc_feature 1..30: note = IPTG inducible Lac promoter cassette
  source 1..30: mol_type = other DNA
ttgtgagcggataacaagatactgagcaca

SEQ ID NO: 640 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 641 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 642 - DNA, 30 bp, synthetic construct
  misc_feature 1..30: note = M13K07 gene I promoter
  source 1..30: mol_type = other DNA
cctgtttttatgttattctctctgtaaagg

SEQ ID NO: 643 - DNA, 30 bp, synthetic construct
  misc_feature 1..30: note = M13K07 gene II promoter
  source 1..30: mol_type = other DNA
aaatatttgcttatacaatcttcctgtttt

SEQ ID NO: 644 - DNA, 30 bp, synthetic construct
  misc_feature 1..30: note = M13K07 gene III promoter
  source 1..30: mol_type = other DNA
gctgataaaccgatacaattaaaggctcct

SEQ ID NO: 645 - DNA, 30 bp, synthetic construct
  misc_feature 1..30: note = M13K07 gene IV promoter
  source 1..30: mol_type = other DNA
ctcttctcagcgtcttaatctaagctatcg

SEQ ID NO: 646 - DNA, 30 bp, synthetic construct
  misc_feature 1..30: note = M13K07 gene V promoter
  source 1..30: mol_type = other DNA
atgagccagttcttaaaatcgcataaggta

SEQ ID NO: 647 - DNA, 30 bp, synthetic construct
  misc_feature 1..30: note = M13K07 gene VI promoter
  source 1..30: mol_type = other DNA
ctattgattgtgacaaaataaacttattcc

SEQ ID NO: 648 - DNA, 30 bp, synthetic construct
  misc_feature 1..30: note = M13K07 gene VIII promoter
  source 1..30: mol_type = other DNA
gtttcgcgcttggtataatcgctgggggtc

SEQ ID NO: 649 - DNA, 30 bp, synthetic construct
  misc_feature 1..30: note = M13110
  source 1..30: mol_type = other DNA
ctttgcttctgactataatagtcagggtaa

SEQ ID NO: 650 - DNA, 30 bp, synthetic construct
  misc_feature 1..30: note = Modified promoter sequence of g3.
  source 1..30: mol_type = other DNA
aaaccgatacaattaaaggctcctgctagc

SEQ ID NO: 651 - DNA, 30 bp, synthetic construct
  misc_feature 1..30: note = Constitutive Promoter I
  source 1..30: mol_type = other DNA
caccacactgatagtgctagtgtagatcac

SEQ ID NO: 652 - DNA, 30 bp, synthetic construct
  misc_feature 1..30: note = Constitutive Promoter II
  source 1..30: mol_type = other DNA
gccggaataactccctataatgcgccacca

SEQ ID NO: 653 - DNA, 30 bp, synthetic construct
  misc_feature 1..30: note = --Specify Parts List--
  source 1..30: mol_type = other DNA
ttgacaagcttttcctcagctccgtaaact

SEQ ID NO: 654 - DNA, 30 bp, synthetic construct
  misc_feature 1..30: note = Full-length stationary phase osmY promoter
  source 1..30: mol_type = other DNA
ggtttcaaaattgtgatctatatttaacaa

SEQ ID NO: 655 - DNA, 30 bp, synthetic construct; identical to SEQ ID NO: 654
  misc_feature 1..30: note = Minimal stationary phase osmY promoter
  source 1..30: mol_type = other DNA

SEQ ID NO: 656 - DNA, 30 bp, synthetic construct
  misc_feature 1..30: note = htpG Heat Shock Promoter
  source 1..30: mol_type = other DNA
tctattccaataaagaaatcttcctgcgtg

SEQ ID NO: 657 - DNA, 30 bp, synthetic construct
  misc_feature 1..30: note = Promoter veg a constitutive promoter for B.subtilis
  source 1..30: mol_type = other DNA
aaaaatgggctcgtgttgtacaataaatgt

SEQ ID NO: 658 - DNA, 30 bp, synthetic construct
  misc_feature 1..30: note = Promoter 43 a constitutive promoter for B.subtilis
  source 1..30: mol_type = other DNA
aaaaaaagcgcgcgattatgtaaaatataa

SEQ ID NO: 659 - DNA, 30 bp, synthetic construct
  misc_feature 1..30: note = Strong constitutive promoter for Bacillus subtilis
  source 1..30: mol_type = other DNA
aattgcagtaggcatgacaaaatggactca

SEQ ID NO: 660 - DNA, 30 bp, synthetic construct
  misc_feature 1..30: note = PliaG
  source 1..30: mol_type = other DNA
caagcttttcctttataatagaatgaatga

SEQ ID NO: 661 - DNA, 30 bp, synthetic construct
  misc_feature 1..30: note = PlepA
  source 1..30: mol_type = other DNA
tctaagctagtgtattttgcgtttaatagt

SEQ ID NO: 662 - DNA, 30 bp, synthetic construct
  misc_feature 1..30: note = Pveg
  source 1..30: mol_type = other DNA
aatgggctcgtgttgtacaataaatgtagt

SEQ ID NO: 663 - DNA, 30 bp, synthetic construct
  misc_feature 1..30: note = Promoter ctc for B. subtilis
  source 1..30: mol_type = other DNA
atccttatcgttatgggtattgtttgtaat

SEQ ID NO: 664 - DNA, 30 bp, synthetic construct
  misc_feature 1..30: note = Promoter gsiB for B. subtilis
  source 1..30: mol_type = other DNA
taaaagaattgtgagcgggaatacaacaac

SEQ ID NO: 665 - DNA, 30 bp, synthetic construct; identical to SEQ ID NO: 658
  misc_feature 1..30: note = Promoter 43 a constitutive promoter for B.subtilis
  source 1..30: mol_type = other DNA

SEQ ID NO: 666 - DNA, 30 bp, synthetic construct
  misc_feature 1..30: note = Pspv2 from Salmonella
  source 1..30: mol_type = other DNA
tacaaaataattcccctgcaaacattatca

SEQ ID NO: 667 - DNA, 30 bp, synthetic construct
  misc_feature 1..30: note = Pspv from Salmonella
  source 1..30: mol_type = other DNA
tacaaaataattcccctgcaaacattatcg

SEQ ID NO: 668 - DNA, 30 bp, synthetic construct
  misc_feature 1..30: note = T7 promoter (strong promoter from T7bacteriophage)
  source 1..30: mol_type = other DNA
agggaatacaagctacttgttctttttgca

SEQ ID NO: 669 - DNA, 23 bp, synthetic construct
  misc_feature 1..23: note = T7 Promoter
  source 1..23: mol_type = other DNA
taatacgactcactatagggaga

SEQ ID NO: 670 - DNA, 28 bp, synthetic construct
  misc_feature 1..28: note = T7 Promoter
  source 1..28: mol_type = other DNA
gaatttaatacgactcactatagggaga

SEQ ID NO: 671 - DNA, 19 bp, synthetic construct
  misc_feature 1..19: note = T7 consensus -10 and rest
  source 1..19: mol_type = other DNA
taatacgactcactatagg

SEQ ID NO: 672 - DNA, 30 bp, synthetic construct
  misc_feature 1..30: note = overlapping T7 promoter
  source 1..30: mol_type = other DNA
gagtcgtattaatacgactcactatagggg

SEQ ID NO: 673 - DNA, 30 bp, synthetic construct
  misc_feature 1..30: note = more overlapping T7 promoter
  source 1..30: mol_type = other DNA
agtgagtcgtactacgactcactatagggg

SEQ ID NO: 674 - DNA, 30 bp, synthetic construct
  misc_feature 1..30: note = weaken overlapping T7 promoter
  source 1..30: mol_type = other DNA
gagtcgtattaatacgactctctatagggg

SEQ ID NO: 675 - DNA, 23 bp, synthetic construct
  misc_feature 1..23: note = T7 Consensus Promoter Sequence
  source 1..23: mol_type = other DNA
taatacgactcactatagggaga

SEQ ID NO: 676 - DNA, 23 bp, synthetic construct
  misc_feature 1..23: note = T7 RNAP promoter
  source 1..23: mol_type = other DNA
ttatacgactcactatagggaga

SEQ ID NO: 677 - DNA, 23 bp, synthetic construct
  misc_feature 1..23: note = T7 RNAP promoter
  source 1..23: mol_type = other DNA
gaatacgactcactatagggaga

SEQ ID NO: 678 - DNA, 23 bp, synthetic construct
  misc_feature 1..23: note = T7 RNAP promoter
  source 1..23: mol_type = other DNA
taatacgtctcactatagggaga

SEQ ID NO: 679 - DNA, 23 bp, synthetic construct
  misc_feature 1..23: note = T7 RNAP promoter
  source 1..23: mol_type = other DNA
tcatacgactcactatagggaga

SEQ ID NO: 680 - DNA, 30 bp, synthetic construct
  misc_feature 1..30: note = T7 strong promoter
  source 1..30: mol_type = other DNA
taatacgactcactatagggagaccacaac

SEQ ID NO: 681 - DNA, 30 bp, synthetic construct
  misc_feature 1..30: note = T7 weak binding and processivity
  source 1..30: mol_type = other DNA
taattgaactcactaaagggagaccacagc

SEQ ID NO: 682 - DNA, 30 bp, synthetic construct
  misc_feature 1..30: note = T7 weak binding promoter
  source 1..30: mol_type = other DNA
cgaagtaatacgactcactattagggaaga

SEQ ID NO: 683 - DNA, 30 bp, synthetic construct
  misc_feature 1..30: note = pCyc (Medium) Promoter
  source 1..30: mol_type = other DNA
acaaacacaaatacacacactaaattaata

SEQ ID NO: 684 - DNA, 30 bp, synthetic construct
  misc_feature 1..30: note = pAdh (Strong) Promoter
  source 1..30: mol_type = other DNA
ccaagcatacaatcaactatctcatataca

SEQ ID NO: 685 - DNA, 30 bp, synthetic construct
  misc_feature 1..30: note = pSte5 (Weak) Promoter
  source 1..30: mol_type = other DNA
gatacaggatacagcggaaacaacttttaa

SEQ ID NO: 686 - DNA, 30 bp, synthetic construct
  misc_feature 1..30: note = yeast ADH1 promoter
  source 1..30: mol_type = other DNA
tttcaagctataccaagcatacaatcaact

SEQ ID NO: 687 - DNA, 30 bp, synthetic construct
  misc_feature 1..30: note = cyc100 minimal promoter
  source 1..30: mol_type = other DNA
cctttgcagcataaattactatacttctat

SEQ ID NO: 688 - DNA, 30 bp, synthetic construct; identical to SEQ ID NO: 687
  misc_feature 1..30: note = cyc70 minimal promoter
  source 1..30: mol_type = other DNA

SEQ ID NO: 689 - DNA, 30 bp, synthetic construct; identical to SEQ ID NO: 687
  misc_feature 1..30: note = cyc43 minimal promoter
  source 1..30: mol_type = other DNA

SEQ ID NO: 690 - DNA, 30 bp, synthetic construct; identical to SEQ ID NO: 687
  misc_feature 1..30: note = cyc28 minimal promoter
  source 1..30: mol_type = other DNA

SEQ ID NO: 691 - DNA, 30 bp, synthetic construct; identical to SEQ ID NO: 687
  misc_feature 1..30: note = cyc16 minimal promoter
  source 1..30: mol_type = other DNA

SEQ ID NO: 692 - DNA, 30 bp, synthetic construct
  misc_feature 1..30: note = pPGK1
  source 1..30: mol_type = other DNA
ttatctactttttacaacaaatataaaaca

SEQ ID NO: 693 - DNA, 30 bp, synthetic construct; identical to SEQ ID NO: 683
  misc_feature 1..30: note = pCYC Yeast Promoter
  source 1..30: mol_type = other DNA

SEQ ID NO: 694 - DNA, 30 bp, synthetic construct
  misc_feature 1..30: note = Yeast GPD (TDH3) Promoter
  source 1..30: mol_type = other DNA
gtttcgaataaacacacataaacaaacaaa

SEQ ID NO: 695 - DNA, 30 bp, synthetic construct; identical to SEQ ID NO: 684
  misc_feature 1..30: note = yeast mid-length ADH1 promoter
  source 1..30: mol_type = other DNA

SEQ ID NO: 696 - DNA, 30 bp, synthetic construct
  misc_feature 1..30: note = Yeast CLB1 promoter region, G2/M cell cyclespecific
  source 1..30: mol_type = other DNA
accatcaaaggaagctttaatcttctcata

SEQ ID NO: 697 - DNA, 30 bp, synthetic construct
  misc_feature 1..30: note = CMV promoter
  source 1..30: mol_type = other DNA
agaacccactgcttactggcttatcgaaat

SEQ ID NO: 698 - DNA, 30 bp, synthetic construct
  misc_feature 1..30: note = Ubc Promoter
  source 1..30: mol_type = other DNA
ggccgtttttggcttttttgttagacgaag

SEQ ID NO: 699 - protein, 9 aa, Lactobacillus curvatus
NIPQLTPTP

SEQ ID NO: 700 - DNA, 27 bp, Lactobacillus curvatus
  source 1..27: mol_type = genomic DNA
aacattccgcagctgaccccgaccccg

SEQ ID NO: 701 - protein, 18 aa, Bacillus thuringiensis
  VARIANT 4..10: note = X= any amino acid
DWTXWSXLVXAACSVELL

SEQ ID NO: 702 - DNA, 54 bp, Bacillus thuringiensis; identical to SEQ ID NO: 389
  misc_feature 10..30: note = n = A,T,C or G
  source 1..54: mol_type = genomic DNA

SEQ ID NO: 703 - protein, 30 aa, Lactobacillus curvatus; identical to SEQ ID NO: 390
  source 1..30: note = L442

SEQ ID NO: 704 - DNA, 90 bp, Lactobacillus curvatus; identical to SEQ ID NO: 391
  source 1..90: mol_type = genomic DNA; note = L442

SEQ ID NO: 705 - protein, 43 aa, Carnobacterium divergens; identical to SEQ ID NO: 392

SEQ ID NO: 706 - DNA, 129 bp, Carnobacterium divergens; identical to SEQ ID NO: 393
  source 1..129: mol_type = genomic DNA

SEQ ID NO: 707 - protein, 24 aa, Microbispora sp.
  source 1..24: note = strain 107891
VTSWSLCTPGCTSPGGGSNCSFCC

SEQ ID NO: 708 - DNA, 72 bp, Microbispora sp.
  source 1..72: mol_type = genomic DNA; note = strain 107891
gtgaccagctggagcctgtgcaccccgggctgcaccagcccgggcggcggcagcaactgcagcttttgctgc

SEQ ID NO: 709 - protein, 62 aa, Enterococcus sp.; identical to SEQ ID NO: 394

SEQ ID NO: 710 - DNA, 186 bp, Enterococcus sp.; identical to SEQ ID NO: 395
  source 1..186: mol_type = genomic DNA

SEQ ID NO: 711 - protein, 39 aa, Enterococcus faecium; identical to SEQ ID NO: 396

SEQ ID NO: 712 - DNA, 117 bp, Enterococcus faecium; identical to SEQ ID NO: 397
  source 1..117: mol_type = genomic DNA

SEQ ID NO: 713 - protein, 30 aa, Paenibacillus polymyxa; identical to SEQ ID NO: 398

SEQ ID NO: 714 - DNA, 90 bp, Paenibacillus polymyxa; identical to SEQ ID NO: 399
  source 1..90: mol_type = genomic DNA

SEQ ID NO: 715 - protein, 31 aa, Staphylococcus epidermidis; identical to SEQ ID NO: 400

SEQ ID NO: 716 - DNA, 93 bp, Staphylococcus epidermidis; identical to SEQ ID NO: 401
  source 1..93: mol_type = genomic DNA

SEQ ID NO: 717 - protein, 58 aa, Enterococcus faecium
MEKLTVKEMSQVVGGKYYGNGVSCNKKGCSVDWGKAIGIIGNNAAANLTTGGKAGWKG

SEQ ID NO: 718 - DNA, 178 bp, Enterococcus faecium
  source 1..178: mol_type = genomic DNA
atggaaaaattaactgtgaaagaaatgtcgcaagtagttggcggaaagtactatggtaacggagtatcatgtaataaaaagggatgtagtgttgattggggaaaagctattggtattattggaaataatgctgctgctaatttaactactggcggaaaagcagggtggaaaggttaac

SEQ ID NO: 719 - protein, 39 aa, Paenibacillus polymyxa; identical to SEQ ID NO: 404

SEQ ID NO: 720 - DNA, 93 bp, Paenibacillus polymyxa; identical to SEQ ID NO: 401
  source 1..93: mol_type = genomic DNA

SEQ ID NO: 721 - protein, 35 aa, Bacillus circulans; identical to SEQ ID NO: 406

SEQ ID NO: 722 - DNA, 105 bp, Bacillus circulans; identical to SEQ ID NO: 407
  source 1..105: mol_type = genomic DNA

SEQ ID NO: 723 - protein, 30 aa, Paenibacillus polymyxa; identical to SEQ ID NO: 408

SEQ ID NO: 724 - DNA, 90 bp, Paenibacillus polymyxa; identical to SEQ ID NO: 409
  source 1..90: mol_type = genomic DNA

SEQ ID NO: 725 - protein, 13 aa, Lactobacillus rhamnosus
AVPAVRKTNETLD

SEQ ID NO: 726 - DNA, 39 bp, Lactobacillus rhamnosus; identical to SEQ ID NO: 411
  source 1..39: mol_type = genomic DNA

SEQ ID NO: 727 - protein, 60 aa, Bacillus licheniformis
MKNSAAREAFKGANHPAGMVSEEELKALVGGNDVNPETTPATTSSWTCITAGVTVSASLC

SEQ ID NO: 728 - DNA, 180 bp, Bacillus licheniformis
  source 1..180: mol_type = genomic DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgc

SEQ ID NO: 729 - protein, 689 aa, Pseudomonas aeruginosa; identical to SEQ ID NO: 276

SEQ ID NO: 730 - DNA, 2070 bp, Pseudomonas aeruginosa; identical to SEQ ID NO: 277
  source 1..2070: mol_type = genomic DNA

SEQ ID NO: 731 - protein, 36 aa, Lactobacillus plantarum; identical to SEQ ID NO: 414

SEQ ID NO: 732 - DNA, 108 bp, Lactobacillus plantarum; identical to SEQ ID NO: 415
  source 1..108: mol_type = genomic DNA

SEQ ID NO: 733 - protein, 30 aa, Lactococcus lactis
MKTILRFVAGYDIASHKKKTGGYPWERGKA

SEQ ID NO: 734 - DNA, 93 bp, Lactococcus lactis
  source 1..93: mol_type = genomic DNA
atgaaaacaatcctacgttttgttgctggctacgatattgctagtcataaaaagaaaactggcggctatccatgggaacgtggaaaagcttaa

SEQ ID NO: 735 - protein, 24 aa, Lactobacillus acidophilus
GNPKVAHCASQIGRSTAWGAVSGA

SEQ ID NO: 736 - DNA, 72 bp, Lactobacillus acidophilus; identical to SEQ ID NO: 417
  source 1..72: mol_type = genomic DNA

SEQ ID NO: 737 - protein, 53 aa, Lactobacillus salivarius
  source 1..53: note = cp400
MFFNFMKKVDVKKNFGYKEVSRKDLAKVNGGKRKKHRCRVYNNGMPTGMYRWC

SEQ ID NO: 738 - DNA, 162 bp, Lactobacillus salivarius
  source 1..162: mol_type = genomic DNA; note = cp400
atgttttttaattttatgaaaaaagtagatgtgaagaagaattttggatataaagaagtttctagaaaagatctagctaaagtaaatggtggaaagagaaagaaacatcgttgcagagtttataataatggaatgcctacaggaatgtatcgttggtgctaa

SEQ ID NO: 739 - protein, 5 aa, synthetic construct
  REGION 1..5: note = Caspase 2 consensus sequence
DVADL

SEQ ID NO: 740 - protein, 5 aa, synthetic construct
  REGION 1..5: note = Caspase 2 consensus sequence
DVADI

SEQ ID NO: 741 - protein, 12 aa, synthetic construct
  REGION 1..12: note = Synthetic RAP-binding peptide, RBP
FHWWQTSPAHFS

SEQ ID NO: 742 - protein, 12 aa, synthetic construct
  REGION 1..12: note = Synthetic RAP-binding peptide, RBP
WPFAHWPWQYPR

SEQ ID NO: 743 - protein, 9 aa, synthetic construct
  REGION 1..9: note = Synthetic AgrC ligand
  VARIANT 5..9: note = Thiolacton linkage between C5 and F9
GDSVCASYF

SEQ ID NO: 744 - protein, 7 aa, synthetic construct
  REGION 1..7: note = Synthetic AgrC ligand
  VARIANT 3..7: note = Thiolacton linkage between C3 and F7
SVCASYF

SEQ ID NO: 745 - protein, 5 aa, synthetic construct
  REGION 1..5: note = Synthetic Cry1Aa ligand
SKADT

SEQ ID NO: 746 - protein, 5 aa, synthetic construct
  REGION 1..5: note = Synthetic Cry1Aa ligand
SKPAD

SEQ ID NO: 747 - protein, 11 aa, synthetic construct
  REGION 1..11: note = Synthetic Fsr ligand
  VARIANT 3..11: note = Lacton linkage between S3 and A11
QNSAAAFAAWA

SEQ ID NO: 748 - protein, 11 aa, synthetic construct
  REGION 1..11: note = Synthetic Fsr ligand
  VARIANT 3..11: note = Lacton linkage between S3 and A11
QNSAAAFGQWA

SEQ ID NO: 749 - protein, 7 aa, synthetic construct
  REGION 1..7: note = Synthetic AgrC1, AgrC2
  VARIANT 4..7: note = Thiolacton linkage between C4 and M7)
YSTCFIM

SEQ ID NO: 750 - protein, 7 aa, synthetic construct
  REGION 1..7: note = Synthetic AgrC1, AgrC2
  VARIANT 3..7: note = Thiolacton linkage between C3 and M7
STCAFIM